SEQUENCE LISTING (888 sequences)

SEQ ID NO: 1 - DNA, 22 bp, Artificial Sequence
  Synthetic: Slu1
gatggagggccttttattcgcg

SEQ ID NO: 2 - DNA, 22 bp, Artificial Sequence
  Synthetic: Slu2
ggccttttattcgcgagggtcg

SEQ ID NO: 3 - DNA, 22 bp, Artificial Sequence
  Synthetic: Slu3
cagttcacaaccgctccgagcg

SEQ ID NO: 4 - DNA, 22 bp, Artificial Sequence
  Synthetic: Slu4
aatatccaaaccgccgaagcgg

SEQ ID NO: 5 - DNA, 22 bp, Artificial Sequence
  Synthetic: Slu5
aggacccttcgagccccgttcg

SEQ ID NO: 6 - DNA, 22 bp, Artificial Sequence
  Synthetic: Slu6
ccacgctcggagcggttgtgaa

SEQ ID NO: 7 - DNA, 22 bp, Artificial Sequence
  Synthetic: Slu7
ctccacgcacccccacctatcg

SEQ ID NO: 8 - DNA, 22 bp, Artificial Sequence
  Synthetic: Slu8
cacccccgaccctcgcgaataa

SEQ ID NO: 9 - DNA, 22 bp, Artificial Sequence
  Synthetic: Slu9
accctagaactgtcttcgactc

SEQ ID NO: 10 - DNA, 22 bp, Artificial Sequence
  Synthetic: Slu10
ctttgcgaaccaacgataggtg

SEQ ID NO: 11 - DNA, 22 bp, Artificial Sequence
  Synthetic: Slu11
gagggccttttattcgcgaggg

SEQ ID NO: 12 - DNA, 22 bp, Artificial Sequence
  Synthetic: Slu12
gggccttttattcgcgagggtc

SEQ ID NO: 13 - DNA, 22 bp, Artificial Sequence
  Synthetic: Slu13
acctcgtcctccgactcgctga

SEQ ID NO: 14 - DNA, 22 bp, Artificial Sequence
  Synthetic: Slu14
tttgcactttgcgaaccaacga

SEQ ID NO: 15 - DNA, 22 bp, Artificial Sequence
  Synthetic: Slu15
cgggatccccgaaaaagcgggt

SEQ ID NO: 16 - DNA, 22 bp, Artificial Sequence
  Synthetic: Slu16
ccagttcacaaccgctccgagc

SEQ ID NO: 17 - DNA, 22 bp, Artificial Sequence
  Synthetic: Slu17
actttgcgaaccaacgataggt

SEQ ID NO: 18 - DNA, 22 bp, Artificial Sequence
  Synthetic: Slu18
ataaatatccaaaccgccgaag

SEQ ID NO: 19 - DNA, 22 bp, Artificial Sequence
  Synthetic: Slu19
agatggagggccttttattcgc

SEQ ID NO: 20 - DNA, 22 bp, Artificial Sequence
  Synthetic: Slu20
cggctccgcccgcttcggcggt

SEQ ID NO: 21 - DNA, 22 bp, Artificial Sequence
  Synthetic: Slu21
ccccggagtcgaagacagttct

SEQ ID NO: 22 - DNA, 22 bp, Artificial Sequence
  Synthetic: Slu22
tgggcggagacccacgctcgga

SEQ ID NO: 23 - DNA, 22 bp, Artificial Sequence
  Synthetic: Slu23
gcgcgatctctgcctgcttact

SEQ ID NO: 24 - DNA, 22 bp, Artificial Sequence
  Synthetic: Slu24
cactttgcgaaccaacgatagg

SEQ ID NO: 25 - DNA, 22 bp, Artificial Sequence
  Synthetic: Slu25
gcggccggcgaacggggctcga

SEQ ID NO: 26 - DNA, 22 bp, Artificial Sequence
  Synthetic: Slu26
atccgggcccgccccctagcgg

SEQ ID NO: 27 - DNA, 22 bp, Artificial Sequence
  Synthetic: Slu27
cctgcagtttgcccatccacgt

SEQ ID NO: 28 - DNA, 22 bp, Artificial Sequence
  Synthetic: Slu28
ggcgcgatctctgcctgcttac

SEQ ID NO: 29 - DNA, 22 bp, Artificial Sequence
  Synthetic: Slu29
caaaccgccgaagcgggcggag

SEQ ID NO: 30 - DNA, 22 bp, Artificial Sequence
  Synthetic: Slu30
tgtcttcgactccggggccccg

SEQ ID NO: 31 - DNA, 22 bp, Artificial Sequence
  Synthetic: Slu31
cccaacaaccccaatccacgtt

SEQ ID NO: 32 - DNA, 22 bp, Artificial Sequence
  Synthetic: Slu32
gggcgcgggatccccgaaaaag

SEQ ID NO: 33 - DNA, 22 bp, Artificial Sequence
  Synthetic: Slu33
agggccttttattcgcgagggt

SEQ ID NO: 34 - DNA, 22 bp, Artificial Sequence
  Synthetic: Slu34
aataaatatccaaaccgccgaa

SEQ ID NO: 35 - DNA, 22 bp, Artificial Sequence
  Synthetic: Slu35
ggggcgcgggatccccgaaaaa

SEQ ID NO: 36 - DNA, 22 bp, Artificial Sequence
  Synthetic: Slu36
tgtgatccgggcccgcccccta

SEQ ID NO: 37 - DNA, 22 bp, Artificial Sequence
  Synthetic: Slu37
cctccgactcgctgacaggcta

SEQ ID NO: 38 - DNA, 22 bp, Artificial Sequence
  Synthetic: Slu38
cttcgagccccgttcgccggcc

SEQ ID NO: 39 - DNA, 22 bp, Artificial Sequence
  Synthetic: Slu39
gggctcgaagggtccttgtagc

SEQ ID NO: 40 - DNA, 22 bp, Artificial Sequence
  Synthetic: Slu40
gctcggagcggttgtgaactgg

SEQ ID NO: 41 - DNA, 22 bp, Artificial Sequence
  Synthetic: Slu41
ccagccggctccgcccgcttcg

SEQ ID NO: 42 - DNA, 22 bp, Artificial Sequence
  Synthetic: Slu42
ctgcagtttgcccatccacgtc

SEQ ID NO: 43 - DNA, 22 bp, Artificial Sequence
  Synthetic: Slu43
ggtcctgtagcctgtcagcgag

SEQ ID NO: 44 - DNA, 22 bp, Artificial Sequence
  Synthetic: Slu44
ctcagtgcatccaaaacgtgga

SEQ ID NO: 45 - DNA, 22 bp, Artificial Sequence
  Synthetic: Slu45
tcagtgcatccaaaacgtggat

SEQ ID NO: 46 - DNA, 22 bp, Artificial Sequence
  Synthetic: Slu46
gccccgttggaagactgagtgc

SEQ ID NO: 47 - DNA, 22 bp, Artificial Sequence
  Synthetic: Slu47
ttcttgtgcatgacgccctgct

SEQ ID NO: 48 - DNA, 22 bp, Artificial Sequence
  Synthetic: Slu48
tcttgtgcatgacgccctgctc

SEQ ID NO: 49 - DNA, 22 bp, Artificial Sequence
  Synthetic: Slu49
tggaggatggaacacggacggc

SEQ ID NO: 50 - DNA, 22 bp, Artificial Sequence
  Synthetic: Slu50
tcgcgccagacgctccccagag

SEQ ID NO: 51 - DNA, 22 bp, Artificial Sequence
  Synthetic: Slu51
ccccgttggaagactgagtgcc

SEQ ID NO: 52 - skipped

SEQ ID NO: 53 - DNA, 22 bp, Artificial Sequence
  Synthetic: Slu53
gccgggtccgcggccggcgaac

SEQ ID NO: 54 - skipped

SEQ ID NO: 55 - DNA, 22 bp, Artificial Sequence
  Synthetic: Slu55
gctagggggcgggcccggatca

SEQ ID NO: 56 - DNA, 22 bp, Artificial Sequence
  Synthetic: Slu56
gccccggagtcgaagacagttc

SEQ ID NO: 57 - skipped

SEQ ID NO: 58 - DNA, 22 bp, Artificial Sequence
  Synthetic: Slu58
gccccgttcgccggccgcggac

SEQ ID NO: 59 - DNA, 22 bp, Artificial Sequence
  Synthetic: Slu59
ccctagaactgtcttcgactcc

SEQ ID NO: 60 - skipped

SEQ ID NO: 61 - DNA, 22 bp, Artificial Sequence
  Synthetic: Slu61
ggggctcgaagggtccttgtag

SEQ ID NO: 62 - DNA, 22 bp, Artificial Sequence
  Synthetic: Slu62
cccgggcactcagtcttccaac

SEQ ID NO: 63 - skipped

SEQ ID NO: 64 - DNA, 22 bp, Artificial Sequence
  Synthetic: Slu64
agcggttgtgaactggcaggcg

SEQ ID NO: 65 - skipped

SEQ ID NO: 66 - DNA, 22 bp, Artificial Sequence
  Synthetic: Slu66
ggcgcggcttctgtgccgtgcc

SEQ ID NO: 67-69 - skipped (3 entries)

SEQ ID NO: 70 - DNA, 22 bp, Artificial Sequence
  Synthetic: Slu70
cggagcggttgtgaactggcag

SEQ ID NO: 71-96 - skipped (26 entries)

SEQ ID NO: 97 - RNA, 77 nt, Artificial Sequence
  Synthetic: crRNA and tracrRNA used for gRNAs with SaCas9
guuuuaguacucuggaaacagaaucuacuaaaacaaggcaaaaugccguguuuaucucgucaacuuguuggcgagau

SEQ ID NO: 98-100 - skipped (3 entries)

SEQ ID NO: 101 - DNA, 19 bp, Artificial Sequence
  Synthetic: TIDE sequencing Forward Primer
gagtcccaggagccaatca

SEQ ID NO: 102 - DNA, 18 bp, Artificial Sequence
  Synthetic: TIDE sequencing Reverse Primer
cccctcttctcgacgctc

SEQ ID NO: 103 - DNA, 15 bp, Artificial Sequence
  Synthetic: 5′ LOS ddPCR Forward Primer
ctagcggccggggag

SEQ ID NO: 104 - DNA, 17 bp, Artificial Sequence
  Synthetic: 5′ LOS ddPCR Reverse Primer
agcagcattcccggcta

SEQ ID NO: 105 - DNA, 24 bp, Artificial Sequence
  Synthetic: 5′ LOS ddPCR Probe
cgaacggggctcgaagggtccttg

SEQ ID NO: 106 - DNA, 21 bp, Artificial Sequence
  Synthetic: 3′ LOS ddPCR Forward Primer
gggggatcacagaccatttct

SEQ ID NO: 107 - DNA, 20 bp, Artificial Sequence
  Synthetic: 3′ LOS ddPCR Reverse Primer
cgaaccaacgataggtgggg

SEQ ID NO: 108 - DNA, 20 bp, Artificial Sequence
  Synthetic: 3′ LOS ddPCR Probe
cctgggaaggcagcaagccg

SEQ ID NO: 109-199 - skipped (91 entries)

SEQ ID NO: 200 - DNA, 22 bp, Artificial Sequence
  Synthetic: Sa1
gcgggatgcgaagcggccgaat

SEQ ID NO: 201 - DNA, 22 bp, Artificial Sequence
  Synthetic: Sa2
gccccggagtcgaagacagttc

SEQ ID NO: 202 - DNA, 22 bp, Artificial Sequence
  Synthetic: Sa3
cgcggccggcgaacggggctcg

SEQ ID NO: 203 - DNA, 22 bp, Artificial Sequence
  Synthetic: Sa4
ccagttcacaaccgctccgagc

SEQ ID NO: 204 - DNA, 22 bp, Artificial Sequence
  Synthetic: Sa5
gggccttttattcgcgagggtc

SEQ ID NO: 205 - DNA, 22 bp, Artificial Sequence
  Synthetic: Sa6
agatggagggccttttattcgc

SEQ ID NO: 206 - DNA, 22 bp, Artificial Sequence
  Synthetic: Sa7
gagctagcgggatgcgaagcgg

SEQ ID NO: 207 - DNA, 22 bp, Artificial Sequence
  Synthetic: Sa8
cggctccgcccgcttcggcggt

SEQ ID NO: 208 - DNA, 22 bp, Artificial Sequence
  Synthetic: Sa9
caacgataggtgggggtgcgtg

SEQ ID NO: 209 - DNA, 22 bp, Artificial Sequence
  Synthetic: Sa10
tggggacagacaataaataccg

SEQ ID NO: 210 - DNA, 22 bp, Artificial Sequence
  Synthetic: Sa11
cccaacaaccccaatccacgtt

SEQ ID NO: 211 - DNA, 22 bp, Artificial Sequence
  Synthetic: Sa12
actcagtcttccaacggggccc

SEQ ID NO: 212 - DNA, 22 bp, Artificial Sequence
  Synthetic: Sa13
ggggtctcagtgcatccaaaac

SEQ ID NO: 213 - DNA, 22 bp, Artificial Sequence
  Synthetic: Sa14
acaacgcaaaccgcggacactg

SEQ ID NO: 214 - DNA, 22 bp, Artificial Sequence
  Synthetic: Sa15
cttcggccgcctccacacgcct

SEQ ID NO: 215 - DNA, 22 bp, Artificial Sequence
  Synthetic: Sa16
ccccggccgctagggggcgggc

SEQ ID NO: 216 - DNA, 22 bp, Artificial Sequence
  Synthetic: Sa17
ggggcgcgggatccccgaaaaa

SEQ ID NO: 217 - DNA, 22 bp, Artificial Sequence
  Synthetic: Sa18
caaaacgtggattggggttgtt

SEQ ID NO: 218 - DNA, 22 bp, Artificial Sequence
  Synthetic: Sa19
ttgggggtcctgtagcctgtca

SEQ ID NO: 219 - DNA, 22 bp, Artificial Sequence
  Synthetic: Sa20
tcagtgcatccaaaacgtggat

SEQ ID NO: 220 - DNA, 22 bp, Artificial Sequence
  Synthetic: Sa21
actccggggccccgttggaaga

SEQ ID NO: 221 - DNA, 22 bp, Artificial Sequence
  Synthetic: Sa22
gacaataaataccgaggaatgt

SEQ ID NO: 222 - DNA, 22 bp, Artificial Sequence
  Synthetic: Sa23
tcggccaggctgaggccctgac

SEQ ID NO: 223 - DNA, 22 bp, Artificial Sequence
  Synthetic: Sa24
actttgcgaaccaacgataggt

SEQ ID NO: 224 - DNA, 22 bp, Artificial Sequence
  Synthetic: Sa25
cttttgccaaacccgctttttc

SEQ ID NO: 225 - DNA, 22 bp, Artificial Sequence
  Synthetic: Sa26
ggctcgaagggtccttgtagcc

SEQ ID NO: 226 - DNA, 22 bp, Artificial Sequence
  Synthetic: Sa27
tttattcgcgagggtcgggggt

SEQ ID NO: 227 - DNA, 22 bp, Artificial Sequence
  Synthetic: Sa28
ccgaaggtctgggaggagctag

SEQ ID NO: 228 - DNA, 22 bp, Artificial Sequence
  Synthetic: Sa29
aggacccccacccccgaccctc

SEQ ID NO: 229 - DNA, 22 bp, Artificial Sequence
  Synthetic: Sa30
gggtttggcaaaagcaaatttc

SEQ ID NO: 230 - DNA, 22 bp, Artificial Sequence
  Synthetic: Sa31
agcgcaagtgaggaggggggcg

SEQ ID NO: 231 - DNA, 22 bp, Artificial Sequence
  Synthetic: Sa32
ctagcggccggggagggagggg

SEQ ID NO: 232 - DNA, 22 bp, Artificial Sequence
  Synthetic: Sa33
ctgctgctgctgctgctgctgg

SEQ ID NO: 233 - DNA, 22 bp, Artificial Sequence
  Synthetic: NSa1
ccaggctgaggccctgacgtgg

SEQ ID NO: 234 - DNA, 22 bp, Artificial Sequence
  Synthetic: NSa3
aaccaacgataggtgggggtgc

SEQ ID NO: 235 - DNA, 22 bp, Artificial Sequence
  Synthetic: NSa4
tgtcttcgactccggggccccg

SEQ ID NO: 236 - DNA, 22 bp, Artificial Sequence
  Synthetic: NSa5
aggtggggacagacaataaata

SEQ ID NO: 237 - DNA, 22 bp, Artificial Sequence
  Synthetic: NSa6
gcgggcggagccggctggggct

SEQ ID NO: 238 - DNA, 22 bp, Artificial Sequence
  Synthetic: NSa7
cgcctgccagttcacaaccgct

SEQ ID NO: 239 - DNA, 22 bp, Artificial Sequence
  Synthetic: NSa8
tcgcgccagacgctccccagag

SEQ ID NO: 240 - DNA, 22 bp, Artificial Sequence
  Synthetic: NSa12
gccccgttggaagactgagtgc

SEQ ID NO: 241 - DNA, 22 bp, Artificial Sequence
  Synthetic: NSa14
cgcccagctccagtcctgtgat

SEQ ID NO: 242 - DNA, 22 bp, Artificial Sequence
  Synthetic: NSa16
ggcgcggcttctgtgccgtgcc

SEQ ID NO: 243 - DNA, 22 bp, Artificial Sequence
  Synthetic: NSa17
ggggcgggcccggatcacagga

SEQ ID NO: 244 - DNA, 22 bp, Artificial Sequence
  Synthetic: NSa18
ggggctcgaagggtccttgtag

SEQ ID NO: 245 - DNA, 22 bp, Artificial Sequence
  Synthetic: NSa24
cattcccggctacaaggaccct

SEQ ID NO: 246 - DNA, 22 bp, Artificial Sequence
  Synthetic: NSa34
cggcccctccctccccggccgc

SEQ ID NO: 247 - DNA, 22 bp, Artificial Sequence
  Synthetic: NSa40
cgggcccgccccctagcggccg

SEQ ID NO: 248 - DNA, 22 bp, Artificial Sequence
  Synthetic: NSa41
cccgccccctagcggccgggga

SEQ ID NO: 249 - DNA, 22 bp, Artificial Sequence
  Synthetic: NSa42
cactcagtcttccaacggggcc

SEQ ID NO: 250 - DNA, 22 bp, Artificial Sequence
  Synthetic: NSa45
ggagctgggcggagacccacgc

SEQ ID NO: 251 - DNA, 22 bp, Artificial Sequence
  Synthetic: NSa49
gcccctccctccccggccgcta

SEQ ID NO: 252 - DNA, 22 bp, Artificial Sequence
  Synthetic: NSa51
actgagtgcccggggcacggca

SEQ ID NO: 253 - DNA, 22 bp, Artificial Sequence
  Synthetic: NSa54
gtccgcggccggcgaacggggc

SEQ ID NO: 254 - DNA, 22 bp, Artificial Sequence
  Synthetic: NSa55
gtcttccaacggggccccggag

SEQ ID NO: 255 - DNA, 22 bp, Artificial Sequence
  Synthetic: NSa58
gagacccacgctcggagcggtt

SEQ ID NO: 256 - DNA, 22 bp, Artificial Sequence
  Synthetic: NSa59
gtcttcgactccggggccccgt

SEQ ID NO: 257 - DNA, 22 bp, Artificial Sequence
  Synthetic: NSa63
accctagaactgtcttcgactc

SEQ ID NO: 258 - DNA, 22 bp, Artificial Sequence
  Synthetic: NSa64
ccccgttggaagactgagtgcc

SEQ ID NO: 259 - DNA, 22 bp, Artificial Sequence
  Synthetic: NSa65
ggccgggtccgcggccggcgaa

SEQ ID NO: 260-499 - skipped (240 entries)

SEQ ID NO: 500 - DNA, 86 bp, Artificial Sequence
  Synthetic: exemplary scaffold sequence suitable for use with SaCas9 to follow the guide sequence at its 3′ end
gtttaagtactctgtgctggaaacagcacagaatctacttaaacaaggcaaaatgccgtgtttatctcgtcaacttgttggcgaga

SEQ ID NO: 501-599 - skipped (99 entries)

SEQ ID NO: 600 - DNA, 76 bp, Artificial Sequence
  Synthetic: guide RNA
gttttagtactctggaaacagaatctactgaaacaagacaatatgtcgtgtttatcccatcaatttattggtggga

SEQ ID NO: 601 - DNA, 86 bp, Artificial Sequence
  Synthetic: guide RNA
gtttaagtactctgtgctggaaacagcacagaatctactgaaacaagacaatatgtcgtgtttatcccatcaatttattggtggga

SEQ ID NO: 602 - RNA, 77 nt, Artificial Sequence
  Synthetic: crRNA and tracrRNA used for gRNAs with SluCas9
guuuuaguacucuggaaacagaaucuacugaaacaagacaauaugucguguuuaucccaucaauuuauuggugggau

SEQ ID NO: 603 - DNA, 156 bp, Artificial Sequence
  Synthetic: guide RNA
cttgtacttatacctaaaattacagaatctactgaaacaagacaatatgtcgtgtttatcccatcaatttattggtgggatttttttatgtttttagcaaaaagtaataccatactttatatttttaaattataataaagatataaataaaggtgg

SEQ ID NO: 604 - DNA, 77 bp, Artificial Sequence
  Synthetic: V5 scaffold for SluCas9
gtttcagtactctggaaacagaatctactgaaacaagacaatatgtcgtgtttatcccatcaatttattggtgggat

SEQ ID NO: 605-699 - skipped (95 entries)

SEQ ID NO: 700 - DNA, 450 bp, Artificial Sequence
  Synthetic: CK8 promoter
ctagactagcatgctgcccatgtaaggaggcaaggcctggggacacccgagatgcctggttataattaacccagacatgtggctgcccccccccccccaacacctgctgcctctaaaaataaccctgcatgccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagc

SEQ ID NO: 701 - DNA, 436 bp, Artificial Sequence
  Synthetic: CK8e promoter
tgcccatgtaaggaggcaaggcctggggacacccgagatgcctggttataattaacccagacatgtggctgcccccccccccccaacacctgctgcctctaaaaataaccctgcatgccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagc

SEQ ID NO: 702-710 - skipped (9 entries)

SEQ ID NO: 711 - protein, 1052 aa, Artificial Sequence
  Synthetic: SaCas9
KRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 712 - protein, 1053 aa, Artificial Sequence
  Synthetic: SluCas9 protein
NQKFILGLDIGITSVGYGLIDYETKNIIDAGVRLFPEANVENNEGRRSKRGSRRLKRRRIHRLERVKKLLEDYNLLDQSQIPQSTNPYAIRVKGLSEALSKDELVIALLHIAKRRGIHKIDVIDSNDDVGNELSTKEQLNKNSKLLKDKFVCQIQLERMNEGQVRGEKNRFKTADIIKEIIQLLNVQKNFHQLDENFINKYIELVEMRREYFEGPGKGSPYGWEGDPKAWYETLMGHCTYFPDELRSVKYAYSADLFNALNDLNNLVIQRDGLSKLEYHEKYHIIENVFKQKKKPTLKQIANEINVNPEDIKGYRITKSGKPQFTEFKLYHDLKSVLFDQSILENEDVLDQIAEILTIYQDKDSIKSKLTELDILLNEEDKENIAQLTGYTGTHRLSLKCIRLVLEEQWYSSRNQMEIFTHLNIKPKKINLTAANKIPKAMIDEFILSPVVKRTFGQAINLINKIIEKYGVPEDIIIELARENNSKDKQKFINEMQKKNENTRKRINEIIGKYGNQNAKRLVEKIRLHDEQEGKCLYSLESIPLEDLLNNPNHYEVDHIIPRSVSFDNSYHNKVLVKQSENSKKSNLTPYQYFNSGKSKLSYNQFKQHILNLSKSQDRISKKKKEYLLEERDINKFEVQKEFINRNLVDTRYATRELTNYLKAYFSANNMNVKVKTINGSFTDYLRKVWKFKKERNHGYKHHAEDALIIANADFLFKENKKLKAVNSVLEKPEIETKQLDIQVDSEDNYSEMFIIPKQVQDIKDFRNFKYSHRVDKKPNRQLINDTLYSTRKKDNSTYIVQTIKDIYAKDNTTLKKQFDKSPEKFLMYQHDPRTFEKLEVIMKQYANEKNPLAKYHEETGEYLTKYSKKNNGPIVKSLKYIGNKLGSHLDVTHQFKSSTKKLVKLSIKPYRFDVYLTDKGYKFITISYLDVLKKDNYYYIPEQKYDKLKLGKAIDKNAKFIASFYKNDLIKLDGEIYKIIGVNSDTRNMIELDLPDIRYKEYCELNNIKGEPRIKKTIGKKVNSIEKLTTDVLGNVFTNTQYTKPQLLFKRGN

SEQ ID NO: 713 - protein, 1053 aa, Artificial Sequence
  Synthetic: SluCas9-KH
NQKFILGLDIGITSVGYGLIDYETKNIIDAGVRLFPEANVENNEGRRSKRGSRRLKRRRIHRLERVKKLLEDYNLLDQSQIPQSTNPYAIRVKGLSEALSKDELVIALLHIAKRRGIHKIDVIDSNDDVGNELSTKEQLNKNSKLLKDKFVCQIQLERMNEGQVRGEKNRFKTADIIKEIIQLLNVQKNFHQLDENFINKYIELVEMRREYFEGPGKGSPYGWEGDPKAWYETLMGHCTYFPDELRSVKYAYSADLFNALNDLNNLVIQRDGLSKLEYHEKYHIIENVFKQKKKPTLKQIANEINVNPEDIKGYRITKSGKPQFTEFKLYHDLKSVLFDQSILENEDVLDQIAEILTIYQDKDSIKSKLTELDILLNEEDKENIAQLTGYTGTHRLSLKCIRLVLEEQWYSSRNQMEIFTHLNIKPKKINLTAANKIPKAMIDEFILSPVVKRTFGQAINLINKIIEKYGVPEDIIIELARENNSKDKQKFINEMQKKNENTRKRINEIIGKYGNQNAKRLVEKIRLHDEQEGKCLYSLESIPLEDLLNNPNHYEVDHIIPRSVSFDNSYHNKVLVKQSENSKKSNLTPYQYFNSGKSKLSYNQFKQHILNLSKSQDRISKKKKEYLLEERDINKFEVQKEFINRNLVDTRYATRELTNYLKAYFSANNMNVKVKTINGSFTDYLRKVWKFKKERNHGYKHHAEDALIIANADFLFKENKKLKAVNSVLEKPEIETKQLDIQVDSEDNYSEMFIIPKQVQDIKDFRNFKYSHRVDKKPNRKLINDTLYSTRKKDNSTYIVQTIKDIYAKDNTTLKKQFDKSPEKFLMYQHDPRTFEKLEVIMKQYANEKNPLAKYHEETGEYLTKYSKKNNGPIVKSLKYIGNKLGSHLDVTHQFKSSTKKLVKLSIKPYRFDVYLTDKGYKFITISYLDVLKKDNYYYIPEQKYDKLKLGKAIDKNAKFIASFYKNDLIKLDGEIYKIIGVNSDTRNMIELDLPDIRYKEYCELNNIKGEPHIKKTIGKKVNSIEKLTTDVLGNVFTNTQYTKPQLLFKRGN

SEQ ID NO: 714 - protein, 1053 aa, Artificial Sequence
  Synthetic: SluCas9-HF
NQKFILGLDIGITSVGYGLIDYETKNIIDAGVRLFPEANVENNEGRRSKRGSRRLKRRRIHRLERVKKLLEDYNLLDQSQIPQSTNPYAIRVKGLSEALSKDELVIALLHIAKRRGIHKIDVIDSNDDVGNELSTKEQLNKNSKLLKDKFVCQIQLERMNEGQVRGEKNRFKTADIIKEIIQLLNVQKNFHQLDENFINKYIELVEMRREYFEGPGKGSPYGWEGDPKAWYETLMGHCTYFPDELASVKYAYSADLFNALNDLNNLVIQRDGLSKLEYHEKYHIIENVFKQKKKPTLKQIANEINVNPEDIKGYRITKSGKPQFTEFKLYHDLKSVLFDQSILENEDVLDQIAEILTIYQDKDSIKSKLTELDILLNEEDKENIAQLTGYTGTHRLSLKCIRLVLEEQWYSSRAQMEIFAHLNIKPKKINLTAANKIPKAMIDEFILSPVVKRTFGQAINLINKIIEKYGVPEDIIIELARENNSKDKQKFINEMQKKNENTRKRINEIIGKYGNQNAKRLVEKIRLHDEQEGKCLYSLESIPLEDLLNNPNHYEVDHIIPRSVSFDNSYHNKVLVKQSENSKKSNLTPYQYFNSGKSKLSYNQFKQHILNLSKSQDRISKKKKEYLLEERDINKFEVQKEFINRNLVDTRYATAELTNYLKAYFSANNMNVKVKTINGSFTDYLRKVWKFKKERNHGYKHHAEDALIIANADFLFKENKKLKAVNSVLEKPEIETKQLDIQVDSEDNYSEMFIIPKQVQDIKDFRNFKYSHRVDKKPNRQLINDTLYSTRKKDNSTYIVQTIKDIYAKDNTTLKKQFDKSPEKFLMYQHDPRTFEKLEVIMKQYANEKNPLAKYHEETGEYLTKYSKKNNGPIVKSLKYIGNKLGSHLDVTHQFKSSTKKLVKLSIKPYRFDVYLTDKGYKFITISYLDVLKKDNYYYIPEQKYDKLKLGKAIDKNAKFIASFYKNDLIKLDGEIYKIIGVNSDTRNMIELDLPDIRYKEYCELNNIKGEPRIKKTIGKKVNSIEKLTTDVLGNVFTNTQYTKPQLLFKRGN

SEQ ID NO: 715 - protein, 1053 aa, Artificial Sequence
  Synthetic: SluCas9-HF-KH
NQKFILGLDIGITSVGYGLIDYETKNIIDAGVRLFPEANVENNEGRRSKRGSRRLKRRRIHRLERVKKLLEDYNLLDQSQIPQSTNPYAIRVKGLSEALSKDELVIALLHIAKRRGIHKIDVIDSNDDVGNELSTKEQLNKNSKLLKDKFVCQIQLERMNEGQVRGEKNRFKTADIIKEIIQLLNVQKNFHQLDENFINKYIELVEMRREYFEGPGKGSPYGWEGDPKAWYETLMGHCTYFPDELASVKYAYSADLFNALNDLNNLVIQRDGLSKLEYHEKYHIIENVFKQKKKPTLKQIANEINVNPEDIKGYRITKSGKPQFTEFKLYHDLKSVLFDQSILENEDVLDQIAEILTIYQDKDSIKSKLTELDILLNEEDKENIAQLTGYTGTHRLSLKCIRLVLEEQWYSSRAQMEIFAHLNIKPKKINLTAANKIPKAMIDEFILSPVVKRTFGQAINLINKIIEKYGVPEDIIIELARENNSKDKQKFINEMQKKNENTRKRINEIIGKYGNQNAKRLVEKIRLHDEQEGKCLYSLESIPLEDLLNNPNHYEVDHIIPRSVSFDNSYHNKVLVKQSENSKKSNLTPYQYFNSGKSKLSYNQFKQHILNLSKSQDRISKKKKEYLLEERDINKFEVQKEFINRNLVDTRYATAELTNYLKAYFSANNMNVKVKTINGSFTDYLRKVWKFKKERNHGYKHHAEDALIIANADFLFKENKKLKAVNSVLEKPEIETKQLDIQVDSEDNYSEMFIIPKQVQDIKDFRNFKYSHRVDKKPNRKLINDTLYSTRKKDNSTYIVQTIKDIYAKDNTTLKKQFDKSPEKFLMYQHDPRTFEKLEVIMKQYANEKNPLAKYHEETGEYLTKYSKKNNGPIVKSLKYIGNKLGSHLDVTHQFKSSTKKLVKLSIKPYRFDVYLTDKGYKFITISYLDVLKKDNYYYIPEQKYDKLKLGKAIDKNAKFIASFYKNDLIKLDGEIYKIIGVNSDTRNMIELDLPDIRYKEYCELNNIKGEPHIKKTIGKKVNSIEKLTTDVLGNVFTNTQYTKPQLLFKRGN

SEQ ID NO: 716 - protein, 1057 aa, Artificial Sequence
  Synthetic: sRGN1
MNQKFILGLDIGITSVGYGLIDYETKNIIDAGVRLFPEANVENNEGRRSKRGSRRLKRRRIHRLDRVKHLLAEYDLLDLTNIPKSTNPYQTRVKGLNEKLSKDELVIALLHIAKRRGIHNVDVAADKEETASDSLSTKDQINKNAKFLESRYVCELQKERLENEGHVRGVENRFLTKDIVREAKKIIDTQMQYYPEIDETFKEKYISLVETRREYFEGPGKGSPFGWEGNIKKWFEQMMGHCTYFPEELRSVKYSYSAELFNALNDLNNLVITRDEDAKLNYGEKFQIIENVFKQKKTPNLKQIAIEIGVHETEIKGYRVNKSGTPEFTEFKLYHDLKSIVFDKSILENEAILDQIAEILTIYQDEQSIKEELNKLPEILNEQDKAEIAKLIGYNGTHRLSLKCIHLINEELWQTSRNQMEIFNYLNIKPNKVDLSEQNKIPKDMVNDFILSPVVKRTFIQSINVINKVIEKYGIPEDIIIELARENNSDDRKKFINNLQKKNEATRKRINEIIGQTGNQNAKRIVEKIRLHDQQEGKCLYSLKDIPLEDLLRNPNNYDIDHIIPRSVSFDDSMHNKVLVRREQNAKKNNQTPYQYLTSGYADIKYSVFKQHVLNLAENKDRMTKKKREYLLEERDINKFEVQKEFINRNLVDTRYATRELTNYLKAYFSANNMNVKVKTINGSFTDYLRKVWKFKKERNHGYKHHAEDALIIANADFLFKENKKLKAVNSVLEKPEIETKQLDIQVDSEDNYSEMFIIPKQVQDIKDFRNFKYSHRVDKKPNRQLINDTLYSTRKKDNSTYIVQTIKDIYAKDNTTLKKQFDKSPEKFLMYQHDPRTFEKLEVIMKQYANEKNPLAKYHEETGEYLTKYSKKNNGPIVKSLKYIGNKLGSHLDVTHQFKSSTKKLVKLSIKPYRFDVYLTDKGYKFITISYLDVLKKDNYYYIPEQKYDKLKLGKAIDKNAKFIASFYKNDLIKLDGEIYKIIGVNSDTRNMIELDLPDIRYKEYCELNNIKGEPRIKKTIGKKVNSIEKLTTDVLGNVFTNTQYTKPQLLFKRGN

SEQ ID NO: 717 - protein, 1053 aa, Artificial Sequence
  Synthetic: sRGN2
MNQKFILGLDIGITSVGYGLIDYETKNIIDAGVRLFPEANVENNEGRRSKRGSRRLKRRRIHRLERVKSLLSEYKIISGLAPTNNQPYNIRVKGLTEQLTKDELAVALLHIAKRRGIHKIDVIDSNDDVGNELSTKEQLNKNSKLLKDKFVCQIQLERMNEGQVRGEKNRFKTADIIKEIIQLLNVQKNFHQLDENFINKYIELVEMRREYFEGPGQGSPFGWNGDLKKWYEMLMGHCTYFPQELRSVKYAYSADLFNALNDLNNLIIQRDNSEKLEYHEKYHIIENVFKQKKKPTLKQIAKEIGVNPEDIKGYRITKSGTPEFTEFKLYHDLKSVLFDQSILENEDVLDQIAEILTIYQDKDSIKSKLTELDILLNEEDKENIAQLTGYNGTHRLSLKCIRLVLEEQWYSSRNQMEIFTHLNIKPKKINLTAANKIPKAMIDEFILSPVVKRTFIQSINVINKVIEKYGIPEDIIIELARENNSDDRKKFINNLQKKNEATRKRINEIIGQTGNQNAKRIVEKIRLHDQQEGKCLYSLESIALMDLLNNPQNYEVDHIIPRSVAFDNSIHNKVLVKQIENSKKGNRTPYQYLNSSDAKLSYNQFKQHILNLSKSKDRISKKKKDYLLEERDINKFEVQKEFINRNLVDTRYATRELTSYLKAYFSANNMDVKVKTINGSFTNHLRKVWRFDKYRNHGYKHHAEDALIIANADFLFKENKKLKAVNSVLEKPEIETKQLDIQVDSEDNYSEMFIIPKQVQDIKDFRNFKYSHRVDKKPNRQLINDTLYSTRKKDNSTYIVQTIKDIYAKDNTTLKKQFDKSPEKFLMYQHDPRTFEKLEVIMKQYANEKNPLAKYHEETGEYLTKYSKKNNGPIVKSLKYIGNKLGSHLDVTHQFKSSTKKLVKLSIKPYRFDVYLTDKGYKFITISYLDVLKKDNYYYIPEQKYDKLKLGKAIDKNAKFIASFYKNDLIKLDGEIYKIIGVNSDTRNMIELDLPDIRYKEYCELNNIKGEPRIKKTIGKKVNSIEKLTTDVLGNVFTNTQYTKPQLLFKRGN

SEQ ID NO: 718 - protein, 1057 aa, Artificial Sequence
  Synthetic: sRGN3
MNQKFILGLDIGITSVGYGLIDYETKNIIDAGVRLFPEANVENNEGRRSKRGSRRLKRRRIHRLERVKLLLTEYDLINKEQIPTSNNPYQIRVKGLSEILSKDELAIALLHLAKRRGIHNVDVAADKEETASDSLSTKDQINKNAKFLESRYVCELQKERLENEGHVRGVENRFLTKDIVREAKKIIDTQMQYYPEIDETFKEKYISLVETRREYFEGPGQGSPFGWNGDLKKWYEMLMGHCTYFPQELRSVKYAYSADLFNALNDLNNLIIQRDNSEKLEYHEKYHIIENVFKQKKKPTLKQIAKEIGVNPEDIKGYRITKSGTPEFTSFKLFHDLKKVVKDHAILDDIDLLNQIAEILTIYQDKDSIVAELGQLEYLMSEADKQSISELTGYTGTHSLSLKCMNMIIDELWHSSMNQMEVFTYLNMRPKKYELKGYQRIPTDMIDDAILSPVVKRTFIQSINVINKVIEKYGIPEDIIIELARENNSDDRKKFINNLQKKNEATRKRINEIIGQTGNQNAKRIVEKIRLHDQQEGKCLYSLESIPLEDLLNNPNHYEVDHIIPRSVSFDNSYHNKVLVKQSENSKKSNLTPYQYFNSGKSKLSYNQFKQHILNLSKSQDRISKKKKEYLLEERDINKFEVQKEFINRNLVDTRYATRELTNYLKAYFSANNMNVKVKTINGSFTDYLRKVWKFKKERNHGYKHHAEDALIIANADFLFKENKKLKAVNSVLEKPEIETKQLDIQVDSEDNYSEMFIIPKQVQDIKDFRNFKYSHRVDKKPNRQLINDTLYSTRKKDNSTYIVQTIKDIYAKDNTTLKKQFDKSPEKFLMYQHDPRTFEKLEVIMKQYANEKNPLAKYHEETGEYLTKYSKKNNGPIVKSLKYIGNKLGSHLDVTHQFKSSTKKLVKLSIKPYRFDVYLTDKGYKFITISYLDVLKKDNYYYIPEQKYDKLKLGKAIDKNAKFIASFYKNDLIKLDGEIYKIIGVNSDTRNMIELDLPDIRYKEYCELNNIKGEPRIKKTIGKKVNSIEKLTTDVLGNVFTNTQYTKPQLLFKRGN

SEQ ID NO: 719 - protein, 1057 aa, Artificial Sequence
  Synthetic: sRGN3.1
MNQKFILGLDIGITSVGYGLIDYETKNIIDAGVRLFPEANVENNEGRRSKRGSRRLKRRRIHRLERVKLLLTEYDLINKEQIPTSNNPYQIRVKGLSEILSKDELAIALLHLAKRRGIHNVDVAADKEETASDSLSTKDQINKNAKFLESRYVCELQKERLENEGHVRGVENRFLTKDIVREAKKIIDTQMQYYPEIDETFKEKYISLVETRREYFEGPGQGSPFGWNGDLKKWYEMLMGHCTYFPQELRSVKYAYSADLFNALNDLNNLIIQRDNSEKLEYHEKYHIIENVFKQKKKPTLKQIAKEIGVNPEDIKGYRITKSGTPEFTSFKLFHDLKKVVKDHAILDDIDLLNQIAEILTIYQDKDSIVAELGQLEYLMSEADKQSISELTGYTGTHSLSLKCMNMIIDELWHSSMNQMEVFTYLNMRPKKYELKGYQRIPTDMIDDAILSPVVKRTFIQSINVINKVIEKYGIPEDIIIELARENNSDDRKKFINNLQKKNEATRKRINEIIGQTGNQNAKRIVEKIRLHDQQEGKCLYSLESIPLEDLLNNPNHYEVDHIIPRSVSFDNSYHNKVLVKQSENSKKSNLTPYQYFNSGKSKLSYNQFKQHILNLSKSQDRISKKKKEYLLEERDINKFEVQKEFINRNLVDTRYATRELTNYLKAYFSANNMNVKVKTINGSFTDYLRKVWKFKKERNHGYKHHAEDALIIANADFLFKENKKLKAVNSVLEKPEIETKQLDIQVDSEDNYSEMFIIPKQVQDIKDFRNFKYSHRVDKKPNRQLINDTLYSTRKKDNSTYIVQTIKDIYAKDNTTLKKQFDKSPEKFLMYQHDPRTFEKLEVIMKQYANEKNPLAKYHEETGEYLTKYSKKNNGPIVKSLKYIGNKLGSHLDVTHQFKSSTKKLVKLSIKNYRFDVYLTEKGYKFVTIAYLNVFKKDNYYYIPKDKYQELKEKKKIKDTDQFIASFYKNDLIKLNGDLYKIIGVNSDDRNIIELDYYDIKYKDYCEINNIKGEPRIKKTIGKKTESIEKFTTDVLGNLYLHSTEKAPQLIFKRGL

SEQ ID NO: 720 - protein, 1057 aa, Artificial Sequence
  Synthetic: sRGN3.2
MNQKFILGLDIGITSVGYGLIDYETKNIIDAGVRLFPEANVENNEGRRSKRGSRRLKRRRIHRLERVKLLLTEYDLINKEQIPTSNNPYQIRVKGLSEILSKDELAIALLHLAKRRGIHNVDVAADKEETASDSLSTKDQINKNAKFLESRYVCELQKERLENEGHVRGVENRFLTKDIVREAKKIIDTQMQYYPEIDETFKEKYISLVETRREYFEGPGQGSPFGWNGDLKKWYEMLMGHCTYFPQELRSVKYAYSADLFNALNDLNNLIIQRDNSEKLEYHEKYHIIENVFKQKKKPTLKQIAKEIGVNPEDIKGYRITKSGTPEFTSFKLFHDLKKVVKDHAILDDIDLLNQIAEILTIYQDKDSIVAELGQLEYLMSEADKQSISELTGYTGTHSLSLKCMNMIIDELWHSSMNQMEVFTYLNMRPKKYELKGYQRIPTDMIDDAILSPVVKRTFIQSINVINKVIEKYGIPEDIIIELARENNSDDRKKFINNLQKKNEATRKRINEIIGQTGNQNAKRIVEKIRLHDQQEGKCLYSLESIPLEDLLNNPNHYEVDHIIPRSVSFDNSYHNKVLVKQSENSKKSNLTPYQYFNSGKSKLSYNQFKQHILNLSKSQDRISKKKKEYLLEERDINKFEVQKEFINRNLVDTRYATRELTNYLKAYFSANNMNVKVKTINGSFTDYLRKVWKFKKERNHGYKHHAEDALIIANADFLFKENKKLKAVNSVLEKPEIETKQLDIQVDSEDNYSEMFIIPKQVQDIKDFRNFKFSHRVDKKPNRQLINDTLYSTRMKDEHDYIVQTITDIYGKDNTNLKKQFNKNPEKFLMYQNDPKTFEKLSIIMKQYSDEKNPLAKYYEETGEYLTKYSKKNNGPIVKKIKLLGNKVGNHLDVTNKYENSTKKLVKLSIKNYRFDVYLTEKGYKFVTIAYLNVFKKDNYYYIPKDKYQELKEKKKIKDTDQFIASFYKNDLIKLNGDLYKIIGVNSDDRNIIELDYYDIKYKDYCEINNIKGEPRIKKTIGKKTESIEKFTTDVLGNLYLHSTEKAPQLIFKRGL

SEQ ID NO: 721 - protein, 1057 aa, Artificial Sequence
  Synthetic: sRGN3.3
MNQKFILGLDIGITSVGYGLIDYETKNIIDAGVRLFPEANVENNEGRRSKRGSRRLKRRRIHRLERVKLLLTEYDLINKEQIPTSNNPYQIRVKGLSEILSKDELAIALLHLAKRRGIHNVDVAADKEETASDSLSTKDQINKNAKFLESRYVCELQKERLENEGHVRGVENRFLTKDIVREAKKIIDTQMQYYPEIDETFKEKYISLVETRREYFEGPGQGSPFGWNGDLKKWYEMLMGHCTYFPQELRSVKYAYSADLFNALNDLNNLIIQRDNSEKLEYHEKYHIIENVFKQKKKPTLKQIAKEIGVNPEDIKGYRITKSGTPEFTSFKLFHDLKKVVKDHAILDDIDLLNQIAEILTIYQDKDSIVAELGQLEYLMSEADKQSISELTGYTGTHSLSLKCMNMIIDELWHSSMNQMEVFTYLNMRPKKYELKGYQRIPTDMIDDAILSPVVKRTFIQSINVINKVIEKYGIPEDIIIELARENNSDDRKKFINNLQKKNEATRKRINEIIGQTGNQNAKRIVEKIRLHDQQEGKCLYSLESIPLEDLLNNPNHYEVDHIIPRSVSFDNSYHNKVLVKQSENSKKSNLTPYQYFNSGKSKLSYNQFKQHILNLSKSQDRISKKKKEYLLEERDINKFEVQKEFINRNLVDTRYATRELTSYLKAYFSANNMDVKVKTINGSFTNHLRKVWRFDKYRNHGYKHHAEDALIIANADFLFKENKKLQNTNKILEKPTIENNTKKVTVEKEEDYNNVFETPKLVEDIKQYRDYKFSHRVDKKPNRQLINDTLYSTRMKDEHDYIVQTITDIYGKDNTNLKKQFNKNPEKFLMYQNDPKTFEKLSIIMKQYSDEKNPLAKYYEETGEYLTKYSKKNNGPIVKKIKLLGNKVGNHLDVTNKYENSTKKLVKLSIKNYRFDVYLTEKGYKFVTIAYLNVFKKDNYYYIPKDKYQELKEKKKIKDTDQFIASFYKNDLIKLNGDLYKIIGVNSDDRNIIELDYYDIKYKDYCEINNIKGEPRIKKTIGKKTESIEKFTTDVLGNLYLHSTEKAPQLIFKRGL

SEQ ID NO: 722 - protein, 1054 aa, Artificial Sequence
  Synthetic: sRGN4
MNQKFILGLDIGITSVGYGLIDYETKNIIDAGVRLFPEANVENNEGRRSKRGSRRLKRRRIHRLERVKKLLEDYNLLDQSQIPQSTNPYAIRVKGLSEALSKDELVIALLHIAKRRGIHNINVSSEDEDASNELSTKEQINRNNKLLKDKYVCEVQLQRLKEGQIRGEKNRFKTTDILKEIDQLLKVQKDYHNLDIDFINQYKEIVETRREYFEGPGKGSPYGWEGDPKAWYETLMGHCTYFPDELRSVKYAYSADLFNALNDLNNLVIQRDGLSKLEYHEKYHIIENVFKQKKKPTLKQIANEINVNPEDIKGYRITKSGKPEFTSFKLFHDLKKVVKDHAILDDIDLLNQIAEILTIYQDKDSIVAELGQLEYLMSEADKQSISELTGYTGTHSLSLKCMNMIIDELWHSSMNQMEVFTYLNMRPKKYELKGYQRIPTDMIDDAILSPVVKRTFIQSINVINKVIEKYGIPEDIIIELARENNSDDRKKFINNLQKKNEATRKRINEIIGQTGNQNAKRIVEKIRLHDQQEGKCLYSLESIPLEDLLNNPNHYEVDHIIPRSVSFDNSYHNKVLVKQSENSKKSNLTPYQYFNSGKSKLSYNQFKQHILNLSKSQDRISKKKKEYLLEERDINKFEVQKEFINRNLVDTRYATRELTNYLKAYFSANNMNVKVKTINGSFTDYLRKVWKFKKERNHGYKHHAEDALIIANADFLFKENKKLKAVNSVLEKPEIETKQLDIQVDSEDNYSEMFIIPKQVQDIKDFRNFKYSHRVDKKPNRQLINDTLYSTRKKDNSTYIVQTIKDIYAKDNTTLKKQFDKSPEKFLMYQHDPRTFEKLEVIMKQYANEKNPLAKYHEETGEYLTKYSKKNNGPIVKSLKYIGNKLGSHLDVTHQFKSSTKKLVKLSIKPYRFDVYLTDKGYKFITISYLDVLKKDNYYYIPEQKYDKLKLGKAIDKNAKFIASFYKNDLIKLDGEIYKIIGVNSDTRNMIELDLPDIRYKEYCELNNIKGEPRIKKTIGKKVNSIEKLTTDVLGNVFTNTQYTKPQLLFKRGN

SEQ ID NO: 723-799 - skipped (77 entries)

SEQ ID NO: 800 - protein, 1055 aa, Artificial Sequence
  Synthetic: M-SluCas9_X
  misc_feature (239)..(239): Xaa can be any naturally occurring amino acid
  misc_feature (401)..(401): Xaa can be any naturally occurring amino acid
  misc_feature (408)..(408): Xaa can be any naturally occurring amino acid
  misc_feature (414)..(414): Xaa can be any naturally occurring amino acid
  misc_feature (418)..(418): Xaa can be any naturally occurring amino acid
  misc_feature (422)..(422): Xaa can be any naturally occurring amino acid
MNQKFILGLDIGITSVGYGLIDYETKNIIDAGVRLFPEANVENNEGRRSKRGSRRLKRRRIHRLERVKKLLEDYNLLDQSQIPQSTNPYAIRVKGLSEALSKDELVIALLHIAKRRGIHKIDVIDSNDDVGNELSTKEQLNKNSKLLKDKFVCQIQLERMNEGQVRGEKNRFKTADIIKEIIQLLNVQKNFHQLDENFINKYIELVEMRREYFEGPGKGSPYGWEGDPKAWYETLMGHXTYFPDELRSVKYAYSADLFNALNDLNNLVIQRDGLSKLEYHEKYHIIENVFKQKKKPTLKQIANEINVNPEDIKGYRITKSGKPQFTEFKLYHDLKSVLFDQSILENEDVLDQIAEILTIYQDKDSIKSKLTELDILLNEEDKENIAQLTGYTGTHRLSLKXIRLVLEXQWYSSXNQMXIFTXLNIKPKKINLTAANKIPKAMIDEFILSPWKRTFGQAINLINKIIEKYGVPEDIIIELARENNSKDKQKFINEMQKKNENTRKRINEIIGKYGNQNAKRLVEKIRLHDEQEGKCLYSLESIPLEDLLNNPNHYEVDHIIPRSVSFDNSYHNKVLVKQSENSKKSNLTPYQYFNSGKSKLSYNQFKQHILNLSKSQDRISKKKKEYLLEERDINKFEVQKEFINRNLVDTRYATRELTNYLKAYFSANNMNVKVKTINGSFTDYLRKVWKFKKERNHGYKHHAEDALIIANADFLFKENKKLKAVNSVLEKPEIETKQLDIQVDSEDNYSEMFIIPKQVQDIKDFRNFKYSHRVDKKPNRQLINDTLYSTRKKDNSTYIVQTIKDIYAKDNTTLKKQFDKSPEKFLMYQHDPRTFEKLEVIMKQYANEKNPLAKYHEETGEYLTKYSKKNNGPIVKSLKYIGNKLGSHLDVTHQFKSSTKKLVKLSIKPYRFDVYLTDKGYKFITISYLDVLKKDNYYYIPEQKYDKLKLGKAIDKNAKFIASFYKNDLIKLDGEIYKIIGVNSDTRNMIELDLPDIRYKEYCELNNIKGEPRIKKTIGKKVNSIEKLTTDVLGNVFTNTQYTKPQLLFKRGNGG

SEQ ID NO: 801 - protein, 1055 aa, Artificial Sequence
  Synthetic: M-SluCas9-R414A
MNQKFILGLDIGITSVGYGLIDYETKNIIDAGVRLFPEANVENNEGRRSKRGSRRLKRRRIHRLERVKKLLEDYNLLDQSQIPQSTNPYAIRVKGLSEALSKDELVIALLHIAKRRGIHKIDVIDSNDDVGNELSTKEQLNKNSKLLKDKFVCQIQLERMNEGQVRGEKNRFKTADIIKEIIQLLNVQKNFHQLDENFINKYIELVEMRREYFEGPGKGSPYGWEGDPKAWYETLMGHCTYFPDELRSVKYAYSADLFNALNDLNNLVIQRDGLSKLEYHEKYHIIENVFKQKKKPTLKQIANEINVNPEDIKGYRITKSGKPQFTEFKLYHDLKSVLFDQSILENEDVLDQIAEILTIYQDKDSIKSKLTELDILLNEEDKENIAQLTGYTGTHRLSLKCIRLVLEEQWYSSANQMEIFTHLNIKPKKINLTAANKIPKAMIDEFILSPWKRTFGQAINLINKIIEKYGVPEDIIIELARENNSKDKQKFINEMQKKNENTRKRINEIIGKYGNQNAKRLVEKIRLHDEQEGKCLYSLESIPLEDLLNNPNHYEVDHIIPRSVSFDNSYHNKVLVKQSENSKKSNLTPYQYFNSGKSKLSYNQFKQHILNLSKSQDRISKKKKEYLLEERDINKFEVQKEFINRNLVDTRYATRELTNYLKAYFSANNMNVKVKTINGSFTDYLRKVWKFKKERNHGYKHHAEDALIIANADFLFKENKKLKAVNSVLEKPEIETKQLDIQVDSEDNYSEMFIIPKQVQDIKDFRNFKYSHRVDKKPNRQLINDTLYSTRKKDNSTYIVQTIKDIYAKDNTTLKKQFDKSPEKFLMYQHDPRTFEKLEVIMKQYANEKNPLAKYHEETGEYLTKYSKKNNGPIVKSLKYIGNKLGSHLDVTHQFKSSTKKLVKLSIKPYRFDVYLTDKGYKFITISYLDVLKKDNYYYIPEQKYDKLKLGKAIDKNAKFIASFYKNDLIKLDGEIYKIIGVNSDTRNMIELDLPDIRYKRYCELNNIKGEPRIKKTIGKKVNSIEKLTTDVLGNVFTNTQYTKPQLLFKRGNGG

SEQ ID NO: 802 - protein, 1056 aa, Artificial Sequence
  Synthetic: SluCas9 in WO2019/ 118935
MNQKFILGLDIGITSVGYGLIDYETKNIIDAGVRLFPEANVENNEGRRSKRGSRRLKRRRIHRLERVKKLLEDYNLLDQSQIPQSTNPYAIRVKGLSEALSKDELVIALLHIAKRRGIHKIDVIDSNDDVGNELSTKEQLNKNSKLLKDKFVCQIQLERMNEGQVRGEKNRFKTADIIKEIIQLLNVQKNFHQLDENFINKYIELVEMRREYFEGPGKGSPYGWEGDPKAWYETLMGHCTYFPDELRSVKYAYSADLFNALNDLNNLVIQRDGLSKLEYHEKYHIIENVFKQKKKPTLKQIANEINVNPEDIKGYRITKSGKPQFTEFKLYHDLKSVLFDQSILENEDVLDQIAEILTIYQDKDSIKSKLTELDILLNEEDKENIAQLTGYTGTHRLSLKCIRLVLEEQWYSSRNQMEIFTHLNIKPKKINLTAANKIPKAMIDEFILSPVVKRTFGQAINLINKIIEKYGVPEDIIIELARENNSKDKQKFINEMQKKNENTRKRINEIIGKYGNQNAKRLVEKIRLHDEQEGKCLYSLESIPLEDLLNNPNHYEVDHIIPRSVSFDNSYHNKVLVKQSENSKKSNLTPYQYFNSGKSKLSYNQFKQHILNLSKSQDRISKKKKEYLLEERDINKFEVQKEFINRNLVDTRYATRELTNYLKAYFSANNMNVKVKTINGSFTDYLRKVWKFKKERNHGYKHHAEDALIIANADFLFKENKKLKAVNSVLEKPEIETKQLDIQVDSEDNYSEMFIIPKQVQDIKDFRNFKYSHRVDKKPNRQLINDTLYSTRKKDNSTYIVQTIKDIYAKDNTTLKKQFDKSPEKFLMYQHDPRTFEKLEVIMKQYANEKNPLAKYHEETGEYLTKYSKKNNGPIVKSLKYIGNKLGSHLDVTHQFKSSTKKLVKLSIKPYRFDVYLTDKGYKFITISYLDVLKKDNYYYIPEQKYDKLKLGKAIDKNAKFIASFYKNDLIKLDGEIYKIIGVNSDTRNMIELDLPDIRYKEYCELNNIKGEPRIKKTIGKKVNSIEKLTTDVLGNVFTNTQYTKPQLLFKRGNGG

SEQ ID NO: 803 - DNA, 3315 bp, Artificial Sequence
  Synthetic: Codon optimized version of a polynucleotide sequence shown from position 61 to 3225 (SEQ ID NO: 3 in WO2019/ 183150)
atgaaacgtccggcagcaaccaaaaaagcaggtcaggccaagaaaaaaaaaggtggtggttcaggtaaccagaaatttatcctgggtctggatattggtattaccagcgttggttatggcctgattgattacgaaaccaaaaacattattgatgccggtgttcgtctgtttccggaagcaaatgttgaaaataatgaaggtcgtcgtagcaaacgtggtagccgtcgtctgaaacgtcgtcgtattcatcgtctggaacgtgttaaaaaactgctggaagattataacctgctggatcagagccagattccgcagagcaccaatccgtatgcaattcgtgttaaaggtctgagcgaagcactgagcaaagatgaactggttattgcactgctgcatattgcaaaacgccgtggcattcataaaatcgatgtgattgatagcaatgacgatgtgggtaatgaactgagcaccaaagaacagctgaacaaaaatagcaaactgctgaaagacaaattcgtgtgtcagattcagctggaacgtatgaatgaaggccaggttcgtggtgaaaagaatcgctttaaaaccgcagacatcatcaaagaaattatccagctgctgaacgtgcagaaaaacttccatcagctggatgaaaacttcatcaacaaatacatcgagctggttgaaatgcgtcgcgaatattttgaaggtccgggtaaaggtagcccgtatggttgggaaggtgatccgaaagcatggtatgaaaccctgatgggtcattgtacctattttccggatgaactgcgtagcgttaaatatgcctatagcgcagacctgtttaatgcactgaatgatctgaataacctggtgattcagcgtgatggtctgagcaaactggaatatcatgagaaatatcacatcatcgaaaacgtgttcaaacagaagaagaaaccgaccctgaaacaaatcgccaacgaaattaatgtgaacccggaagatattaaaggctaccgtattaccaaaagcggtaaaccgcagttcaccgaatttaaactgtatcacgatctgaaaagcgtgctgtttgatcagagcattctggaaaatgaagatgtgctggaccagattgcagaaattctgaccatttatcaggacaaagacagcatcaaaagcaaactgaccgaactggatattctgctgaatgaagaagataaagagaacattgcacagctgaccggttataccggcacccatcgtctgagcctgaaatgtattcgtctggtactggaagaacagtggtatagcagccgtaatcagatggaaatctttacccatctgaacattaaaccgaagaaaatcaatctgaccgcagccaacaaaattccgaaagccatgattgatgagtttattctgagtccggttgtgaaacgtacctttggtcaggcaattaacctgatcaacaaaatcattgaaaaatatggcgtgcctgaggatatcattattgaactggcacgtgaaaacaacagcaaagataaacagaaattcatcaacgagatgcagaagaagaacgaaaatacccgcaaacggattaacgagatcattggcaaatatggtaatcagaatgccaaacgcctggtggaaaaaattcgtctgcatgatgaacaagagggcaaatgtctgtatagcctggaaagcattcctctggaagatctgctgaacaatccgaatcattatgaagtggatcacattattccgcgtagcgtgagctttgataattcctatcataataaagtgctggtgaaacagagcgaaaactccaaaaaatccaacctgacaccgtatcagtatttcaatagcggcaaatccaaactgagctacaaccagtttaaacagcatattctgaacctgagcaaaagccaggatcgcatcagcaagaagaagaaggagtacctgctggaagaacgcgacatcaacaaatttgaagtgcagaaagaatttatcaaccgcaacctggttgatacccgttatgcaacccgtgaactgaccaattatctgaaagcatatttcagcgccaacaacatgaacgtgaaagtgaaaacgattaacggcagctttaccgattatctgcgtaaagtgtggaaattcaaaaaagaacgcaaccacggctataaacatcatgccgaagatgccctgattattgcaaatgcagatttcctgtttaaagaaaacaaaaaactgaaagccgtcaacagcgtgctggaaaaaccggaaattgagacaaaacagctggacattcaggttgatagcgaagataattacagcgaaatgtttatcatcccgaaacaggtgcaggatatcaaagattttcgcaacttcaaatatagccaccgcgttgacaaaaaacctaatcgtcagctgattaacgataccctgtatagcacccgcaaaaaagataacagcacctatattgtgcagaccattaaagacatctacgccaaagataataccaccctgaaaaaacagttcgacaaaagcccagaaaaatttctgatgtatcagcatgatccgcgtaccttcgaaaaactggaagttattatgaaacagtatgccaacgagaaaaatccgctggccaaatatcacgaagaaaccggtgaatatctgaccaaatattccaagaagaacaacggtccgatcgttaaatccctgaaatatatcggtaataaactgggcagccatctggatgttacccatcagtttaaaagctccacaaagaagctggttaaactgtccatcaaaccgtatcgctttgatgtgtatctgaccgacaaaggctataaattcattaccatcagctatctggacgtgctgaaaaaagacaactattattatatcccggaacagaaatatgataaactgaaactgggtaaagccatcgataaaaacgccaaatttatcgccagcttctacaaaaacgacctgattaaactggatggcgagatctataaaatcatcggtgttaatagcgacacccgcaatatgattgagctggatctgccggatattcgctataaagaatattgcgaactgaacaacattaaaggcgaaccgcgtatcaaaaagaccatcggcaaaaaagtgaatagcatcgagaaactgaccaccgatgttctgggtaatgtgtttaccaatacccagtataccaaacctcagctgctgttcaaacgcggtaatggcggaggatctggcccccctaagaaaaagcggaaggtgggtggaagcggaggcagcgggggatcaggccatcatcatcaccatcattaa

SEQ ID NO: 804 - DNA, 3162 bp, Artificial Sequence
  Synthetic: Codon optimized version of a polynucleotide sequence shown from position 61 to 3225 (SEQ ID NO: 44 in WO2019/ 183150)
atgaaccaaaagttcattctggggctcgatatcggcatcacctccgtgggatatggtctgatcgactacgagactaagaacatcatcgacgctggagtgcgactgttcccggaagcgaacgtggagaacaacgaaggccgcagatccaagcgcgggtccagaaggctcaagaggcggaggatccatagactcgaaagagtgaagaagctccttgaagattacaatctgttggaccagagccagattccccaaagcaccaacccgtacgccatcagagtgaagggcctgtccgaagccctgtcgaaagatgaactggtcattgccctgctgcatattgccaaacggcgcggaatccataagatcgacgtgatagactccaacgatgacgtgggcaacgaactgtcaaccaaggagcagctgaacaagaactcgaaactgctgaaggacaagttcgtctgccaaattcaactggaacggatgaacgagggacaagtcaggggagagaaaaaccggttcaagaccgcggacatcatcaaggagatcatccaactcctgaatgtgcagaagaactttcaccagctggatgaaaacttcattaacaagtacattgaactggtggaaatgcggagggagtacttcgagggacctggaaagggatccccttacggctgggaaggggaccccaaggcttggtacgaaacgctcatgggccattgcacttactttccggacgaactccggtccgtgaagtacgcatactctgccgatctgttcaatgcactcaacgaccttaacaacttggtgatccagcgcgatggcctgtccaagttggaataccacgaaaagtatcacatcatcgagaacgtgttcaagcagaaaaagaagccaactctgaagcagattgccaacgaaattaacgtgaaccccgaggatatcaagggataccggatcactaagtccggcaaaccacagttcaccgagttcaagctgtaccacgatctgaagtcggtgctctttgaccagtccatcctggaaaacgaagatgtgctggaccagattgctgagatcctgaccatctaccaggacaaggactcgattaagagcaagctcacggagctggacattctgctgaacgaagaggataaggagaacatcgcgcagctcactggttacaccggtacccaccgcttgtcccttaagtgcatccggctggtcctcgaggaacaatggtactccagccggaaccagatggagatcttcacgcacttgaacatcaagccgaagaagattaacctgaccgctgcgaacaagatacccaaggccatgatcgacgagtttatcctctcaccggtggtcaagcgcaccttcggacaagccatcaacctcatcaacaagattatcgagaagtacggcgtgcctgaggatatcatcatcgagctggctcgggagaacaactcaaaggataagcagaagttcattaacgagatgcagaaaaagaacgagaacactcgcaagcggattaatgagatcatcggtaaatacgggaaccagaacgccaagcggcttgtggaaaagattcggctccacgacgagcaggagggaaagtgtctgtactcgctggagagcattcccctggaggacctcctgaacaacccaaaccactacgaagtggatcacataatcccccgcagcgtgtcattcgacaattcctaccataacaaggtcctcgtgaagcagtccgagaatagcaagaagtccaacctgactccgtaccagtacttcaactccggcaaatccaagctgtcctacaaccagttcaaacagcacatcctcaacctgtcaaagagccaggacaggatctcgaagaagaagaaggaataccttctcgaggaacgggatatcaataagttcgaggtgcagaaggagtttatcaatagaaacctggtggacactcgctatgccacccgcgaactgaccaactacctgaaggcgtacttctccgccaacaacatgaacgtgaaggtcaaaactattaacggcagcttcaccgactatctgcgcaaggtctggaagttcaagaaggaacgcaaccacggttacaagcaccacgcggaagatgcgctgattatcgccaacgctgacttcctgttcaaggaaaacaagaagctcaaggccgtgaactcagtgctcgagaagcctgaaatcgagactaagcagctggacatccaggtcgattcggaagataactactccgaaatgttcatcatccctaagcaagtgcaggacatcaaggacttcaggaatttcaagtacagccatcgcgtggacaagaagccaaacagacagctgatcaacgatacactgtattccacccggaagaaggacaactccacctacatcgtccaaaccattaaggacatctacgcaaaggacaacaccacgcttaagaagcagttcgacaagagccccgaaaagttcctcatgtaccagcacgaccccagaaccttcgagaagcttgaagtgatcatgaagcagtacgccaacgaaaagaacccactggctaagtaccacgaggaaaccggcgaatacctgaccaagtactccaaaaagaacaacggaccgatcgtcaagtccctgaagtacattgggaacaagctcggctcgcacctcgatgtgacccaccagttcaagtcctcgaccaaaaagctcgtgaagctgtccatcaagccgtaccggttcgacgtgtacctgactgacaagggatataagttcatcaccatttcctacctcgacgtgttgaagaaggataactactactacattccggaacagaagtacgacaagctcaagctcggaaaggccatcgacaaaaatgcgaagttcatcgcgagcttctacaagaatgacttgatcaagctggatggcgaaatctacaagatcatcggggtcaactccgatacccgcaacatgattgagctggatctgcccgacattcggtacaaggaatactgcgagctgaacaacatcaagggagaaccgcggatcaagaaaaccatcggaaagaaagtgaacagcatcgagaaactgactactgacgtcctgggaaacgtgttcaccaacacacaatacaccaaaccccagctgctgtttaagcgcgggaac

SEQ ID NO: 805 - DNA, 3162 bp, Artificial Sequence
  Synthetic: Codon optimized version of a polynucleotide sequence shown from position 61 to 3225 (SEQ ID NO: 45 in WO2019/ 183150)
atgaaccagaagttcatcctgggcctcgacatcggcatcacctctgttggctacggcctgatcgactacgagacaaagaacatcatcgatgccggcgtgcggctgttccctgaggccaacgtggaaaacaacgagggccgcagaagcaagagaggcagcagaaggctgaagcggcggagaatccaccggctggaaagagtgaagaagctgctcgaggactacaacctgctggaccagtctcagatccctcagagcacaaacccctacgccatcagagtgaagggcctgtctgaggccctgagcaaggacgagctggttatcgccctgctgcacattgccaagcggagaggcatccacaagatcgacgtgatcgacagcaacgacgacgtgggcaatgagctgagcaccaaagagcagctgaacaagaacagcaagctgctgaaggacaagttcgtgtgccagattcagctggaacggatgaatgagggccaagtgcggggcgagaagaacagattcaagaccgccgacatcatcaaagagatcatccagctgctcaacgtgcagaagaacttccaccagctggacgagaacttcatcaacaagtacatcgagctggtcgagatgcggcgcgagtactttgaaggccctggaaagggcagcccttatggctgggaaggcgatcccaaggcttggtacgagacactgatgggccactgcacctactttcccgacgagctgagaagcgtgaagtacgcctacagcgccgacctgttcaacgccctgaacgacctgaacaacctcgtgatccagagagatggcctgtccaagctggaataccacgagaagtaccacatcattgagaacgtgttcaagcagaagaagaagcccacactgaagcagatcgccaacgagatcaacgtgaaccccgaggacatcaagggctacagaatcaccaagagcggcaagccccagttcaccgagttcaagctgtaccacgatctgaagtccgtgctgttcgaccagagcatcctggaaaacgaggacgtgctggatcagatcgccgagatcctgaccatctaccaggacaaggacagcatcaagagcaagctgaccgagctggacatcctgctgaacgaagaggacaaagagaatatcgcccagctgaccggctacaccggcacacatagactgagcctgaagtgcatccggctggtgctggaagaacagtggtactccagccggaaccagatggaaatcttcacccacctgaacatcaagcccaagaagatcaacctgaccgccgccaacaagatccccaaggccatgatcgacgagttcattctgagccccgtggtcaagagaaccttcggccaggccatcaatctgatcaacaagattatcgagaagtatggcgtgcccgaggatatcatcatcgaactggccagagagaacaacagcaaggacaagcaaaagttcatcaacgagatgcagaaaaagaacgagaacacccggaagcggatcaacgaaatcatcgggaagtacggcaaccagaacgccaagagactggtggaaaagatccggctgcacgacgagcaagagggcaagtgtctgtacagcctggaatctatccctctcgaggatctgctgaacaatcccaaccactacgaggtggaccacattatccccagaagcgtgtccttcgacaacagctaccacaacaaggtgctggtcaagcagagcgagaactccaagaagtccaatctgaccccttaccagtacttcaacagcggcaagtctaagctgagctacaaccagtttaagcagcacatcctgaacctcagcaagagccaggaccggatcagcaagaagaagaaagagtacctgctcgaagagagggacattaacaagttcgaggtgcagaaagagtttatcaaccggaacctggtggacaccagatacgccaccagagagctgaccaactacctgaaggcctacttcagcgccaacaacatgaacgtgaaagtcaagaccatcaacggcagcttcaccgactacctgcggaaagtgtggaagtttaagaaagagcggaaccacggctacaagcaccacgccgaagatgccctgattatcgccaatgccgacttcctgttcaaagagaacaagaaactgaaggccgtgaacagcgtgctggaaaagcccgagatcgagacaaaacagctcgacatccaggtggacagcgaggacaactacagcgagatgttcatcatccccaaacaggtgcaggatatcaaggacttccggaacttcaagtacagccaccgcgtggacaagaagcctaaccggcagctgatcaatgacaccctgtacagcacccgcaagaaggacaacagcacctacatcgtgcagacgatcaaggacatctacgccaaggacaatacgaccctgaagaagcagttcgacaagagccccgagaagttcctgatgtaccagcacgaccccaggaccttcgagaagctggaagtgatcatgaagcagtacgctaatgagaagaacccgctggccaagtaccacgaggaaaccggcgagtacctgaccaagtactctaagaagaacaacggccccatcgtgaagtccctgaagtatatcggcaacaagctgggcagccacctggacgtgacacaccagttcaagagcagcaccaagaagctggtcaaactgtccatcaagccataccgcttcgacgtgtacctgacagacaaggggtacaagtttatcaccatcagctacctcgacgtgctgaagaaggataactactactacatccccgagcagaagtacgacaagctgaagctgggaaaagccatcgacaagaatgccaagttcattgccagcttctacaagaacgacctcatcaagctggacggcgagatctacaagatcatcggcgtgaactccgacacacggaacatgattgagctggacctgcctgacatccggtacaaagagtactgcgaactgaacaatatcaagggcgagccccggatcaaaaagacgatcggcaagaaagtgaacagcattgagaagctgaccaccgatgtgctgggcaatgtgttcaccaacacacagtacaccaagcctcagctgctgttcaagcggggcaat

SEQ ID NO: 806 - protein, 1052 aa, Staphylococcus pasteuri
  SITE (1)..(1052): Staphylococcus pasteuri Cas9
MKEKYILGLDLGITSVGYGIINFETKKIIDAGVRLFPEANVDNNEGRRSKRGSRRLKRRRIHRLERVKLLLTEYDLINKEQIPTSNNPYQIRVKGLSEILSKDELAIALLHLAKRRGIHNINVSSEDEDASNELSTKEQINRNNKLLKDKYVCEVQLQRLKEGQIRGEKNRFKTTDILKEIDQLLKVQKDYHNLDIDFINQYKEIVETRREYFEGPGQGSPFGWNGDLKKWYEMLMGHCTYFPQELRSVKYAYSADLFNALNDLNNLIIQRDNSEKLEYHEKYHIIENVFKQKKKPTLKQIAKEIGVNPEDIKGYRITKSGTPQEFKLYHDLKSIVFDKSILENEAILDQIAEILTIYQDEQSIKEELNKLPEILNEQDKAEIAKLIGYNGTHRLSLKCIHLINEELWQTSRNQMEIFNYLNIKPNKVDLSEQNKIPKDMVNDFILSPVVKRTFIQSINVINKVIEKYGIPEDIIIELARENNSDDRKKFINNLQKKNEATRKRINEIIGQTGNQNAKRIVEKIRLHDQQEGKCLYSLESIALMDLLNNPQNYEVDHIIPRSVAFDNSIHNKVLVKQIENSKKGNRTPYQYLNSSDAKLSYNQFKQHILNLSKSKDRISKKKKDYLLEERDINKFEVQKEFINRNLVDTRYATRELTSYLKAYFSANNMDVKVKTINGSFTNHLRKVWRFDKYRNHGYKHHAEDALIIANADFLFKENKKLQNTNKILEKPTIENNTKKVTVEKEEDYNNVFETPKLVEDIKQYRDYKFSHRVDKKPNRQLINDTLYSTRMKDEHDYIVQTITDIYGKDNTNLKKQFNKNPEKFLMYQNDPKTFEKLSIIMKQYSDEKNPLAKYYEETGEYLTKYSKKNNGPIVKKIKLLGNKVGNHLDVTNKYENSTKKLVKLSIKNYRFDVYLTEKGYKFVTIAYLNVFKKDNYYYIPKDKYQELKEKKKIKDTDQFIASFYKNDLIKLNGDLYKIIGVNSDDRNIIELDYYDIKYKDYCEINNIKGEPRIKKTIGKKTESIEKFTTDVLGNLYLHSTEKAPQLIFKRGL

SEQ ID NO: 807 - protein, 1052 aa, Staphylococcus microti
  SITE (1)..(1052): Staphylococcus microti Cas9
MEKDYILGLDIGIGSVGYGLIDYDTKSIIDAGVRLFPEANADNNLGRRAKRGARRLKRRRIHRLERVKSLLSEYKIISGLAPTNNQPYNIRVKGLTEQLTKDELAVALLHIAKRRGIHNVDVAADKEETASDSLSTKDQINKNAKFLESRYVCELQKERLENEGHVRGVENRFLTKDIVREAKKIIDTQMQYYPEIDETFKEKYISLVETRREYYEGPGKGSPYGWDADVKKWYQLMMGHCTYFPVEFRSVKYAYTADLYNALNDLNNLTIARDDNPKLEYHEKYHIIENVFKQKRNPTLKQIAKEIGVNDINISGYRVTKSGKPQFTSFKLFHDLKKVVKDHAILDDIDLLNQIAEILTIYQDKDSIVAELGQLEYLMSEADKQSISELTGYTGTHSLSLKCMNMIIDELWHSSMNQMEVFTYLNMRPKKYELKGYQRIPTDMIDDAILSPVVKRSFKQAIGVVNAIIKKYGLPKDIIIELARESNSAEKSRYLRAIQKKNEKTRERIEAIIKEYGNENAKGLVQKIKLHDAQEGKCLYSLKDIPLEDLLRNPNNYDIDHIIPRSVSFDDSMHNKVLVRREQNAKKNNQTPYQYLTSGYADIKYSVFKQHVLNLAENKDRMTKKKREYLLEERNINKYDVQKEFINRNLVDTRYTTRELTTLLKTYFTINNLDVKVKTINGSFTDFLRKRWGFKKNRDEGYKHHAEDALIIANADYLFKEHKLLKEIKDVSDLAGDERNSNVKDEDQYEEVFGGYFKIEDIKKYKIKKFSHRVDKKPNRQLINDTIYSTRVKDDKRYLINTLKNLYDKSNGDLKERMQKDPESLLMYHHDPQTFEKLKIVMSQYENEKNPLAKYFEETGQYLTKYAKHDNGPAIHKIKYYGNKLYEHLDITKNYHNPQNKVVQLSQKSFRFDVYQTDKGYKFISIAYLTLKNEKNYYAISQEKYDQLKSEKKISNNAVFIGSFYTSDIIEINNEKFRVIGVNSDKNNLIEVDRIDIRQKEFIELEEEKKNNRIKVTIGRKTTNIEKFHTDILGNMYKSKRPKAPQLVFKKG

SEQ ID NO: 808 - protein, 1051 aa, Staphylococcus hyicus
  SITE (1)..(1051): Staphylococcus hyicus Cas9
MNNYILGLDIGITSVGYGIVDSDTREIKDAGVRLFPEANVDNNEGRRSKRGARRLKRRRIHRLDRVKHLLAEYDLLDLTNIPKSTNPYQTRVKGLNEKLSKDELVIALLHIAKRRGIHNVNVMMDDNDSGNELSTKDQLKKNAKALSDKYVCELQLERFEQDYKVRGEKNRFKTEDFVREARKLLETQSKFFEIDQTFIMRYIELIETRREYFEGPGKGSPFGWEGNIKKWFEQMMGHCTYFPEELRSVKYSYSAELFNALNDLNNLVITRDEDAKLNYGEKFQIIENVFKQKKTPNLKQIAIEIGVHETEIKGYRVNKSGKPEFTQFKLYHDLKNIFKDPKYLNDIQLMDNIAEIITIYQDAESIIKELNQLPELLSEREKEKISALSGYSGTHRLSLKCINLLLDDLWESSLNQMELFTKLNLKPKKIDLSQQHKIPSKLVDDFILSPVVKRAFIQSIQVVNAIIDKYGLPEDIIIELARENNSDDRRKFLNQLQKQNEETRKQVEKVLREYGNDNAKRIVQKIKLHNMQEGKCLYSLKDIPLEDLLRNPHHYEVDHIIPRSVAFDNSMHNKVLVRADENSKKGNRTPYQYLNSSESSLSYNEFKQHILNLSKTKDRITKKKREYLLEERDINKFDVQKEFINRNLVDTRYATRELTSLLKAYFSANNLDVKVKTINGSFTNYLRKVWKFDKDRNKGYKHHAEDALIIANADFLFKHNKKLRNINKVLDAPSKEVDKKRVTVQSEDEYNQIFEDTQKAQAIKKFEIRKFSHRVDKKPNRQLINDTLYSTRNIDGIEYVVESIKDIYSVNNDKVKTKFKKDPHRLLMYRNDPQTFEKFEKVFKQYESEKNPFAKYYEETGEKIRKFSKTGQGPYINKIKYLRERLGRHCDVTNKYINSRNKIVQLKIYSYRFDIYQYGNNYKMITISYIDLEQKSNYYYISREKYEQKKKDKQIDDSYKFIGSFYKNDIINYNGEMYRVIGVNDSEKNKIQLDMIDISIKDYMELNNIKKTGVIYKTIGKSTTHIEKYTTDILGNLYKAAPPKKPQLIFK

SEQ ID NO: 809 - protein, 67 aa, Staphylococcus lugdunensis
  SITE (1)..(67): Staphylococcus lugdunensis mini-domain 1 from 8 mini-domain library
MNQKFILGLDIGITSVGYGLIDYETKNIIDAGVRLFPEANVENNEGRRSKRGSRRLKRRRIHRLERV

SEQ ID NO: 810 - protein, 67 aa, Staphylococcus pasteuri
  SITE (1)..(67): Staphylococcus pasteuri mini-domain 1 from 8 mini-domain library
MKEKYILGLDLGITSVGYGIINFETKKIIDAGVRLFPEANVDNNEGRRSKRGSRRLKRRRIHRLERV

SEQ ID NO: 811 - protein, 66 aa, Staphylococcus hyicus
  SITE (1)..(66): Staphylococcus hyicus mini-domain 1 from 8 mini-domain library
MNNYILGLDIGITSVGYGIVDSDTREIKDAGVRLFPEANVDNNEGRRSKRGARRLKRRRIHRLDRV

SEQ ID NO: 812 - protein, 67 aa, Staphylococcus microti
  SITE (1)..(67): Staphylococcus microti mini-domain 1 from 8 mini-domain library
MEKDYILGLDIGIGSVGYGLIDYDTKSIIDAGVRLFPEANADNNLGRRAKRGARRLKRRRIHRLERV

SEQ ID NO: 813 - protein, 52 aa, Staphylococcus lugdunensis
  SITE (1)..(52): Staphylococcus lugdunensis mini-domain 2 from 8 mini-domain library
KKLLEDYNLLDQSQIPQSTNPYAIRVKGLSEALSKDELVIALLHIAKRRGIH

SEQ ID NO: 814 - protein, 52 aa, Staphylococcus pasteuri
  SITE (1)..(52): Staphylococcus pasteuri mini-domain 2 from 8 mini-domain library
KLLLTEYDLINKEQIPTSNNPYQIRVKGLSEILSKDELAIALLHLAKRRGIH

SEQ ID NO: 815 - protein, 52 aa, Staphylococcus hyicus
  SITE (1)..(52): Staphylococcus hyicus mini-domain 2 from 8 mini-domain library
KHLLAEYDLLDLTNIPKSTNPYQTRVKGLNEKLSKDELVIALLHIAKRRGIH

SEQ ID NO: 816 - protein, 51 aa, Staphylococcus microti
  SITE (1)..(51): Staphylococcus microti mini-domain 2 from 8 mini-domain library
KSLLSEYKIISGLAPTNNQPYNIRVKGLTEQLTKDELAVALLHIAKRRGIH

SEQ ID NO: 817 - protein, 92 aa, Staphylococcus lugdunensis
  SITE (1)..(92): Staphylococcus lugdunensis mini-domain 3 from 8 mini-domain library
KIDVIDSNDDVGNELSTKEQLNKNSKLLKDKFVCQIQLERMNEGQVRGEKNRFKTADIIKEIIQLLNVQKNFHQLDENFINKYIELVEMRRE

SEQ ID NO: 818 - protein, 92 aa, Staphylococcus pasteuri
  SITE (1)..(92): Staphylococcus pasteuri mini-domain 3 from 8 mini-domain library
NINVSSEDEDASNELSTKEQINRNNKLLKDKYVCEVQLQRLKEGQIRGEKNRFKTTDILKEIDQLLKVQKDYHNLDIDFINQYKEIVETRRE

SEQ ID NO: 819 - protein, 93 aa, Staphylococcus hyicus
  SITE (1)..(93): Staphylococcus hyicus mini-domain 3 from 8 mini-domain library
NVNVMMDDNDSGNELSTKDQLKKNAKALSDKYVCELQLERFEQDYKVRGEKNRFKTEDFVREARKLLETQSKFFEIDQTFIMRYIELIETRRE

SEQ ID NO: 820 - protein, 95 aa, Staphylococcus microti
  SITE (1)..(95): Staphylococcus microti mini-domain 3 from 8 mini-domain library
NVDVAADKEETASDSLSTKDQINKNAKFLESRYVCELQKERLENEGHVRGVENRFLTKDIVREAKKIIDTQMQYYPEIDETFKEKYISLVETRRE

SEQ ID NO: 821 - protein, 111 aa, Staphylococcus lugdunensis
  SITE (1)..(111): Staphylococcus lugdunensis mini-domain 4 from 8 mini-domain library
YFEGPGKGSPYGWEGDPKAWYETLMGHCTYFPDELRSVKYAYSADLFNALNDLNNLVIQRDGLSKLEYHEKYHIIENVFKQKKKPTLKQIANEINVNPEDIKGYRITKSGK

SEQ ID NO: 822 - protein, 111 aa, Staphylococcus pasteuri
  SITE (1)..(111): Staphylococcus pasteuri mini-domain 4 from 8 mini-domain library
YFEGPGQGSPFGWNGDLKKWYEMLMGHCTYFPQELRSVKYAYSADLFNALNDLNNLIIQRDNSEKLEYHEKYHIIENVFKQKKKPTLKQIAKEIGVNPEDIKGYRITKSGT

SEQ ID NO: 823 - protein, 111 aa, Staphylococcus hyicus
  SITE (1)..(111): Staphylococcus hyicus mini-domain 4 from 8 mini-domain library
YFEGPGKGSPFGWEGNIKKWFEQMMGHCTYFPEELRSVKYSYSAELFNALNDLNNLVITRDEDAKLNYGEKFQIIENVFKQKKTPNLKQIAIEIGVHETEIKGYRVNKSGK

SEQ ID NO: 824 - protein, 111 aa, Staphylococcus microti
  SITE (1)..(111): Staphylococcus microti mini-domain 4 from 8 mini-domain library
YYEGPGKGSPYGWDADVKKWYQLMMGHCTYFPVEFRSVKYAYTADLYNALNDLNNLTIARDDNPKLEYHEKYHIIENVFKQKRNPTLKQIAKEIGVNDINISGYRVTKSGK

SEQ ID NO: 825 - protein, 131 aa, Staphylococcus lugdunensis
  SITE (1)..(131): Staphylococcus lugdunensis mini-domain 5 from 8 mini-domain library
PQFTEFKLYHDLKSVLFDQSILENEDVLDQIAEILTIYQDKDSIKSKLTELDILLNEEDKENIAQLTGYTGTHRLSLKCIRLVLEEQWYSSRNQMEIFTHLNIKPKKINLTAANKIPKAMIDEFILSPVVK

SEQ ID NO: 826 - protein, 131 aa, Staphylococcus pasteuri
  SITE (1)..(131): Staphylococcus pasteuri mini-domain 5 from 8 mini-domain library
PQFTEFKLYHDLKSIVFDKSILENEAILDQIAEILTIYQDEQSIKEELNKLPEILNEQDKAEIAKLIGYNGTHRLSLKCIHLINEELWQTSRNQMEIFNYLNIKPNKVDLSEQNKIPKDMVNDFILSPVVK

SEQ ID NO: 827 - protein, 131 aa, Staphylococcus hyicus
  SITE (1)..(131): Staphylococcus hyicus mini-domain 5 from 8 mini-domain library
PEFTQFKLYHDLKNIFKDPKYLNDIQLMDNIAEIITIYQDAESIIKELNQLPELLSEREKEKISALSGYSGTHRLSLKCINLLLDDLWESSLNQMELFTKLNLKPKKIDLSQQHKIPSKLVDDFILSPVVK

SEQ ID NO: 828 - protein, 131 aa, Staphylococcus microti
  SITE (1)..(131): Staphylococcus microti mini-domain 5 from 8 mini-domain library
PQFTSFKLFHDLKKVVKDHAILDDIDLLNQIAEILTIYQDKDSIVAELGQLEYLMSEADKQSISELTGYTGTHSLSLKCMNMIIDELWHSSMNQMEVFTYLNMRPKKYELKGYQRIPTDMIDDAILSPVVK

SEQ ID NO: 829 - protein, 89 aa, Staphylococcus lugdunensis
  SITE (1)..(89): Staphylococcus lugdunensis mini-domain 6 from 8 mini-domain library
RTFGQAINLINKIIEKYGVPEDIIIELARENNSKDKQKFINEMQKKNENTRKRINEIIGKYGNQNAKRLVEKIRLHDEQEGKCLYSLES

SEQ ID NO: 830 - protein, 89 aa, Staphylococcus pasteuri
  SITE (1)..(89): Staphylococcus pasteuri mini-domain 6 from 8 mini-domain library
RTFIQSINVINKVIEKYGIPEDIIIELARENNSDDRKKFINNLQKKNEATRKRINEIIGQTGNQNAKRIVEKIRLHDQQEGKCLYSLES

SEQ ID NO: 831 - protein, 89 aa, Staphylococcus hyicus
  SITE (1)..(89): Staphylococcus hyicus mini-domain 6 from 8 mini-domain library
RAFIQSIQVVNAIIDKYGLPEDIIIELARENNSDDRRKFLNQLQKQNEETRKQVEKVLREYGNDNAKRIVQKIKLHNMQEGKCLYSLKD

SEQ ID NO: 832 - protein, 89 aa, Staphylococcus microti
  SITE (1)..(89): Staphylococcus microti mini-domain 6 from 8 mini-domain library
RSFKQAIGVVNAIIKKYGLPKDIIIELARESNSAEKSRYLRAIQKKNEKTRERIEAIIKEYGNENAKGLVQKIKLHDAQEGKCLYSLKD

SEQ ID NO: 833 - protein, 116 aa, Staphylococcus lugdunensis
  SITE (1)..(116): Staphylococcus lugdunensis mini-domain 7 from 8 mini-domain library
IPLEDLLNNPNHYEVDHIIPRSVSFDNSYHNKVLVKQSENSKKSNLTPYQYFNSGKSKLSYNQFKQHILNLSKSQDRISKKKKEYLLEERDINKFEVQKEFINRNLVDTRYATREL

SEQ ID NO: 834 - protein, 116 aa, Staphylococcus pasteuri
  SITE (1)..(116): Staphylococcus pasteuri mini-domain 7 from 8 mini-domain library
IALMDLLNNPQNYEVDHIIPRSVAFDNSIHNKVLVKQIENSKKGNRTPYQYLNSSDAKLSYNQFKQHILNLSKSKDRISKKKKDYLLEERDINKFEVQKEFINRNLVDTRYATREL

SEQ ID NO: 835 - protein, 116 aa, Staphylococcus hyicus
  SITE (1)..(116): Staphylococcus hyicus mini-domain 7 from 8 mini-domain library
IPLEDLLRNPHHYEVDHIIPRSVAFDNSMHNKVLVRADENSKKGNRTPYQYLNSSESSLSYNEFKQHILNLSKTKDRITKKKREYLLEERDINKFDVQKEFINRNLVDTRYATREL

SEQ ID NO: 836 - protein, 115 aa, Staphylococcus microti
  SITE (1)..(115): Staphylococcus microti mini-domain 7 from 8 mini-domain library
IPLEDLLRNPNNYDIDHIIPRSVSFDDSMHNKVLVRREQAKKNNQTPYQYLTSGYADIKYSVFKQHVLNLAENKDRMTKKKREYLLEERNINKYDVQKEFINRNLVDTRYTTREL

SEQ ID NO: 837 - protein, 396 aa, Staphylococcus lugdunensis
  SITE (1)..(396): Staphylococcus lugdunensis mini-domain 8 from 8 mini-domain library
TNYLKAYFSANNMNVKVKTINGSFTDYLRKVWKFKKERNHGYKHHAEDALIIANADFLFKENKKLKAVNSVLEKPEIETKQLDIQVDSEDNYSEMFIIPKQVQDIKDFRNFKYSHRVDKKPNRQLINDTLYSTRKKDNSTYIVQTIKDIYAKDNTTLKKQFDKSPEKFLMYQHDPRTFEKLEVIMKQYANEKNPLAKYHEETGEYLTKYSKKNNGPIVKSLKYIGNKLGSHLDVTHQFKSSTKKLVKLSIKPYRFDVYLTDKGYKFITISYLDVLKKDNYYYIPEQKYDKLKLGKAIDKNAKFIASFYKNDLIKLDGEIYKIIGVNSDTRNMIELDLPDIRYKEYCELNNIKGEPRIKKTIGKKVNSIEKLTTDVLGNVFTNTQYTKPQLLFKRGN

SEQ ID NO: 838 - protein, 396 aa, Staphylococcus pasteuri
  SITE (1)..(396): Staphylococcus pasteuri mini-domain 8 from 8 mini-domain library
TSYLKAYFSANNMDVKVKTINGSFTNHLRKVWRFDKYRNHGYKHHAEDALIIANADFLFKENKKLQNTNKILEKPTIENNTKKVTVEKEEDYNNVFETPKLVEDIKQYRDYKFSHRVDKKPNRQLINDTLYSTRMKDEHDYIVQTITDIYGKDNTNLKKQFNKNPEKFLMYQNDPKTFEKLSIIMKQYSDEKNPLAKYYEETGEYLTKYSKKNNGPIVKKIKLLGNKVGNHLDVTNKYENSTKKLVKLSIKNYRFDVYLTEKGYKFVTIAYLNVFKKDNYYYIPKDKYQELKEKKKIKDTDQFIASFYKNDLIKLNGDLYKIIGVNSDDRNIIELDYYDIKYKDYCEINNIKGEPRIKKTIGKKTESIEKFTTDVLGNLYLHSTEKAPQLIFKRGL

SEQ ID NO: 839 - protein, 393 aa, Staphylococcus hyicus
  SITE (1)..(393): Staphylococcus hyicus mini-domain 8 from 8 mini-domain library
TSLLKAYFSANNLDVKVKTINGSFTNYLRKVWKFDKDRNKGYKHHAEDALIIANADFLFKHNKKLRNINKVLDAPSKEVDKKRVTVQSEDEYNQIFEDTQKAQAIKKFEIRKFSHRVDKKPNRQLINDTLYSTRNIDGIEYVVESIKDIYSVNNDKVKTKFKKDPHRLLMYRNDPQTFEKFEKVFKQYESEKNPFAKYYEETGEKIRKFSKTGQGPYINKIKYLRERLGRHCDVTNKYINSRNKIVQLKIYSYRFDIYQYGNNYKMITISYIDLEQKSNYYYISREKYEQKKKDKQIDDSYKFIGSFYKNDIINYNGEMYRVIGVNDSEKNKIQLDMIDISIKDYMELNNIKKTGVIYKTIGKSTTHIEKYTTDILGNLYKAAPPKKPQLIFK

SEQ ID NO: 840 - protein, 392 aa, Staphylococcus  microti
  SITE (1)..(392): Staphylococcus  microti mini-domain 8 from 8 mini-domain library
TTLLKTYFTINNLDVKVKTINGSFTDFLRKRWGFKKNRDEGYKHHAEDALIIANADYLFKEHKLLKEIKDVSDLAGDERNSNVKDEDQYEEVFGGYFKIEDIKKYKIKKFSHRVDKKPNRQLINDTIYSTRVKDDKRYLINTLKNLYDKSNGDLKERMQKDPESLLMYHHDPQTFEKLKIVMSQYENEKNPLAKYFEETGQYLTKYAKHDNGPAIHKIKYYGNKLVEHLDITKNYHNPQNKVVQLSQKSFRFDVYQTDKGYKFISIAYLTLKNEKNYYAISQEKYDQLKSEKKISNNAVFIGSFYTSDIIEINNEKFRVIGVNSDKNNLIEVDRIDIRQKEFIELEEEKKNNRIKVTIGRKTTNIEKFHTDILGNMYKSKRPKAPQLVFKKG

SEQ ID NO: 841 - protein, 41 aa, Staphylococcus lugdunensis
  SITE (1)..(41): Staphylococcus lugdunensis mini-domain 1 from 12 mini-domain library
MNQKFILGLDIGITSVGYGLIDYETKNIIDAGVRLFPEANV

SEQ ID NO: 842 - protein, 41 aa, Staphylococcus pasteuri
  SITE (1)..(41): Staphylococcus pasteuri mini-domain 1 from 12 mini-domain library
MKEKYILGLDLGITSVGYGIINFETKKIIDAGVRLFPEANV

SEQ ID NO: 843 - protein, 40 aa, Staphylococcus hyicus
  SITE (1)..(40): Staphylococcus hyicus mini-domain 1 from 12 mini-domain library
MNNYILGLDIGITSVGYGIVDSDTREIKDAGVRLFPEANV

SEQ ID NO: 844 - protein, 41 aa, Staphylococcus microti
  SITE (1)..(41): Staphylococcus microti mini-domain 1 from 12 mini-domain library
MEKDYILGLDIGIGSVGYGLIDYDTKSIIDAGVRLFPEANA

SEQ ID NO: 845 - protein, 23 aa, Staphylococcus lugdunensis
  SITE (1)..(23): Staphylococcus lugdunensis mini-domain 2 from 12 mini-domain library
ENNEGRRSKRGSRRLKRRRIHRL

SEQ ID NO: 846 - protein, 23 aa, Staphylococcus pasteuri
  SITE (1)..(23): Staphylococcus pasteuri mini-domain 2from 12 mini-domain library
DNNEGRRSKRGSRRLKRRRIHRL

SEQ ID NO: 847 - protein, 23 aa, Staphylococcus hyicus
  SITE (1)..(23): Staphylococcus hyicus mini-domain 2 from 12 mini-domain library
DNNEGRRSKRGARRLKRRRIHRL

SEQ ID NO: 848 - protein, 23 aa, Staphylococcus microti
  SITE (1)..(23): Staphylococcus microti mini-domain 2 from 12 mini-domain library
DNNLGRRAKRGARRLKRRRIHRL

SEQ ID NO: 849 - protein, 55 aa, Staphylococcus lugdunensis
  SITE (1)..(55): Staphylococcus lugdunensis mini-domain 3 from 12 mini-domain library
ERVKKLLEDYNLLDQSQIPQSTNPYAIRVKGLSEALSKDELVIALLHIAKRRGIH

SEQ ID NO: 850 - protein, 55 aa, Staphylococcus pasteuri; identical to SEQ ID NO: 849
  SITE (1)..(55): Staphylococcus pasteuri mini-domain 3 from 12 mini-domain library

SEQ ID NO: 851 - protein, 55 aa, Staphylococcus hyicus
  SITE (1)..(55): Staphylococcus hyicus mini-domain 3 from 12 mini-domain library
DRVKHLLAEYDLLDLTNIPKSTNPYQTRVKGLNEKLSKDELVIALLHIAKRRGIH

SEQ ID NO: 852 - protein, 54 aa, Staphylococcus microti
  SITE (1)..(54): Staphylococcus microti mini-domain 3 from 12 mini-domain library
ERVKSLLSEYKIISGLAPTNNQPYNIRVKGLTEQLTKDELAVALLHIAKRRGIH

SEQ ID NO: 853 - protein, 93 aa, Staphylococcus lugdunensis
  SITE (1)..(93): Staphylococcus lugdunensis mini-domain 4 from 12 mini-domain library
KIDVIDSNDDVGNELSTKEQLNKNSKLLKDKFVCQIQLERMNEGQVRGEKNRFKTADIIKEIIQLLNVQKNFHQLDENFINKYIELVEMRREY

SEQ ID NO: 854 - protein, 92 aa, Staphylococcus pasteuri
  SITE (1)..(92): Staphylococcus pasteuri mini-domain 4 from 12 mini-domain library
INVSSEDEDASNELSTKEQINRNNKLLKDKYVCEVQLQRLKEGQIRGEKNRFKTTDILKEIDQLLKVQKDYHNLDIDFINQYKEIVETRREY

SEQ ID NO: 855 - protein, 94 aa, Staphylococcus hyicus
  SITE (1)..(94): Staphylococcus hyicus mini-domain 4 from 12 mini-domain library
NVNVMMDDNDSGNELSTKDQLKKNAKALSDKYVCELQLERFEQDYKVRGEKNRFKTEDFVREARKLLETQSKFFEIDQTFIMRYIELIETRREY

SEQ ID NO: 856 - protein, 96 aa, Staphylococcus microti
  SITE (1)..(96): Staphylococcus microti mini-domain 4 from 12 mini-domain library
NVDVAADKEETASDSLSTKDQINKNAKFLESRYVCELQKERLENEGHVRGVENRFLTKDIVREAKKIIDTQMQYYPEIDETFKEKYISLVETRREY

SEQ ID NO: 857 - protein, 114 aa, Staphylococcus lugdunensis
  SITE (1)..(114): Staphylococcus lugdunensis mini-domain 5 from 12 mini-domain library
FEGPGKGSPYGWEGDPKAWYETLMGHCTYFPDELRSVKYAYSADLFNALNDLNNLVIQRDGLSKLEYHEKYHIIENVFKQKKKPTLKQIANEINVNPEDIKGYRITKSGKPQFT

SEQ ID NO: 858 - protein, 114 aa, Staphylococcus pasteuri
  SITE (1)..(114): Staphylococcus pasteuri mini-domain 5 from 12 mini-domain library
FEGPGQGSPFGWNGDLKKWYEMLMGHCTYFPQELRSVKYAYSADLFNALNDLNNLIIQRDNSEKLEYHEKYHIIENVFKQKKKPTLKQIAKEIGVNPEDIKGYRITKSGTPQFF

SEQ ID NO: 859 - protein, 114 aa, Staphylococcus hyicus
  SITE (1)..(114): Staphylococcus hyicus mini-domain 5 from 12 mini-domain library
FEGPGKGSPFGWEGNIKKWFEQMMGHCTYFPEELRSVKYSYSAELFNALNDLNNLVITRDEDAKLNYGEKFQIIENVFKQKKTPNLKQIAIEIGVHETEIKGYRVNKSGKPEFT

SEQ ID NO: 860 - protein, 114 aa, Staphylococcus microti
  SITE (1)..(114): Staphylococcus microti mini-domain 5 from 12 mini-domain library
YEGPGKGSPYGWDADVKKWYQLMMGHCTYFPVEFRSVKYAYTADLYNALNDLNNLTIARDDNPKLEYHEKYHIIENVFKQKRNPTLKQIAKEIGVNDINISGYRVTKSGKPQFT

SEQ ID NO: 861 - protein, 128 aa, Staphylococcus lugdunensis
  SITE (1)..(128): Staphylococcus lugdunensis mini-domain 6 from 12 mini-domain library
EFKLYHDLKSVLFDQSILENEDVLDQIAEILTIYQDKDSIKSKLTELDILLNEEDKENIAQLTGYTGTHRLSLKCIRLVLEEQWYSSRNQMEIFTHLNIKPKKINLTAANKIPKAMIDEFILSPVVKR

SEQ ID NO: 862 - protein, 128 aa, Staphylococcus pasteuri
  SITE (1)..(128): Staphylococcus pasteuri mini-domain 6 from 12 mini-domain library
EFKLYHDLKSIVFDKSILENEAILDQIAEILTIYQDEQSIKEELNKLPEILNEQDKAEIAKLIGYNGTHRLSLKCIHLINEELWQTSRNQMEIFNYLNIKPNKVDLSEQNKIPKDMVNDFILSPVVKR

SEQ ID NO: 863 - protein, 128 aa, Staphylococcus hyicus
  SITE (1)..(128): Staphylococcus hyicus mini-domain 6 from 12 mini-domain
QFKLYHDLKNIFKDPKYLNDIQLMDNIAEIITIYQDAESIIKELNQLPELLSEREKEKISALSGYSGTHRLSLKCINLLLDDLWESSLNQMELFTKLNLKPKKIDLSQQHKIPSKLVDDFILSPVVKR

SEQ ID NO: 864 - protein, 128 aa, Staphylococcus microti
  SITE (1)..(128): Staphylococcus microti mini-domain 6 from 12 mini-domain library
SFKLFHDLKKVVKDHAILDDIDLLNQIAEILTIYQDKDSIVAELGQLEYLMSEADKQSISELTGYTGTHSLSLKCMNMIIDELWHSSMNQMEVFTYLNMRPKKYELKGYQRIPTDMIDDAILSPVVKR

SEQ ID NO: 865 - protein, 86 aa, Staphylococcus lugdunensis
  SITE (1)..(86): Staphylococcus lugdunensis mini-domain 7 from 12 mini-domain library
TFGQAINLINKIIEKYGVPEDIIIELARENNSKDKQKFINEMQKKNENTRKRINEIIGKYGNQNAKRLVEKIRLHDEQEGKCLYSL

SEQ ID NO: 866 - protein, 86 aa, Staphylococcus pasteuri
  SITE (1)..(86): Staphylococcus pasteuri mini-domain 7 from 12 mini-domain library
TFIQSINVINKVIEKYGIPEDIIIELARENNSDDRKKFINNLQKKNEATRKRINEIIGQTGNQNAKRIVEKIRLHDQQEGKCLYSL

SEQ ID NO: 867 - protein, 86 aa, Staphylococcus hyicus
  SITE (1)..(86): Staphylococcus hyicus mini-domain 7 from 12 mini-domain library
AFIQSIQVVNAIIDKYGLPEDIIIELARENNSDDRRKFLNQLQKQNEETRKQVEKVLREYGNDNAKRIVQKIKLHNMQEGKCLYSL

SEQ ID NO: 868 - protein, 86 aa, Staphylococcus microti
  SITE (1)..(86): Staphylococcus microti mini-domain 7 from 12 mini-domain library
SFKQAIGVVNAIIKKYGLPKDIIIELARESNSAEKSRYLRAIQKKNEKTRERIEAIIKEYGNENAKGLVQKIKLHDAQEGKCLYSL

SEQ ID NO: 869 - protein, 92 aa, Staphylococcus lugdunensis
  SITE (1)..(92): Staphylococcus lugdunensis mini-domain 8 from 12 mini-domain library
ESIPLEDLLNNPNHYEVDHIIPRSVSFDNSYHNKVLVKQSENSKKSNLTPYQYFNSGKSKLSYNQFKQHILNLSKSQDRISKKKKEYLLEER

SEQ ID NO: 870 - protein, 92 aa, Staphylococcus pasteuri
  SITE (1)..(92): Staphylococcus pasteuri mini-domain 8 from 12 mini-domain library
ESIALMDLLNNPQNYEVDHIIPRSVAFDNSIHNKVLVKQIENSKKGNRTPYQYLNSSDAKLSYNQFKQHILNLSKSKDRISKKKKDYLLEER

SEQ ID NO: 871 - protein, 92 aa, Staphylococcus hyicus
  SITE (1)..(92): Staphylococcus hyicus mini-domain 8 from 12 mini-domain library
KDIPLEDLLRNPHHYEVDHIIPRSVAFDNSMHNKVLVRADENSKKGNRTPYQYLNSSESSLSYNEFKQHILNLSKTKDRITKKKREYLLEER

SEQ ID NO: 872 - protein, 92 aa, Staphylococcus microti
  SITE (1)..(92): Staphylococcus microti mini-domain 8 from 12 mini-domain library
KDIPLEDLLRNPNNYDIDHIIPRSVSFDDSMHNKVLVRREQNAKKNNQTPYQYLTSGYADIKYSVFKQHVLNLAENKDRMTKKKREYLLEER

SEQ ID NO: 873 - protein, 27 aa, Staphylococcus lugdunensis
  SITE (1)..(27): Staphylococcus lugdunensis mini-domain 9 from 12 mini-domain library
DINKFEVQKEFINRNLVDTRYATRELT

SEQ ID NO: 874 - protein, 27 aa, Staphylococcus pasteuri
  SITE (1)..(27): Staphylococcus pasteuri mini-domain 9 from 12 mini-domain library
DINKFEVQKEFINRNLVDTRYATRELT

SEQ ID NO: 875 - protein, 27 aa, Staphylococcus hyicus
  SITE (1)..(27): Staphylococcus hyicus mini-domain 9 from 12 mini-domain library
DINKFDVQKEFINRNLVDTRYATRELT

SEQ ID NO: 876 - protein, 27 aa, Staphylococcus microti
  SITE (1)..(27): Staphylococcus microti mini-domain 9 from 12 mini-domain library
NINKYDVQKEFINRNLVDTRYTTRELT

SEQ ID NO: 877 - protein, 64 aa, Staphylococcus lugdunensis
  SITE (1)..(64): Staphylococcus lugdunensis mini-domain 10 from 12 mini-domain library
NYLKAYFSANNMNVKVKTINGSFTDYLRKVWKFKKERNHGYKHHAEDALIIANADFLFKENKKL

SEQ ID NO: 878 - protein, 64 aa, Staphylococcus pasteuri
  SITE (1)..(64): Staphylococcus pasteuri mini-domain 10 from 12 mini-domain library
SYLKAYFSANNMDVKVKTINGSFTNHLRKVWRFDKYRNHGYKHHAEDALIIANADFLFKENKKL

SEQ ID NO: 879 - protein, 64 aa, Staphylococcus hyicus
  SITE (1)..(64): Staphylococcus hyicus mini-domain 10 from 12 mini-domain library
SLLKAYFSANNLDVKVKTINGSFTNYLRKVWKFDKDRNKGYKHHAEDALIIANADFLFKHNKKL

SEQ ID NO: 880 - protein, 64 aa, Staphylococcus microti
  SITE (1)..(64): Staphylococcus microti mini-domain 10 from 12 mini-domain library
TLLKTYFTINNLDVKVKTINGSFTDFLRKRWGFKKNRDEGYKHHAEDALIIANADYLFKEHKLL

SEQ ID NO: 881 - protein, 186 aa, Staphylococcus lugdunensis
  SITE (1)..(186): Staphylococcus lugdunensis mini-domain 11 from 12 mini-domain library
KAVNSVLEKPEIETKQLDIQVDSEDNYSEMFIIPKQVQDIKDFRNFKYSHRVDKKPNRQLINDTLYSTRKKDNSTYIVQTIKDIYAKDNTTLKKQFDKSPEKFLMYQHDPRTFEKLEVIMKQYANEKNPLAKYHEETGEYLTKYSKKNNGPIVKSLKYIGNKLGSHLDVTHQFKSSTKKLVKLSIK

SEQ ID NO: 882 - protein, 186 aa, Staphylococcus pasteuri
  SITE (1)..(186): Staphylococcus pasteuri mini-domain 11 from 12 mini-domain library
QNTNKILEKPTIENNTKKVTVEKEEDYNNVFETPKLVEDIKQYRDYKFSHRVDKKPNRQLINDTLYSTRMKDEHDYIVQTITDIYGKDNTNLKKQFNKNPEKFLMYQNDPKTFEKLSIIMKQYSDEKNPLAKYYEETGEYLTKYSKKNNGPIVKKIKLLGNKVGNHLDVTNKYENSTKKLVKLSIK

SEQ ID NO: 883 - protein, 184 aa, Staphylococcus hyicus
  SITE (1)..(184): Staphylococcus hyicus mini-domain 11 from 12 mini-domain library
RNINKVLDAPSKEVDKKRVTVQSEDEYNQIFEDTQKAQAIKKFEIRKFSHRVDKKPNRQLINDTLYSTRNIDGIEYVVESIKDIYSVNNDKVKTKFKKDPHRLLMYRNDPQTFEKFEKVFKQYESEKNPFAKYYEETGEKIRKFSKTGQGPYINKIKYLRERLGRHCDVTNKYINSRNKIVQLK

SEQ ID NO: 884 - protein, 183 aa, Staphylococcus microti
  SITE (1)..(183): Staphylococcus microti mini-domain 11 from 12 mini-domain library
KEIKDVSDLAGDERNSNVKDEDQYEEVFGGYFKIEDIKKYKIKKFSHRVDKKPNRQLINDTIYSTRVKDDKRYLINTLKNLYDKSNGDLKERMQKDPESLLMYHHDPQTFEKLKIVMSQYENEKNPLAKYFEETGQYLTKYAKHDNGPAIHKIKYYGNKLVEHLDITKNYHNPQNKVVQLSQK

SEQ ID NO: 885 - protein, 145 aa, Staphylococcus lugdunensis
  SITE (1)..(145): Staphylococcus lugdunensis mini-domain 12 from 12 mini-domain library
PYRFDVYLTDKGYKFITISYLDVLKKDNYYYIPEQKYDKLKLGKAIDKNAKFIASFYKNDLIKLDGEIYKIIGVNSDTRNMIELDLPDIRYKEYCELNNIKGEPRIKKTIGKKVNSIEKLTTDVLGNVFTNTQYTKPQLLFKRGN

SEQ ID NO: 886 - protein, 145 aa, Staphylococcus pasteuri
  SITE (1)..(145): Staphylococcus pasteuri mini-domain 12 from 12 mini-domain library
NYRFDVYLTEKGYKFVTIAYLNVFKKDNYYYIPKDKYQELKEKKKIKDTDQFIASFYKNDLIKLNGDLYKIIGVNSDDRNIIELDYYDIKYKDYCEINNIKGEPRIKKTIGKKTESIEKFTTDVLGNLYLHSTEKAPQLIFKRGL

SEQ ID NO: 887 - protein, 144 aa, Staphylococcus hyicus
  SITE (1)..(144): Staphylococcus hyicus mini-domain 12 from 12 mini-domain library
IYSYRFDIYQYGNNYKMITISYIDLEQKSNYYYISREKYEQKKKDKQIDDSYKFIGSFYKNDIINYNGEMYRVIGVNDSEKNKIQLDMIDISIKDYMELNNIKKTGVIYKTIGKSTTHIEKYTTDILGNLYKAAPPKKPQLIFK

SEQ ID NO: 888 - protein, 144 aa, Staphylococcus microti
  SITE (1)..(144): Staphylococcus microti mini-domain 12 from 12 mini-domain library
SFRFDVYQTDKGYKFISIAYLTLKNEKNYYAISQEKYDQLKSEKKISNNAVFIGSFYTSDIIEINNEKFRVIGVNSDKNNLIEVDRIDIRQKEFIELEEEKKNNRIKVTIGRKTTNIEKFHTDILGNMYKSKRPKAPQLVFKKG

(19) United States
(12) Patent Application Publication
(10) Pub. No.: US 2023/0414648 A1
(43) Pub. Date: Dec. 28, 2023

(54) Compositions and Methods for Treatment of DM1 with SLUCAS9 and SACAS9

(71) Applicant: Vertex Pharmaceuticals Incorporated, Boston, MA (US)

(72) Inventors: John Patrick Leonard, Cambridge, MA (US); Zachary Michael Detwiler, Cambridge, MA (US); Peter Thomas Hallock, Cambridge, MA (US); Eric Gunnar Anderson, Cambridge, MA (US)

(73) Assignee: Vertex Pharmaceuticals Incorporated, Boston, MA (US)

(21) Appl. No.: 18/035,631

(22) PCT Filed: Nov. 5, 2021
(86) PCT No.: PCT/US2021/058157

§ 371 Date: May 5, 2023

Related U.S. Application Data
(60) Provisional application No. 63/110,579, filed on Nov. 6, 2020.

(51) Int. Cl. A61K 31/7105; C12N 15/11; C12N 9/22; C12N 15/90; A61K 38/46

(52) U.S. Cl. CPC A61K 31/7105; C12N 15/11; C12N 9/22; C12N 15/907; A61K 38/465; C12N 2310/20; C12N 2800/80

(57) ABSTRACT

Compositions and methods for treating myotonic dystrophy type 1 (DM1) with SluCas9 and SaCas9 are encompassed.

### CROSS-REFERENCE TO RELATED APPLICATIONS

This application claims the benefit of priority to U.S. Provisional Application No. 63/110,579, filed Nov. 6, 2020, which is incorporated by reference in its entirety.

### SEQUENCE LISTING STATEMENT

The instant application contains a Sequence Listing which has been submitted electronically in ASCII format and is hereby incorporated by reference in its entirety. Said ASCII copy, created on Nov. 4, 2021, is named 2021-11-04_01245-0020-00PCT_ST25.txt and is 316,037 bytes in size.

### INTRODUCTION AND SUMMARY

Myotonic dystrophy type 1 (DM1) is a disorder caused by expansion of a CTG trinucleotide repeat in the noncoding region of the DMPK gene. The protein encoded by the DMPK gene is called myotonic dystrophy protein kinase and is believed to play a role in communication between cells. The DMPK protein is also important for the maintenance of skeletal muscle. If the number of CTG repeats in the DMPK gene is greater than normal, a longer and toxic RNA is produced, preventing cells in muscles and other tissues from functioning normally.

DM1 affects muscle and other body systems with patients typically experiencing muscle weakness and wasting. Adults may become disabled and have a shortened life span. A diagnosis of DM1 is confirmed by molecular genetic testing of DMPK.

CRISPR-based genome editing can provide sequence-specific cleavage of genomic DNA using an RNA-targeted endonuclease and a guide RNA. Providing a pair of guide RNAs that cut on either side of the trinucleotide repeat may result in excision to some extent, but the breaks may simply be resealed without loss of the intervening repeats in a significant number of cells. Accordingly, there is a need for improved compositions and methods for excision of the CTG repeat region in DMPK to treat DM1.

Adeno-associated virus (AAV) administration of the CRISPR-Cas components in vivo or in vitro is attractive due to the early and ongoing successes of AAV vector design, manufacturing, and clinical stage administration for gene therapy. See, e.g., Wang et al. (2019) Nature Reviews Drug Discovery 18:358-378; Ran et al. (2015a) Nature 520: 186-101. However, the commonly used Streptococcus pyogenes (SpCas9) is very large, and when used in AAV-based CRISPR/Cas systems, requires two AAV vectors—one vector carrying the nucleic acid encoding the spCas9, and the other carrying the nucleic acid encoding the guide RNA. One possible way to overcome this technical hurdle is to take advantage of the smaller orthologs of Cas9 derived from different prokaryotic species. Smaller Cas9s such as Staphylococcus aureus (SaCas9) and Staphylococcus lugdunensis (SluCas9) may be able to be manufactured on a single AAV vector together with a nucleic acid encoding one or more guide RNAs. One advantage of incorporating one or more guide RNAs on a single vector together with the smaller SaCas9 or SluCas9 is that doing so allows extreme design flexibility in situations where more than one guide RNA is desired for optimal performance. For example, one vector may be utilized to express SaCas9 or SluCas9 and one or more guide RNAs targeting one or more genomic targets, and a second vector may be utilized to express multiple copies of the same or different guide RNAs targeting the same or different genomic targets. Alternatively, one vector may be utilized to express SaCas9 or SluCas9, and a second vector may be utilized to express one or more guide RNAs targeting one or more genomic targets. Compositions and methods utilizing these dual vector configurations have the benefit of reducing manufacturing costs, reducing complexity of administration routes and protocols, and allowing maximum flexibility with regard to using multiple copies of the same or different guide RNAs targeting the same or different genomic target sequences. In some instances, providing multiple copies of the same guide RNA improves the efficiency of the guide, improving an already successful system. Another benefit to using a endonucleases such as SaCas9 or SluCas9 is that a vector (e.g., AAV) may accommodate a nucleic acid encoding these nucleases more easily than a nucleic acid encoding the much larger SpCas9.

Disclosed herein are compositions and methods using guide RNAs particularly suitable for use with the smaller Cas9 from Staphylococcus lugdunensis (SluCas9) and Staphylococcus aureus (SaCas9).

Accordingly, the following embodiments are provided:
[Embodiment 01] A composition comprising:
   a. one or more guide RNAs (gRNAs), or a vector encoding one or more gRNAs, wherein each gRNA comprises:
      i. a spacer sequence selected from any one of SEQ ID NOs: 1-51, 53, 55-56, 58-59, 61-62, 64, 66, and 70; or
      ii. a spacer sequence that is at least 20 contiguous nucleotides of any one of SEQ ID NOs: 1-51, 53, 55-56, 58-59, 61-62, 64, 66, or 70; or
      iii. a spacer sequence that is at least 90% identical to any one of SEQ ID NOs: 1-51, 53, 55-56, 58-59, 61-62, 64, 66, or 70;
   b. wherein the gRNAs are for use with a SluCas9; and
   c. optionally a Staphylococcus lugdunensis Cas9 (SluCas9) or a nucleic acid encoding a SluCas9; or
   d. one or more guide RNAs (gRNAs), or a vector encoding one or more gRNAs, wherein each gRNA comprises:
      i. a spacer sequence selected from any one of SEQ ID NOs: 200-259; or
      ii. a spacer sequence that is at least 20 contiguous nucleotides of any one of SEQ ID NOs: 200-259; or
      iii. a spacer sequence that is at least 90% identical to any one of SEQ ID NOs: 200-259;
   e. wherein the gRNAs are for use with a Staphylococcus aureus Cas9 (SaCas9); and
   f. optionally a SaCas9 or a nucleic acid encoding a SaCas9.
[Embodiment 02] The composition of embodiment 1, comprising a SluCas9 or a nucleic acid encoding a SluCas9.
[Embodiment 03] The composition of embodiment 1, comprising a SaCas9 or a nucleic acid encoding a SaCas9.
[Embodiment 04] A composition comprising:
   a pair of guide RNAs comprising a pair of spacer sequences, or one or more vectors encoding the pair of guide RNAs, wherein the pair of spacer sequences comprise:
      i. a first spacer sequence selected from SEQ ID NOs: 3, 5, 6, 9, 16, 21, 22, 25, 26, 30, 36, 38, 39, 40, 46, 51, 53, 55, 56, 58, 59, 61, 62, 64, 66, and 70, and a second spacer sequence selected from SEQ ID NOs: 1, 2, 4, 7, 8, 10, 11, 12, 13, 14, 15, 17, 18, 19, 20, 23, 24, 27, 28, 29, 31, 32, 33, 34, 35, 37, 41, 42, 43, 44, 45, 47, 48, 49, and 50; and/or
      ii. a first spacer sequence having at least 20 contiguous nucleotides of a sequence selected from SEQ ID NOs: 3, 5, 6, 9, 16, 21, 22, 25, 26, 30, 36, 38, 39, 40, 46, 51, 53, 55, 56, 58, 59, 61, 62, 64, 66, and 70, and a second spacer sequence having at least 20 contiguous nucleotides of a sequence selected from SEQ ID NOs: 1, 2, 4, 7, 8, 10, 11, 12, 13, 14, 15, 17, 18, 19, 20, 23, 24, 27, 28, 29, 31, 32, 33, 34, 35, 37, 41, 42, 43, 44, 45, 47, 48, 49, and 50; and/or
      iii. a first spacer sequence that is at least 99%, 98%, 97%, 96%, 95%, 94%, 93%, 92%, 91%, or 90% identical to a sequence selected from SEQ ID NOs: 3, 5, 6, 9, 16, 21, 22, 25, 26, 30, 36, 38, 39, 40, 46, 51, 53, 55, 56, 58, 59, 61, 62, 64, 66, and 70, and a second spacer sequence that is at least 99%, 98%, 97%, 96%, 95%, 94%, 93%, 92%, 91%, or 90% identical to a sequence selected from SEQ ID NOs: 1, 2, 4, 7, 8, 10, 11, 12, 13, 14, 15, 17, 18, 19, 20, 23, 24, 27, 28, 29, 31, 32, 33, 34, 35, 37, 41, 42, 43, 44, 45, 47, 48, 49, and 50, wherein the gRNAs are for use with a SluCas9.
[Embodiment 05] A composition comprising:
   a pair of guide RNAs comprising a pair of spacer sequences, or one or more vectors encoding the pair of guide RNAs, wherein the pair of spacer sequences comprise:
      i. a first spacer sequence selected from SEQ ID NOs: 201-203, 211, 215, 220, 225, 231, 235, 238, and 240-259 and a second spacer sequence selected from SEQ ID NOs: 200, 204-210, 212-214, 216-219, 221-224, 226-230, 232-234, 236-237, and 239; and/or
      ii. a first spacer sequence having at least 20 contiguous nucleotides of a sequence selected from SEQ ID NOs: 201-203, 211, 215, 220, 225, 231, 235, 238, and 240-259, and a second spacer sequence having at least 20 contiguous nucleotides of a sequence selected from SEQ ID NOs: 1200, 204-210, 212-214, 216-219, 221-224, 226-230, 232-234, 236-237, and 239; and/or
      iii. a first spacer sequence that is at least 99%, 98%, 97%, 96%, 95%, 94%, 93%, 92%, 91%, or 90% identical to a sequence selected from SEQ ID NOs: 201-203, 211, 215, 220, 225, 231, 235, 238, and 240-259, and a second spacer sequence that is at least 99%, 98%, 97%, 96%, 95%, 94%, 93%, 92%, 91%, or 90% identical to a sequence selected from SEQ ID NOs: 200, 204-210, 212-214, 216-219, 221-224, 226-230, 232-234, 236-237, and 239, wherein the gRNAs are for use with a SaCas9.
[Embodiment 06] A composition comprising:
   a pair of guide RNAs comprising a pair of spacer sequences, or one or more vectors encoding the pair of guide RNAs, wherein the pair of spacer sequences comprise:
   a first spacer sequence selected from SEQ ID NOs: 5, 21, 46, 55, 59, 61, or 64 and a second spacer sequence selected from SEQ ID NOs: 7, 19, 41, or 47, wherein the gRNAs are for use with a SluCas9;
   a first spacer sequence selected from SEQ ID NOs: 201-202 and a second spacer sequence selected from SEQ ID NOs: 206, 213, 218, or 224, wherein the gRNAs are for use with a SaCas9;
   a first and second spacer sequence of SEQ ID NOs: 5 and 7, wherein the gRNAs are for use with a SluCas9;
   a first and second spacer sequence of SEQ ID NOs: 5 and 10, wherein the gRNAs are for use with a SluCas9;
   a first and second spacer sequence of SEQ ID NOs: 5 and 19, wherein the gRNAs are for use with a SluCas9;
   a first and second spacer sequence of SEQ ID NOs: 5 and 41, wherein the gRNAs are for use with a SluCas9;
   a first and second spacer sequence of SEQ ID NOs: 5 and 47, wherein the gRNAs are for use with a SluCas9;
   a first and second spacer sequence of SEQ ID NOs: 21 and 7, wherein the gRNAs are for use with a SluCas9;
   a first and second spacer sequence of SEQ ID NOs: 21 and 19, wherein the gRNAs are for use with a SluCas9;
   a first and second spacer sequence of SEQ ID NOs: 21 and 41, wherein the gRNAs are for use with a SluCas9;
   a first and second spacer sequence of SEQ ID NOs: 21 and 47, wherein the gRNAs are for use with a SluCas9;
   a first and second spacer sequence of SEQ ID NOs: 46 and 7, wherein the gRNAs are for use with a SluCas9;
   a first and second spacer sequence of SEQ ID NOs: 46 and 10, wherein the gRNAs are for use with a SluCas9;
   a first and second spacer sequence of SEQ ID NOs: 46 and 19 wherein the gRNAs are for use with a SluCas9;
   a first and second spacer sequence of SEQ ID NOs: 46 and 41, wherein the gRNAs are for use with a SluCas9;
   a first and second spacer sequence of SEQ ID NOs: 46 and 47, wherein the gRNAs are for use with a SluCas9;
   a first and second spacer sequence of SEQ ID NOs: 55 and 7, wherein the gRNAs are for use with a SluCas9;
   a first and second spacer sequence of SEQ ID NOs: 55 and 19, wherein the gRNAs are for use with a SluCas9;
   a first and second spacer sequence of SEQ ID NOs: 55 and 41, wherein the gRNAs are for use with a SluCas9;
   a first and second spacer sequence of SEQ ID NOs: 55 and 47, wherein the gRNAs are for use with a SluCas9;
   a first and second spacer sequence of SEQ ID NOs: 59 and 7, wherein the gRNAs are for use with a SluCas9;
   a first and second spacer sequence of SEQ ID NOs: 59 and 19, wherein the gRNAs are for use with a SluCas9;
   a first and second spacer sequence of SEQ ID NOs: 59 and 41, wherein the gRNAs are for use with a SluCas9;
   a first and second spacer sequence of SEQ ID NOs: 59 and 47, wherein the gRNAs are for use with a SluCas9;
   a first and second spacer sequence of SEQ ID NOs: 61 and 7, wherein the gRNAs are for use with a SluCas9;
   a first and second spacer sequence of SEQ ID NOs: 61 and 10, wherein the gRNAs are for use with a SluCas9;
   a first and second spacer sequence of SEQ ID NOs: 61 and 19, wherein the gRNAs are for use with a SluCas9;
   a first and second spacer sequence of SEQ ID NOs: 61 and 41, wherein the gRNAs are for use with a SluCas9;
   a first and second spacer sequence of SEQ ID NOs: 61 and 47, wherein the gRNAs are for use with a SluCas9;
   a first and second spacer sequence of SEQ ID NOs: 64 and 7, wherein the gRNAs are for use with a SluCas9;
   a first and second spacer sequence of SEQ ID NOs: 64 and 19, wherein the gRNAs are for use with a SluCas9;
   a first and second spacer sequence of SEQ ID NOs: 64 and 41, wherein the gRNAs are for use with a SluCas9;
   a first and second spacer sequence of SEQ ID NOs: 64 and 47, wherein the gRNAs are for use with a SluCas9;
   a first and second spacer sequence of SEQ ID NOs: 202 and 218, wherein the gRNAs are for use with a SaCas9;
   a first and second spacer sequence of SEQ ID NOs: 201 and 224, wherein the gRNAs are for use with a SaCas9;
   a first and second spacer sequence of SEQ ID NOs: 202 and 213, wherein the gRNAs are for use with a SaCas9; or a first and second spacer sequence of SEQ ID NOs: 202 and 206, wherein the gRNAs are for use with a SaCas9.
[Embodiment 07] The composition of embodiment 4, further comprising a SluCas9, or a nucleic acid encoding the SluCas9.
[Embodiment 08] The composition of embodiment 5, further comprising a SaCas9, or a nucleic acid encoding the SaCas9.
[Embodiment 09] The composition of any one of the preceding embodiments, wherein the guide RNA comprises a crRNA and/or a tracrRNA sequence.
[Embodiment 10] The composition of any one of embodiments 1a, 4, 6a, and 6c-6gg, wherein the guide RNA comprises any one of SEQ ID NOs: 1-51, 53, 55-56, 58-59, 61-62, 64, 66, and and further comprises:
   a. a sequence selected from SEQ ID NOs: 600-604;
   b. a sequence that is at least 80%, 85%, 90%, 91%, 92%, 93%, 94%, 95%, 96%, 97%, 98%, 99% or 100% identical to SEQ ID NOs: 600-604; or
   c. a sequence that differs from SEQ ID NOs: 600-604 by no more than 1, 2, 3, 4, 5, 10, 20, or 25 nucleotides.
[Embodiment 11] The composition of any one of embodiments 1a, 4, 6a, and 6c-6gg, wherein the SluCas9 comprises SEQ ID NO: 712.
[Embodiment 12] The composition of any one of embodiments 1a, 4, 6a, and 6c-6gg, wherein the SluCas9 comprises a sequence that is at least 80%, 85%, 90%, 91%, 92%, 93%, 94%, 95%, 96%, 97%, 98%, 99% or 100% identical to the sequence of SEQ ID NO: 712.
[Embodiment 13] The composition of any one of embodiments 1b, 5, 6b, and 6hh-6kk, wherein the SaCas9 comprises SEQ ID NO: 711.
[Embodiment 14] The composition of any one of embodiments 1b, 5, 6b, and 6hh-6kk, wherein the SaCas9 comprises a sequence that is at least 80%, 85%, 90%, 91%, 92%, 93%, 94%, 95%, 96%, 97%, 98%, 99% or 100% identical to the sequence of SEQ ID NO: 711.
[Embodiment 15] The composition of any one of embodiments 1a, 4, 6a, and 6c-6gg, wherein the SluCas9 comprises:
   a. a sequence selected from SEQ ID NOs: 800-805 and 809-888;
   b. a chimeric SluCas9 protein comprising a SluCas9 PAM interacting domain.
[Embodiment 16] The composition of any one of embodiments 1a, 4, 6a, and 6c-6gg, wherein the SluCas9 or nucleic acid encoding SluCas9 comprises one or more of the following mutations to SEQ ID NO: 712:
   a. a mutation at any one of, or combination of, positions R246, N414, T420, or R655;
   b. a mutation at the position corresponding to position R246 of SEQ ID NO: 712 (e.g., R246A);
   c. a mutation at the position corresponding to position N414 of SEQ ID NO: 712 (e.g., N414A);
   d. a mutation at the position corresponding to position T420 of SEQ ID NO: 712 (e.g., T420A);
   e. a mutation at the position corresponding to position R655 of SEQ ID NO: 712 (e.g., R655A);
   f. a combination of mutations at the positions corresponding to position R246 of SEQ ID NO: 712 (e.g., R246A), position N414 of SEQ ID NO: 712 (e.g., N414A), position T420 of SEQ ID NO: 712 (e.g., T420A), and position R655 of SEQ ID NO: 712 (e.g., R655A);
   g. a mutation at the position corresponding to position Q781 of SEQ ID NO: 712 (e.g., Q781K);
   h. a mutation at the position corresponding to position R1013 of SEQ ID NO: 712 (e.g., R1013H); and
   i. a combination of mutations at the positions corresponding to position Q781 of SEQ ID NO: 712 (e.g., Q781K) and position R1013 of SEQ ID NO: 712 (e.g., R1013H).
[Embodiment 17] The composition of any one of the preceding embodiments, wherein the guide RNA is an sgRNA.
[Embodiment 18] The composition of embodiment 17, wherein the sgRNA is modified.
[Embodiment 19] The composition of embodiment 18, wherein the modifications alter one or more 2′ positions and/or phosphodiester linkages.
[Embodiment 20] The composition of any one of embodiments 18-19, wherein the modifications alter one or more, or all, of the first three nucleotides of the sgRNA.
[Embodiment 21] The composition of any one of embodiments 18-20, wherein the modifications alter one or more, or all, of the last three nucleotides of the sgRNA.
[Embodiment 22] The composition of any one of embodiments 18-21, wherein the modifications include one or more of a phosphorothioate modification, a 2′-OMe modification, a 2′-O-MOE modification, a 2′-F modification, a 2′-O-methine-4′ bridge modification, a 3′-thiophosphonoacetate modification, and a 2′-deoxy modification.
[Embodiment 23] The composition of any one of the preceding embodiments, wherein the composition further comprises a pharmaceutically acceptable excipient.
[Embodiment 24] The composition of any one of the preceding embodiments, wherein the guide RNA or nucleic acid encoding the guide RNA is associated with a lipid nanoparticle (LNP).
[Embodiment 25] The composition of any one of the preceding embodiments, wherein the guide RNA or nucleic acid encoding the guide RNA is associated with a viral vector.
[Embodiment 26] The composition of embodiment 25, wherein the viral vector is an adeno-associated virus vector, a lentiviral vector, an integrase-deficient lentiviral vector, an adenoviral vector, a vaccinia viral vector, an alphaviral vector, or a herpes simplex viral vector.
[Embodiment 27] The composition of embodiment 26, wherein the viral vector is an adeno-associated virus (AAV) vector.
[Embodiment 28] The composition of embodiment 27, wherein the AAV vector is an AAV1, AAV2, AAV3, AAV4, AAV5, AAV6, AAV7, AAV8, AAVrh10, AAVrh74, or AAV9 vector, wherein the number following AAV indicates the AAV serotype.
[Embodiment 29] The composition of embodiment 28, wherein the AAV vector is an AAV serotype 9 vector.
[Embodiment 30] The composition of any one of embodiments 25-28, wherein the viral vector comprises a tissue-specific promoter.
[Embodiment 31] The composition of any one of embodiments 25-30, wherein the viral vector comprises a muscle-specific promoter, optionally wherein the muscle-specific promoter is a muscle creatine kinase promoter, a desmin promoter, an MHCK7 promoter, or an SPc5-12 promoter.
[Embodiment 32] The composition of any one of embodiments 25-31, wherein the viral vector comprises a neuron-specific promoter, optionally wherein the neuron-specific promoter is an enolase promoter.
[Embodiment 33] The composition of any one of the preceding embodiments comprising a spacer sequence having the sequence of SEQ ID NO: 1.
[Embodiment 34] The composition of any one of the preceding embodiments comprising a spacer sequence having the sequence of SEQ ID NO: 2.
[Embodiment 35] The composition of any one of the preceding embodiments comprising a spacer sequence having the sequence of SEQ ID NO: 3.
[Embodiment 36] The composition of any one of the preceding embodiments comprising a spacer sequence having the sequence of SEQ ID NO: 4.
[Embodiment 37] The composition of any one of the preceding embodiments comprising a spacer sequence having the sequence of SEQ ID NO: 5.
[Embodiment 38] The composition of any one of the preceding embodiments comprising a spacer sequence having the sequence of SEQ ID NO: 6.
[Embodiment 39] The composition of any one of the preceding embodiments comprising a spacer sequence having the sequence of SEQ ID NO: 7.
[Embodiment 40] The composition of any one of the preceding embodiments comprising a spacer sequence having the sequence of SEQ ID NO: 8.
[Embodiment 41] The composition of any one of the preceding embodiments comprising a spacer sequence having the sequence of SEQ ID NO: 9.
[Embodiment 42] The composition of any one of the preceding embodiments comprising a spacer sequence having the sequence of SEQ ID NO: 10.
[Embodiment 43] The composition of any one of the preceding embodiments comprising a spacer sequence having the sequence of SEQ ID NO: 11.
[Embodiment 44] The composition of any one of the preceding embodiments comprising a spacer sequence having the sequence of SEQ ID NO: 12.
[Embodiment 45] The composition of any one of the preceding embodiments comprising a spacer sequence having the sequence of SEQ ID NO: 13.
[Embodiment 46] The composition of any one of the preceding embodiments comprising a spacer sequence having the sequence of SEQ ID NO: 14.
[Embodiment 47] The composition of any one of the preceding embodiments comprising a spacer sequence having the sequence of SEQ ID NO: 15.
[Embodiment 48] The composition of any one of the preceding embodiments comprising a spacer sequence having the sequence of SEQ ID NO: 16.
[Embodiment 49] The composition of any one of the preceding embodiments comprising a spacer sequence having the sequence of SEQ ID NO: 17.
[Embodiment 50] The composition of any one of the preceding embodiments comprising a spacer sequence having the sequence of SEQ ID NO: 18.
[Embodiment 51] The composition of any one of the preceding embodiments comprising a spacer sequence having the sequence of SEQ ID NO: 19.
[Embodiment 52] The composition of any one of the preceding embodiments comprising a spacer sequence having the sequence of SEQ ID NO: 20.
[Embodiment 53] The composition of any one of the preceding embodiments comprising a spacer sequence having the sequence of SEQ ID NO: 21.
[Embodiment 54] The composition of any one of the preceding embodiments comprising a spacer sequence having the sequence of SEQ ID NO: 22.
[Embodiment 55] The composition of any one of the preceding embodiments comprising a spacer sequence having the sequence of SEQ ID NO: 23.
[Embodiment 56] The composition of any one of the preceding embodiments comprising a spacer sequence having the sequence of SEQ ID NO: 24.
[Embodiment 57] The composition of any one of the preceding embodiments comprising a spacer sequence having the sequence of SEQ ID NO: 25.
[Embodiment 58] The composition of any one of the preceding embodiments comprising a spacer sequence having the sequence of SEQ ID NO: 26.
[Embodiment 59] The composition of any one of the preceding embodiments comprising a spacer sequence having the sequence of SEQ ID NO: 27.
[Embodiment 60] The composition of any one of the preceding embodiments comprising a spacer sequence having the sequence of SEQ ID NO: 28.
[Embodiment 61] The composition of any one of the preceding embodiments comprising a spacer sequence having the sequence of SEQ ID NO: 29.
[Embodiment 62] The composition of any one of the preceding embodiments comprising a spacer sequence having the sequence of SEQ ID NO: 30.
[Embodiment 63] The composition of any one of the preceding embodiments comprising a spacer sequence having the sequence of SEQ ID NO: 31.
[Embodiment 64] The composition of any one of the preceding embodiments comprising a spacer sequence having the sequence of SEQ ID NO: 32.
[Embodiment 65] The composition of any one of the preceding embodiments comprising a spacer sequence having the sequence of SEQ ID NO: 33.
[Embodiment 66] The composition of any one of the preceding embodiments comprising a spacer sequence having the sequence of SEQ ID NO: 34.
[Embodiment 67] The composition of any one of the preceding embodiments comprising a spacer sequence having the sequence of SEQ ID NO: 35.
[Embodiment 68] The composition of any one of the preceding embodiments comprising a spacer sequence having the sequence of SEQ ID NO: 36.
[Embodiment 69] The composition of any one of the preceding embodiments comprising a spacer sequence having the sequence of SEQ ID NO: 37.
[Embodiment 70] The composition of any one of the preceding embodiments comprising a spacer sequence having the sequence of SEQ ID NO: 38.
[Embodiment 71] The composition of any one of the preceding embodiments comprising a spacer sequence having the sequence of SEQ ID NO: 39.
[Embodiment 72] The composition of any one of the preceding embodiments comprising a spacer sequence having the sequence of SEQ ID NO: 40.
[Embodiment 73] The composition of any one of the preceding embodiments comprising a spacer sequence having the sequence of SEQ ID NO: 41.
[Embodiment 74] The composition of any one of the preceding embodiments comprising a spacer sequence having the sequence of SEQ ID NO: 42.
[Embodiment 75] The composition of any one of the preceding embodiments comprising a spacer sequence having the sequence of SEQ ID NO: 43.
[Embodiment 76] The composition of any one of the preceding embodiments comprising a spacer sequence having the sequence of SEQ ID NO: 44.
[Embodiment 77] The composition of any one of the preceding embodiments comprising a spacer sequence having the sequence of SEQ ID NO: 45.
[Embodiment 78] The composition of any one of the preceding embodiments comprising a spacer sequence having the sequence of SEQ ID NO: 46.
[Embodiment 79] The composition of any one of the preceding embodiments comprising a spacer sequence having the sequence of SEQ ID NO: 47.
[Embodiment 80] The composition of any one of the preceding embodiments comprising a spacer sequence having the sequence of SEQ ID NO: 48.
[Embodiment 81] The composition of any one of the preceding embodiments comprising a spacer sequence having the sequence of SEQ ID NO: 49.
[Embodiment 82] The composition of any one of the preceding embodiments comprising a spacer sequence having the sequence of SEQ ID NO: 50.
[Embodiment 83] The composition of any one of the preceding embodiments comprising a spacer sequence having the sequence of SEQ ID NO: 51.
[Embodiment 84] The composition of any one of the preceding embodiments comprising a spacer sequence having the sequence of SEQ ID NO: 53.
[Embodiment 85] The composition of any one of the preceding embodiments comprising a spacer sequence having the sequence of SEQ ID NO: 55.
[Embodiment 86] The composition of any one of the preceding embodiments comprising a spacer sequence having the sequence of SEQ ID NO: 56.
[Embodiment 87] The composition of any one of the preceding embodiments comprising a spacer sequence having the sequence of SEQ ID NO: 58.
[Embodiment 88] The composition of any one of the preceding embodiments comprising a spacer sequence having the sequence of SEQ ID NO: 59.
[Embodiment 89] The composition of any one of the preceding embodiments comprising a spacer sequence having the sequence of SEQ ID NO: 61.
[Embodiment 90] The composition of any one of the preceding embodiments comprising a spacer sequence having the sequence of SEQ ID NO: 62.
[Embodiment 91] The composition of any one of the preceding embodiments comprising a spacer sequence having the sequence of SEQ ID NO: 64.
[Embodiment 92] The composition of any one of the preceding embodiments comprising a spacer sequence having the sequence of SEQ ID NO: 66.
[Embodiment 93] The composition of any one of the preceding embodiments comprising a spacer sequence having the sequence of SEQ ID NO: 70.
[Embodiment 94] Use of a composition of any one of the preceding embodiments for the preparation of a medicament for treating a human subject having DM1.
[Embodiment 95] Use of a composition of any one of the preceding embodiments for treating a human subject having DM1.
[Embodiment 96] A method of treating a muscular dystrophy characterized by a trinucleotide repeat (TNR) in the 3′ UTR of the DMPK gene, the method comprising delivering to a cell that comprises a TNR in the 3′ UTR of the DMPK gene:
   a. the composition of any one of embodiments 1a, 4, 6a, 6c-6gg, 9-12, and 15-95; or
   b. a pair of guide RNAs comprising a pair of spacer sequences, or one or more vectors encoding the pair of guide RNAs, wherein the pair of spacer sequences comprise a first spacer sequence selected from SEQ ID NOs: 3, 5, 6, 9, 16, 21, 22, 25, 26, 30, 36, 38, 39, 40, 46, 51, 53, 55, 56, 58, 59, 61, 62, 64, 66, and 70, and a second spacer sequence selected from SEQ ID NOs: 1, 2, 4, 7, 8, 10, 11, 12, 13, 14, 15, 17, 18, 19, 20, 23, 24, 27, 28, 29, 31, 32, 33, 34, 35, 37, 41, 42, 43, 44, 45, 47, 48, 49, and 50, or a nucleic acid encoding the guide RNA;
   c. a pair of guide RNAs comprising a pair of spacer sequences, or one or more vectors encoding the pair of guide RNAs, wherein the pair of spacer sequences comprises SEQ ID NO: 5 and SEQ ID NO: 10;
   d. a pair of guide RNAs comprising a pair of spacer sequences, or one or more vectors encoding the pair of guide RNAs, wherein the pair of spacer sequences comprises SEQ ID NO: 46 and SEQ ID NO: 10;
   e. a pair of guide RNAs comprising a pair of spacer sequences, or one or more vectors encoding the pair of guide RNAs, wherein the pair of spacer sequences comprises SEQ ID NO: 61 and SEQ ID NO: 10; or
   f. a pair of guide RNAs comprising a pair of spacer sequences, or one or more vectors encoding the pair of guide RNAs, wherein the pair of spacer sequences comprises SEQ ID NO: 64 and SEQ ID NO: 47; and
      i. SluCas9 or a nucleic acid encoding the SluCas9.
[Embodiment 97] A method of excising a trinucleotide repeat (TNR) in the 3′ UTR of the DMPK gene comprising delivering to a cell that comprises the TNR in the 3′ UTR of the DMPK gene a pair of guide RNAs comprising a pair of spacer sequences, or one or more vectors encoding the pair of guide RNAs, wherein the pair of spacer sequences comprise:
   i. a first spacer sequence selected from SEQ ID NOs: 3, 5, 6, 9, 16, 21, 22, 25, 26, 30, 36, 38, 39, 40, 46, 51, 53, 55, 56, 58, 59, 61, 62, 64, 66, and 70, and a second spacer sequence selected from SEQ ID NOs: 1, 2, 4, 7, 8, 10, 11, 12, 13, 14, 15, 17, 18, 19, 20, 23, 24, 27, 28, 29, 31, 32, 33, 34, 35, 37, 41, 42, 43, 44, 45, 47, 48, 49, and 50, or a nucleic acid encoding the guide RNA; and
   ii. SluCas9 or a nucleic acid encoding the SluCas9, wherein at least one TNR is excised.
[Embodiment 98] The method of any one of embodiments 96-97, wherein a pair of guide RNAs that comprises a first and second spacer sequence that guide the SluCas9 to or near a TNR, or one or more vectors encoding the pair of guide RNAs, are delivered to the cell.
[Embodiment 99] The method of any one of embodiments 96-98, wherein the first and second spacer sequences have the sequences of SEQ ID NOs: 5 and 7.
[Embodiment 100] The method of any one of embodiments 96-98, wherein the first and second spacer sequences have the sequences of SEQ ID NOs: 5 and 10.
[Embodiment 101] The method of any one of embodiments 96-98, wherein the first and second spacer sequences have the sequences of SEQ ID NOs: 5 and 19.
[Embodiment 102] The method of any one of embodiments 96-98, wherein the first and second spacer sequences have the sequences of SEQ ID NOs: 5 and 41.
[Embodiment 103] The method of any one of embodiments 96-98, wherein the first and second spacer sequences have the sequences of SEQ ID NOs: 5 and 47.
[Embodiment 104] The method of any one of embodiments 96-98, wherein the first and second spacer sequences have the sequences of SEQ ID NOs: 21 and 7.
[Embodiment 105] The method of any one of embodiments 96-98, wherein the first and second spacer sequences have the sequences of SEQ ID NOs: 21 and 19.
[Embodiment 106] The method of any one of embodiments 96-98, wherein the first and second spacer sequences have the sequences of SEQ ID NOs: 21 and 41.
[Embodiment 107] The method of any one of embodiments 96-98, wherein the first and second spacer sequences have the sequences of SEQ ID NOs: 21 and 47.
[Embodiment 108] The method of any one of embodiments 96-98, wherein the first and second spacer sequences have the sequences of SEQ ID NOs: 46 and 7.
[Embodiment 109] The method of any one of embodiments 96-98, wherein the first and second spacer sequences have the sequences of SEQ ID NOs: 46 and 10.
[Embodiment 110] The method of any one of embodiments 96-98, wherein the first and second spacer sequences have the sequences of SEQ ID NOs: 46 and 19.
[Embodiment 111] The method of any one of embodiments 96-98, wherein the first and second spacer sequences have the sequences of SEQ ID NOs: 46 and 41.
[Embodiment 112] The method of any one of embodiments 96-98, wherein the first and second spacer sequences have the sequences of SEQ ID NOs: 46 and 47.
[Embodiment 113] The method of any one of embodiments 96-98, wherein the first and second spacer sequences have the sequences of SEQ ID NOs: 55 and 7.
[Embodiment 114] The method of any one of embodiments 96-98, wherein the first and second spacer sequences have the sequences of SEQ ID NOs: 55 and 19.
[Embodiment 115] The method of any one of embodiments 96-98, wherein the first and second spacer sequences have the sequences of SEQ ID NOs: 55 and 41.
[Embodiment 116] The method of any one of embodiments 96-98, wherein the first and second spacer sequences have the sequences of SEQ ID NOs: 55 and 47.
[Embodiment 117] The method of any one of embodiments 96-98, wherein the first and second spacer sequences have the sequences of SEQ ID NOs: 59 and 7.
[Embodiment 118] The method of any one of embodiments 96-98, wherein the first and second spacer sequences have the sequences of SEQ ID NOs: 59 and 19.
[Embodiment 119] The method of any one of embodiments 96-98, wherein the first and second spacer sequences have the sequences of SEQ ID NOs: 59 and 41.
[Embodiment 120] The method of any one of embodiments 96-98, wherein the first and second spacer sequences have the sequences of SEQ ID NOs: 59 and 47.
[Embodiment 121] The method of any one of embodiments 96-98, wherein the first and second spacer sequences have the sequences of SEQ ID NOs: 61 and 7.
[Embodiment 122] The method of any one of embodiments 96-98, wherein the first and second spacer sequences have the sequences of SEQ ID NOs: 61 and 10.
[Embodiment 123] The method of any one of embodiments 96-98, wherein the first and second spacer sequences have the sequences of SEQ ID NOs: 61 and 19.
[Embodiment 124] The method of any one of embodiments 96-98, wherein the first and second spacer sequences have the sequences of SEQ ID NOs: 61 and 41.
[Embodiment 125] The method of any one of embodiments 96-98, wherein the first and second spacer sequences have the sequences of SEQ ID NOs: 61 and 47.
[Embodiment 126] The method of any one of embodiments 96-98, wherein the first and second spacer sequences have the sequences of SEQ ID NOs: 64 and 7.
[Embodiment 127] The method of any one of embodiments 96-98, wherein the first and second spacer sequences have the sequences of SEQ ID NOs: 64 and 19.
[Embodiment 128] The method of any one of embodiments 96-98, wherein the first and second spacer sequences have the sequences of SEQ ID NOs: 64 and 41.
[Embodiment 129] The method of any one of embodiments 96-98, wherein the first and second spacer sequences have the sequences of SEQ ID NOs: 64 and 47.
[Embodiment 130] The method of any one of embodiments 96-129, further comprising SluCas9, or a nucleic acid encoding the SluCas9.
[Embodiment 131] The method of any one of embodiment 96-130, wherein the guide RNA further comprises a SluCas9 crRNA and/or a tracrRNA sequence.
[Embodiment 132] The method of any one of embodiments 96-131, wherein the guide RNA further comprises:
   a. a sequence selected from SEQ ID NOs: 600-603;
   b. a sequence that is at least 80%, 85%, 90%, 91%, 92%, 93%, 94%, 95%, 96%, 97%, 98%, 99% or 100% identical to SEQ ID NOs: 600-603; or
   c. a sequence that differs from SEQ ID NOs: 600-603 by no more than 1, 2, 3, 4, 5, 10, 20, or 25 nucleotides.
[Embodiment 133] The method of any one of embodiments 96-132, wherein the SluCas9 or nucleic acid encoding SluCas9 comprises SEQ ID NO: 712.
[Embodiment 134] The method of any one of embodiments 96-133, wherein the SluCas9 or nucleic acid encoding SluCas9 comprises a sequence that is at least 80%, 85%, 90%, 91%, 92%, 93%, 94%, 95%, 96%, 97%, 98%, 99% or 100% identical to the sequence of SEQ ID NO: 712.
[Embodiment 135] The method of any one of embodiments 96-134, wherein the SluCas9 or nucleic acid encoding SluCas9 comprises:
   a. a sequence selected from SEQ ID NOs: 800-805 and 809-888;
   b. a chimeric SaCas9 protein comprising a SluCas9 PAM interacting domain.
[Embodiment 136] The method of any one of the embodiments 96-135, wherein the SluCas9 or nucleic acid encoding SluCas9 comprises one or more of the following mutations to SEQ ID NO: 712:
   a. a mutation at any one of, or combination of, positions R246, N414, T420, or R655;
   b. a mutation at the position corresponding to position R246 of SEQ ID NO: 712 (e.g., R246A);
   c. a mutation at the position corresponding to position N414 of SEQ ID NO: 712 (e.g., N414A);
   d. a mutation at the position corresponding to position T420 of SEQ ID NO: 712 (e.g., T420A);
   e. a mutation at the position corresponding to position R655 of SEQ ID NO: 712 (e.g., R655A);
   f. a combination of mutations at the positions corresponding to position R246 of SEQ ID NO: 712 (e.g., R246A), position N414 of SEQ ID NO: 712 (e.g., N414A), position T420 of SEQ ID NO: 712 (e.g., T420A), and position R655 of SEQ ID NO: 712 (e.g., R655A);
   g. a mutation at the position corresponding to position Q781 of SEQ ID NO: 712 (e.g., Q781K);
   h. a mutation at the position corresponding to position R1013 of SEQ ID NO: 712 (e.g., R1013H); and
   i. a combination of mutations at the positions corresponding to position Q781 of SEQ ID NO: 712 (e.g., Q781K) and position R1013 of SEQ ID NO: 712 (e.g., R1013H).
[Embodiment 137] The method of any one of embodiments 96-136, wherein the guide RNA is an sgRNA.
[Embodiment 138] The method of embodiment 137, wherein the sgRNA is modified.
[Embodiment 139] The method of embodiment 138, wherein the modifications alter one or more 2′ positions and/or phosphodiester linkages.
[Embodiment 140] The method of embodiments 138-139, wherein the modifications alter one or more, or all, of the first three nucleotides of the sgRNA.
[Embodiment 141] The method of embodiments 138-140, wherein the modifications alter one or more, or all, of the last three nucleotides of the sgRNA.
[Embodiment 142] The method of embodiments 138-141, wherein the modifications include one or more of a phosphorothioate modification, a 2′-OMe modification, a 2′-O-MOE modification, a 2′-F modification, a 2′-O-methine-4′ bridge modification, a 3′-thiophosphonoacetate modification, and a 2′-deoxy modification.
[Embodiment 143] The method of any one of embodiments 96-142, wherein the composition further comprises a pharmaceutically acceptable excipient.
[Embodiment 144] The method of any one of embodiments 96-143, wherein the guide RNA is associated with a lipid nanoparticle (LNP), or encoded by a viral vector.
[Embodiment 145] The method of embodiment 144, wherein the viral vector is an adeno-associated virus vector, a lentiviral vector, an integrase-deficient lentiviral vector, an adenoviral vector, a vaccinia viral vector, an alphaviral vector, or a herpes simplex viral vector.
[Embodiment 146] The method of embodiment 145, wherein the viral vector is an adeno-associated virus (AAV) vector.
[Embodiment 147] The method of embodiment 146, wherein the AAV vector is an AAV1, AAV2, AAV3, AAV4, AAV5, AAV6, AAV7, AAV8, AAVrh10, AAVrh74, or AAV9 vector, wherein the number following AAV indicates the AAV serotype.
[Embodiment 148] The method of embodiment 147, wherein the AAV vector is an AAV serotype 9 vector.
[Embodiment 149] The method of any one of embodiments 144-148, wherein the viral vector comprises a tissue-specific promoter.
[Embodiment 150] The method of any one of embodiments 144-147, wherein the viral vector comprises a muscle-specific promoter, optionally wherein the muscle-specific promoter is a muscle creatine kinase promoter, a desmin promoter, an MHCK7 promoter, or an SPc5-12 promoter.
   a. The method of any one of embodiments 135-141, wherein the viral vector comprises a neuron-specific promoter, optionally wherein the neuron-specific promoter is an enolase promoter.
[Embodiment 151] A method of treating a muscular dystrophy characterized by a trinucleotide repeat (TNR) in the 3′ UTR of the DMPK gene, the method comprising delivering to a cell that comprises a TNR in the 3′ UTR of the DMPK gene:
   the composition of any one of 1b, 5, 6b, 6hh-6kk, 13-14, and 15-95; or
   a pair of guide RNAs comprising a pair of spacer sequences, or one or more vectors encoding the pair of guide RNAs, wherein the pair of spacer sequences comprise a first spacer sequence selected from SEQ ID NOs: 201-203, 211, 215, 220, 225, 231, 235, 238, and 240-259 and a second spacer sequence selected from SEQ ID NOs: 200, 204-210, 212-214, 216-219, 221-224, 226-230, 232-234, 236-237, and 239;
      a. a pair of guide RNAs comprising a pair of spacer sequences, or one or more vectors encoding the pair of guide RNAs, wherein the pair of spacer sequences comprises SEQ ID NO: 201 and SEQ ID NO: 206;
      b. a pair of guide RNAs comprising a pair of spacer sequences, or one or more vectors encoding the pair of guide RNAs, wherein the pair of spacer sequences comprises SEQ ID NO: 201 and SEQ ID NO: 224;
      c. a pair of guide RNAs comprising a pair of spacer sequences, or one or more vectors encoding the pair of guide RNAs, wherein the pair of spacer sequences comprises SEQ ID NO: 202 and SEQ ID NO: 213;
      d. a pair of guide RNAs comprising a pair of spacer sequences, or one or more vectors encoding the pair of guide RNAs, wherein the pair of spacer sequences comprises SEQ ID NO: 202 and SEQ ID NO: 218; and
         i. SluCas9 or a nucleic acid encoding the SaCas9.
[Embodiment 152] A method of excising a trinucleotide repeat (TNR) in the 3′ UTR of the DMPK gene comprising delivering to a cell that comprises the TNR in the 3′ UTR of the DMPK gene a pair of guide RNAs comprising a pair of spacer sequences, or one or more vectors encoding the pair of guide RNAs, wherein the pair of spacer sequences comprise:
   i. a first spacer sequence selected from SEQ ID NOs: 201-203, 211, 215, 220, 225, 231, 235, 238, and 240-259, and a second spacer sequence selected from SEQ ID NOs: 200, 204-210, 212-214, 216-219, 221-224, 226-230, 232-234, 236-237, and 239, or a nucleic acid encoding the guide RNA; and
   ii. SaCas9 or a nucleic acid encoding the SaCas9, wherein at least one TNR is excised.
[Embodiment 153] The method of any one of embodiments 151-152, wherein a pair of guide RNAs that comprises a first and second spacer sequence that guide the SaCas9 to or near a TNR, or one or more vectors encoding the pair of guide RNAs, are delivered to the cell.
[Embodiment 154] The method of any one of embodiments 151-153, wherein the first and second spacer sequences have the sequences of SEQ ID NOs: 201 and 206.
[Embodiment 155] The method of any one of embodiments 151-153, wherein the first and second spacer sequences have the sequences of SEQ ID NOs: 201 and 224.
[Embodiment 156] The method of any one of embodiments 151-153, wherein the first and second spacer sequences have the sequences of SEQ ID NOs: 202 and 213.
[Embodiment 157] The method of any one of embodiments 151-153, wherein the first and second spacer sequences have the sequences of SEQ ID NOs: 202 and 218.
[Embodiment 158] The method of any one of embodiments 151-157, further comprising SaCas9, or a nucleic acid encoding the SaCas9.
[Embodiment 159] The method of any one of embodiment 151-158, wherein the guide RNA further comprises a SaCas9 crRNA and/or a tracrRNA sequence.
[Embodiment 160] The method of any one of embodiments 96-128, wherein the guide RNA further comprises:
   a. a sequence selected from SEQ ID NO: 500;
   b. a sequence that is at least 80%, 85%, 90%, 91%, 92%, 93%, 94%, 95%, 96%, 97%, 98%, 99% or 100% identical to SEQ ID NO: 500; or
   c. a sequence that differs from SEQ ID NO: 500 by no more than 1, 2, 3, 4, 5, 10, 15, 20, or 25 nucleotides.
[Embodiment 161] The method of any one of embodiments 151-160, wherein the SaCas9 or nucleic acid encoding SaCas9 comprises SEQ ID NO: 711.
[Embodiment 162] The method of any one of embodiments 151-161, wherein the SaCas9 or nucleic acid encoding SaCas9 comprises a sequence that is at least 80%, 85%, 90%, 91%, 92%, 93%, 94%, 95%, 96%, 97%, 98%, 99% or 100% identical to the sequence of SEQ ID NO: 711.
[Embodiment 163] The method of any one of embodiments 151-162, wherein the guide RNA is an sgRNA.
[Embodiment 164] The method of embodiment 163, wherein the sgRNA is modified.
[Embodiment 165] The method of embodiment 164, wherein the modifications alter one or more 2′ positions and/or phosphodiester linkages.
[Embodiment 166] The method of embodiments 164-165, wherein the modifications alter one or more, or all, of the first three nucleotides of the sgRNA.
[Embodiment 167] The method of embodiments 164-166, wherein the modifications alter one or more, or all, of the last three nucleotides of the sgRNA.
[Embodiment 168] The method of embodiments 164-167, wherein the modifications include one or more of a phosphorothioate modification, a 2′-OMe modification, a 2′-O-MOE modification, a 2′-F modification, a 2′-O-methine-4′ bridge modification, a 3′-thiophosphonoacetate modification, and a 2′-deoxy modification.
[Embodiment 169] The method of any one of embodiments 151-168, wherein the composition further comprises a pharmaceutically acceptable excipient.
[Embodiment 170] The method of any one of embodiments 151-169, wherein the guide RNA is associated with a lipid nanoparticle (LNP), or encoded by a viral vector.
[Embodiment 171] The method of embodiment 170, wherein the viral vector is an adeno-associated virus vector, a lentiviral vector, an integrase-deficient lentiviral vector, an adenoviral vector, a vaccinia viral vector, an alphaviral vector, or a herpes simplex viral vector.
[Embodiment 172] The method of embodiment 171, wherein the viral vector is an adeno-associated virus (AAV) vector.
[Embodiment 173] The method of embodiment 172, wherein the AAV vector is an AAV1, AAV2, AAV3, AAV4, AAV5, AAV6, AAV7, AAV8, AAVrh10, AAVrh74, or AAV9 vector, wherein the number following AAV indicates the AAV serotype.
[Embodiment 174] The method of embodiment 173, wherein the AAV vector is an AAV serotype 9 vector.
[Embodiment 175] The method of any one of embodiments 170-173, wherein the viral vector comprises a tissue-specific promoter.
[Embodiment 176] The method of any one of embodiments 170-175, wherein the viral vector comprises a muscle-specific promoter, optionally wherein the muscle-specific promoter is a muscle creatine kinase promoter, a desmin promoter, an MHCK7 promoter, or an SPc5-12 promoter.
[Embodiment 177] The method of any one of embodiments 170-176, wherein the viral vector comprises a neuron-specific promoter, optionally wherein the neuron-specific promoter is an enolase promoter.

### BRIEF DESCRIPTION OF THE DRAWINGS

FIG. 1 shows characterization of the DM1 iPSC cell line SB1 as compared to a wildtype iPSC cell line by Southern blot analysis following digestion of genomic DNA with Bgl I to confirm the CTG repeat region. The SB1 cells contain a CTG repeat region of ˜300 CTG repeats (CTG repeat allele shown at ˜4.4 kB).

FIG. 2 shows a schematic for the two loss-of-signal (LOS) digital droplet PCR (ddPCR) assays (5′ LOS ddPCR assay and 3′ LOS ddPCR assay) used to detect deletion of the CTG repeat region in the 3′ UTR of the DMPK gene.

FIG. 3 shows the percent editing efficiency results for 61 SluCas9 gRNAs in wildtype iPSC-0052 cells.

FIG. 4 shows percent CTG repeat deletion for SluCas9 gRNAs. The percent repeat deletion data is shown for pairs and individual SluCas9 gRNAs from the 3′ LOS ddPCR assay.

FIG. 5 shows percent CTG repeat deletion in four SluCas9 gRNA pairs in DM1 iPSCs.

FIG. 6 shows percent CTG repeat deletion for three SluCas9 gRNAs pairs in DM1 cardiomyocyte s.

FIG. 7 shows the percent editing efficiency results for 59 SaCas9 gRNAs in wildtype iPSC-0052 cells.

FIG. 8 shows percent CTG repeat deletion for several single SaCas9 gRNAs as well as several pairs of SaCas9 gRNAs. The percent repeat deletion data is shown for pairs and individual SaCas9 gRNAs from the 3′ LOS ddPCR assay.

FIG. 9 shows percent CTG repeat deletion for four SaCas9 gRNAs pairs in DM1 iPSCs.

FIG. 10 shows percent CTG repeat deletion for two SaCas9 gRNAs pairs in DM1 cardiomyocyte s.

### DETAILED DESCRIPTION

Reference will now be made in detail to certain embodiments of the invention, examples of which are illustrated in the accompanying drawings. While the invention is described in conjunction with the illustrated embodiments, it will be understood that they are not intended to limit the invention to those embodiments. On the contrary, the invention is intended to cover all alternatives, modifications, and equivalents, which may be included within the invention as defined by the appended claims and included embodiments.

Before describing the present teachings in detail, it is to be understood that the disclosure is not limited to specific compositions or process steps, as such may vary. It should be noted that, as used in this specification and the appended claims, the singular form “a”, “an” and “the” include plural references unless the context clearly dictates otherwise. Thus, for example, reference to “a guide” includes a plurality of guides and reference to “a cell” includes a plurality of cells and the like.

Numeric ranges are inclusive of the numbers defining the range. Measured and measurable values are understood to be approximate, taking into account significant digits and the error associated with the measurement. Also, the use of “comprise”, “comprises”, “comprising”, “contain”, “contains”, “containing”, “include”, “includes”, and “including” are not intended to be limiting. It is to be understood that both the foregoing general description and detailed description are exemplary and explanatory only and are not restrictive of the teachings.

Unless specifically noted in the specification, embodiments in the specification that recite “comprising” various components are also contemplated as “consisting of” or “consisting essentially of” the recited components; embodiments in the specification that recite “consisting of” various components are also contemplated as “comprising” or “consisting essentially of” the recited components; and embodiments in the specification that recite “consisting essentially of” various components are also contemplated as “consisting of” or “comprising” the recited components (this interchangeability does not apply to the use of these terms in the claims). The term “or” is used in an inclusive sense, i.e., equivalent to “and/or,” unless the context clearly indicates otherwise.

The section headings used herein are for organizational purposes only and are not to be construed as limiting the desired subject matter in any way. In the event that any material incorporated by reference contradicts any term defined in this specification or any other express content of this specification, this specification controls. While the present teachings are described in conjunction with various embodiments, it is not intended that the present teachings be limited to such embodiments. On the contrary, the present teachings encompass various alternatives, modifications, and equivalents, as will be appreciated by those of skill in the art.

### I. Definitions

Unless stated otherwise, the following terms and phrases as used herein are intended to have the following meanings:

“Polynucleotide” and “nucleic acid” are used herein to refer to a multimeric compound comprising nucleosides or nucleoside analogs which have nitrogenous heterocyclic bases or base analogs linked together along a backbone, including conventional RNA, DNA, mixed RNA-DNA, and polymers that are analogs thereof. A nucleic acid “backbone” can be made up of a variety of linkages, including one or more of sugar-phosphodiester linkages, peptide-nucleic acid bonds (“peptide nucleic acids” or PNA; PCT No. WO 95/32305), phosphorothioate linkages, methylphosphonate linkages, or combinations thereof. Sugar moieties of a nucleic acid can be ribose, deoxyribose, or similar compounds with substitutions, e.g., 2′ methoxy or 2′ halide substitutions. Nitrogenous bases can be conventional bases (A, G, C, T, U), analogs thereof (e.g., modified uridines such as 5-methoxyuridine, pseudouridine, or N1-methylpseudouridine, or others); inosine; derivatives of purines or pyrimidines (e.g., N⁴-methyl deoxyguanosine, deaza- or aza-purines, deaza- or aza-pyrimidines, pyrimidine bases with substituent groups at the 5 or 6 position (e.g., 5-methylcytosine), purine bases with a substituent at the 2, 6, or 8 positions, 2-amino-6-methylaminopurine, O⁶-methylguanine, 4-thio-pyrimidines, 4-amino-pyrimidines, 4-dimethylhydrazine-pyrimidines, and O⁴-alkyl-pyrimidines; U.S. Pat. No. 5,378,825 and PCT No. WO 93/13121). For general discussion see The Biochemistry of the Nucleic Acids 5-36, Adams et al., ed., 11^{th} ed., 1992). Nucleic acids can include one or more “abasic” residues where the backbone includes no nitrogenous base for position(s) of the polymer (U.S. Pat. No. 5,585,481). A nucleic acid can comprise only conventional RNA or DNA sugars, bases and linkages, or can include both conventional components and substitutions (e.g., conventional bases with 2′ methoxy linkages, or polymers containing both conventional bases and one or more base analogs). Nucleic acid includes “locked nucleic acid” (LNA), an analogue containing one or more LNA nucleotide monomers with a bicyclic furanose unit locked in an RNA mimicking sugar conformation, which enhance hybridization affinity toward complementary RNA and DNA sequences (Vester and Wengel, 2004, Biochemistry 43(42):13233-41). RNA and DNA have different sugar moieties and can differ by the presence of uracil or analogs thereof in RNA and thymine or analogs thereof in DNA.

“Guide RNA”, “gRNA”, and simply “guide” are used herein interchangeably to refer to either a crRNA (also known as CRISPR RNA), or the combination of a crRNA and a trRNA (also known as tracrRNA). The crRNA and trRNA may be associated as a single RNA molecule (single guide RNA, sgRNA) or in two separate RNA molecules (dual guide RNA, dgRNA). “Guide RNA” or “gRNA” refers to each type. The trRNA may be a naturally-occurring sequence, or a trRNA sequence with modifications or variations compared to naturally-occurring sequences.

As used herein, a “spacer sequence,” sometimes also referred to herein and in the literature as a “guide sequence,” or “targeting sequence” refers to a sequence within a guide RNA that is complementary to a target sequence and functions to direct a guide RNA to a target sequence for cleavage by an RNA-targeted endonuclease. A guide sequence can be 24, 23, 22, 21, 20 or fewer base pairs in length, e.g., in the case of Staphylococcus lugdunensis (SluCas9), Staphylococcus aureus Cas9 (SaCas9), and related, e.g., modified versions, Cas9 homologs/orthologs. Shorter or longer sequences can also be used as guides, e.g., 15-, 16-, 17-, 18-, 19-, 20-, 21-, 22-, 23-, 24-, or 25-nucleotides in length. For example, in some embodiments, the guide sequence comprises at least 17, 18, 19, 20, 21, 22, 23, 24, or 25 contiguous nucleotides of a sequence selected from SEQ ID NOs: 1-51, 53, 55-56, 58-59, 61-62, 64, 66, or 70 (for SluCas9), and 200-259 (for SaCas9). In some embodiments, the guide sequence comprises a sequence selected from SEQ ID NOs: 1-51, 53, 55-56, 58-59, 61-62, 64, 66, 70, or 200-259. In some embodiments, the target sequence is in a gene or on a chromosome, for example, and is complementary to the guide sequence. In some embodiments, the degree of complementarity or identity between a guide sequence and its corresponding target sequence may be about 75%, 80%, 85%, 90%, 95%, 96%, 97%, 98%, 99%, or 100%. For example, in some embodiments, the guide sequence comprises a sequence with about 75%, 80%, 85%, 90%, 95%, 96%, 97%, 98%, 99%, or 100% identity to at least 17, 18, 19, 20, 21, 22, 23, 24, or 25 contiguous nucleotides of a sequence selected from SEQ ID NOs: 1-51, 53, 55-56, 58-59, 61-62, 64, 66, 70, or 200-259. In some embodiments, the guide sequence comprises a sequence with about 75%, 80%, 85%, 90%, 95%, 96%, 97%, 98%, 99%, or 100% identity to a sequence selected from SEQ ID NOs: 1-51, 53, 55-56, 58-59, 61-62, 64, 66, 70, or 200-259. In some embodiments, the guide sequence and the target region may be 100% complementary or identical. In other embodiments, the guide sequence and the target region may contain at least one mismatch. For example, the guide sequence and the target sequence may contain 1, 2, 3, or 4 mismatches, where the total length of the target sequence is at least 17, 18, 19, 20 or more base pairs. In some embodiments, the guide sequence and the target region may contain 1-4 mismatches where the guide sequence comprises at least 17, 18, 19, 20 or more nucleotides. In some embodiments, the guide sequence and the target region may contain 1, 2, 3, or 4 mismatches where the guide sequence comprises 20 nucleotides. In some embodiments, the guide sequence and the target region do not contain any mismatches.

In some embodiments, the guide sequence comprises a sequence selected from SEQ ID NOs: 1-51, 53, 55-56, 58-59, 61-62, 64, 66, 70, or 200-259, wherein if the 5′ terminal nucleotide is not guanine, one or more guanine (g) is added to the sequence at its 5′ end. The 5′ g or gg is required in some instances for transcription, for example, for expression by the RNA polymerase III-dependent U6 promoter or the T7 promoter. In some embodiments, a 5′ guanine is added to any one of the guide sequences or pairs of guide sequences disclosed herein.

Target sequences for RNA-targeted endonucleases include both the positive and negative strands of genomic DNA (i.e., the sequence given and the sequence's reverse compliment), as a nucleic acid substrate for an RNA-targeted endonuclease is a double stranded nucleic acid. Accordingly, where a guide sequence is said to be “complementary to a target sequence,” it is to be understood that the guide sequence may direct a guide RNA to bind to the reverse complement of a target sequence. Thus, in some embodiments, where the guide sequence binds the reverse complement of a target sequence, the guide sequence is identical to certain nucleotides of the target sequence (e.g., the target sequence not including the PAM) except for the substitution of U for T in the guide sequence.

As used herein, “ribonucleoprotein” (RNP) or “RNP complex” refers to a guide RNA together with an RNA-targeted endonuclease, such as a Cas nuclease, e.g., a Cas cleavase or Cas nickase (e.g., Cas9). In some embodiments, the guide RNA guides the RNA-targeted endonuclease such as Cas9 to a target sequence, and the guide RNA hybridizes with and the agent binds to the target sequence, which can be followed by cleaving or nicking.

As used herein, a first sequence is considered to “comprise a sequence with at least X % identity to” a second sequence if an alignment of the first sequence to the second sequence shows that X % or more of the positions of the second sequence in its entirety are matched by the first sequence. For example, the sequence AAGA comprises a sequence with 100% identity to the sequence AAG because an alignment would give 100% identity in that there are matches to all three positions of the second sequence. The differences between RNA and DNA (generally the exchange of uridine for thymidine or vice versa) and the presence of nucleoside analogs such as modified uridines do not contribute to differences in identity or complementarity among polynucleotides as long as the relevant nucleotides (such as thymidine, uridine, or modified uridine) have the same complement (e.g., adenosine for all of thymidine, uridine, or modified uridine; another example is cytosine and 5-methylcytosine, both of which have guanosine or modified guanosine as a complement). Thus, for example, the sequence 5′-AXG where X is any modified uridine, such as pseudouridine, N1-methyl pseudouridine, or 5-methoxyuridine, is considered 100% identical to AUG in that both are perfectly complementary to the same sequence (5′-CAU). Exemplary alignment algorithms are the Smith-Waterman and Needleman-Wunsch algorithms, which are well-known in the art. One skilled in the art will understand what choice of algorithm and parameter settings are appropriate for a given pair of sequences to be aligned; for sequences of generally similar length and expected identity >50% for amino acids or >75% for nucleotides, the Needleman-Wunsch algorithm with default settings of the Needleman-Wunsch algorithm interface provided by the EBI at the www.ebi.ac.uk web server is generally appropriate.

“mRNA” is used herein to refer to a polynucleotide that is not DNA and comprises an open reading frame that can be translated into a polypeptide (i.e., can serve as a substrate for translation by a ribosome and amino-acylated tRNAs). mRNA can comprise a phosphate-sugar backbone including ribose residues or analogs thereof, e.g., 2′-methoxy ribose residues. In some embodiments, the sugars of an mRNA phosphate-sugar backbone consist essentially of ribose residues, 2′-methoxy ribose residues, or a combination thereof.

Guide sequences useful in the guide RNA compositions and methods described herein are shown in Table 2 and throughout the application.

As used herein, a “SluCas9” encompasses wild type and modified versions of Cas9 from Staphylococcus lugdunensis, where the modified versions of SluCas9 maintain their main function to direct a guide RNA to a desired target location in DNA. In some embodiments, the SluCas9 protein comprises an amino acid sequence that is at least 80%, 85%, 90%, 91%, 92%, 93%, 94%, 95%, 96%, 97%, 98%, 99% or 100% identical to the sequence of SEQ ID NO: 712:

NQKFILGLDIGITSVGYGLIDYETKNIIDAGVRLFPEANVENNEGRRSK
RGSRRLKRRRIHRLERVKKLLEDYNLLDQSQIPQSTNPYAIRVKGLSEA
LSKDELVIALLHIAKRRGIHKIDVIDSNDDVGNELSTKEQLNKNSKLLK
DKFVCQIQLERMNEGQVRGEKNRFKTADIIKEIIQLLNVQKNFHQLDEN
FINKYIELVEMRREYFEGPGKGSPYGWEGDPKAWYETLMGHCTYFPDEL
RSVKYAYSADLFNALNDLNNLVIQRDGLSKLEYHEKYHIIENVFKQKKK
PTLKQIANEINVNPEDIKGYRITKSGKPQFTEFKLYHDLKSVLFDQSIL
ENEDVLDQIAEILTIYQDKDSIKSKLTELDILLNEEDKENIAQLTGYTG
THRLSLKCIRLVLEEQWYSSRNQMEIFTHLNIKPKKINLTAANKIPKAM
IDEFILSPVVKRTFGQAINLINKIIEKYGVPEDIIIELARENNSKDKQK
FINEMQKKNENTRKRINEIIGKYGNQNAKRLVEKIRLHDEQEGKCLYSL
ESIPLEDLLNNPNHYEVDHIIPRSVSFDNSYHNKVLVKQSENSKKSNLT
PYQYFNSGKSKLSYNQFKQHILNLSKSQDRISKKKKEYLLEERDINKFE
VQKEFINRNLVDTRYATRELTNYLKAYFSANNMNVKVKTINGSFTDYLR
KVWKFKKERNHGYKHHAEDALIIANADFLFKENKKLKAVNSVLEKPEIE
TKQLDIQVDSEDNYSEMFIIPKQVQDIKDFRNFKYSHRVDKKPNRQLIN
DTLYSTRKKDNSTYIVQTIKDIYAKDNTTLKKQFDKSPEKFLMYQHDPR
TFEKLEVIMKQYANEKNPLAKYHEETGEYLTKYSKKNNGPIVKSLKYIG
NKLGSHLDVTHQFKSSTKKLVKLSIKPYRFDVYLTDKGYKFITISYLDV
LKKDNYYYIPEQKYDKLKLGKAIDKNAKFIASFYKNDLIKLDGEIYKII
GVNSDTRNMIELDLPDIRYKEYCELNNIKGEPRIKKTIGKKVNSIEKLT
TDVLGNVFTNTQYTKPQLLFKRGN.

In some embodiments, the SluCas9 is a modified SluCas9 protein. Exemplary modified versions of SluCas9 include those described in:
(1) WO2020186059, filed 12 Mar. 2020, including, “M-SluCas9_X” wherein it is understood that M-SluCas9_X has a base sequence as shown in SEQ ID NO: 2 in that publication (SEQ ID NO: 800 herein), where any of the amino acid positions shown as “X” in SEQ ID NO: 2 in that publication (SEQ ID NO: 800 herein (see Table of Additional Sequences)) can be substituted as shown in Table 2 of that publication as follows: position E408 can be substituted with G, S, T, A, or D; position R414 can be substituted with G, S, T, A, D, or E; position E418 can be substituted with G, S, T, A, or D; position H422 can be substituted with H, A, G, S, T, D, or E; position C239 can be substituted with S or A; and position C401 can be substituted with S or A. In one embodiment, a SluCas9 is “M-SluCas9-R414A” as shown in SEQ ID NO: 7 of WO2020186059 (SEQ ID NO: 801 herein (see Table of Additional Sequences));
and
(2) WO2019118935, filed 14 Dec. 2017, including, the SluCas9 having the sequence of SEQ ID NO: 2 in that publication (SEQ ID NO: 802 herein (see Table of Additional Sequences)), or a variant thereof that is at least 75%, 80%, 85%, 90%, 95%, 99%, or 100% identical to SEQ ID NO: 2 in that publication (SEQ ID NO: 802 herein (see Table of Additional Sequences)) over its full length or at least 75%, 80%, 85%, 90%, 95%, 99%, or 100% identical over positions 789-1053 of SEQ ID NO: 2 in that publication (SEQ ID NO: 802 herein (see Table of Additional Sequences)), or a variant of such SluCas9 having a D 1 OA, H559A, and/or N582A substitution as compared to SEQ ID NO: 2 in that publication (SEQ ID NO: 802 herein (see Table of Additional Sequences)), as well as a SluCas9 produced from a codon optimized version of a polynucleotide sequence shown from position 61 to 3225 of SEQ ID NO: 3 (SEQ ID NO: 803 herein (see Table of Additional Sequences)), or SEQ ID NO: 44 (SEQ ID NO: 804 herein (see Table of Additional Sequences)), or SEQ ID NO: 45 (SEQ ID NO: 805 herein (see Table of Additional Sequences)) in that publication;
(3) WO2019183150, filed 19 Mar. 2019, including, the synthetic RNA-guided nuclease (sRGN) polypeptide described in paragraphs [009]-[0013] and the claims (e.g., SEQ ID NOs: 809-888 herein (see Table of Additional Sequences)), e.g., a sRGN comprising eight mini-domains, wherein at least 2 or 3 of the mini-domains are derived from parental SluCas9 endonucleases, and wherein at least 2 or 3 of the other mini-domains is derived from a different parental Cas9 endonuclease (including Staphylococcus pasteuri Cas9 (SEQ ID NO: 806 herein (see Table of Additional Sequences)), Staphylococcus microti Cas9 (SEQ ID NO: 807 herein (see Table of Additional Sequences)), and Staphylococcus hyicus Cas9 (SEQ ID NO: 808 (see Table of Additional Sequences));
(4) CN110577969, filed 8 Aug. 2019, including a chimeric SaCas9 protein comprising a SluCas9 PAM interacting domain;
(5) Hu et al., 2020, BioRxiv, https://doi.org/10.1101/2020.09.29.316661; including a SluCas9 protein comprising one or more of the following mutations, or combinations of mutations, as compared to SEQ ID NO: 712:
   (i) A mutation at any one of, or combination of, positions R246, N414, T420, or R655;
   (ii) A mutation at the position corresponding to position R246 of SEQ ID NO: 712 (e.g., R246A);
   (iii) A mutation at the position corresponding to position N414 of SEQ ID NO: 712 (e.g., N414A);
   (iv) A mutation at the position corresponding to position T420 of SEQ ID NO: 712 (e.g., T420A);
   (v) A mutation at the position corresponding to position R655 of SEQ ID NO: 712 (e.g., R655A);
   (vi) A combination of mutations at the positions corresponding to position R246 of SEQ ID NO: 712 (e.g., R246A), position N414 of SEQ ID NO: 712 (e.g., N414A), position T420 of SEQ ID NO: 712 (e.g., T420A), and position R655 of SEQ ID NO: 712 (e.g., R655A);
   (vii) A mutation at the position corresponding to position Q781 of SEQ ID NO: 712 (e.g., Q781K);
   (viii) A mutation at the position corresponding to position R1013 of SEQ ID NO: 712 (e.g., R1013H);
   (ix) A combination of mutations at the positions corresponding to position Q781 of SEQ ID NO: 712 (e.g., Q781K) and position R1013 of SEQ ID NO: 712 (e.g., R1013H);
each of which is incorporated by reference in its entirety.

As used herein, a “SaCas9” encompasses wild type and modified versions of Cas9 from Staphylococcus aureus, where the modified versions of SaCas9 maintain their main function to direct a guide RNA to a desired target location in DNA. A variant of SaCas9 comprises one or more amino acid changes as compared to SEQ ID NO: 711, including insertion, deletion, or substitution of one or more amino acids, or a chemical modification to one or more amino acids. In some embodiments, the nucleic acid encoding SaCas9 encodes an SaCas9 comprising an amino acid sequence that is at least 80%, 85%, 90%, 91%, 92%, 93%, 94%, 95%, 96%, 97%, 98%, 99% or 100% identical to the sequence of SEQ ID NO: 711:

KRNYILGLDIGITSVGYGIIDYETRDVIDAGVRLFKEANVENNEGRRSK
RGARRLKRRRRHRIQRVKKLLFDYNLLTDHSELSGINPYEARVKGLSQK
LSEEEFSAALLHLAKRRGVHNVNEVEEDTGNELSTKEQISRNSKALEEK
YVAELQLERLKKDGEVRGSINRFKTSDYVKEAKQLLKVQKAYHQLDQSF
IDTYIDLLETRRTYYEGPGEGSPFGWKDIKEWYEMLMGHCTYFPEELRS
VKYAYNADLYNALNDLNNLVITRDENEKLEYYEKFQIIENVFKQKKKPT
LKQIAKEILVNEEDIKGYRVTSTGKPEFTNLKVYHDIKDITARKEIIEN
AELLDQIAKILTIYQSSEDIQEELTNLNSELTQEEIEQISNLKGYTGTH
NLSLKAINLILDELWHTNDNQIAIFNRLKLVPKKVDLSQQKEIPTTLVD
DFILSPVVKRSFIQSIKVINAIIKKYGLPNDIIIELAREKNSKDAQKMI
NEMQKRNRQTNERIEEIIRTTGKENAKYLIEKIKLHDMQEGKCLYSLEA
IPLEDLLNNPFNYEVDHIIPRSVSFDNSFNNKVLVKQEENSKKGNRTPF
QYLSSSDSKISYETFKKHILNLAKGKGRISKTKKEYLLEERDINRFSVQ
KDFINRNLVDTRYATRGLMNLLRSYFRVNNLDVKVKSINGGFTSFLRRK
WKFKKERNKGYKHHAEDALIIANADFIFKEWKKLDKAKKVMENQMFEEK
QAESMPEIETEQEYKEIFITPHQIKHIKDFKDYKYSHRVDKKPNRELIN
DTLYSTRKDDKGNTLIVNNLNGLYDKDNDKLKKLINKSPEKLLMYHHDP
QTYQKLKLIMEQYGDEKNPLYKYYEETGNYLTKYSKKDNGPVIKKIKYY
GNKLNAHLDITDDYPNSRNKVVKLSLKPYRFDVYLDNGVYKFVTVKNLD
VIKKENYYEVNSKCYEEAKKLKKISNQAEFIASFYNNDLIKINGELYRV
IGVNNDLLNRIEVNMIDITYREYLENMNDKRPPRIIKTIASKTQSIKKY
STDILGNLYEVKSKKHPQIIKKG.

As used herein, a “target sequence” refers to a sequence of nucleic acid in a target gene that has complementarity to the guide sequence of the gRNA. The interaction of the target sequence and the guide sequence directs an RNA-targeted endonuclease to bind, and potentially nick or cleave (depending on the activity of the agent), within the target sequence.

As used herein, “treatment” refers to any administration or application of a therapeutic for disease or disorder in a subject, and includes inhibiting the disease or development of the disease (which may occur before or after the disease is formally diagnosed, e.g., in cases where a subject has a genotype that has the potential or is likely to result in development of the disease), arresting its development, relieving one or more symptoms of the disease, curing the disease, or preventing reoccurrence of one or more symptoms of the disease. For example, treatment of DM1 may comprise alleviating symptoms of DM1.

As used herein, “ameliorating” refers to any beneficial effect on a phenotype or symptom, such as reducing its severity, slowing, or delaying its development, arresting its development, or partially or completely reversing or eliminating it. In the case of quantitative phenotypes such as expression levels, ameliorating encompasses changing the expression level so that it is closer to the expression level seen in healthy or unaffected cells or individuals.

As used herein, “excision” of a sequence means any process that results in removal of the sequence from nucleic acid (e.g., DNA, such as gDNA) in which it originally occurred, including but not limited to processes comprising two double strand cleavage events or two or more nicking events followed by any repair process that does not include the sequence in the repair product, which may comprise one or more of ligation of distal ends, resection, or secondary structure formation by at least part of the region being excised.

As used herein, an “expanded amino acid repeat” refers to a segment of a given amino acid (e.g., one of glutamine, alanine, etc.) in DMPK that contains more instances of the amino acid than normally appears in wild-type versions of DMPK. In Table 1, the normal range indicates the range of instances of the amino acid than normally appears in wild-type versions of DMPK.

The term “about” or “approximately” means an acceptable error for a particular value as determined by one of ordinary skill in the art, which depends in part on how the value is measured or determined.

### II. Overview of Repetitive DNA Excision in DM1

Methods and compositions provided herein can be used to excise trinucleotide repeats or self-complementary sequences to ameliorate genotypes associated with DM1. Table 1 provides information regarding the trinucleotide repeats associated with DM1.

TABLE 1

| | Genetic Locus; | | | |
|---|---|---|---|---|
| | inheritance | | Normal repeat | Pathological repeat |
| Disorder | pattern | TNR | copy number | copy number |
| DM1/myotonic | DMPK 3′ UTR | CTG | 5-34 | 50-5000 in most |
| dystrophy | Autosomal | | (35-49 = | cells; may be higher |
| type 1 | dominant | | premutation, | in muscle cells |
| | | | children at risk) | |

### III. Methods and Uses for Treating DM1

This disclosure provides methods and uses for treating DM1 comprising administering one or more guide RNAs (gRNAs) or one or more nucleic acids encoding said gRNAs to a subject in need of treatment. In some embodiments, the one or more gRNA, or nucleic acid encoding the one or more gRNA, is administered in combination (e.g., at or near the same time as) a SluCas9, or a nucleic acid encoding a SluCas9, or a SaCas9, or a nucleic acid encoding an SaCas9. The one or more gRNA comprises a spacer sequence of Table 2. In some embodiments, a vector is provided comprising a nucleic acid encoding one or more gRNA comprising a spacer sequence of Table 2 and a nucleic acid encoding a SluCas9 (for SEQ ID NOs: 1-51, 53, 55-56, 58-59, 61-62, 64, 66, or 70) or SaCas9 (for SEQ ID NOs: 200-259). In some embodiments, one vector is administered, wherein the vector comprises a nucleic acid encoding the one or more gRNA and a nucleic acid encoding a SluCas9 or SaCas9. In some embodiments, two or more vectors are administered, where one vector comprises a nucleic acid encoding one or more gRNA and does not comprise an endonuclease such as SluCas9 or SaCas9, and the other vector comprises a nucleic acid encoding a SluCas9 or SaCas9 and optionally one or more gRNAs, wherein the gRNAs may be the same or different than the gRNAs on the other vector not encoding the SluCas9 or SaCas9. In some embodiments, two or more vectors are administered, where one vector comprises a nucleic acid encoding one or more gRNA and a nucleic acid encoding a SluCas9 or SaCas9, and the other vector may comprise one or more nucleic acids encoding one or more gRNAs and not a SluCas9 or SaCas9. In some embodiments, two or more vectors are administered, where each vector comprises a nucleic acid encoding one or more gRNA and a nucleic acid encoding a SluCas9 or SaCas9. In some embodiments, any of the compositions described herein is administered to a subject in need thereof for use in treating DM1. In some embodiments, the composition administered comprises one or more guide RNAs (gRNAs) comprising any one or more of the guide sequences of Table 2, or a vector encoding any one or more of the gRNAs.

In some embodiments, methods of excising trinucleotide repeats in the DMPK gene are provided comprising administering two or more guide RNAs (gRNAs), each gRNA comprising any one of the spacer sequences of Table 2, or administering a vector encoding two or more gRNAs. In some embodiments, two or more gRNAs described herein (e.g., a pair of gRNAs) or a vector encoding the gRNAs are delivered to a cell in combination (e.g., at or near the same time) with SluCas9 or a nucleic acid encoding the SluCas9 (for SEQ ID NOs: 1-51, 53, 55-56, 58-59, 61-62, 64, 66, or 70) or SaCas9 or a nucleic acid encoding SaCas9 (for SEQ ID NOs: 200-259). Exemplary gRNAs, vectors, and SluCas9s for treating DM1 are described herein.

In some embodiments, a method of treating DM1 is provided, the method comprising delivering to a cell a guide RNA comprising a spacer sequence selected from any one of SEQ ID NOs: 1-51, 53, 55-56, 58-59, 61-62, 64, 66, or 70, or a nucleic acid encoding the guide RNA, and optionally a Staphylococcus lugdunensis (SluCas9) or a nucleic acid encoding a SluCas9. In some embodiments, a method of treating DM1 is provided, the method comprising delivering to a cell a guide RNA comprising a spacer sequence that is at least 20 contiguous nucleotides of any one of SEQ ID NOs: 1-51, 53, 55-56, 58-59, 61-62, 64, 66, or 70 and optionally a Staphylococcus lugdunensis (SluCas9) or a nucleic acid encoding a SluCas9. In some embodiments, a method of treating DM1 is provided, the method comprising delivering to a cell a guide RNA comprising a spacer sequence that is at least 90% or 100% identical to any one of SEQ ID NOs: 1-51, 53, 55-56, 58-59, 61-62, 64, 66, or and optionally a Staphylococcus lugdunensis (SluCas9) or a nucleic acid encoding a SluCas9.

Also provided is a method of treating a disease or disorder characterized by a trinucleotide repeat (TNR) in the 3′ UTR of the DMPK gene, the method comprising delivering to a cell that comprises a TNR in the 3′ UTR of the DMPK gene
i) a guide RNA comprising a spacer having a sequence of any one of SEQ ID NOs: 3, 5, 6, 9, 16, 21, 22, 25, 26, 30, 36, 38, 39, 40, 46, 51, 53, 55, 56, 58, 59, 61, 62, 64, 66, and 70 or a nucleic acid encoding the guide RNA; ii) a guide RNA comprising a spacer having a sequence of any one of SEQ ID NOs: 1, 2, 4, 7, 8, 10, 11, 12, 13, 14, 15, 17, 18, 19, 20, 23, 24, 27, 28, 29, 31, 32, 33, 34, 35, 37, 41, 42, 43, 44, 45, 47, 48, 49, and 50 or a nucleic acid encoding the guide RNA; and iii) SluCas9 or a nucleic acid encoding the SluCas9.

Also provided is a method of excising a trinucleotide repeat (TNR) in the 3′ UTR of the DMPK gene comprising delivering to a cell that comprises the TNR in the 3′ UTR of the DMPK a pair of guide RNAs comprising a pair of spacer sequences, or one or more vectors encoding the pair of guide RNAs, wherein the pair of spacer sequences comprise: i) a first spacer sequence selected from SEQ ID NOs: 3, 5, 6, 9, 16, 21, 22, 25, 26, 30, 36, 38, 39, 40, 46, 51, 53, 55, 56, 58, 59, 61, 62, 64, 66, and 70, and a second spacer sequence selected from SEQ ID NOs: 1, 2, 4, 7, 8, 10, 11, 12, 13, 14, 15, 17, 18, 19, 20, 23, 24, 27, 28, 29, 31, 32, 33, 34, 35, 37, 41, 42, 43, 44, 45, 47, 48, 49, and 50; and ii) a SluCas9 or a nucleic acid encoding the SluCas9, wherein at least one TNR is excised.

Also provided is a method of treating DM1, the method comprising administering to a subject having DM1:
i) a guide RNA comprising a spacer having a sequence of any one of SEQ ID NOs: 3, 5, 6, 9, 16, 21, 22, 25, 26, 30, 36, 38, 39, 40, 46, 51, 53, 55, 56, 58, 59, 61, 62, 64, 66, and 70 or a nucleic acid encoding the guide RNA;
ii) a guide RNA comprising a spacer having a sequence of any one of SEQ ID NOs: 1, 2, 4, 7, 8, 10, 11, 12, 13, 14, 15, 17, 18, 19, 20, 23, 24, 27, 28, 29, 31, 32, 33, 34, 35, 37, 41, 42, 43, 44, 47, 48, 49, and 50 or a nucleic acid encoding the guide RNA; and
iii) SluCas9 or a nucleic acid encoding the SluCas9.

In some embodiments of methods described herein, a pair of guide RNAs that comprise a first and second spacer that deliver the SluCas9 to or near the TNR, or one or more vectors encoding the pair of guide RNAs, are provided, administered, or delivered to a cell. For example, where the TNR is in the 3′ UTR of the DMPK gene, the first and second spacer sequences may have the sequences of any one of the following pairs of SEQ ID NOs: 5 and 7, 5 and 10, 5 and 19, 5 and 41, 5 and 47, 21 and 7, 21 and 19, 21 and 41, 21 and 47, 46 and 7, 46 and 10, 46 and 19, 46 and 41, 46 and 47, 55 and 7, 55 and 19, 55 and 41, 55 and 47, 59 and 7, 59 and 19, 59 and 41, 59 and 47, 61 and 7, 61 and 10, 61 and 19, 61 and 41, 61 and 47, 64 and 7, 64 and 19, 64 and 41, or 64 and 47.

In some embodiments, methods of treating DM1, excising a trinucleotide repeat (TNR) in the 3′ UTR of the DMPK gene, or treating a disease or disorder characterized by a trinucleotide repeat (TNR) in the 3′ UTR of the DMPK gene are provided comprising administering to a subject in need:
a. a pair of guide RNAs comprising a pair of spacer sequences, or one or more vectors encoding the pair of guide RNAs, wherein the pair of spacer sequences comprises SEQ ID NO: 5 and SEQ ID NO: 10;
b. a pair of guide RNAs comprising a pair of spacer sequences, or one or more vectors encoding the pair of guide RNAs, wherein the pair of spacer sequences comprises SEQ ID NO: 46 and SEQ ID NO: 10;
c. a pair of guide RNAs comprising a pair of spacer sequences, or one or more vectors encoding the pair of guide RNAs, wherein the pair of spacer sequences comprises SEQ ID NO: 61 and SEQ ID NO: 10; or
d. a pair of guide RNAs comprising a pair of spacer sequences, or one or more vectors encoding the pair of guide RNAs, wherein the pair of spacer sequences comprises SEQ ID NO: 64 and SEQ ID NO: 47; and SluCas9 or a nucleic acid encoding the SluCas9.

Any of the foregoing methods and any other method described herein may be combined to the extent feasible with any of the additional features described herein, including in the sections above, the following discussion, the examples, and the claims.

In some embodiments, at least a pair of gRNAs are provided which direct a SluCas9 to a pair of sites flanking (i.e., on opposite sides of) a TNR. For example, the pair of sites flanking a TNR may each be within 10, 20, 30, 40, or 50 nucleotides of the TNR but on opposite sides thereof.

In some embodiments, trinucleotide repeats are excised from a locus or gene associated with DM1.

The number of repeats that is excised may be at least 1, 2, 3, 4, 5, 6, 7, 8, 9, 10, 15, 20, 30, 40, 50, 60, 70, 80, 90, 100, 150, 200, 300, 400, 500, 600, 700, 800, 900, 1000, 1500, 2000, 3000, 4000, 5000, 6000, 7000, 8000, 9000, or 10,000, or in a range bounded by any two of the foregoing numbers, inclusive, or in any of the ranges listed herein. In some embodiments, the number of repeats that is excised is in a range listed in Table 1, e.g., as a pathological, premutation, at-risk, or intermediate range.

In some embodiments, excision of a repeat region ameliorates at least one phenotype or symptom associated with the repeat region. This may include ameliorating aberrant expression of the DMPK gene encompassing or near the repeat region, or ameliorating aberrant activity of a gene product (noncoding RNA, mRNA, or polypeptide) encoded by the DMPK gene encompassing the repeat region.

For example, excision of the TNRs may ameliorate one or more phenotypes associated with an expanded-repeat DMPK gene, e.g., one or more of increasing myotonic dystrophy protein kinase activity; increasing phosphorylation of phospholemman, dihydropyridine receptor, myogenin, L-type calcium channel beta subunit, and/or myosin phosphatase targeting subunit; increasing inhibition of myosin phosphatase; and/or ameliorating muscle loss, muscle weakness, hypersomnia, one or more executive function deficiencies, insulin resistance, cataract formation, balding, or male infertility or low fertility.

In some embodiments, any one or more of the gRNAs, pairs of gRNAs, vectors, compositions, or pharmaceutical formulations described herein is for use in a method disclosed herein or in preparing a medicament for treating or preventing DM1 in a subject. In some embodiments, treatment and/or prevention is accomplished with a single dose, e.g., one-time treatment, of medicament/composition.

In some embodiments, a method of treating or preventing DM1 in subject comprising administering a pair of gRNAs, vectors, compositions, or pharmaceutical formulations described herein is provided. In some embodiments, the gRNAs, vectors, compositions, or pharmaceutical formulations described herein are administered as a single dose, e.g., at one time. In some embodiments, the single dose achieves durable treatment and/or prevention. In some embodiments, the method achieves durable treatment and/or prevention. Durable treatment and/or prevention, as used herein, includes treatment and/or prevention that extends at least i) 3, 4, 5, 6, 7, 8, 9, 10, 11, 12, 13, 14, or 15 weeks; ii) 1, 2, 3, 4, 5, 6, 7, 8, 9, 10, 11, 12, 18, 24, 30, or 36 months; or iii) 1, 2, 3, 4, 5, 6, 7, 8, 9, or 10 years. In some embodiments, a single dose of the gRNAs, vectors, compositions, or pharmaceutical formulations described herein is sufficient to treat and/or prevent any of the indications described herein for the duration of the subject's life.

In some embodiments, a method of excising a TNR of DMPK is provided comprising administering a composition comprising a pair of guide RNAs comprising a pair of spacer sequences, or one or more vectors encoding the pair of guide RNAs, wherein the pair of spacer sequences comprise: a first spacer sequence selected from SEQ ID NOs: 3, 5, 6, 9, 16, 21, 22, 25, 26, 30, 36, 38, 39, 40, 46, 51, 53, 55, 56, 58, 59, 61, 62, 64, 66, and 70, and a second spacer sequence selected from SEQ ID NOs: 1, 2, 4, 7, 8, 10, 11, 12, 13, 14, 15, 17, 18, 19, 20, 23, 24, 27, 28, 29, 31, 32, 33, 34, 35, 37, 41, 42, 43, 44, 45, 47, 48, 49, and 50 together with SluCas9 or an mRNA or vector encoding SluCas9. In some embodiments, the pair of spacer sequences comprises SEQ ID NO: 5 and SEQ ID NO: 10, SEQ ID NO: 46 and SEQ ID NO: 10, SEQ ID NO: 61 and SEQ ID NO: 10, or SEQ ID NO: 64 and SEQ ID NO: 47.

In some embodiments, a pair of gRNAs comprising a first spacer sequence selected from SEQ ID NOs: 3, 5, 6, 9, 16, 21, 22, 25, 26, 30, 36, 38, 39, 40, 46, 51, 53, 55, 56, 58, 59, 61, 62, 64, 66, and 70, and a second spacer sequence selected from SEQ ID NOs: 1, 2, 4, 7, 8, 10, 11, 12, 13, 14, 17, 18, 19, 20, 23, 24, 27, 28, 29, 31, 32, 33, 34, 35, 37, 41, 42, 43, 44, 45, 47, 48, 49, and 50 are administered to excise a TNR in DMPK and SluCas9 or an mRNA or vector encoding SluCas9. In some embodiments, the pair of spacer sequences comprises SEQ ID NO: 5 and SEQ ID NO: 10, SEQ ID NO: 46 and SEQ ID NO: 10, SEQ ID NO: 61 and SEQ ID NO: 10, or SEQ ID NO: 64 and SEQ ID NO: 47.

In some embodiments, a method of treating DM1 is provided comprising administering a composition comprising a pair of guide RNAs comprising a first spacer sequence selected from SEQ ID NOs: 3, 5, 6, 9, 16, 21, 22, 25, 26, 30, 36, 38, 39, 40, 46, 51, 53, 55, 56, 58, 59, 61, 62, 64, 66, and and a second spacer sequence selected from SEQ ID NOs: 1, 2, 4, 7, 8, 10, 11, 12, 13, 14, 15, 17, 18, 19, 20, 23, 24, 27, 28, 29, 31, 32, 33, 34, 35, 37, 41, 42, 43, 44, 45, 47, 48, 49, and 50 and SluCas9 or an mRNA or vector encoding SluCas9. In some embodiments, the pair of spacer sequences comprises SEQ ID NO: 5 and SEQ ID NO: 10, SEQ ID NO: 46 and SEQ ID NO: 10, SEQ ID NO: 61 and SEQ ID NO: 10, or SEQ ID NO: 64 and SEQ ID NO: 47.

In some embodiments, a method of decreasing or eliminating production of an mRNA comprising an expanded trinucleotide repeat in the 3′ UTR of the DMPK gene is provided comprising administering a pair of guide RNAs comprising a first spacer sequence selected from SEQ ID NOs: 3, 6, 9, 16, 21, 22, 25, 26, 30, 36, 38, 39, 40, 46, 51, 53, 55, 56, 58, 59, 61, 62, 64, 66, and 70, and a second spacer sequence selected from SEQ ID NOs: 1, 2, 4, 7, 8, 10, 11, 12, 13, 14, 15, 17, 18, 19, 20, 23, 24, 27, 28, 29, 31, 32, 33, 34, 35, 37, 41, 42, 43, 44, 45, 47, 48, 49, and 50 and SluCas9 or an mRNA or vector encoding SluCas9. In some embodiments, the pair of spacer sequences comprises SEQ ID NO: 5 and SEQ ID NO: 10, SEQ ID NO: 46 and SEQ ID NO: 10, SEQ ID NO: 61 and SEQ ID NO: 10, or SEQ ID NO: 64 and SEQ ID NO: 47.

In some embodiments, a method of decreasing or eliminating production of a protein comprising an expanded amino acid repeat in DMPK is provided comprising administering two or more guide RNAs comprising a first spacer sequence selected from SEQ ID NOs: 3, 5, 6, 9, 16, 21, 22, 25, 26, 30, 36, 38, 39, 40, 46, 51, 53, 55, 56, 58, 59, 61, 62, 64, 66, and 70, and one or more second spacer sequence selected from SEQ ID NOs: 1, 2, 4, 7, 8, 10, 11, 12, 13, 14, 15, 17, 18, 19, 20, 23, 24, 27, 28, 29, 31, 32, 33, 34, 35, 37, 41, 42, 43, 44, 45, 47, 48, 49, and 50 and SluCas9 or an mRNA or vector encoding SluCas9. In some embodiments, the pair of spacer sequences comprises SEQ ID NO: 5 and SEQ ID NO: 10, SEQ ID NO: 46 and SEQ ID NO: 10, SEQ ID NO: 61 and SEQ ID NO: 10, or SEQ ID NO: 64 and SEQ ID NO: 47.

In some embodiments, gRNAs comprising any two of the guide sequences of (i) SEQ ID NOs: 1-51, 53, 55-56, 58-59, 61-62, 64, 66, or 70 are administered to reduce expression of a polypeptide comprising an expanded amino acid repeat in DMPK together with SluCas9 or an mRNA or vector encoding SluCas9.

In some embodiments, the pair of gRNAs comprise two of the guide sequences of Table 2 together with SluCas9 (for SEQ ID NOs: 1-51, 53, 55-56, 58-59, 61-62, 64, 66, or 70) or SaCas9 (for SEQ ID NOs: 200-259) to induce DSBs, and microhomology-mediated end joining (MMEJ) during repair leads to a mutation in the targeted gene. In some embodiments, MMEJ leads to excision of trinucleotide repeats.

In some embodiments, methods of excising trinucleotide repeats in the DMPK gene are provided comprising administering two or more SaCas9-specific guide RNAs (gRNAs), each gRNA comprising any one of the spacer sequences of SEQ ID NO: 200-259 in Table 2, or administering a vector encoding two or more gRNAs. In some embodiments, two or more gRNAs described herein (e.g., a pair of gRNAs) or a vector encoding the gRNAs are delivered to a cell in combination (e.g., at or near the same time) with SaCas9 or a nucleic acid encoding the SaCas9. Exemplary gRNAs, vectors, and SaCas9 for treating DM1 are described herein.

In some embodiments, a method of treating DM1 is provided, the method comprising delivering to a cell a guide RNA comprising a spacer sequence selected from any one of SEQ ID NOs: 200-259, or a nucleic acid encoding the guide RNA, and optionally a Staphylococcus aureus Cas9 (SaCas9) or a nucleic acid encoding a SaCas9. In some embodiments, a method of treating DM1 is provided, the method comprising delivering to a cell a guide RNA comprising a spacer sequence that is at least 20 contiguous nucleotides of any one of SEQ ID NOs: 200-259 and optionally a Staphylococcus aureus Cas9 (SaCas9) or a nucleic acid encoding a SaCas9. In some embodiments, a method of treating DM1 is provided, the method comprising delivering to a cell a guide RNA comprising a spacer sequence that is at least 90% identical to any one of SEQ ID NOs: 200-259 and optionally a Staphylococcus aureus Cas9 (SaCas9) or a nucleic acid encoding a SaCas9.

Also provided is a method of treating a disease or disorder characterized by a trinucleotide repeat (TNR) in the 3′ UTR of the DMPK gene, the method comprising delivering to a cell that comprises a TNR in the 3′ UTR of the DMPK gene
i) a guide RNA comprising a spacer having a sequence of any one of SEQ ID NOs: 201-203, 211, 215, 220, 225, 231, 235, 238, and 240-259 or a nucleic acid encoding the guide RNA; ii) a guide RNA comprising a spacer having a sequence of any one of SEQ ID NOs: 200, 204-210, 212-214, 216-219, 221-224, 226-230, 232-234, 236-237, and 239 or a nucleic acid encoding the guide RNA; and iii) SaCas9 or a nucleic acid encoding the SaCas9. In some embodiments, a pair of gRNAs is delivered to a cell that comprises a TNR in the 3′ UTR of the DMPK gene, wherein the pair comprises any one of the SEQ ID NO: 202 and SEQ ID NO: 218, SEQ ID NO: 202 and SEQ ID NO: 213, SEQ ID NO: 201 and SEQ ID NO: 224, or SEQ ID NO: 201 and SEQ ID NO: 206.

Also provided is a method of excising a trinucleotide repeat (TNR) in the 3′ UTR of the DMPK gene comprising delivering to a cell that comprises the TNR in the 3′ UTR of the DMPK a pair of guide RNAs comprising a pair of spacer sequences, or one or more vectors encoding the pair of guide RNAs, wherein the pair of spacer sequences comprise: i) a first spacer sequence selected from SEQ ID NOs: 200, 204-210, 212-214, 216-219, 221-224, 226-230, 232-234, 236-237, and 239, and a second spacer sequence selected from SEQ ID NOs: 200, 204-210, 212-214, 216-219, 221-224, 226-230, 232-234, 236-237, and 239; and ii) a SaCas9 or a nucleic acid encoding the SaCas9, wherein at least one TNR is excised. In some embodiments, a pair of gRNAs is delivered to a cell, wherein the pair comprises any one of the SEQ ID NO: 202 and SEQ ID NO: 218, SEQ ID NO: 202 and SEQ ID NO: 213, SEQ ID NO: 201 and SEQ ID NO: 224, or SEQ ID NO: 201 and SEQ ID NO: 206.

In some embodiments of methods described herein, a pair of guide RNAs that comprise a first and second spacer that deliver the SaCas9 to or near the TNR, or one or more vectors encoding the pair of guide RNAs, are provided or delivered to a cell. For example, where the TNR is in the 3′ UTR of the DMPK gene, the first and second spacer sequences may have the sequences of any one of the following pairs of SEQ ID NOs: 202 and 218, 201 and 224, 202 and 213, or 202 and 206.

Any of the foregoing methods and any other method described herein may be combined to the extent feasible with any of the additional features described herein, including in the sections above, the following discussion, the examples, and the claims.

In some embodiments, at least a pair of gRNAs are provided which direct a SaCas9 to a pair of sites flanking (i.e., on opposite sides of) a TNR. For example, the pair of sites flanking a TNR may each be within 10, 20, 30, 40, or 50 nucleotides of the TNR but on opposite sides thereof.

In some embodiments, a method of excising a TNR of DMPK is provided comprising administering a composition comprising a pair of guide RNAs comprising a pair of spacer sequences, or one or more vectors encoding the pair of guide RNAs, wherein the pair of spacer sequences comprise: a first spacer sequence selected from SEQ ID NOs: 201-203, 211, 215, 220, 225, 231, 235, 238, and 240-259, and a second spacer sequence selected from SEQ ID NOs: 200, 204-210, 212-214, 216-219, 221-224, 226-230, 232-234, 236-237, and 239.

In some embodiments, a pair of gRNAs comprising a first spacer sequence selected from SEQ ID NOs: 201-203, 211, 215, 220, 225, 231, 235, 238, and 240-259, and a second spacer sequence selected from SEQ ID NOs: 200, 204-210, 212-214, 216-219, 221-224, 226-230, 232-234, 236-237, and 239 are administered to excise a TNR in DMPK. The guide RNAs may be administered together with SaCas9 or an mRNA or vector encoding SaCas9.

In some embodiments, a method of treating DM1 is provided comprising administering a composition comprising a pair of guide RNAs comprising a first spacer sequence selected from SEQ ID NOs: 201-203, 211, 215, 220, 225, 231, 235, 238, and 240-259, and a second spacer sequence selected from SEQ ID NOs: 200, 204-210, 212-214, 216-219, 221-224, 226-230, 232-234, 236-237, and 239; and SaCas9 or an mRNA or vector encoding SaCas9. In some embodiments, the pair of gRNAs comprises any one of the SEQ ID NO: 202 and SEQ ID NO: 218, SEQ ID NO: 202 and SEQ ID NO: 213, SEQ ID NO: 201 and SEQ ID NO: 224, or SEQ ID NO: 201 and SEQ ID NO: 206.

In some embodiments, a method of decreasing or eliminating production of an mRNA comprising an expanded trinucleotide repeat in the 3′ UTR of the DMPK gene is provided comprising administering a pair of guide RNAs comprising a first spacer sequence selected from SEQ ID NOs: 201-203, 211, 215, 220, 225, 231, 235, 238, and 240-259, and a second spacer sequence selected from SEQ ID NOs: 200, 204-210, 212-214, 216-219, 221-224, 226-230, 232-234, 236-237, and 239; and SaCas9 or an mRNA or vector encoding SaCas9. In some embodiments, the pair of gRNAs comprises any one of the SEQ ID NO: 202 and SEQ ID NO: 218, SEQ ID NO: 202 and SEQ ID NO: 213, SEQ ID NO: 201 and SEQ ID NO: 224, or SEQ ID NO: 201 and SEQ ID NO: 206.

In some embodiments, a method of decreasing or eliminating production of a protein comprising an expanded amino acid repeat in DMPK is provided comprising administering two or more guide RNAs comprising a first spacer sequence selected from SEQ ID NOs: 201-203, 211, 215, 220, 225, 231, 235, 238, and 240-259, and one or more second spacer sequence selected from SEQ ID NOs: 200, 204-210, 212-214, 216-219, 221-224, 226-230, 232-234, 236-237, and 239; and SaCas9 or an mRNA or vector encoding SaCas9. In some embodiments, the pair of gRNAs comprises any one of the SEQ ID NO: 202 and SEQ ID NO: 218, SEQ ID NO: 202 and SEQ ID NO: 213, SEQ ID NO: 201 and SEQ ID NO: 224, or SEQ ID NO: 201 and SEQ ID NO: 206.

In some embodiments, gRNAs comprising any two of the guide sequences of (i) SEQ ID NOs: 200-259 are administered to reduce expression of a polypeptide comprising an expanded amino acid repeat in DMPK. The gRNAs may be administered together with SaCas9 or an mRNA or vector encoding SaCas9.

In some embodiments, the pair of gRNAs comprise two of the guide sequences of SEQ ID NO: 200-259 in Table 2 together with SaCas9 to induce DSBs, and microhomology-mediated end joining (MMEJ) during repair leads to a mutation in the targeted gene. In some embodiments, MMEJ leads to excision of trinucleotide repeats.

In some embodiments, the subject is mammalian. In some embodiments, the subject is human. In some embodiments, the subject is cow, pig, monkey, sheep, dog, cat, fish, or poultry.

In some embodiments, the use of a pair of guide RNAs comprising any two of the guide sequences in Table 2 (e.g., in a composition provided herein) is provided for the preparation of a medicament for treating a human subject having DM1.

For treatment of a subject (e.g., a human), any of the compositions disclosed herein may be administered in a manner compatible with the dosage formulation and in such amount as is therapeutically effective. The compositions may be readily administered in a variety of dosage forms, such as injectable solutions. For parenteral administration in an aqueous solution, for example, the solution will generally be suitably buffered and the liquid diluent first rendered isotonic with, for example, sufficient saline or glucose. Such aqueous solutions may be used, for example, for intravenous, intramuscular, subcutaneous, and/or intraperitoneal administration. In some embodiments, the guide RNAs, compositions, and formulations are administered intravenously. In some embodiments, the guide RNAs, compositions, and formulations are administered intramuscularly. In some embodiments, the guide RNAs, compositions, and formulations are administered intracranially. In some embodiments, the guide RNAs, compositions, and formulations are administered to cells ex vivo.

In some embodiments, a single administration of a composition comprising a pair of guide RNAs provided herein is sufficient to excise TNRs. In other embodiments, more than one administration of a composition comprising a pair of guide RNAs provided herein may be beneficial to maximize therapeutic effects.

### Combination Therapy

In some embodiments, the invention comprises combination therapies comprising any of the methods described herein (e.g., two or more gRNAs comprising any two or more of the guide sequences disclosed in Table 2 (e.g., in a composition provided herein)) together with an additional therapy suitable for ameliorating DM1 and/or one or more symptoms thereof. Suitable additional therapies for use in ameliorating DM1, and/or one or more symptoms thereof are known in the art.

Delivery of gRNA Compositions

The compositions may be administered via any suitable approach for delivering gRNAs and compositions described herein. Exemplary delivery approaches include vectors, such as viral vectors; lipid nanoparticles; transfection; and electroporation. In some embodiments, vectors or LNPs associated with the gRNAs disclosed herein are for use in preparing a medicament for treating DM1.

Where a vector is used, it may be a viral vector, such as a non-integrating viral vector. In some embodiments, the viral vector is an adeno-associated virus vector, a lentiviral vector, an integrase-deficient lentiviral vector, an adenoviral vector, a vaccinia viral vector, an alphaviral vector, or a herpes simplex viral vector. In some embodiments, the viral vector is an adeno-associated virus (AAV) vector. In some embodiments, the AAV vector is an AAV1, AAV2, AAV3, AAV4, AAV5, AAV6, AAV7, AAV8, AAVrh10 (see, e.g., SEQ ID NO: 81 of U.S. Pat. No. 9,790,472, which is incorporated by reference herein in its entirety), AAVrh74 (see, e.g., SEQ ID NO: 1 of US 2015/0111955, which is incorporated by reference herein in its entirety), or AAV9 vector, wherein the number following AAV indicates the AAV serotype. In some embodiments, the AAV vector is a single-stranded AAV (ssAAV). In some embodiments, the AAV vector is a double-stranded AAV (dsAAV). Any variant of an AAV vector or serotype thereof, such as a self-complementary AAV (scAAV) vector, is encompassed within the general terms AAV vector, AAV1 vector, etc. See, e.g., McCarty et al., Gene Ther. 2001; 8:1248-54, Naso et al., BioDrugs 2017; 31:317-334, and references cited therein for detailed discussion of various AAV vectors. In some embodiments, the AAV vector size is measured in length of nucleotides from ITR to ITR, inclusive of both ITRs. In some embodiments, the AAV vector is less than 5 kb in size from ITR to ITR, inclusive of both ITRs. In particular embodiments, the AAV vector is less than 4.9 kb from ITR to ITR in size, inclusive of both ITRs. In further embodiments, the AAV vector is less than 4.85 kb in size from ITR to ITR, inclusive of both ITRs. In further embodiments, the AAV vector is less than 4.8 kb in size from ITR to ITR, inclusive of both ITRs. In further embodiments, the AAV vector is less than 4.75 kb in size from ITR to ITR, inclusive of both ITRs. In further embodiments, the AAV vector is less than 4.7 kb in size from ITR to ITR, inclusive of both ITRs.

In some embodiments, the vector is an AAV9 vector. In some embodiments, the vector (e.g., viral vector, such as an adeno-associated viral vector) comprises a tissue-specific (e.g., muscle-specific) promoter, e.g., which is operatively linked to a sequence encoding the gRNA. In some embodiments, the muscle-specific promoter is a muscle creatine kinase promoter, a desmin promoter, an MHCK7 promoter, or an SPc5-12 promoter. In some embodiments, the muscle-specific promoter is a CK8 promoter. In some embodiments, the muscle-specific promoter is a CK8e promoter. Muscle-specific promoters are described in detail, e.g., in US2004/0175727 A1; Wang et al., Expert Opin Drug Deliv. (2014) 11, 345-364; Wang et al., Gene Therapy (2008) 15, 1489-1499. In some embodiments, the tissue-specific promoter is a neuron-specific promoter, such as an enolase promoter. See, e.g., Naso et al., BioDrugs 2017; 31:317-334; Dashkoff et al., Mol Ther Methods Clin Dev. 2016; 3:16081, and references cited therein for detailed discussion of tissue-specific promoters including neuron-specific promoters.

In some embodiments, in addition to guide RNA sequences, the vectors further comprise nucleic acids that do not encode guide RNAs. Nucleic acids that do not encode guide RNA include, but are not limited to, promoters, enhancers, regulatory sequences, and nucleic acids encoding an RNA-guided DNA nuclease, which can be a nuclease such as Cas9. In some embodiments, the vector comprises one or more nucleotide sequence(s) encoding a crRNA, a trRNA, or a crRNA and trRNA.

Lipid nanoparticles (LNPs) are a known means for delivery of nucleotide and protein cargo, and may be used for delivery of the guide RNAs, compositions, or pharmaceutical formulations disclosed herein. In some embodiments, the LNPs deliver nucleic acid, protein, or nucleic acid together with protein.

In some embodiments, the invention comprises a method for delivering any one of the gRNAs disclosed herein to a subject, wherein the gRNA is associated with an LNP. In some embodiments, the gRNA/LNP is also associated with SluCas9 or an mRNA encoding SluCas9.

In some embodiments, the invention comprises a composition comprising any one of the gRNAs disclosed and an LNP. In some embodiments, the composition further comprises SluCas9 or an mRNA encoding SluCas9.

Electroporation is a well-known means for delivery of cargo, and any electroporation methodology may be used for delivery of any one of the gRNAs disclosed herein. In some embodiments, electroporation may be used to deliver any one of the gRNAs disclosed herein and SluCas9 or an mRNA encoding SluCas9.

In some embodiments, the invention comprises a method for delivering any one of the gRNAs disclosed herein to an ex vivo cell, wherein the gRNA is encoded by a vector, associated with an LNP, or in aqueous solution. In some embodiments, the gRNA/LNP or gRNA is also associated with SluCas9 or sequence encoding SluCas9 (e.g., in the same vector, LNP, or solution).

In some embodiments, the invention comprises a method for delivering any one of the gRNAs disclosed herein to a subject, wherein the gRNA is associated with an LNP. In some embodiments, the gRNA/LNP is also associated with or SaCas9 an mRNA encoding SaCas9.

In some embodiments, the invention comprises a composition comprising any one of the gRNAs disclosed and an LNP. In some embodiments, the composition further comprises SaCas9 or an mRNA encoding SaCas9.

Electroporation is a well-known means for delivery of cargo, and any electroporation methodology may be used for delivery of any one of the gRNAs disclosed herein. In some embodiments, electroporation may be used to deliver any one of the gRNAs disclosed herein and SaCas9 or an mRNA encoding SaCas9.

In some embodiments, the invention comprises a method for delivering any one of the gRNAs disclosed herein to an ex vivo cell, wherein the gRNA is encoded by a vector, associated with an LNP, or in aqueous solution. In some embodiments, the gRNA/LNP or gRNA is also associated with SaCas9 or sequence encoding SaCas9 (e.g., in the same vector, LNP, or solution).

### IV. Compositions

Compositions Comprising Guide RNA (gRNAs)

Provided herein are compositions useful for treating DM1, e.g., comprising 1) one or more guide RNAs comprising one or more guide sequences of Table 2, or nucleic acids encoding same; and optionally 2) SluCas9 or a nucleic acid encoding SluCas9 (for SEQ ID Nos: 1-51, 53, 55-56, 58-59, 61-62, 64, 66, or 70) or SaCas9 or a nucleic acid encoding SaCas9 (for SEQ ID Nos: 200-259). Such compositions may be administered to subjects having or suspected of having DM1.

Also provided herein are compositions useful for excising trinucleotide repeats from DNA of DMPK, e.g., using two or more guide RNAs with SluCas9 or SaCas9. Pairs of guide RNAs are contemplated for use in excision methods and therefore any composition described below that comprises one guide RNA can be used in combination with another to achieve the intended purpose. Further, compositions comprising two or more guide RNAs are contemplated.

The compositions may comprise one or more guide RNAs or a vector(s) encoding one or more guide RNAs comprising a spacer sequence of any one of SEQ ID NOs: 1-51, 53, 55-56, 58-59, 61-62, 64, 66, 70, or 200-259 and may be administered to subjects having or suspected of having DM1, optionally with SluCas9 or a nucleic acid encoding SluCas9 (for SEQ ID NOs: 1-51, 53, 55-56, 58-59, 61-62, 64, 66, or 70) or a SaCas9 or a nucleic acid encoding SaCas9 (for SEQ ID NOs: 200-259).

In some embodiments, a guide RNA is provided wherein the gRNA comprises a guide sequence of any one of SEQ ID NOs 5, 21, 46, 55, 59, 61, 64, 7, 19, 41, or 47.

In some embodiments, one or more gRNAs direct a SluCas9 to a site in or near a TNR. For example, the SluCas9 may be directed to cut within 10, 20, 30, 40, or 50 nucleotides of the TNR based on the sequence of the spacer sequence.

In some embodiments, a composition is provided comprising a guide RNA comprising a spacer sequence comprising a sequence selected from any one of SEQ ID NOs: 1-51, 53, 55-56, 58-59, 61-62, 64, 66, or 70 or a nucleic acid encoding same, and optionally, a nucleic acid encoding a Staphylococcus lugdunensis (SluCas9). In some embodiments, a composition is provided comprising a gRNA encoding a spacer sequence comprising a sequence that is at least 20 contiguous nucleotides of a spacer sequence selected from any one of SEQ ID NOs: 1-51, 53, 55-56, 58-59, 61-62, 64, 66, or or a nucleic acid encoding same, and optionally a gRNA encoding a Staphylococcus lugdunensis (SluCas9). In some embodiments, a composition is provided comprising a first nucleic acid encoding a spacer sequence comprising a sequence that is at least 90% identical to any one of SEQ ID NOs: 1-51, 53, 55-56, 58-59, 61-62, 64, 66, or 70 and optionally a second nucleic acid encoding a Staphylococcus lugdunensis (SluCas9). In some embodiments, the composition comprises the second nucleic acid encoding a Staphylococcus lugdunensis (SluCas9).

In some embodiments, one or more guide RNAs and SluCas9 are provided on a single nucleic acid molecule. In some embodiments, the single nucleic acid molecule is a vector. In some embodiments, the vector expresses the guide RNA(s) and SluCas9. In some embodiments, the guide RNA(s) and SluCas9 are expressed from the same vector, but with different promoters. In some embodiments, the guide RNA(s) and SluCas9 are provided on two separate nucleic acid molecules. In some embodiments, two separate nucleic acid molecules are provided wherein the first comprises one or more sequences encoding a spacer sequence of a guide RNA (e.g., one or more copies of one or more different spacer sequences) and does not comprise a sequence encoding an endonuclease, and the second comprises a sequence encoding a SluCas9 or SaCas9 and optionally sequence(s) encoding one or more guide RNAs. In some embodiments, the nucleic acid molecules are vectors. In some embodiments, the vectors express one or more guide RNA and SluCas9.

In some embodiments, at least a pair of gRNAs are provided which direct a SluCas9 to a pair of sites flanking (i.e., on opposite sides of) a TNR in DMPK. For example, the pair of sites flanking a TNR may each be within 10, 20, 30, 40, or 50 nucleotides of the TNR but on opposite sides thereof. In some embodiments, a pair of gRNAs is provided that comprise SluCas9 guide sequences selected from SEQ ID NOs: 1-51, 53, 55-56, 58-59, 61-62, 64, 66, and 70 and direct a SluCas9 to a pair of sites according to any of the foregoing embodiments. In some embodiments, the pair of spacer sequences comprises SEQ ID NO: 5 and SEQ ID NO: 10, SEQ ID NO: 46 and SEQ ID NO: 10, SEQ ID NO: 61 and SEQ ID NO: 10, or SEQ ID NO: 64 and SEQ ID NO: 47.

In some embodiments, a composition is provided comprising a pair of guide RNAs comprising a pair of spacer sequences, or one or more vectors encoding the pair of guide RNAs, wherein the pair of spacer sequences comprise: a) a first spacer sequence selected from SEQ ID NOs: 21, 46, 55, 59, 61, or 64, and a second spacer sequence selected from SEQ ID NOs: 7, 19, 41, or 47; b) a first and second spacer sequence of SEQ ID NOs: 5 and 7; c) a first and second spacer sequence of SEQ ID NOs: 5 and 10; d) a first and second spacer sequence of SEQ ID NOs: 5 and 19; e) a first and second spacer sequence of SEQ ID NOs: 5 and 41; f) a first and second spacer sequence of SEQ ID NOs: 5 and 47; g) a first and second spacer sequence of SEQ ID NOs: 21 and 7; h) a first and second spacer sequence of SEQ ID NOs: 21 and 19; i) a first and second spacer sequence of SEQ ID NOs: 21 and 41; j) a first and second spacer sequence of SEQ ID NOs: 21 and 47; k) a first and second spacer sequence of SEQ ID NOs: 46 and 7; 1) a first and second spacer sequence of SEQ ID NOs: 46 and 10; m) a first and second spacer sequence of SEQ ID NOs: 46 and 19; n) a first and second spacer sequence of SEQ ID NOs: 46 and 41; o) a first and second spacer sequence of SEQ ID NOs: 46 and 47; p) a first and second spacer sequence of SEQ ID NOs: 55 and 7; q) a first and second spacer sequence of SEQ ID NOs: 55 and 19; r) a first and second spacer sequence of SEQ ID NOs: 55 and 41; s) a first and second spacer sequence of SEQ ID NOs: 55 and 47; t) a first and second spacer sequence of SEQ ID NOs: 59 and 7; u) a first and second spacer sequence of SEQ ID NOs: 59 and 19; v) a first and second spacer sequence of SEQ ID NOs: 59 and 41; w) a first and second spacer sequence of SEQ ID NOs: 59 and 47; x) a first and second spacer sequence of SEQ ID NOs: 61 and 7; y) a first and second spacer sequence of SEQ ID NOs: 61 and 10; z) a first and second spacer sequence of SEQ ID NOs: 61 and 19; aa) a first and second spacer sequence of SEQ ID NOs: 61 and 41; bb) a first and second spacer sequence of SEQ ID NOs: 61 and 47; cc) a first and second spacer sequence of SEQ ID NOs: 64 and 7; dd) a first and second spacer sequence of SEQ ID NOs: 64 and 19; ee) a first and second spacer sequence of SEQ ID NOs: 64 and 41; or ff) a first and second spacer sequence of SEQ ID NOs: 64 and 47.

In some embodiments, a composition is provided comprising a pair of gRNAs comprising a pair of spacer sequences, or one or more vectors encoding the pair of guide RNAs, wherein the pair of spacer sequences comprise a first spacer sequence selected from SEQ ID NOs: 3, 5, 6, 9, 16, 21, 22, 25, 26, 30, 36, 38, 39, 40, 46, 51, 53, 55, 56, 58, 59, 61, 62, 64, 66, and 70, and a second spacer sequence selected from SEQ ID NOs: 1, 2, 4, 7, 8, 10, 11, 12, 13, 14, 15, 17, 18, 19, 20, 23, 24, 27, 28, 29, 31, 32, 33, 34, 35, 37, 41, 42, 43, 44, 45, 47, 48, 49, 50. In some embodiments, the pair of spacer sequences comprises SEQ ID NO: 5 and SEQ ID NO: 10, SEQ ID NO: 46 and SEQ ID NO: SEQ ID NO: 61 and SEQ ID NO: 10, or SEQ ID NO: 64 and SEQ ID NO: 47. In one embodiment, a composition is provided comprising a pair of gRNAs comprising a pair of spacer sequences, or one or more vectors encoding the pair of guide RNAs, wherein the pair of spacer sequences comprise a first spacer sequence that is SEQ ID NO: 3 and a second spacer sequence selected from any one of SEQ ID NOs: 1, 2, 4, 7, 8, 10, 11, 12, 13, 14, 15, 17, 18, 19, 20, 23, 24, 27, 28, 29, 31, 32, 33, 34, 35, 37, 41, 42, 43, 44, 45, 47, 48, 49, 50, respectively. In one embodiment, a composition is provided comprising a pair of gRNAs comprising a pair of spacer sequences, or one or more vectors encoding the pair of guide RNAs, wherein the pair of spacer sequences comprise a first spacer sequence that is SEQ ID NO: 5 and a second spacer sequence selected from any one of SEQ ID NOs: 1, 2, 4, 7, 8, 10, 11, 12, 13, 14, 15, 17, 18, 19, 20, 23, 24, 27, 28, 29, 31, 32, 33, 34, 35, 37, 41, 42, 43, 44, 45, 47, 48, 49, 50, respectively. In one embodiment, a composition is provided comprising a pair of gRNAs comprising a pair of spacer sequences, or one or more vectors encoding the pair of guide RNAs, wherein the pair of spacer sequences comprise a first spacer sequence that is SEQ ID NO: 6 and a second spacer sequence selected from any one of SEQ ID NOs: 1, 2, 4, 7, 8, 10, 11, 12, 13, 14, 15, 17, 18, 19, 20, 23, 24, 27, 28, 29, 31, 32, 33, 34, 35, 37, 41, 42, 43, 44, 45, 47, 48, 49, 50, respectively. In one embodiment, a composition is provided comprising a pair of gRNAs comprising a pair of spacer sequences, or one or more vectors encoding the pair of guide RNAs, wherein the pair of spacer sequences comprise a first spacer sequence that is SEQ ID NO: 9 and a second spacer sequence selected from any one of SEQ ID NOs: 1, 2, 4, 7, 8, 10, 11, 12, 13, 14, 15, 17, 18, 19, 20, 23, 24, 27, 28, 29, 31, 32, 33, 34, 35, 37, 41, 42, 43, 44, 45, 47, 48, 49, 50, respectively. In one embodiment, a composition is provided comprising a pair of gRNAs comprising a pair of spacer sequences, or one or more vectors encoding the pair of guide RNAs, wherein the pair of spacer sequences comprise a first spacer sequence that is SEQ ID NO: 10 and a second spacer sequence selected from any one of SEQ ID NOs: 3, 5, 6, 9, 16, 21, 22, 25, 26, 30, 36, 38, 39, 40, 46, 51, 53, 55, 56, 58, 59, 61, 62, 64, 66, and 70, respectively. In one embodiment, a composition is provided comprising a pair of gRNAs comprising a pair of spacer sequences, or one or more vectors encoding the pair of guide RNAs, wherein the pair of spacer sequences comprise a first spacer sequence that is SEQ ID NO: 16 and a second spacer sequence selected from any one of SEQ ID NOs: 1, 2, 4, 7, 8, 10, 11, 12, 13, 14, 15, 17, 18, 19, 20, 23, 24, 27, 28, 29, 31, 32, 33, 34, 35, 37, 41, 42, 43, 44, 45, 47, 48, 49, 50, respectively. In one embodiment, a composition is provided comprising a pair of gRNAs comprising a pair of spacer sequences, or one or more vectors encoding the pair of guide RNAs, wherein the pair of spacer sequences comprise a first spacer sequence that is SEQ ID NO: 21 and a second spacer sequence selected from any one of SEQ ID NOs: 1, 2, 4, 7, 8, 10, 11, 12, 13, 14, 15, 17, 18, 19, 20, 23, 24, 27, 28, 29, 31, 32, 33, 34, 35, 37, 41, 42, 43, 44, 45, 47, 48, 49, 50, respectively. In one embodiment, a composition is provided comprising a pair of gRNAs comprising a pair of spacer sequences, or one or more vectors encoding the pair of guide RNAs, wherein the pair of spacer sequences comprise a first spacer sequence that is SEQ ID NO: 22 and a second spacer sequence selected from any one of SEQ ID NOs: 1, 2, 4, 7, 8, 10, 11, 12, 13, 14, 15, 17, 18, 19, 20, 23, 24, 27, 28, 29, 31, 32, 33, 34, 35, 37, 41, 42, 43, 44, 45, 47, 48, 49, 50, respectively. In one embodiment, a composition is provided comprising a pair of gRNAs comprising a pair of spacer sequences, or one or more vectors encoding the pair of guide RNAs, wherein the pair of spacer sequences comprise a first spacer sequence that is SEQ ID NO: 25 and a second spacer sequence selected from any one of SEQ ID NOs: 1, 2, 4, 7, 8, 10, 11, 12, 13, 14, 15, 17, 18, 19, 20, 23, 24, 27, 28, 29, 31, 32, 33, 34, 35, 37, 41, 42, 43, 44, 45, 47, 48, 49, 50, respectively. In one embodiment, a composition is provided comprising a pair of gRNAs comprising a pair of spacer sequences, or one or more vectors encoding the pair of guide RNAs, wherein the pair of spacer sequences comprise a first spacer sequence that is SEQ ID NO: 26 and a second spacer sequence selected from any one of SEQ ID NOs: 1, 2, 4, 7, 8, 10, 11, 12, 13, 14, 15, 17, 18, 19, 20, 23, 24, 27, 28, 29, 31, 32, 33, 34, 35, 37, 41, 42, 43, 44, 45, 47, 48, 49, 50, respectively. In one embodiment, a composition is provided comprising a pair of gRNAs comprising a pair of spacer sequences, or one or more vectors encoding the pair of guide RNAs, wherein the pair of spacer sequences comprise a first spacer sequence that is SEQ ID NO: 30 and a second spacer sequence selected from any one of SEQ ID NOs: 1, 2, 4, 7, 8, 10, 11, 12, 13, 14, 15, 17, 18, 19, 20, 23, 24, 27, 28, 29, 31, 32, 33, 34, 35, 37, 41, 42, 43, 44, 45, 47, 48, 49, 50, respectively. In one embodiment, a composition is provided comprising a pair of gRNAs comprising a pair of spacer sequences, or one or more vectors encoding the pair of guide RNAs, wherein the pair of spacer sequences comprise a first spacer sequence that is SEQ ID NO: 36 and a second spacer sequence selected from any one of SEQ ID NOs: 1, 2, 4, 7, 8, 10, 11, 12, 13, 14, 15, 17, 18, 19, 20, 23, 24, 27, 28, 29, 31, 32, 33, 34, 35, 37, 41, 42, 43, 44, 45, 47, 48, 49, 50, respectively. In one embodiment, a composition is provided comprising a pair of gRNAs comprising a pair of spacer sequences, or one or more vectors encoding the pair of guide RNAs, wherein the pair of spacer sequences comprise a first spacer sequence that is SEQ ID NO: 38 and a second spacer sequence selected from any one of SEQ ID NOs: 1, 2, 4, 7, 8, 10, 11, 12, 13, 14, 15, 17, 18, 19, 20, 23, 24, 27, 28, 29, 31, 32, 33, 34, 35, 37, 41, 42, 43, 44, 45, 47, 48, 49, 50, respectively. In one embodiment, a composition is provided comprising a pair of gRNAs comprising a pair of spacer sequences, or one or more vectors encoding the pair of guide RNAs, wherein the pair of spacer sequences comprise a first spacer sequence that is SEQ ID NO: 39 and a second spacer sequence selected from any one of SEQ ID NOs: 1, 2, 4, 7, 8, 10, 11, 12, 13, 14, 15, 17, 18, 19, 20, 23, 24, 27, 28, 29, 31, 32, 33, 34, 35, 37, 41, 42, 43, 44, 45, 47, 48, 49, 50, respectively. In one embodiment, a composition is provided comprising a pair of gRNAs comprising a pair of spacer sequences, or one or more vectors encoding the pair of guide RNAs, wherein the pair of spacer sequences comprise a first spacer sequence that is SEQ ID NO: 40 and a second spacer sequence selected from any one of SEQ ID NOs: 1, 2, 4, 7, 8, 10, 11, 12, 13, 14, 15, 17, 18, 19, 20, 23, 24, 27, 28, 29, 31, 32, 33, 34, 35, 37, 41, 42, 43, 44, 45, 47, 48, 49, 50, respectively. In one embodiment, a composition is provided comprising a pair of gRNAs comprising a pair of spacer sequences, or one or more vectors encoding the pair of guide RNAs, wherein the pair of spacer sequences comprise a first spacer sequence that is SEQ ID NO: 46 and a second spacer sequence selected from any one of SEQ ID NOs: 1, 2, 4, 7, 8, 10, 11, 12, 13, 14, 15, 17, 18, 19, 20, 23, 24, 27, 28, 29, 31, 32, 33, 34, 35, 37, 41, 42, 43, 44, 45, 47, 48, 49, 50, respectively. In one embodiment, a composition is provided comprising a pair of gRNAs comprising a pair of spacer sequences, or one or more vectors encoding the pair of guide RNAs, wherein the pair of spacer sequences comprise a first spacer sequence that is SEQ ID NO: 51 and a second spacer sequence selected from any one of SEQ ID NOs: 1, 2, 4, 7, 8, 10, 11, 12, 13, 14, 15, 17, 18, 19, 20, 23, 24, 27, 28, 29, 31, 32, 33, 34, 35, 37, 41, 42, 43, 44, 45, 47, 48, 49, 50, respectively. In one embodiment, a composition is provided comprising a pair of gRNAs comprising a pair of spacer sequences, or one or more vectors encoding the pair of guide RNAs, wherein the pair of spacer sequences comprise a first spacer sequence that is SEQ ID NO: 53 and a second spacer sequence selected from any one of SEQ ID NOs: 1, 2, 4, 7, 8, 10, 11, 12, 13, 14, 15, 17, 18, 19, 20, 23, 24, 27, 28, 29, 31, 32, 33, 34, 35, 37, 41, 42, 43, 44, 45, 47, 48, 49, 50, respectively. In one embodiment, a composition is provided comprising a pair of gRNAs comprising a pair of spacer sequences, or one or more vectors encoding the pair of guide RNAs, wherein the pair of spacer sequences comprise a first spacer sequence that is SEQ ID NO: 55 and a second spacer sequence selected from any one of SEQ ID NOs: 1, 2, 4, 7, 8, 10, 11, 12, 13, 14, 15, 17, 18, 19, 20, 23, 24, 27, 28, 29, 31, 32, 33, 34, 35, 37, 41, 42, 43, 44, 45, 47, 48, 49, 50, respectively. In one embodiment, a composition is provided comprising a pair of gRNAs comprising a pair of spacer sequences, or one or more vectors encoding the pair of guide RNAs, wherein the pair of spacer sequences comprise a first spacer sequence that is SEQ ID NO: 56 and a second spacer sequence selected from any one of SEQ ID NOs: 1, 2, 4, 7, 8, 10, 11, 12, 13, 14, 15, 17, 18, 19, 20, 23, 24, 27, 28, 29, 31, 32, 33, 34, 35, 37, 41, 42, 43, 44, 45, 47, 48, 49, 50, respectively. In one embodiment, a composition is provided comprising a pair of gRNAs comprising a pair of spacer sequences, or one or more vectors encoding the pair of guide RNAs, wherein the pair of spacer sequences comprise a first spacer sequence that is SEQ ID NO: 58 and a second spacer sequence selected from any one of SEQ ID NOs: 1, 2, 4, 7, 8, 10, 11, 12, 13, 14, 15, 17, 18, 19, 20, 23, 24, 27, 28, 29, 31, 32, 33, 34, 35, 37, 41, 42, 43, 44, 45, 47, 48, 49, 50, respectively. In one embodiment, a composition is provided comprising a pair of gRNAs comprising a pair of spacer sequences, or one or more vectors encoding the pair of guide RNAs, wherein the pair of spacer sequences comprise a first spacer sequence that is SEQ ID NO: 59 and a second spacer sequence selected from any one of SEQ ID NOs: ID 1, 2, 4, 7, 8, 10, 11, 12, 13, 14, 15, 17, 18, 19, 20, 23, 24, 27, 28, 29, 31, 32, 33, 34, 35, 37, 41, 42, 43, 44, 45, 47, 48, 49, 50, respectively. In one embodiment, a composition is provided comprising a pair of gRNAs comprising a pair of spacer sequences, or one or more vectors encoding the pair of guide RNAs, wherein the pair of spacer sequences comprise a first spacer sequence that is SEQ ID NO: 61 and a second spacer sequence selected from any one of SEQ ID NOs: 1, 2, 4, 7, 8, 10, 11, 12, 13, 14, 15, 17, 18, 19, 20, 23, 24, 27, 28, 29, 31, 32, 33, 34, 35, 37, 41, 42, 43, 44, 45, 47, 48, 49, 50, respectively. In one embodiment, a composition is provided comprising a pair of gRNAs comprising a pair of spacer sequences, or one or more vectors encoding the pair of guide RNAs, wherein the pair of spacer sequences comprise a first spacer sequence that is SEQ ID NO: 62 and a second spacer sequence selected from any one of SEQ ID NOs: 1, 2, 4, 7, 8, 10, 11, 12, 13, 14, 15, 17, 18, 19, 20, 23, 24, 27, 28, 29, 31, 32, 33, 34, 35, 37, 41, 42, 43, 44, 45, 47, 48, 49, 50, respectively. In one embodiment, a composition is provided comprising a pair of gRNAs comprising a pair of spacer sequences, or one or more vectors encoding the pair of guide RNAs, wherein the pair of spacer sequences comprise a first spacer sequence that is SEQ ID NO: 64 and a second spacer sequence selected from any one of SEQ ID NOs: 1, 2, 4, 7, 8, 10, 11, 12, 13, 14, 15, 17, 18, 19, 20, 23, 24, 27, 28, 29, 31, 32, 33, 34, 35, 37, 41, 42, 43, 44, 45, 47, 48, 49, 50, respectively. In one embodiment, a composition is provided comprising a pair of gRNAs comprising a pair of spacer sequences, or one or more vectors encoding the pair of guide RNAs, wherein the pair of spacer sequences comprise a first spacer sequence that is SEQ ID NO: 66 and a second spacer sequence selected from any one of SEQ ID NOs: 1, 2, 4, 7, 8, 10, 11, 12, 13, 14, 15, 17, 18, 19, 20, 23, 24, 27, 28, 29, 31, 32, 33, 34, 35, 37, 41, 42, 43, 44, 45, 47, 48, 49, 50, respectively. In one embodiment, a composition is provided comprising a pair of gRNAs comprising a pair of spacer sequences, or one or more vectors encoding the pair of guide RNAs, wherein the pair of spacer sequences comprise a first spacer sequence that is SEQ ID NO: 70 and a second spacer sequence selected from any one of SEQ ID NOs: 1, 2, 4, 7, 8, 10, 11, 12, 13, 14, 15, 17, 18, 19, 20, 23, 24, 27, 28, 29, 31, 32, 33, 34, 35, 37, 41, 42, 43, 44, 45, 47, 48, 49, 50, respectively.

In some embodiments, nucleotide sequences encoding two guide RNAs and a nucleotide sequence encoding SluCas9 are provided on a single nucleic acid molecule. In some embodiments, the single nucleic acid molecule is a vector. In some embodiments, the vector expresses the two guide RNAs and SluCas9. In some embodiments, the two guide RNAs are identical. In some embodiments, the two guide RNAs are not identical. In some embodiments, the two guide RNAs and SluCas9 are separately expressed, e.g., from their own promoters.

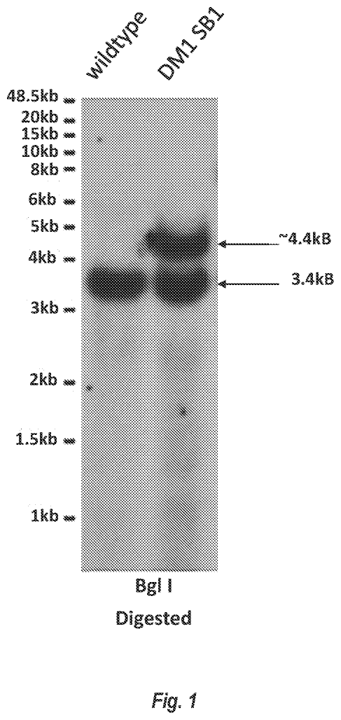

Each of the guide sequences shown in Table 2 at SEQ ID NOs: 1-51, 53, 55-56, 58-59, 61-62, 64, 66, or 70 may further comprise additional nucleotides to form or encode a crRNA, e.g., using any known sequence appropriate for the SluCas9 being used. In some embodiments, the crRNA comprises (5′ to 3′) at least a spacer sequence and a first complementarity domain. The first complementary domain is sufficiently complementary to a second complementarity domain, which may be part of the same molecule in the case of an sgRNA or in a tracrRNA in the case of a dual or modular gRNA, to form a duplex. See, e.g., US 2017/0007679 for detailed discussion of crRNA and gRNA domains, including first and second complementarity domains. For sgRNA, a spacer sequence is typically followed (5′ to 3′) by a crRNA, a linker (e.g., GAAA), and a tracrRNA. The crRNA, linker, and tracrRNA is sometimes referred to herein and in the art as a “scaffold” sequence. See, for example, Briner et al. (2014) Mol. Cell 56: 333-339, incorporated herein in its entirety, and in particular, the generalized structure of a sgRNA at FIG. 1A. For example, exemplary scaffold sequences suitable for use with SluCas9 to follow the guide sequence at its 3′ end is:

(SEQ ID NO: 600)
GTTTTAGTACTCTGGAAACAGAATCTACTGAAACAAGACAATATGTCGT
GTTTATCCCATCAATTTATTGGTGGGA;
(SEQ ID NO: 601)
GTTTAAGTACTCTGTGCTGGAAACAGCACAGAATCTACTGAAACAAGAC
AATATGTCGTGTTTATCCCATCAATTTATTGGTGGGA;
(SEQ ID NO: 602)
GUUUUAGUACUCUGGAAACAGAAUCUACUGAAACAAGACAAUAUGUCGU
GUUUAUCCCAUCAAUUUAUUGGUGGGAU;
(SEQ ID NO: 603)
CTTGTACTTATACCTAAAATTACAGAATCTACTGAAACAAGACAATATG
TCGTGTTTATCCCATCAATTTATTGGTGGGATTTTTTTATGTTTTTAGC
AAAAAGTAATACCATACTTTATATTTTTAAATTATAATAAAGATATAAA
TAAAGGTGG;
or
(SEQ ID NO: 604)
GTTTCAGTACTCTGGAAACAGAATCTACTGAAACAAGACAATATGTCGT
GTTTATCCCATCAATTTATTGGTGGGAT

in 5′ to 3′ orientation. Note that in these sequences, T's are representative of the DNA version, and with U's in an RNA version. In some embodiments, an exemplary sequence for use with SluCas9 to follow the 3′ end of the guide sequence is a sequence that is at least 80%, 85%, 90%, 91%, 92%, 93%, 94%, 95%, 96%, 97%, 98%, 99% or 100% identical to SEQ ID NO: 600, SEQ ID NO: 601, SEQ ID NO: 602, SEQ ID NO: 603, or SEQ ID NO: 604, or a sequence that differs from SEQ ID NO: 600 or SEQ ID NO: 601 or SEQ ID NO: 602, SEQ ID NO: 603, or SEQ ID NO: 604 by no more than 1, 2, 3, 4, 5, 10, 15, 20, or 25 nucleotides.

In some embodiments, a guide RNA is provided wherein the gRNA comprises a guide sequence of any one of SEQ ID Nos: 200-259.

In some embodiments, one or more gRNAs direct a SaCas9 to a site in or near a TNR. For example, the SaCas9 may be directed to cut within 10, 20, 30, 40, or 50 nucleotides of the TNR based on the sequence of the spacer sequence.

In some embodiments, a composition is provided comprising a guide RNA comprising a spacer sequence comprising a sequence selected from any one of SEQ ID NOs: 200-259, or a nucleic acid encoding same, and optionally a nucleic acid encoding a Staphylococcus aureus (SaCas9). In some embodiments, a composition is provided comprising a gRNA encoding a spacer sequence comprising a sequence that is at least 20 contiguous nucleotides of a spacer sequence selected from any one of SEQ ID NOs: 200-259, or a nucleic acid encoding same, and optionally a gRNA encoding a Staphylococcus aureus (SaCas9). In some embodiments, a composition is provided comprising a first nucleic acid encoding a spacer sequence comprising a sequence that is at least 90% identical to any one of SEQ ID NOs: 200-259 and optionally a second nucleic acid encoding a Staphylococcus aureus (SaCas9). In some embodiments, the composition comprises the second nucleic acid encoding a Staphylococcus aureus (SaCas9).

In some embodiments, one or more guide RNAs and SaCas9 are provided on a single nucleic acid molecule. In some embodiments, the single nucleic acid molecule is a vector. In some embodiments, the vector expresses the guide RNA(s) and SaCas9. In some embodiments, the guide RNA and SaCas9 are expressed from the same vector, but with different promoters. In some embodiments, a guide RNA and SaCas9 are provided on two separate nucleic acid molecules. In some embodiments, two separate nucleic acid molecules are provided wherein the first comprises one or more sequences encoding a spacer sequence of a guide RNA and does not comprise a sequence encoding an endonuclease, and the second comprises a sequence encoding a SluCas9 or SaCas9 and optionally sequence(s) encoding one or more guide RNAs. In some embodiments, the nucleic acid molecules are vectors. In some embodiments, the vectors express one or more guide RNAs and SaCas9.

In some embodiments, at least a pair of gRNAs are provided which direct a SaCas9 to a pair of sites flanking (i.e., on opposite sides of) a TNR in DMPK. For example, the pair of sites flanking a TNR may each be within 10, 20, 30, 40, or 50 nucleotides of the TNR but on opposite sides thereof. In some embodiments, a pair of gRNAs is provided that comprise SaCas9 guide sequences selected from SEQ ID NOs: 200-259 and direct a SaCas9 to a pair of sites according to any of the foregoing embodiments. In some embodiments, the pair of gRNAs comprises any one of the SEQ ID NO: 202 and SEQ ID NO: 218, SEQ ID NO: 202 and SEQ ID NO: 213, SEQ ID NO: 201 and SEQ ID NO: 224, or SEQ ID NO: 201 and SEQ ID NO: 206.

In some embodiments, a composition is provided comprising a pair of guide RNAs comprising a pair of spacer sequences, or one or more vectors encoding the pair of guide RNAs, wherein the pair of spacer sequences comprise: a) a first spacer sequence selected from SEQ ID NOs: 201 and 202, and a second spacer sequence selected from SEQ ID NOs: 206, 213, 218, and 224. In some embodiments, the pair of gRNAs comprises any one of the SEQ ID NO: 202 and SEQ ID NO: 218, SEQ ID NO: 202 and SEQ ID NO: 213, SEQ ID NO: 201 and SEQ ID NO: 224, or SEQ ID NO: 201 and SEQ ID NO: 206.

In some embodiments, a composition is provided comprising a pair of gRNAs comprising a pair of spacer sequences, or one or more vectors encoding the pair of guide RNAs, wherein the pair of spacer sequences comprise a first spacer sequence selected from SEQ ID NOs: 201-203, 211, 215, 220, 225, 231, 235, 238, and 240-259, and a second spacer sequence selected from SEQ ID NOs: 200, 204-210, 212-214, 216-219, 221-224, 226-230, 232-234, 236-237, and 239. In one embodiment, a composition is provided comprising a pair of gRNAs comprising a pair of spacer sequences, or one or more vectors encoding the pair of guide RNAs, wherein the pair of spacer sequences comprise a first spacer sequence that is SEQ ID NO: 201 and a second spacer sequence selected from any one of SEQ ID NOs: 200, 204-210, 212-214, 216-219, 221-224, 226-230, 232-234, 236-237, and 239, respectively. In one embodiment, a composition is provided comprising a pair of gRNAs comprising a pair of spacer sequences, or one or more vectors encoding the pair of guide RNAs, wherein the pair of spacer sequences comprise a first spacer sequence that is SEQ ID NO: 202 and a second spacer sequence selected from any one of SEQ ID NOs: 200, 204-210, 212-214, 216-219, 221-224, 226-230, 232-234, 236-237, and 239, respectively. In one embodiment, a composition is provided comprising a pair of gRNAs comprising a pair of spacer sequences, or one or more vectors encoding the pair of guide RNAs, wherein the pair of spacer sequences comprise a first spacer sequence that is SEQ ID NO: 203 and a second spacer sequence selected from any one of SEQ ID NOs: 200, 204-210, 212-214, 216-219, 221-224, 226-230, 232-234, 236-237, and 239, respectively. In one embodiment, a composition is provided comprising a pair of gRNAs comprising a pair of spacer sequences, or one or more vectors encoding the pair of guide RNAs, wherein the pair of spacer sequences comprise a first spacer sequence that is SEQ ID NO: 211 and a second spacer sequence selected from any one of SEQ ID NOs: 200, 204-210, 212-214, 216-219, 221-224, 226-230, 232-234, 236-237, and 239, respectively. In one embodiment, a composition is provided comprising a pair of gRNAs comprising a pair of spacer sequences, or one or more vectors encoding the pair of guide RNAs, wherein the pair of spacer sequences comprise a first spacer sequence that is SEQ ID NO: 215 and a second spacer sequence selected from any one of SEQ ID NOs: 200, 204-210, 212-214, 216-219, 221-224, 226-230, 232-234, 236-237, and 239, respectively. In one embodiment, a composition is provided comprising a pair of gRNAs comprising a pair of spacer sequences, or one or more vectors encoding the pair of guide RNAs, wherein the pair of spacer sequences comprise a first spacer sequence that is SEQ ID NO: 220 and a second spacer sequence selected from any one of SEQ ID NOs: 200, 204-210, 212-214, 216-219, 221-224, 226-230, 232-234, 236-237, and 239, respectively. In one embodiment, a composition is provided comprising a pair of gRNAs comprising a pair of spacer sequences, or one or more vectors encoding the pair of guide RNAs, wherein the pair of spacer sequences comprise a first spacer sequence that is SEQ ID NO: 225 and a second spacer sequence selected from any one of SEQ ID NOs: 200, 204-210, 212-214, 216-219, 221-224, 226-230, 232-234, 236-237, and 239, respectively. In one embodiment, a composition is provided comprising a pair of gRNAs comprising a pair of spacer sequences, or one or more vectors encoding the pair of guide RNAs, wherein the pair of spacer sequences comprise a first spacer sequence that is SEQ ID NO: 231 and a second spacer sequence selected from any one of SEQ ID NOs: 200, 204-210, 212-214, 216-219, 221-224, 226-230, 232-234, 236-237, and 239, respectively. In one embodiment, a composition is provided comprising a pair of gRNAs comprising a pair of spacer sequences, or one or more vectors encoding the pair of guide RNAs, wherein the pair of spacer sequences comprise a first spacer sequence that is SEQ ID NO: 235 and a second spacer sequence selected from any one of SEQ ID NOs: 200, 204-210, 212-214, 216-219, 221-224, 226-230, 232-234, 236-237, and 239, respectively. In one embodiment, a composition is provided comprising a pair of gRNAs comprising a pair of spacer sequences, or one or more vectors encoding the pair of guide RNAs, wherein the pair of spacer sequences comprise a first spacer sequence that is SEQ ID NO: 238 and a second spacer sequence selected from any one of SEQ ID NOs: 200, 204-210, 212-214, 216-219, 221-224, 226-230, 232-234, 236-237, and 239, respectively. In one embodiment, a composition is provided comprising a pair of gRNAs comprising a pair of spacer sequences, or one or more vectors encoding the pair of guide RNAs, wherein the pair of spacer sequences comprise a first spacer sequence that is SEQ ID NO: 240 and a second spacer sequence selected from any one of SEQ ID NOs: 200, 204-210, 212-214, 216-219, 221-224, 226-230, 232-234, 236-237, and 239, respectively. In one embodiment, a composition is provided comprising a pair of gRNAs comprising a pair of spacer sequences, or one or more vectors encoding the pair of guide RNAs, wherein the pair of spacer sequences comprise a first spacer sequence that is SEQ ID NO: 240 and a second spacer sequence selected from any one of SEQ ID NOs: 200, 204-210, 212-214, 216-219, 221-224, 226-230, 232-234, 236-237, and 239, respectively. In one embodiment, a composition is provided comprising a pair of gRNAs comprising a pair of spacer sequences, or one or more vectors encoding the pair of guide RNAs, wherein the pair of spacer sequences comprise a first spacer sequence that is SEQ ID NO: 241 and a second spacer sequence selected from any one of SEQ ID NOs: 200, 204-210, 212-214, 216-219, 221-224, 226-230, 232-234, 236-237, and 239, respectively. In one embodiment, a composition is provided comprising a pair of gRNAs comprising a pair of spacer sequences, or one or more vectors encoding the pair of guide RNAs, wherein the pair of spacer sequences comprise a first spacer sequence that is SEQ ID NO: 242 and a second spacer sequence selected from any one of SEQ ID NOs: 200, 204-210, 212-214, 216-219, 221-224, 226-230, 232-234, 236-237, and 239, respectively. In one embodiment, a composition is provided comprising a pair of gRNAs comprising a pair of spacer sequences, or one or more vectors encoding the pair of guide RNAs, wherein the pair of spacer sequences comprise a first spacer sequence that is SEQ ID NO: 243 and a second spacer sequence selected from any one of SEQ ID NOs: 200, 204-210, 212-214, 216-219, 221-224, 226-230, 232-234, 236-237, and 239, respectively. In one embodiment, a composition is provided comprising a pair of gRNAs comprising a pair of spacer sequences, or one or more vectors encoding the pair of guide RNAs, wherein the pair of spacer sequences comprise a first spacer sequence that is SEQ ID NO: 244 and a second spacer sequence selected from any one of SEQ ID NOs: 200, 204-210, 212-214, 216-219, 221-224, 226-230, 232-234, 236-237, and 239, respectively. In one embodiment, a composition is provided comprising a pair of gRNAs comprising a pair of spacer sequences, or one or more vectors encoding the pair of guide RNAs, wherein the pair of spacer sequences comprise a first spacer sequence that is SEQ ID NO: 245 and a second spacer sequence selected from any one of SEQ ID NOs: 200, 204-210, 212-214, 216-219, 221-224, 226-230, 232-234, 236-237, and 239, respectively. In one embodiment, a composition is provided comprising a pair of gRNAs comprising a pair of spacer sequences, or one or more vectors encoding the pair of guide RNAs, wherein the pair of spacer sequences comprise a first spacer sequence that is SEQ ID NO: 246 and a second spacer sequence selected from any one of SEQ ID NOs: 200, 204-210, 212-214, 216-219, 221-224, 226-230, 232-234, 236-237, and 239, respectively. In one embodiment, a composition is provided comprising a pair of gRNAs comprising a pair of spacer sequences, or one or more vectors encoding the pair of guide RNAs, wherein the pair of spacer sequences comprise a first spacer sequence that is SEQ ID NO: 247 and a second spacer sequence selected from any one of SEQ ID NOs: 200, 204-210, 212-214, 216-219, 221-224, 226-230, 232-234, 236-237, and 239, respectively. In one embodiment, a composition is provided comprising a pair of gRNAs comprising a pair of spacer sequences, or one or more vectors encoding the pair of guide RNAs, wherein the pair of spacer sequences comprise a first spacer sequence that is SEQ ID NO: 248 and a second spacer sequence selected from any one of SEQ ID NOs: 200, 204-210, 212-214, 216-219, 221-224, 226-230, 232-234, 236-237, and 239, respectively. In one embodiment, a composition is provided comprising a pair of gRNAs comprising a pair of spacer sequences, or one or more vectors encoding the pair of guide RNAs, wherein the pair of spacer sequences comprise a first spacer sequence that is SEQ ID NO: 249 and a second spacer sequence selected from any one of SEQ ID NOs: ID 200, 204-210, 212-214, 216-219, 221-224, 226-230, 232-234, 236-237, and 239, respectively. In one embodiment, a composition is provided comprising a pair of gRNAs comprising a pair of spacer sequences, or one or more vectors encoding the pair of guide RNAs, wherein the pair of spacer sequences comprise a first spacer sequence that is SEQ ID NO: 250 and a second spacer sequence selected from any one of SEQ ID NOs: 200, 204-210, 212-214, 216-219, 221-224, 226-230, 232-234, 236-237, and 239, respectively. In one embodiment, a composition is provided comprising a pair of gRNAs comprising a pair of spacer sequences, or one or more vectors encoding the pair of guide RNAs, wherein the pair of spacer sequences comprise a first spacer sequence that is SEQ ID NO: 251 and a second spacer sequence selected from any one of SEQ ID NOs: 200, 204-210, 212-214, 216-219, 221-224, 226-230, 232-234, 236-237, and 239, respectively. In one embodiment, a composition is provided comprising a pair of gRNAs comprising a pair of spacer sequences, or one or more vectors encoding the pair of guide RNAs, wherein the pair of spacer sequences comprise a first spacer sequence that is SEQ ID NO: 252 and a second spacer sequence selected from any one of SEQ ID NOs: 200, 204-210, 212-214, 216-219, 221-224, 226-230, 232-234, 236-237, and 239, respectively. In one embodiment, a composition is provided comprising a pair of gRNAs comprising a pair of spacer sequences, or one or more vectors encoding the pair of guide RNAs, wherein the pair of spacer sequences comprise a first spacer sequence that is SEQ ID NO: 253 and a second spacer sequence selected from any one of SEQ ID NOs: 200, 204-210, 212-214, 216-219, 221-224, 226-230, 232-234, 236-237, and 239, respectively. In one embodiment, a composition is provided comprising a pair of gRNAs comprising a pair of spacer sequences, or one or more vectors encoding the pair of guide RNAs, wherein the pair of spacer sequences comprise a first spacer sequence that is SEQ ID NO: 254 and a second spacer sequence selected from any one of SEQ ID NOs: 200, 204-210, 212-214, 216-219, 221-224, 226-230, 232-234, 236-237, and 239, respectively. In one embodiment, a composition is provided comprising a pair of gRNAs comprising a pair of spacer sequences, or one or more vectors encoding the pair of guide RNAs, wherein the pair of spacer sequences comprise a first spacer sequence that is SEQ ID NO: 255 and a second spacer sequence selected from any one of SEQ ID NOs: 200, 204-210, 212-214, 216-219, 221-224, 226-230, 232-234, 236-237, and 239, respectively. In one embodiment, a composition is provided comprising a pair of gRNAs comprising a pair of spacer sequences, or one or more vectors encoding the pair of guide RNAs, wherein the pair of spacer sequences comprise a first spacer sequence that is SEQ ID NO: 256 and a second spacer sequence selected from any one of SEQ ID NOs: 200, 204-210, 212-214, 216-219, 221-224, 226-230, 232-234, 236-237, and 239, respectively. In one embodiment, a composition is provided comprising a pair of gRNAs comprising a pair of spacer sequences, or one or more vectors encoding the pair of guide RNAs, wherein the pair of spacer sequences comprise a first spacer sequence that is SEQ ID NO: 257 and a second spacer sequence selected from any one of SEQ ID NOs: 200, 204-210, 212-214, 216-219, 221-224, 226-230, 232-234, 236-237, and 239, respectively. In one embodiment, a composition is provided comprising a pair of gRNAs comprising a pair of spacer sequences, or one or more vectors encoding the pair of guide RNAs, wherein the pair of spacer sequences comprise a first spacer sequence that is SEQ ID NO: 258 and a second spacer sequence selected from any one of SEQ ID NOs: 200, 204-210, 212-214, 216-219, 221-224, 226-230, 232-234, 236-237, and 239, respectively. In one embodiment, a composition is provided comprising a pair of gRNAs comprising a pair of spacer sequences, or one or more vectors encoding the pair of guide RNAs, wherein the pair of spacer sequences comprise a first spacer sequence that is SEQ ID NO: 259 and a second spacer sequence selected from any one of SEQ ID NOs: 200, 204-210, 212-214, 216-219, 221-224, 226-230, 232-234, 236-237, and 239, respectively.

In some embodiments, nucleotide sequences encoding two guide RNAs and a nucleotide sequence encoding SaCas9 are provided on a single nucleic acid molecule. In some embodiments, the single nucleic acid molecule is a vector. In some embodiments, the vector expresses the two guide RNAs and SaCas9. In some embodiments, the two guide RNAs are identical. In some embodiments, the two guide RNAs are not identical. In some embodiments, the two guide RNAs and SaCas9 are separately expressed, e.g., from their own promoters.

Each of the guide sequences shown in Table 2 at SEQ ID NOs: 200-259 may further comprise additional nucleotides to form or encode a crRNA, e.g., using any known sequence appropriate for the SaCas9 being used. In some embodiments, the crRNA comprises (5′ to 3′) at least a spacer sequence and a first complementarity domain. The first complementary domain is sufficiently complementary to a second complementarity domain, which may be part of the same molecule in the case of an sgRNA or in a tracrRNA in the case of a dual or modular gRNA, to form a duplex. See, e.g., US 2017/0007679 for detailed discussion of crRNA and gRNA domains, including first and second complementarity domains. For sgRNA, a spacer sequence is typically followed (5′ to 3′) by a crRNA, a linker (e.g., GAAA), and a tracrRNA. The crRNA, linker, and tracrRNA is sometimes referred to herein and in the art as a “scaffold” sequence. See, for example, Briner et al. (2014) Mol. Cell 56: 333-339, incorporated herein in its entirety, and in particular, the generalized structure of a sgRNA at FIG. 1A.

An exemplary scaffold sequence suitable for use with SaCas9 to follow the guide sequence at its 3′ end is: GTTTAAGTACTCTGTGCTGGAAACAGCACAGAATCTACTTAAACAAGGCAAAATGCCGT GTTTATCTCGTCAACTTGTTGGCGAGA (SEQ ID NO: 500) in 5′ to 3′ orientation. In some embodiments, an exemplary scaffold sequence for use with SaCas9 to follow the 3′ end of the guide sequence is a sequence that is at least 80%, 85%, 90%, 91%, 92%, 93%, 94%, 95%, 96%, 97%, 98%, 99% or 100% identical to SEQ ID NO: 500, or a sequence that differs from SEQ ID NO: 500 by no more than 1, 2, 3, 4, 5, 10, 15, 20, or 25 nucleotides.

In some embodiments, if the composition comprises one or more nucleic acids encoding an RNA-targeted endonuclease and one or more guide RNAs, the one or more nucleic acids are designed such that they express the one or more guide RNAs at an equivalent or higher level (e.g., a greater number of expressed transgene copies) as compared to the expression level of the RNA-targeted endonuclease. In some embodiments, the one or more nucleic acids are designed such that they express (e.g., on average in 100 cells) the one or more guide RNAs at at least a 1.1, 1.2, 1.3, 1.4, or 1.5 times higher level (e.g., a greater number of expressed transgene copies) as compared to the expression level of the RNA-targeted endonuclease. In some embodiments, the one or more nucleic acids are designed such that they express the one or more guide RNAs at 1.01-1.5, 1.01-1.4, 1.01-1.3, 1.01-1.2, 1.01-1.1, 1.1-2.0, 1.1-1.8, 1.1-1.6, 1.1-1.4, 1.1-1.3, 1.2-2.0, 1.2-1.8, 1.2-1.6, 1.2-1.4, 1.4-2.0, 1.4-1.8, 1.4-1.6, 1.6-2.0, 1.6-1.8, or 1.8-2.0 times higher level (e.g., a greater number of expressed transgene copies) as compared to the expression level of the RNA-targeted endonuclease. In some embodiments, the one or more guide RNAs are designed to express a higher level than the RNA-targeted endonuclease by: a) utilizing one or more regulatory elements (e.g., promoters or enhancers) that express the one or more guide RNAs at a higher level as compared to the regulatory elements (e.g., promoters or enhancers) for expression of the RNA-targeted endonuclease; and/or b) expressing more copies of one or more of the guide RNAs as compared to the number of copies of the RNA-targeted endonuclease (e.g., 2× or 3× as many copies of the nucleotide sequences encoding the one or more guide RNAs as compared to the number of copies of the nucleotide sequences encoding the RNA-targeted endonuclease). For example, in some embodiments, the composition comprises multiple nucleic acid molecules (e.g., in multiple vectors), wherein for every nucleotide sequence encoding an RNA-targeted endonuclease in the nucleic acid molecules in the composition, there are two or three copies of the nucleotide sequence encoding the guide RNA in the nucleic acid molecules in the composition. In some embodiments, the composition comprises a first guide RNA and a second guide RNA, wherein the first guide RNA and the second guide RNA are not the same (e.g., any of the guide RNA pairs disclosed herein), and for every nucleotide sequence encoding an RNA-targeted endonuclease in the nucleic acid molecules in the composition, there are two or three copies of the nucleotide sequence encoding the first guide RNA and/or the second guide RNA.

In some embodiments, the disclosure provides for specific nucleic acid sequence encoding one or more guide RNA components (e.g., any of the spacer and or scaffold sequences disclosed herein). The disclosure contemplates RNA equivalents of any of the DNA sequences provided herein (i.e., in which “T”s are replaced with “U”s), as well as complements (including reverse complements) of any of the sequences disclosed herein. In general, in the case of a DNA vector encoding a gRNA, the U residues in any of the RNA sequences described herein may be replaced with T residues. In general, in the case of a given DNA sequence, the T residues may be replaced with U residues to depict the same sequence as a RNA sequence.

Provided herein are compositions comprising one or more guide RNAs or one or more nucleic acids encoding one or more guide RNAs comprising a guide sequence disclosed herein in Table 2.

TABLE 2
Exemplary spacer sequences

| SEQ | | | |
|---|---|---|---|
| ID | Guide | | |
| NO | RNA | Spacer Sequences (22 mer) | 5′ or 3′ |
| 1 | Slu1 | GATGGAGGGCCTTTTATTCGCG | 3′ |
| 2 | Slu2 | GGCCTTTTATTCGCGAGGGTCG | 3′ |
| 3 | Slu3 | CAGTTCACAACCGCTCCGAGCG | 5′ |
| 4 | Slu4 | AATATCCAAACCGCCGAAGCGG | 3′ |
| 5 | Slu5 | AGGACCCTTCGAGCCCCGTTCG | 5′ |
| 6 | Slu6 | CCACGCTCGGAGCGGTTGTGAA | 5′ |
| 7 | Slu7 | CTCCACGCACCCCCACCTATCG | 3′ |
| 8 | Slu8 | CACCCCCGACCCTCGCGAATAA | 3′ |
| 9 | Slu9 | ACCCTAGAACTGTCTTCGACTC | 5′ |
| 10 | Slu10 | CTTTGCGAACCAACGATAGGTG | 3′ |
| 11 | Slu11 | GAGGGCCTTTTATTCGCGAGGG | 3′ |
| 12 | Slu12 | GGGCCTTTTATTCGCGAGGGTC | 3′ |
| 13 | Slu13 | ACCTCGTCCTCCGACTCGCTGA | 3′ |
| 14 | Slu14 | TTTGCACTTTGCGAACCAACGA | 3′ |
| 15 | Slu15 | CGGGATCCCCGAAAAAGCGGGT | 3′ |
| 16 | Slu16 | CCAGTTCACAACCGCTCCGAGC | 5′ |
| 17 | Slu17 | ACTTTGCGAACCAACGATAGGT | 3′ |
| 18 | Slu18 | ATAAATATCCAAACCGCCGAAG | 3′ |
| 19 | Slu19 | AGATGGAGGGCCTTTTATTCGC | 3′ |
| 20 | Slu20 | CGGCTCCGCCCGCTTCGGCGGT | 3′ |
| 21 | Slu21 | CCCCGGAGTCGAAGACAGTTCT | 5′ |
| 22 | Slu22 | TGGGCGGAGACCCACGCTCGGA | 5′ |
| 23 | Slu23 | GCGCGATCTCTGCCTGCTTACT | 3′ |
| 24 | Slu24 | CACTTTGCGAACCAACGATAGG | 3′ |
| 25 | Slu25 | GCGGCCGGCGAACGGGGCTCGA | 5′ |
| 26 | Slu26 | ATCCGGGCCCGCCCCCTAGCGG | 5′ |
| 27 | Slu27 | CCTGCAGTTTGCCCATCCACGT | 3′ |
| 28 | Slu28 | GGCGCGATCTCTGCCTGCTTAC | 3′ |
| 29 | Slu29 | CAAACCGCCGAAGCGGGCGGAG | 3′ |
| 30 | Slu30 | TGTCTTCGACTCCGGGGCCCCG | 5′ |
| 31 | Slu31 | CCCAACAACCCCAATCCACGTT | 3′ |
| 32 | Slu32 | GGGCGCGGGATCCCCGAAAAAG | 3′ |
| 33 | Slu33 | AGGGCCTTTTATTCGCGAGGGT | 3′ |
| 34 | Slu34 | AATAAATATCCAAACCGCCGAA | 3′ |
| 35 | Slu35 | GGGGCGCGGGATCCCCGAAAAA | 3′ |
| 36 | Slu36 | TGTGATCCGGGCCCGCCCCCTA | 5′ |
| 37 | Slu37 | CCTCCGACTCGCTGACAGGCTA | 3′ |
| 38 | Slu38 | CTTCGAGCCCCGTTCGCCGGCC | 5′ |
| 39 | Slu39 | GGGCTCGAAGGGTCCTTGTAGC | 5′ |
| 40 | Slu40 | GCTCGGAGCGGTTGTGAACTGG | 5′ |
| 41 | Slu41 | CCAGCCGGCTCCGCCCGCTTCG | 3′ |
| 42 | Slu42 | CTGCAGTTTGCCCATCCACGTC | 3′ |
| 43 | Slu43 | GGTCCTGTAGCCTGTCAGCGAG | 3′ |
| 44 | Slu44 | CTCAGTGCATCCAAAACGTGGA | 3′ |
| 45 | Slu45 | TCAGTGCATCCAAAACGTGGAT | 3′ |
| 46 | Slu46 | GCCCCGTTGGAAGACTGAGTGC | 5′ |
| 47 | Slu47 | TTCTTGTGCATGACGCCCTGCT | 3′ |
| 48 | Slu48 | TCTTGTGCATGACGCCCTGCTC | 3′ |
| 49 | Slu49 | TGGAGGATGGAACACGGACGGC | 3′ |
| 50 | Slu50 | TCGCGCCAGACGCTCCCCAGAG | 3′ |
| 51 | Slu51 | CCCCGTTGGAAGACTGAGTGCC | 5′ |
| 53 | Slu53 | GCCGGGTCCGCGGCCGGCGAAC | 5′ |
| 55 | Slu55 | GCTAGGGGGCGGGCCCGGATCA | 5′ |
| 56 | Slu56 | GCCCCGGAGTCGAAGACAGTTC | 5′ |
| 58 | Slu58 | GCCCCGTTCGCCGGCCGCGGAC | 5′ |
| 59 | Slu59 | CCCTAGAACTGTCTTCGACTCC | 5′ |
| 61 | Slu61 | GGGGCTCGAAGGGTCCTTGTAG | 5′ |
| 62 | Slu62 | CCCGGGCACTCAGTCTTCCAAC | 5′ |
| 64 | Slu64 | AGCGGTTGTGAACTGGCAGGCG | 5′ |
| 66 | Slu66 | GGCGCGGCTTCTGTGCCGTGCC | 5′ |
| 70 | Slu70 | CGGAGCGGTTGTGAACTGGCAG | 5′ |
| 200 | Sa1 | GCGGGATGCGAAGCGGCCGAAT | 3′ |
| 201 | Sa2 | GCCCCGGAGTCGAAGACAGTTC | 5′ |
| 202 | Sa3 | CGCGGCCGGCGAACGGGGCTCG | 5′ |
| 203 | Sa4 | CCAGTTCACAACCGCTCCGAGC | 5′ |
| 204 | Sa5 | GGGCCTTTTATTCGCGAGGGTC | 3′ |
| 205 | Sa6 | AGATGGAGGGCCTTTTATTCGC | 3′ |
| 206 | Sa7 | GAGCTAGCGGGATGCGAAGCGG | 3′ |
| 207 | Sa8 | CGGCTCCGCCCGCTTCGGCGGT | 3′ |
| 208 | Sa9 | CAACGATAGGTGGGGGTGCGTG | 3′ |
| 209 | Sa10 | TGGGGACAGACAATAAATACCG | 3′ |
| 210 | Sa11 | CCCAACAACCCCAATCCACGTT | 3′ |
| 211 | Sa12 | ACTCAGTCTTCCAACGGGGCCC | 5′ |
| 212 | Sa13 | GGGGTCTCAGTGCATCCAAAAC | 3′ |
| 213 | Sa14 | ACAACGCAAACCGCGGACACTG | 3′ |
| 214 | Sa15 | CTTCGGCCGCCTCCACACGCCT | 3′ |
| 215 | Sa16 | CCCCGGCCGCTAGGGGGGGGC | 5′ |
| 216 | Sa17 | GGGGCGCGGGATCCCCGAAAAA | 3′ |
| 217 | Sa18 | CAAAACGTGGATTGGGGTTGTT | 3′ |
| 218 | Sa19 | TTGGGGGTCCTGTAGCCTGTCA | 3′ |
| 219 | Sa20 | TCAGTGCATCCAAAACGTGGAT | 3′ |
| 220 | Sa21 | ACTCCGGGGCCCCGTTGGAAGA | 5′ |
| 221 | Sa22 | GACAATAAATACCGAGGAATGT | 3′ |
| 222 | Sa23 | TCGGCCAGGCTGAGGCCCTGAC | 3′ |
| 223 | Sa24 | ACTTTGCGAACCAACGATAGGT | 3′ |
| 224 | Sa25 | CTTTTGCCAAACCCGCTTTTTC | 3′ |
| 225 | Sa26 | GGCTCGAAGGGTCCTTGTAGCC | 5′ |
| 226 | Sa27 | TTTATTCGCGAGGGTCGGGGGT | 3′ |
| 227 | Sa28 | CCGAAGGTCTGGGAGGAGCTAG | 3′ |
| 228 | Sa29 | AGGACCCCCACCCCCGACCCTC | 3′ |
| 229 | Sa30 | GGGTTTGGCAAAAGCAAATTTC | 3′ |
| 230 | Sa31 | AGCGCAAGTGAGGAGGGGGGCG | 3′ |
| 231 | Sa32 | CTAGCGGCCGGGGAGGGAGGGG | 5′ |
| 232 | Sa33 | CTGCTGCTGCTGCTGCTGCTGG | 3′ |
| 233 | NSa1 | CCAGGCTGAGGCCCTGACGTGG | 3′ |
| 234 | NSa3 | AACCAACGATAGGTGGGGGTGC | 3′ |
| 235 | NSa4 | TGTCTTCGACTCCGGGGCCCCG | 5′ |
| 236 | NSa5 | AGGTGGGGACAGACAATAAATA | 3′ |
| 237 | NSa6 | GCGGGCGGAGCCGGCTGGGGCT | 3′ |
| 238 | NSa7 | CGCCTGCCAGTTCACAACCGCT | 5′ |
| 239 | NSa8 | TCGCGCCAGACGCTCCCCAGAG | 3′ |
| 240 | NSa12 | GCCCCGTTGGAAGACTGAGTGC | 5′ |
| 241 | NSa14 | CGCCCAGCTCCAGTCCTGTGAT | 5′ |
| 242 | NSa16 | GGCGCGGCTTCTGTGCCGTGCC | 5′ |
| 243 | NSa17 | GGGGCGGGCCCGGATCACAGGA | 5′ |
| 244 | NSa18 | GGGGCTCGAAGGGTCCTTGTAG | 5′ |
| 245 | NSa24 | CATTCCCGGCTACAAGGACCCT | 5′ |
| 246 | NSa34 | CGGCCCCTCCCTCCCCGGCCGC | 5′ |
| 247 | NSa40 | CGGGCCCGCCCCCTAGCGGCCG | 5′ |
| 248 | NSa41 | CCCGCCCCCTAGCGGCCGGGGA | 5′ |
| 249 | NSa42 | CACTCAGTCTTCCAACGGGGCC | 5′ |
| 250 | NSa45 | GGAGCTGGGCGGAGACCCACGC | 5′ |
| 251 | NSa49 | GCCCCTCCCTCCCCGGCCGCTA | 5′ |
| 252 | NSa51 | ACTGAGTGCCCGGGGCACGGCA | 5′ |
| 253 | NSa54 | GTCCGCGGCCGGCGAACGGGGC | 5′ |
| 254 | NSa55 | GTCTTCCAACGGGGCCCCGGAG | 5′ |
| 255 | NSa58 | GAGACCCACGCTCGGAGCGGTT | 5′ |
| 256 | NSa59 | GTCTTCGACTCCGGGGCCCCGT | 5′ |
| 257 | NSa63 | ACCCTAGAACTGTCTTCGACTC | 5′ |
| 258 | NSa64 | CCCCGTTGGAAGACTGAGTGCC | 5′ |
| 259 | NSa65 | GGCCGGGTCCGCGGCCGGCGAA | 5′ |

SID means SEQ ID NO. In Table 2, the descriptions have the following meaning. A 5 or 3 indicates whether the guide directs cleavage 5′ or 3′ of the repeat region (in the orientation of the forward strand), followed by the genomic coordinates of the sequence (version GRCh38 of the human genome). Where a combination of guides is to be used to direct cleavage 5′ and 3′ of a repeat region, one skilled in the art can select a combination of a 5′ guide disclosed herein and a 3′ guide disclosed herein for a given target such as DMPK.

The following are guide sequences directed to DMPK: SEQ ID NOs: 1-51, 53, 55-56, 58-59, 61-62, 64, 66, 70, and 200-259.

In some embodiments, the disclosure provides a composition comprising one or more guide RNAs (gRNAs) comprising a guide sequence that directs SluCas9 to a target DNA sequence in or near the CTG repeat region in the myotonic dystrophy protein kinase gene (DMPK) associated with myotonic dystrophy type 1. In some embodiments, the invention provides two or more compositions each comprising a guide RNA (gRNA) comprising a guide sequence that directs SluCas9 or SaCas9 to a target DNA sequence in or near the CTG repeat region in the myotonic dystrophy protein kinase gene (DMPK) associated with myotonic dystrophy type 1. The gRNA may comprise a crRNA comprising a DMPK guide sequence shown in Table 2. The gRNA may comprise a crRNA comprising 20 contiguous nucleotides of a DMPK guide sequence shown in Table 2. In some embodiments, the gRNA comprises a crRNA comprising a sequence with about 75%, 80%, 85%, 90%, 95%, 96%, 97%, 98%, 99%, or 100% identity to at least 20 contiguous nucleotides of a DMPK guide sequence shown in Table 2. In some embodiments, the gRNA comprises a crRNA comprising a sequence with about 75%, 80%, 85%, 90%, 95%, 96%, 97%, 98%, 99%, or 100% identity to a guide sequence shown in Table 2. The gRNA may further comprise a trRNA. In each composition and method embodiment described herein, the crRNA and trRNA may be associated as a single RNA (sgRNA) or may be on separate RNAs (dgRNA). In the context of sgRNAs, the crRNA and trRNA components may be covalently linked, e.g., via a phosphodiester bond or other covalent bond.

In each of the composition, use, and method embodiments described herein, the guide RNA may comprise two RNA molecules as a “dual guide RNA” or “dgRNA.” The dgRNA comprises a first RNA molecule comprising a crRNA comprising, e.g., a guide sequence shown in Table 2, and a second RNA molecule comprising a trRNA. The first and second RNA molecules may not be covalently linked, but may form an RNA duplex via the base pairing between portions of the crRNA and the trRNA.

In each of the composition, use, and method embodiments described herein, the guide RNA may comprise a single RNA molecule as a “single guide RNA” or “sgRNA”. The sgRNA may comprise a crRNA (or a portion thereof) comprising a guide sequence shown in Table 2 covalently linked to a trRNA. The sgRNA may comprise 20 contiguous nucleotides of a guide sequence shown in Table 2. In some embodiments, the crRNA and the trRNA are covalently linked via a linker. In some embodiments, the sgRNA forms a stem-loop structure via the base pairing between portions of the crRNA and the trRNA. In some embodiments, the crRNA and the trRNA are covalently linked via one or more bonds that are not a phosphodiester bond.

In some embodiments, the trRNA may comprise all or a portion of a trRNA sequence derived from a naturally-occurring CRISPR/Cas system. In some embodiments, the trRNA comprises a truncated or modified wild type trRNA. The length of the trRNA depends on the CRISPR/Cas system used. In some embodiments, the trRNA comprises or consists of 5, 6, 7, 8, 9, 10, 11, 12, 13, 14, 15, 16, 17, 18, 19, 20, 25, 30, 40, 50, 60, 70, 80, 90, 100, or more than 100 nucleotides. In some embodiments, the trRNA may comprise certain secondary structures, such as, for example, one or more hairpin or stem-loop structures, or one or more bulge structures.

In some embodiments, a composition comprising one or more guide RNAs (or one or more vectors encoding one or more guide RNAs) is provided wherein the one or more gRNAs comprise a guide sequence of any one of SEQ ID NOs: 3, 5, 6, 9, 16, 21, 22, 25, 26, 30, 36, 38, 39, 46, 51, 53, 55, 56, 58, 59, 61, 62, 64, 66, and 70; and a composition comprising one or more guide RNAs (or one or more vectors encoding one or more guide RNAs) wherein the one or more gRNAs comprise a guide sequence of any one of SEQ ID NOs 1, 2, 4, 7, 8, 10, 11, 12, 13, 14, 15, 17, 18, 19, 23, 24, 27, 28, 29, 31, 32, 33, 34, 35, 37, 41, 42, 43, 44, 45, 47, 48, 49, and 50. In some embodiments, the pair of spacer sequences comprises SEQ ID NO: 5 and SEQ ID NO: 10, SEQ ID NO: 46 and SEQ ID NO: 10, SEQ ID NO: 61 and SEQ ID NO: 10, or SEQ ID NO: 64 and SEQ ID NO: 47.

In one aspect, the disclosure provides a composition comprising a gRNA or a vector encoding a gRNA that comprises a guide sequence that is at least 99%, 98%, 97%, 96%, 95%, 94%, 93%, 92%, 91%, or 90% identical to any of the nucleic acids of SEQ ID NOs: 3, 5, 6, 9, 16, 21, 22, 26, 30, 36, 38, 39, 40, 46, 51, 53, 55, 56, 58, 59, 61, 62, 64, 66, and 70; and a composition comprising a gRNA or a vector encoding a gRNA that comprises a guide sequence that is at least 99%, 98%, 97%, 96%, 95%, 94%, 93%, 92%, 91%, or 90% identical to any of the nucleic acids of SEQ ID NOs 1, 2, 4, 7, 8, 10, 11, 12, 13, 14, 15, 17, 18, 19, 20, 23, 24, 27, 28, 29, 31, 32, 33, 34, 35, 37, 41, 42, 43, 44, 45, 47, 48, 49, and 50. In some embodiments, the pair of spacer sequences comprises SEQ ID NO: 5 and SEQ ID NO: 10, SEQ ID NO: 46 and SEQ ID NO: 10, SEQ ID NO: 61 and SEQ ID NO: 10, or SEQ ID NO: 64 and SEQ ID NO: 47.

In other embodiments, the composition comprises at least two gRNAs, or one or more vectors encoding at least two gRNAs, wherein the gRNAs comprise guide sequences selected from any two or more of the guide sequences of SEQ ID NOs: 1-51, 53, 55-56, 58-59, 61-62, 64, 66, or 70. In some embodiments, the composition comprises at least two gRNAs that each comprise a guide sequence at least 99%, 98%, 97%, 96%, 95%, 94%, 93%, 92%, 91%, or 90% identical to any of the nucleic acids of SEQ ID NOs: 1-51, 53, 55-56, 58-59, 61-62, 64, 66, or 70.

Any type of vector, such as any of those described herein, may be used. In some embodiments, the composition comprises one or more vectors encoding one or more gRNAs described herein. In some embodiments, the vector is a viral vector. In some embodiments, the viral vector is a non-integrating viral vector (i.e., that does not insert sequence from the vector into a host chromosome). In some embodiments, the viral vector is an adeno-associated virus vector, a lentiviral vector, an integrase-deficient lentiviral vector, an adenoviral vector, a vaccinia viral vector, an alphaviral vector, or a herpes simplex viral vector. In some embodiments, the vector comprises a muscle-specific promoter. Exemplary muscle-specific promoters include a muscle creatine kinase promoter, a desmin promoter, an MHCK7 promoter, or an SPc5-12 promoter. See US 2004/0175727 A1; Wang et al., Expert Opin Drug Deliv. (2014) 11, 345-364; Wang et al., Gene Therapy (2008) 15, 1489-1499. In some embodiments, the muscle-specific promoter is a CK8 promoter. In some embodiments, the muscle-specific promoter is a CK8e promoter. In any of the foregoing embodiments, the vector may be an adeno-associated virus vector.

In some embodiments, the muscle specific promoter is the CK8 promoter. The CK8 promoter has the following sequence (SEQ ID NO. 700):

| | |
|---|---|
| 1 | CTAGACTAGC ATGCTGCCCA TGTAAGGAGG CAAGGCCTGG GGACACCCGA GATGCCTGGT |
| 61 | TATAATTAAC CCAGACATGT GGCTGCCCCC CCCCCCCCAA CACCTGCTGC CTCTAAAAAT |
| 121 | AACCCTGCAT GCCATGTTCC CGGCGAAGGG CCAGCTGTCC CCCGCCAGCT AGACTCAGCA |
| 181 | CTTAGTTTAG GAACCAGTGA GCAAGTCAGC CCTTGGGGCA GCCCATACAA GGCCATGGGG |
| 241 | CTGGGCAAGC TGCACGCCTG GGTCCGGGGT GGGCACGGTG CCCGGGCAAC GAGCTGAAAG |
| 301 | CTCATCTGCT CTCAGGGGCC CCTCCCTGGG GACAGCCCCT CCTGGCTAGT CACACCCTGT |
| 361 | AGGCTCCTCT ATATAACCCA GGGGCACAGG GGCTGCCCTC ATTCTACCAC CACCTCCACA |
| 421 | GCACAGACAG ACACTCAGGA GCCAGCCAGC |

In some embodiments, the muscle-cell cell specific promoter is a variant of the CK8 promoter, called CK8e. The CK8e promoter has the following sequence (SEQ ID NO. 701):

| | |
|---|---|
| 1 | TGCCCATGTA AGGAGGCAAG GCCTGGGGAC ACCCGAGATG CCTGGTTATA ATTAACCCAG |
| 61 | ACATGTGGCT GCCCCCCCCC CCCCAACACC TGCTGCCTCT AAAAATAACC CTGCATGCCA |
| 121 | TGTTCCCGGC GAAGGGCCAG CTGTCCCCCG CCAGCTAGAC TCAGCACTTA GTTTAGGAAC |
| 181 | CAGTGAGCAA GTCAGCCCTT GGGGCAGCCC ATACAAGGCC ATGGGGCTGG GCAAGCTGCA |
| 241 | CGCCTGGGTC CGGGGTGGGC ACGGTGCCCG GGCAACGAGC TGAAAGCTCA TCTGCTCTCA |
| 301 | GGGGCCCCTC CCTGGGGACA GCCCCTCCTG GCTAGTCACA CCCTGTAGGC TCCTCTATAT |
| 361 | AACCCAGGGG CACAGGGGCT GCCCTCATTC TACCACCACC TCCACAGCAC AGACAGACAC |
| 421 | TCAGGAGCCA GCCAGC |

The guide RNA compositions of the present invention are designed to recognize (e.g., hybridize to) a target sequence in or near a trinucleotide repeat, such as a trinucleotide repeat region in the DMPK gene. For example, the target sequence may be recognized and cleaved by SluCas9. In some embodiments, SluCas9 may be directed by a guide RNA to the target sequence, where the guide sequence of the guide RNA hybridizes with the target sequence and the SluCas9 cleaves the target sequence.

In some embodiments, the guide sequence is at least 99%, 98%, 97%, 96%, 95%, 94%, 93%, 92%, 91%, or 90% identical to a target sequence present in the human gene of interest. In some embodiments, the target sequence may be complementary to the guide sequence of the guide RNA. In some embodiments, the degree of complementarity or identity between a guide sequence of a guide RNA and its corresponding target sequence may be at least 80%, 85%, 90%, 95%, 96%, 97%, 98%, 99%, or 100%. In some embodiments, the target sequence and the guide sequence of the gRNA may be 100% complementary or identical. In other embodiments, the target sequence and the guide sequence of the gRNA may contain at least one mismatch. For example, the target sequence and the guide sequence of the gRNA may contain 1, 2, 3, or 4 mismatches, where the total length of the guide sequence is 20. In some embodiments, the target sequence and the guide sequence of the gRNA may contain 1-4 mismatches where the guide sequence is 20 nucleotides.

In some embodiments, a composition or formulation disclosed herein comprises an mRNA comprising an open reading frame (ORF) encoding an RNA-targeted endonuclease, such as a Cas nuclease as described herein. In some embodiments, an mRNA comprising an ORF encoding an RNA-targeted endonuclease, such as a Cas nuclease, is provided, used, or administered.

In some embodiments, the SluCas9 protein comprises an amino acid sequence that is at least 80%, 85%, 90%, 91%, 92%, 93%, 94%, 95%, 96%, 97%, 98%, 99% or 100% identical to the sequence of SEQ ID NO: 712:

NQKFILGLDIGITSVGYGLIDYETKNIIDAGVRLFPEANVENNEGRRSKR
GSRRLKRRRIHRLERVKKLLEDYNLLDQSQIPQSTNPYAIRVKGLSEALS
KDELVIALLHIAKRRGIHKIDVIDSNDDVGNELSTKEQLNKNSKLLKDKF
VCQIQLERMNEGQVRGEKNRFKTADIIKEIIQLLNVQKNFHQLDENFINK
YIELVEMRREYFEGPGKGSPYGWEGDPKAWYETLMGHCTYFPDELRSVKY
AYSADLFNALNDLNNLVIQRDGLSKLEYHEKYHIIENVFKQKKKPTLKQI
ANEINVNPEDIKGYRITKSGKPQFTEFKLYHDLKSVLFDQSILENEDVLD
QIAEILTIYQDKDSIKSKLTELDILLNEEDKENIAQLTGYTGTHRLSLKC
IRLVLEEQWYSSRNQMEIFTHLNIKPKKINLTAANKIPKAMIDEFILSPV
VKRTFGQAINLINKIIEKYGVPEDIIIELARENNSKDKQKFINEMQKKNE
NTRKRINEIIGKYGNQNAKRLVEKIRLHDEQEGKCLYSLESIPLEDLLNN
PNHYEVDHIIPRSVSFDNSYHNKVLVKQSENSKKSNLTPYQYFNSGKSKL
SYNQFKQHILNLSKSQDRISKKKKEYLLEERDINKFEVQKEFINRNLVDT
RYATRELTNYLKAYFSANNMNVKVKTINGSFTDYLRKVWKFKKERNHGYK
HHAEDALIIANADFLFKENKKLKAVNSVLEKPEIETKQLDIQVDSEDNYS
EMFIIPKQVQDIKDFRNFKYSHRVDKKPNRQLINDTLYSTRKKDNSTYIV
QTIKDIYAKDNTTLKKQFDKSPEKFLMYQHDPRTFEKLEVIMKQYANEKN
PLAKYHEETGEYLTKYSKKNNGPIVKSLKYIGNKLGSHLDVTHQFKSSTK
KLVKLSIKPYRFDVYLTDKGYKFITISYLDVLKKDNYYYIPEQKYDKLKL
GKAIDKNAKFIASFYKNDLIKLDGEIYKIIGVNSDTRNMIELDLPDIRYK
EYCELNNIKGEPRIKKTIGKKVNSIEKLTTDVLGNVFTNTQYTKPQLLFK
RGN.

In some embodiments, the SluCas9 is a variant of the amino acid sequence of SEQ ID NO: 712. In some embodiments, the SluCas9 comprises an amino acid other than an Q at the position corresponding to position 781 of SEQ ID NO: 712. In some embodiments, the SluCas9 comprises an amino acid other than an R at the position corresponding to position 1013 of SEQ ID NO: 712. In some embodiments, the SluCas9 comprises a K at the position corresponding to position 781 of SEQ ID NO: 712. In some embodiments, the SluCas9 comprises a K at the position corresponding to position 966 of SEQ ID NO: 712. In some embodiments, the SluCas9 comprises an H at the position corresponding to position 1013 of SEQ ID NO: 712. In some embodiments, the SluCas9 comprises an amino acid other than an Q at the position corresponding to position 781 of SEQ ID NO: 712; and an amino acid other than an R at the position corresponding to position 1013 of SEQ ID NO: 712. In some embodiments, the SluCas9 comprises a K at the position corresponding to position 781 of SEQ ID NO: 712; a K at the position corresponding to position 966 of SEQ ID NO: 712; and an H at the position corresponding to position 1013 of SEQ ID NO: 712.

In some embodiments, the SluCas9 comprises an amino acid other than an R at the position corresponding to position 246 of SEQ ID NO: 712. In some embodiments, the SluCas9 comprises an amino acid other than an N at the position corresponding to position 414 of SEQ ID NO: 712. In some embodiments, the SluCas9 comprises an amino acid other than a T at the position corresponding to position 420 of SEQ ID NO: 712. In some embodiments, the SluCas9 comprises an amino acid other than an R at the position corresponding to position 655 of SEQ ID NO: 712. In some embodiments, the SluCas9 comprises an amino acid other than an R at the position corresponding to position 246 of SEQ ID NO: 712; an amino acid other than an N at the position corresponding to position 414 of SEQ ID NO: 712; an amino acid other than a T at the position corresponding to position 420 of SEQ ID NO: 712; and an amino acid other than an R at the position corresponding to position 655 of SEQ ID NO: 712. In some embodiments, the SluCas9 comprises an A at the position corresponding to position 246 of SEQ ID NO: 712. In some embodiments, the SluCas9 comprises an A at the position corresponding to position 414 of SEQ ID NO: 712. In some embodiments, the SluCas9 comprises an A at the position corresponding to position 420 of SEQ ID NO: 712. In some embodiments, the SluCas9 comprises an A at the position corresponding to position 655 of SEQ ID NO: 712. In some embodiments, the SluCas9 comprises an A at the position corresponding to position 246 of SEQ ID NO: 712; an A at the position corresponding to position 414 of SEQ ID NO: 712; an A at the position corresponding to position 420 of SEQ ID NO: 712; and an A at the position corresponding to position 655 of SEQ ID NO: 712.

In some embodiments, the SluCas9 comprises an amino acid other than an R at the position corresponding to position 246 of SEQ ID NO: 712; an amino acid other than an N at the position corresponding to position 414 of SEQ ID NO: 712; an amino acid other than a T at the position corresponding to position 420 of SEQ ID NO: 712; an amino acid other than an R at the position corresponding to position 655 of SEQ ID NO: 712; an amino acid other than an Q at the position corresponding to position 781 of SEQ ID NO: 712; a K at the position corresponding to position 966 of SEQ ID NO: 712; and an amino acid other than an R at the position corresponding to position 1013 of SEQ ID NO: 712. In some embodiments, the SluCas9 comprises an A at the position corresponding to position 246 of SEQ ID NO: 712; an A at the position corresponding to position 414 of SEQ ID NO: 712; an A at the position corresponding to position 420 of SEQ ID NO: 712; an A at the position corresponding to position 655 of SEQ ID NO: 712; a K at the position corresponding to position 781 of SEQ ID NO: 712; a K at the position corresponding to position 966 of SEQ ID NO: 712; and an H at the position corresponding to position 1013 of SEQ ID NO: 712.

In some embodiments, the SluCas9 comprises an amino acid sequence that is at least 80%, 85%, 90%, 91%, 92%, 93%, 94%, 95%, 96%, 97%, 98%, 99% or 100% identical to the sequence of SEQ ID NO: 713 (designated herein as SluCas9-KH or SLUCAS9KH):

NQKFILGLDIGITSVGYGLIDYETKNIIDAGVRLFPEANVENNEGRRSKR
GSRRLKRRRIHRLERVKKLLEDYNLLDQSQIPQSTNPYAIRVKGLSEALS
KDELVIALLHIAKRRGIHKIDVIDSNDDVGNELSTKEQLNKNSKLLKDKF
VCQIQLERMNEGQVRGEKNRFKTADIIKEIIQLLNVQKNFHQLDENFINK
YIELVEMRREYFEGPGKGSPYGWEGDPKAWYETLMGHCTYFPDELRSVKY
AYSADLFNALNDLNNLVIQRDGLSKLEYHEKYHIIENVFKQKKKPTLKQI
ANEINVNPEDIKGYRITKSGKPQFTEFKLYHDLKSVLFDQSILENEDVLD
QIAEILTIYQDKDSIKSKLTELDILLNEEDKENIAQLTGYTGTHRLSLKC
IRLVLEEQWYSSRNQMEIFTHLNIKPKKINLTAANKIPKAMIDEFILSPV
VKRTFGQAINLINKIIEKYGVPEDIIIELARENNSKDKQKFINEMQKKNE
NTRKRINEIIGKYGNQNAKRLVEKIRLHDEQEGKCLYSLESIPLEDLLNN
PNHYEVDHIIPRSVSFDNSYHNKVLVKQSENSKKSNLTPYQYFNSGKSKL
SYNQFKQHILNLSKSQDRISKKKKEYLLEERDINKFEVQKEFINRNLVDT
RYATRELTNYLKAYFSANNMNVKVKTINGSFTDYLRKVWKFKKERNHGYK
HHAEDALIIANADFLFKENKKLKAVNSVLEKPEIETKQLDIQVDSEDNYS
EMFIIPKQVQDIKDFRNFKYSHRVDKKPNRKLINDTLYSTRKKDNSTYIV
QTIKDIYAKDNTTLKKQFDKSPEKFLMYQHDPRTFEKLEVIMKQYANEKN
PLAKYHEETGEYLTKYSKKNNGPIVKSLKYIGNKLGSHLDVTHQFKSSTK
KLVKLSIKPYRFDVYLTDKGYKFITISYLDVLKKDNYYYIPEQKYDKLKL
GKAIDKNAKFIASFYKNDLIKLDGEIYKIIGVNSDTRNMIELDLPDIRYK
EYCELNNIKGEPHIKKTIGKKVNSIEKLTTDVLGNVFTNTQYTKPQLLFK
RGN.

In some embodiments, the SluCas9 comprises an amino acid sequence that is at least 80%, 85%, 90%, 91%, 92%, 93%, 94%, 95%, 96%, 97%, 98%, 99% or 100% identical to the sequence of SEQ ID NO: 714 (designated herein as SluCas9-HF):

NQKFILGLDIGITSVGYGLIDYETKNIIDAGVRLFPEANVENNEGRRSKR
GSRRLKRRRIHRLERVKKLLEDYNLLDQSQIPQSTNPYAIRVKGLSEALS
KDELVIALLHIAKRRGIHKIDVIDSNDDVGNELSTKEQLNKNSKLLKDKF
VCQIQLERMNEGQVRGEKNRFKTADIIKEIIQLLNVQKNFHQLDENFINK
YIELVEMRREYFEGPGKGSPYGWEGDPKAWYETLMGHCTYFPDELASVKY
AYSADLFNALNDLNNLVIQRDGLSKLEYHEKYHIIENVFKQKKKPTLKQI
ANEINVNPEDIKGYRITKSGKPQFTEFKLYHDLKSVLFDQSILENEDVLD
QIAEILTIYQDKDSIKSKLTELDILLNEEDKENIAQLTGYTGTHRLSLKC
IRLVLEEQWYSSRAQMEIFAHLNIKPKKINLTAANKIPKAMIDEFILSPV
VKRTFGQAINLINKIIEKYGVPEDIIIELARENNSKDKQKFINEMQKKNE
NTRKRINEIIGKYGNQNAKRLVEKIRLHDEQEGKCLYSLESIPLEDLLNN
PNHYEVDHIIPRSVSFDNSYHNKVLVKQSENSKKSNLTPYQYFNSGKSKL
SYNQFKQHILNLSKSQDRISKKKKEYLLEERDINKFEVQKEFINRNLVDT
RYATAELTNYLKAYFSANNMNVKVKTINGSFTDYLRKVWKFKKERNHGYK
HHAEDALIIANADFLFKENKKLKAVNSVLEKPEIETKQLDIQVDSEDNYS
EMFIIPKQVQDIKDFRNFKYSHRVDKKPNRQLINDTLYSTRKKDNSTYIV
QTIKDIYAKDNTTLKKQFDKSPEKFLMYQHDPRTFEKLEVIMKQYANEKN
PLAKYHEETGEYLTKYSKKNNGPIVKSLKYIGNKLGSHLDVTHQFKSSTK
KLVKLSIKPYRFDVYLTDKGYKFITISYLDVLKKDNYYYIPEQKYDKLKL
GKAIDKNAKFIASFYKNDLIKLDGEIYKIIGVNSDTRNMIELDLPDIRYK
EYCELNNIKGEPRIKKTIGKKVNSIEKLTTDVLGNVFTNTQYTKPQLLFK
RGN.

In some embodiments, the SluCas9 comprises an amino acid sequence that is at least 80%, 85%, 90%, 91%, 92%, 93%, 94%, 95%, 96%, 97%, 98%, 99% or 100% identical to the sequence of SEQ ID NO: 715 (designated herein as SluCas9-HF-KH):

NQKFILGLDIGITSVGYGLIDYETKNIIDAGVRLFPEANVENNEGRRSKR
GSRRLKRRRIHRLERVKKLLEDYNLLDQSQIPQSTNPYAIRVKGLSEALS
KDELVIALLHIAKRRGIHKIDVIDSNDDVGNELSTKEQLNKNSKLLKDKF
VCQIQLERMNEGQVRGEKNRFKTADIIKEIIQLLNVQKNFHQLDENFINK
YIELVEMRREYFEGPGKGSPYGWEGDPKAWYETLMGHCTYFPDELASVKY
AYSADLFNALNDLNNLVIQRDGLSKLEYHEKYHIIENVFKQKKKPTLKQI
ANEINVNPEDIKGYRITKSGKPQFTEFKLYHDLKSVLFDQSILENEDVLD
QIAEILTIYQDKDSIKSKLTELDILLNEEDKENIAQLTGYTGTHRLSLKC
IRLVLEEQWYSSRAQMEIFAHLNIKPKKINLTAANKIPKAMIDEFILSPV
VKRTFGQAINLINKIIEKYGVPEDIIIELARENNSKDKQKFINEMQKKNE
NTRKRINEIIGKYGNQNAKRLVEKIRLHDEQEGKCLYSLESIPLEDLLNN
PNHYEVDHIIPRSVSFDNSYHNKVLVKQSENSKKSNLTPYQYFNSGKSKL
SYNQFKQHILNLSKSQDRISKKKKEYLLEERDINKFEVQKEFINRNLVDT
RYATAELTNYLKAYFSANNMNVKVKTINGSFTDYLRKVWKFKKERNHGYK
HHAEDALIIANADFLFKENKKLKAVNSVLEKPEIETKQLDIQVDSEDNYS
EMFIIPKQVQDIKDFRNFKYSHRVDKKPNRKLINDTLYSTRKKDNSTYIV
QTIKDIYAKDNTTLKKQFDKSPEKFLMYQHDPRTFEKLEVIMKQYANEKN
PLAKYHEETGEYLTKYSKKNNGPIVKSLKYIGNKLGSHLDVTHQFKSSTK
KLVKLSIKPYRFDVYLTDKGYKFITISYLDVLKKDNYYYIPEQKYDKLKL
GKAIDKNAKFIASFYKNDLIKLDGEIYKIIGVNSDTRNMIELDLPDIRYK
EYCELNNIKGEPHIKKTIGKKVNSIEKLTTDVLGNVFTNTQYTKPQLLFK
RGN.

In some embodiments, the Cas protein is any of the engineered Cas proteins disclosed in Schmidt et al., 2021, Nature Communications, “Improved CRISPR genome editing using small highly active and specific engineered RNA-guided nucleases.”

In some embodiments, the Cas9 comprises an amino acid sequence that is at least 80%, 85%, 90%, 91%, 92%, 93%, 94%, 95%, 96%, 97%, 98%, 99% or 100% identical to the sequence of SEQ ID NO: 716 (designated herein as sRGN1):

MNQKFILGLDIGITSVGYGLIDYETKNIIDAGVRLFPEANVENNEGRRSK
RGSRRLKRRRIHRLDRVKHLLAEYDLLDLTNIPKSTNPYQTRVKGLNEKL
SKDELVIALLHIAKRRGIHNVDVAADKEETASDSLSTKDQINKNAKFLES
RYVCELQKERLENEGHVRGVENRFLTKDIVREAKKIIDTQMQYYPEIDET
FKEKYISLVETRREYFEGPGKGSPFGWEGNIKKWFEQMMGHCTYFPEELR
SVKYSYSAELFNALNDLNNLVITRDEDAKLNYGEKFQIIENVFKQKKTPN
LKQIAIEIGVHETEIKGYRVNKSGTPEFTEFKLYHDLKSIVFDKSILENE
AILDQIAEILTIYQDEQSIKEELNKLPEILNEQDKAEIAKLIGYNGTHRL
SLKCIHLINEELWQTSRNQMEIFNYLNIKPNKVDLSEQNKIPKDMVNDFI
LSPVVKRTFIQSINVINKVIEKYGIPEDIIIELARENNSDDRKKFINNLQ
KKNEATRKRINEIIGQTGNQNAKRIVEKIRLHDQQEGKCLYSLKDIPLED
LLRNPNNYDIDHIIPRSVSFDDSMHNKVLVRREQNAKKNNQTPYQYLTSG
YADIKYSVFKQHVLNLAENKDRMTKKKREYLLEERDINKFEVQKEFINRN
LVDTRYATRELTNYLKAYFSANNMNVKVKTINGSFTDYLRKVWKFKKERN
HGYKHHAEDALIIANADFLFKENKKLKAVNSVLEKPEIETKQLDIQVDSE
DNYSEMFIIPKQVQDIKDFRNFKYSHRVDKKPNRQLINDTLYSTRKKDNS
TYIVQTIKDIYAKDNTTLKKQFDKSPEKFLMYQHDPRTFEKLEVIMKQYA
NEKNPLAKYHEETGEYLTKYSKKNNGPIVKSLKYIGNKLGSHLDVTHQFK
SSTKKLVKLSIKPYRFDVYLTDKGYKFITISYLDVLKKDNYYYIPEQKYD
KLKLGKAIDKNAKFIASFYKNDLIKLDGEIYKIIGVNSDTRNMIELDLPD
IRYKEYCELNNIKGEPRIKKTIGKKVNSIEKLTTDVLGNVFTNTQYTKPQ
LLFKRGN.

In some embodiments, the Cas9 comprises an amino acid sequence that is at least 80%, 85%, 90%, 91%, 92%, 93%, 94%, 95%, 96%, 97%, 98%, 99% or 100% identical to the sequence of SEQ ID NO: 717 (designated herein as sRGN2):

MNQKFILGLDIGITSVGYGLIDYETKNIIDAGVRLFPEANVENNEGRRSK
RGSRRLKRRRIHRLERVKSLLSEYKIISGLAPTNNQPYNIRVKGLTEQLT
KDELAVALLHIAKRRGIHKIDVIDSNDDVGNELSTKEQLNKNSKLLKDKF
VCQIQLERMNEGQVRGEKNRFKTADIIKEIIQLLNVQKNFHQLDENFINK
YIELVEMRREYFEGPGQGSPFGWNGDLKKWYEMLMGHCTYFPQELRSVKY
AYSADLFNALNDLNNLIIQRDNSEKLEYHEKYHIIENVFKQKKKPTLKQI
AKEIGVNPEDIKGYRITKSGTPEFTEFKLYHDLKSVLFDQSILENEDVLD
QIAEILTIYQDKDSIKSKLTELDILLNEEDKENIAQLTGYNGTHRLSLKC
IRLVLEEQWYSSRNQMEIFTHLNIKPKKINLTAANKIPKAMIDEFILSPV
VKRTFIQSINVINKVIEKYGIPEDIIIELARENNSDDRKKFINNLQKKNE
ATRKRINEIIGQTGNQNAKRIVEKIRLHDQQEGKCLYSLESIALMDLLNN
PQNYEVDHIIPRSVAFDNSIHNKVLVKQIENSKKGNRTPYQYLNSSDAKL
SYNQFKQHILNLSKSKDRISKKKKDYLLEERDINKFEVQKEFINRNLVDT
RYATRELTSYLKAYFSANNMDVKVKTINGSFTNHLRKVWRFDKYRNHGYK
HHAEDALIIANADFLFKENKKLKAVNSVLEKPEIETKQLDIQVDSEDNYS
EMFIIPKQVQDIKDFRNFKYSHRVDKKPNRQLINDTLYSTRKKDNSTYIV
QTIKDIYAKDNTTLKKQFDKSPEKFLMYQHDPRTFEKLEVIMKQYANEKN
PLAKYHEETGEYLTKYSKKNNGPIVKSLKYIGNKLGSHLDVTHQFKSSTK
KLVKLSIKPYRFDVYLTDKGYKFITISYLDVLKKDNYYYIPEQKYDKLKL
GKAIDKNAKFIASFYKNDLIKLDGEIYKIIGVNSDTRNMIELDLPDIRYK
EYCELNNIKGEPRIKKTIGKKVNSIEKLTTDVLGNVFTNTQYTKPQLLFK
RGN.

In some embodiments, the Cas9 comprises an amino acid sequence that is at least 80%, 85%, 90%, 91%, 92%, 93%, 94%, 95%, 96%, 97%, 98%, 99% or 100% identical to the sequence of SEQ ID NO: 718 (designated herein as sRGN3):

MNQKFILGLDIGITSVGYGLIDYETKNIIDAGVRLFPEANVENNEGRRSK
RGSRRLKRRRIHRLERVKLLLTEYDLINKEQIPTSNNPYQIRVKGLSEIL
SKDELAIALLHLAKRRGIHNVDVAADKEETASDSLSTKDQINKNAKFLES
RYVCELQKERLENEGHVRGVENRFLTKDIVREAKKIIDTQMQYYPEIDET
FKEKYISLVETRREYFEGPGQGSPFGWNGDLKKWYEMLMGHCTYFPQELR
SVKYAYSADLFNALNDLNNLIIQRDNSEKLEYHEKYHIIENVFKQKKKPT
LKQIAKEIGVNPEDIKGYRITKSGTPEFTSFKLFHDLKKVVKDHAILDDI
DLLNQIAEILTIYQDKDSIVAELGQLEYLMSEADKQSISELTGYTGTHSL
SLKCMNMIIDELWHSSMNQMEVFTYLNMRPKKYELKGYQRIPTDMIDDAI
LSPVVKRTFIQSINVINKVIEKYGIPEDIIIELARENNSDDRKKFINNLQ
KKNEATRKRINEIIGQTGNQNAKRIVEKIRLHDQQEGKCLYSLESIPLED
LLNNPNHYEVDHIIPRSVSFDNSYHNKVLVKQSENSKKSNLTPYQYFNSG
KSKLSYNQFKQHILNLSKSQDRISKKKKEYLLEERDINKFEVQKEFINRN
LVDTRYATRELTNYLKAYFSANNMNVKVKTINGSFTDYLRKVWKFKKERN
HGYKHHAEDALIIANADFLFKENKKLKAVNSVLEKPEIETKQLDIQVDSE
DNYSEMFIIPKQVQDIKDFRNFKYSHRVDKKPNRQLINDTLYSTRKKDNS
TYIVQTIKDIYAKDNTTLKKQFDKSPEKFLMYQHDPRTFEKLEVIMKQYA
NEKNPLAKYHEETGEYLTKYSKKNNGPIVKSLKYIGNKLGSHLDVTHQFK
SSTKKLVKLSIKPYRFDVYLTDKGYKFITISYLDVLKKDNYYYIPEQKYD
KLKLGKAIDKNAKFIASFYKNDLIKLDGEIYKIIGVNSDTRNMIELDLPD
IRYKEYCELNNIKGEPRIKKTIGKKVNSIEKLTTDVLGNVFTNTQYTKPQ
LLFKRGN.

In some embodiments, the Cas9 comprises an amino acid sequence that is at least 80%, 85%, 90%, 91%, 92%, 93%, 94%, 95%, 96%, 97%, 98%, 99% or 100% identical to the sequence of SEQ ID NO: 719 (designated herein as sRGN3.1):

MNQKFILGLDIGITSVGYGLIDYETKNIIDAGVRLFPEANVENNEGRRSK
RGSRRLKRRRIHRLERVKLLLTEYDLINKEQIPTSNNPYQIRVKGLSEIL
SKDELAIALLHLAKRRGIHNVDVAADKEETASDSLSTKDQINKNAKFLES
RYVCELQKERLENEGHVRGVENRFLTKDIVREAKKIIDTQMQYYPEIDET
FKEKYISLVETRREYFEGPGQGSPFGWNGDLKKWYEMLMGHCTYFPQELR
SVKYAYSADLFNALNDLNNLIIQRDNSEKLEYHEKYHIIENVFKQKKKPT
LKQIAKEIGVNPEDIKGYRITKSGTPEFTSFKLFHDLKKVVKDHAILDDI
DLLNQIAEILTIYQDKDSIVAELGQLEYLMSEADKQSISELTGYTGTHSL
SLKCMNMIIDELWHSSMNQMEVFTYLNMRPKKYELKGYQRIPTDMIDDAI
LSPVVKRTFIQSINVINKVIEKYGIPEDIIIELARENNSDDRKKFINNLQ
KKNEATRKRINEIIGQTGNQNAKRIVEKIRLHDQQEGKCLYSLESIPLED
LLNNPNHYEVDHIIPRSVSFDNSYHNKVLVKQSENSKKSNLTPYQYFNSG
KSKLSYNQFKQHILNLSKSQDRISKKKKEYLLEERDINKFEVQKEFINRN
LVDTRYATRELTNYLKAYFSANNMNVKVKTINGSFTDYLRKVWKFKKERN
HGYKHHAEDALIIANADFLFKENKKLKAVNSVLEKPEIETKQLDIQVDSE
DNYSEMFIIPKQVQDIKDFRNFKYSHRVDKKPNRQLINDTLYSTRKKDNS
TYIVQTIKDIYAKDNTTLKKQFDKSPEKFLMYQHDPRTFEKLEVIMKQYA
NEKNPLAKYHEETGEYLTKYSKKNNGPIVKSLKYIGNKLGSHLDVTHQFK
SSTKKLVKLSIKNYRFDVYLTEKGYKFVTIAYLNVFKKDNYYYIPKDKYQ
ELKEKKKIKDTDQFIASFYKNDLIKLNGDLYKIIGVNSDDRNIIELDYYD
IKYKDYCEINNIKGEPRIKKTIGKKTESIEKFTTDVLGNLYLHSTEKAPQ
LIFKRGL.

In some embodiments, the Cas9 comprises an amino acid sequence that is at least 80%, 85%, 90%, 91%, 92%, 93%, 94%, 95%, 96%, 97%, 98%, 99% or 100% identical to the sequence of SEQ ID NO: 720 (designated herein as sRGN3.2):

MNQKFILGLDIGITSVGYGLIDYETKNIIDAGVRLFPEANVENNEGRRSK
RGSRRLKRRRIHRLERVKLLLTEYDLINKEQIPTSNNPYQIRVKGLSEIL
SKDELAIALLHLAKRRGIHNVDVAADKEETASDSLSTKDQINKNAKFLES
RYVCELQKERLENEGHVRGVENRFLTKDIVREAKKIIDTQMQYYPEIDET
FKEKYISLVETRREYFEGPGQGSPFGWNGDLKKWYEMLMGHCTYFPQELR
SVKYAYSADLFNALNDLNNLIIQRDNSEKLEYHEKYHIIENVFKQKKKPT
LKQIAKEIGVNPEDIKGYRITKSGTPEFTSFKLFHDLKKVVKDHAILDDI
DLLNQIAEILTIYQDKDSIVAELGQLEYLMSEADKQSISELTGYTGTHSL
SLKCMNMIIDELWHSSMNQMEVFTYLNMRPKKYELKGYQRIPTDMIDDAI
LSPVVKRTFIQSINVINKVIEKYGIPEDIIIELARENNSDDRKKFINNLQ
KKNEATRKRINEIIGQTGNQNAKRIVEKIRLHDQQEGKCLYSLESIPLED
LLNNPNHYEVDHIIPRSVSFDNSYHNKVLVKQSENSKKSNLTPYQYFNSG
KSKLSYNQFKQHILNLSKSQDRISKKKKEYLLEERDINKFEVQKEFINRN
LVDTRYATRELTNYLKAYFSANNMNVKVKTINGSFTDYLRKVWKFKKERN
HGYKHHAEDALIIANADFLFKENKKLKAVNSVLEKPEIETKQLDIQVDSE
DNYSEMFIIPKQVQDIKDFRNFKFSHRVDKKPNRQLINDTLYSTRMKDEH
DYIVQTITDIYGKDNTNLKKQFNKNPEKFLMYQNDPKTFEKLSIIMKQYS
DEKNPLAKYYEETGEYLTKYSKKNNGPIVKKIKLLGNKVGNHLDVTNKYE
NSTKKLVKLSIKNYRFDVYLTEKGYKFVTIAYLNVFKKDNYYYIPKDKYQ
ELKEKKKIKDTDQFIASFYKNDLIKLNGDLYKIIGVNSDDRNIIELDYYD
IKYKDYCEINNIKGEPRIKKTIGKKTESIEKFTTDVLGNLYLHSTEKAPQ
LIFKRGL.

In some embodiments, the Cas9 comprises an amino acid sequence that is at least 80%, 85%, 90%, 91%, 92%, 93%, 94%, 95%, 96%, 97%, 98%, 99% or 100% identical to the sequence of SEQ ID NO: 721 (designated herein as sRGN3.3):

MNQKFILGLDIGITSVGYGLIDYETKNIIDAGVRLFPEANVENNEGRRSK
RGSRRLKRRRIHRLERVKLLLTEYDLINKEQIPTSNNPYQIRVKGLSEIL
SKDELAIALLHLAKRRGIHNVDVAADKEETASDSLSTKDQINKNAKFLES
RYVCELQKERLENEGHVRGVENRFLTKDIVREAKKIIDTQMQYYPEIDET
FKEKYISLVETRREYFEGPGQGSPFGWNGDLKKWYEMLMGHCTYFPQELR
SVKYAYSADLFNALNDLNNLIIQRDNSEKLEYHEKYHIIENVFKQKKKPT
LKQIAKEIGVNPEDIKGYRITKSGTPEFTSFKLFHDLKKVVKDHAILDDI
DLLNQIAEILTIYQDKDSIVAELGQLEYLMSEADKQSISELTGYTGTHSL
SLKCMNMIIDELWHSSMNQMEVFTYLNMRPKKYELKGYQRIPTDMIDDAI
LSPVVKRTFIQSINVINKVIEKYGIPEDIIIELARENNSDDRKKFINNLQ
KKNEATRKRINEIIGQTGNQNAKRIVEKIRLHDQQEGKCLYSLESIPLED
LLNNPNHYEVDHIIPRSVSFDNSYHNKVLVKQSENSKKSNLTPYQYFNSG
KSKLSYNQFKQHILNLSKSQDRISKKKKEYLLEERDINKFEVQKEFINRN
LVDTRYATRELTSYLKAYFSANNMDVKVKTINGSFTNHLRKVWRFDKYRN
HGYKHHAEDALIIANADFLFKENKKLQNTNKILEKPTIENNTKKVTVEKE
EDYNNVFETPKLVEDIKQYRDYKFSHRVDKKPNRQLINDTLYSTRMKDEH
DYIVQTITDIYGKDNTNLKKQFNKNPEKFLMYQNDPKTFEKLSIIMKQYS
DEKNPLAKYYEETGEYLTKYSKKNNGPIVKKIKLLGNKVGNHLDVTNKYE
NSTKKLVKLSIKNYRFDVYLTEKGYKFVTIAYLNVFKKDNYYYIPKDKYQ
ELKEKKKIKDTDQFIASFYKNDLIKLNGDLYKIIGVNSDDRNIIELDYYD
IKYKDYCEINNIKGEPRIKKTIGKKTESIEKFTTDVLGNLYLHSTEKAPQ
LIFKRGL.

In some embodiments, the Cas9 comprises an amino acid sequence that is at least 80%, 85%, 90%, 91%, 92%, 93%, 94%, 95%, 96%, 97%, 98%, 99% or 100% identical to the sequence of SEQ ID NO: 722 (designated herein as sRGN4):

MNQKFILGLDIGITSVGYGLIDYETKNIIDAGVRLFPEANVENNEGRRSK
RGSRRLKRRRIHRLERVKKLLEDYNLLDQSQIPQSTNPYAIRVKGLSEAL
SKDELVIALLHIAKRRGIHNINVSSEDEDASNELSTKEQINRNNKLLKDK
YVCEVQLQRLKEGQIRGEKNRFKTTDILKEIDQLLKVQKDYHNLDIDFIN
QYKEIVETRREYFEGPGKGSPYGWEGDPKAWYETLMGHCTYFPDELRSVK
YAYSADLFNALNDLNNLVIQRDGLSKLEYHEKYHIIENVFKQKKKPTLKQ
IANEINVNPEDIKGYRITKSGKPEFTSFKLFHDLKKVVKDHAILDDIDLL
NQIAEILTIYQDKDSIVAELGQLEYLMSEADKQSISELTGYTGTHSLSLK
CMNMIIDELWHSSMNQMEVFTYLNMRPKKYELKGYQRIPTDMIDDAILSP
VVKRTFIQSINVINKVIEKYGIPEDIIIELARENNSDDRKKFINNLQKKN
EATRKRINEIIGQTGNQNAKRIVEKIRLHDQQEGKCLYSLESIPLEDLLN
NPNHYEVDHIIPRSVSFDNSYHNKVLVKQSENSKKSNLTPYQYFNSGKSK
LSYNQFKQHILNLSKSQDRISKKKKEYLLEERDINKFEVQKEFINRNLVD
TRYATRELTNYLKAYFSANNMNVKVKTINGSFTDYLRKVWKFKKERNHGY
KHHAEDALIIANADFLFKENKKLKAVNSVLEKPEIETKQLDIQVDSEDNY
SEMFIIPKQVQDIKDFRNFKYSHRVDKKPNRQLINDTLYSTRKKDNSTYI
VQTIKDIYAKDNTTLKKQFDKSPEKFLMYQHDPRTFEKLEVIMKQYANEK
NPLAKYHEETGEYLTKYSKKNNGPIVKSLKYIGNKLGSHLDVTHQFKSST
KKLVKLSIKPYRFDVYLTDKGYKFITISYLDVLKKDNYYYIPEQKYDKLK
LGKAIDKNAKFIASFYKNDLIKLDGEIYKIIGVNSDTRNMIELDLPDIRY
KEYCELNNIKGEPRIKKTIGKKVNSIEKLTTDVLGNVFTNTQYTKPQLLF
KRGN.

Modified gRNAs

In some embodiments, the gRNA is chemically modified. A gRNA comprising one or more modified nucleosides or nucleotides is called a “modified” gRNA or “chemically modified” gRNA, to describe the presence of one or more non-naturally and/or naturally occurring components or configurations that are used instead of or in addition to the canonical A, G, C, and U residues. In some embodiments, a modified gRNA is synthesized with a non-canonical nucleoside or nucleotide, is here called “modified.” Modified nucleosides and nucleotides can include one or more of: (i) alteration, e.g., replacement, of one or both of the non-linking phosphate oxygens and/or of one or more of the linking phosphate oxygens in the phosphodiester backbone linkage (an exemplary backbone modification); (ii) alteration, e.g., replacement, of a constituent of the ribose sugar, e.g., of the 2′ hydroxyl on the ribose sugar (an exemplary sugar modification); (iii) wholesale replacement of the phosphate moiety with “dephospho” linkers (an exemplary backbone modification); (iv) modification or replacement of a naturally occurring nucleobase, including with a non-canonical nucleobase (an exemplary base modification); (v) replacement or modification of the ribose-phosphate backbone (an exemplary backbone modification); (vi) modification of the 3′ end or 5′ end of the oligonucleotide, e.g., removal, modification or replacement of a terminal phosphate group or conjugation of a moiety, cap or linker (such 3′ or 5′ cap modifications may comprise a sugar and/or backbone modification); and (vii) modification or replacement of the sugar (an exemplary sugar modification).

Chemical modifications such as those listed above can be combined to provide modified gRNAs comprising nucleosides and nucleotides (collectively “residues”) that can have two, three, four, or more modifications. For example, a modified residue can have a modified sugar and a modified nucleobase, or a modified sugar and a modified phosphodiester. In some embodiments, every base of a gRNA is modified, e.g., all bases have a modified phosphate group, such as a phosphorothioate group. In certain embodiments, all, or substantially all, of the phosphate groups of an gRNA molecule are replaced with phosphorothioate groups. In some embodiments, modified gRNAs comprise at least one modified residue at or near the 5′ end of the RNA. In some embodiments, modified gRNAs comprise at least one modified residue at or near the 3′ end of the RNA.

In some embodiments, the gRNA comprises one, two, three or more modified residues. In some embodiments, at least 5% (e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%) of the positions in a modified gRNA are modified nucleosides or nucleotides.

Unmodified nucleic acids can be prone to degradation by, e.g., intracellular nucleases or those found in serum. For example, nucleases can hydrolyze nucleic acid phosphodiester bonds. Accordingly, in one aspect the gRNAs described herein can contain one or more modified nucleosides or nucleotides, e.g., to introduce stability toward intracellular or serum-based nucleases. In some embodiments, the modified gRNA molecules described herein can exhibit a reduced innate immune response when introduced into a population of cells, both in vivo and ex vivo. The term “innate immune response” includes a cellular response to exogenous nucleic acids, including single stranded nucleic acids, which involves the induction of cytokine expression and release, particularly the interferons, and cell death.

In some embodiments of a backbone modification, the phosphate group of a modified residue can be modified by replacing one or more of the oxygens with a different substituent. Further, the modified residue, e.g., modified residue present in a modified nucleic acid, can include the wholesale replacement of an unmodified phosphate moiety with a modified phosphate group as described herein. In some embodiments, the backbone modification of the phosphate backbone can include alterations that result in either an uncharged linker or a charged linker with unsymmetrical charge distribution.

Examples of modified phosphate groups include, phosphorothioate, phosphoroselenates, borano phosphates, borano phosphate esters, hydrogen phosphonates, phosphoroamidates, alkyl or aryl phosphonates and phosphotriesters. The phosphorous atom in an unmodified phosphate group is achiral. However, replacement of one of the non-bridging oxygens with one of the above atoms or groups of atoms can render the phosphorous atom chiral. The stereogenic phosphorous atom can possess either the “R” configuration (herein Rp) or the “S” configuration (herein Sp). The backbone can also be modified by replacement of a bridging oxygen, (i.e., the oxygen that links the phosphate to the nucleoside), with nitrogen (bridged phosphoroamidates), sulfur (bridged phosphorothioates) and carbon (bridged methylenephosphonates). The replacement can occur at either linking oxygen or at both of the linking oxygens.

The phosphate group can be replaced by non-phosphorus containing connectors in certain backbone modifications. In some embodiments, the charged phosphate group can be replaced by a neutral moiety. Examples of moieties which can replace the phosphate group can include, without limitation, e.g., methyl phosphonate, hydroxylamino, siloxane, carbonate, carboxymethyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo and methyleneoxymethylimino.

Scaffolds that can mimic nucleic acids can also be constructed wherein the phosphate linker and ribose sugar are replaced by nuclease resistant nucleoside or nucleotide surrogates. Such modifications may comprise backbone and sugar modifications. In some embodiments, the nucleobases can be tethered by a surrogate backbone. Examples can include, without limitation, the morpholino, cyclobutyl, pyrrolidine and peptide nucleic acid (PNA) nucleoside surrogates.

The modified nucleosides and modified nucleotides can include one or more modifications to the sugar group, i.e. at sugar modification. For example, the 2′ hydroxyl group (OH) can be modified, e.g. replaced with a number of different “oxy” or “deoxy” substituents. In some embodiments, modifications to the 2′ hydroxyl group can enhance the stability of the nucleic acid since the hydroxyl can no longer be deprotonated to form a 2′-alkoxide ion.

Examples of 2′ hydroxyl group modifications can include alkoxy or aryloxy (OR, wherein “R” can be, e.g., alkyl, cycloalkyl, aryl, aralkyl, heteroaryl or a sugar); polyethyleneglycols (PEG), wherein R can be, e.g., H or optionally substituted alkyl, and n can be an integer from 0 to 20 (e.g., from 0 to 4, from 0 to 8, from 0 to 10, from 0 to 16, from 1 to 4, from 1 to 8, from 1 to 10, from 1 to 16, from 1 to 20, from 2 to 4, from 2 to 8, from 2 to 10, from 2 to 16, from 2 to 20, from 4 to 8, from 4 to 10, from 4 to 16, and from 4 to 20). In some embodiments, the 2′ hydroxyl group modification can be 2′-O-Me. In some embodiments, the 2′ hydroxyl group modification can be a 2′-fluoro modification, which replaces the 2′ hydroxyl group with a fluoride. In some embodiments, the 2′ hydroxyl group modification can include “locked” nucleic acids (LNA) in which the 2′ hydroxyl can be connected, e.g., by a C₁₋₆ alkylene or C₁₋₆ heteroalkylene bridge, to the 4′ carbon of the same ribose sugar, where exemplary bridges can include methylene, propylene, ether, or amino bridges; O-amino (wherein amino can be, e.g., NH₂; alkylamino, dialkylamino, heterocyclyl, arylamino, diarylamino, heteroarylamino, or diheteroarylamino, ethylenediamine, or polyamino) and aminoalkoxy, O(CH₂)ₙ-amino, (wherein amino can be, e.g., NH₂; alkylamino, dialkylamino, heterocyclyl, arylamino, diarylamino, heteroarylamino, or diheteroarylamino, ethylenediamine, or polyamino) In some embodiments, the 2′ hydroxyl group modification can include “unlocked” nucleic acids (UNA) in which the ribose ring lacks the C2′-C3′ bond. In some embodiments, the 2′ hydroxyl group modification can include the methoxyethyl group (MOE), (OCH₂CH₂OCH₃, e.g., a PEG derivative).

“Deoxy” 2′ modifications can include hydrogen (i.e. deoxyribose sugars, e.g., at the overhang portions of partially dsRNA); halo (e.g., bromo, chloro, fluoro, or iodo); amino (wherein amino can be, e.g., NH₂; alkylamino, dialkylamino, heterocyclyl, arylamino, diarylamino, heteroarylamino, diheteroarylamino, or amino acid); NH(CH₂CH₂NH)ₙCH₂CH₂— amino (wherein amino can be, e.g., as described herein), —NHC(O)R (wherein R can be, e.g., alkyl, cycloalkyl, aryl, aralkyl, heteroaryl or sugar), cyano; mercapto; alkyl-thio-alkyl; thioalkoxy; and alkyl, cycloalkyl, aryl, alkenyl and alkynyl, which may be optionally substituted with e.g., an amino as described herein.

The sugar modification can comprise a sugar group which may also contain one or more carbons that possess the opposite stereochemical configuration than that of the corresponding carbon in ribose. Thus, a modified nucleic acid can include nucleotides containing e.g., arabinose, as the sugar. The modified nucleic acids can also include abasic sugars. These abasic sugars can also be further modified at one or more of the constituent sugar atoms. The modified nucleic acids can also include one or more sugars that are in the L form, e.g. L-nucleosides.

The modified nucleosides and modified nucleotides described herein, which can be incorporated into a modified nucleic acid, can include a modified base, also called a nucleobase. Examples of nucleobases include, but are not limited to, adenine (A), guanine (G), cytosine (C), and uracil (U). These nucleobases can be modified or wholly replaced to provide modified residues that can be incorporated into modified nucleic acids. The nucleobase of the nucleotide can be independently selected from a purine, a pyrimidine, a purine analog, or pyrimidine analog. In some embodiments, the nucleobase can include, for example, naturally-occurring and synthetic derivatives of a base.

In embodiments employing a dual guide RNA, each of the crRNA and the tracr RNA can contain modifications. Such modifications may be at one or both ends of the crRNA and/or tracr RNA. In embodiments comprising an sgRNA, one or more residues at one or both ends of the sgRNA may be chemically modified, and/or internal nucleosides may be modified, and/or the entire sgRNA may be chemically modified. Certain embodiments comprise a 5′ end modification. Certain embodiments comprise a 3′ end modification.

Modifications of 2′-O-methyl are encompassed.

Another chemical modification that has been shown to influence nucleotide sugar rings is halogen substitution. For example, 2′-fluoro (2′-F) substitution on nucleotide sugar rings can increase oligonucleotide binding affinity and nuclease stability. Modifications of 2′-fluoro (2′-F) are encompassed.

Phosphorothioate (PS) linkage or bond refers to a bond where a sulfur is substituted for one nonbridging phosphate oxygen in a phosphodiester linkage, for example in the bonds between nucleotides bases. When phosphorothioates are used to generate oligonucleotides, the modified oligonucleotides may also be referred to as S-oligos.

Abasic nucleotides refer to those which lack nitrogenous bases.

Inverted bases refer to those with linkages that are inverted from the normal 5′ to 3′ linkage (i.e., either a 5′ to 5′ linkage or a 3′ to 3′ linkage).

An abasic nucleotide can be attached with an inverted linkage. For example, an abasic nucleotide may be attached to the terminal 5′ nucleotide via a 5′ to 5′ linkage, or an abasic nucleotide may be attached to the terminal 3′ nucleotide via a 3′ to 3′ linkage. An inverted abasic nucleotide at either the terminal 5′ or 3′ nucleotide may also be called an inverted abasic end cap.

In some embodiments, one or more of the first three, four, or five nucleotides at the 5′ terminus, and one or more of the last three, four, or five nucleotides at the 3′ terminus are modified. In some embodiments, the modification is a 2′-O-Me, 2′-F, inverted abasic nucleotide, PS bond, or other nucleotide modification well known in the art to increase stability and/or performance.

In some embodiments, the first four nucleotides at the 5′ terminus, and the last four nucleotides at the 3′ terminus are linked with phosphorothioate (PS) bonds.

In some embodiments, the first three nucleotides at the 5′ terminus, and the last three nucleotides at the 3′ terminus comprise a 2′-O-methyl (2′-O-Me) modified nucleotide. In some embodiments, the first three nucleotides at the 5′ terminus, and the last three nucleotides at the 3′ terminus comprise a 2′-fluoro (2′-F) modified nucleotide.

### Ribonucleoprotein Complex

In some embodiments, a composition is encompassed comprising one or more gRNAs comprising one or more guide sequences from Table 2 and SluCas9 (for SEQ ID NOs: 1-51, 53, 55-56, 58-59, 61-62, 64, 66, or 70) or SaCas9 (for SEQ ID NOs: 200-259).

In some embodiments, the gRNA together with SluCas9 is called a ribonucleoprotein complex (RNP).

In some embodiments, a chimeric SluCas9 or SaCas9 is used, where one domain or region of the protein is replaced by a portion of a different protein. In some embodiments, a domain may be replaced with a domain from a different nuclease such as Fok1. In some embodiments, SluCas9 or SaCas9 may be a modified nuclease.

In some embodiments, the SluCas9 or SaCas9 is modified to contain only one functional nuclease domain. For example, the agent protein may be modified such that one of the nuclease domains is mutated or fully or partially deleted to reduce its nucleic acid cleavage activity.

In some embodiments, a conserved amino acid within SluCas9 or SaCas9 is substituted to reduce or alter nuclease activity. In some embodiments, SluCas9 or SaCas9 may comprise an amino acid substitution in the RuvC or RuvC-like nuclease domain.

In some embodiments, the SluCas9 or SaCas9 lacks cleavase activity. In some embodiments, the SluCas9 or SaCas9 comprises a dCas DNA-binding polypeptide. A dCas polypeptide has DNA-binding activity while essentially lacking catalytic (cleavase/nickase) activity. In some embodiments, the dCas polypeptide is a dCas9 polypeptide. In some embodiments, the RNA-targeted endonuclease lacking cleavase and nickase activity or the dCas DNA-binding polypeptide is a version of a Cas nuclease (e.g., a Cas nuclease discussed above) in which its endonucleolytic active sites are inactivated, e.g., by one or more alterations (e.g., point mutations) in its catalytic domains. See, e.g., US 2014/0186958 A1; US 2015/0166980 A1 relating to other species of Cas9 that may be used for guidance.

In some embodiments, the SluCas9 or SaCas9 comprises one or more heterologous functional domains (e.g., is or comprises a fusion polypeptide).

In some embodiments, the heterologous functional domain may facilitate transport of the SluCas9 or SaCas9 into the nucleus of a cell. For example, the heterologous functional domain may be a nuclear localization signal (NLS). In some embodiments, the SluCas9 or SaCas9 may be fused with 1-10 NLS(s). In some embodiments, the SluCas9 or SaCas9 may be fused with 1-5 NLS(s). In some embodiments, the SluCas9 or SaCas9 may be fused with one NLS. Where one NLS is used, the NLS may be linked at the N-terminus or the C-terminus of the SluCas9 or SaCas9 sequence. It may also be inserted within the SluCas9 or SaCas9 sequence. In other embodiments, the SluCas9 or SaCas9 may be fused with more than one NLS. In some embodiments, the SluCas9 or SaCas9 may be fused with 2, 3, 4, or 5 NLSs. In some embodiments, the SluCas9 or SaCas9 may be fused with two NLSs. In certain circumstances, the two NLSs may be the same (e.g., two SV40 NLSs) or different. In some embodiments, the SluCas9 or SaCas9 is fused to two SV40 NLS sequences linked at the carboxy terminus. In some embodiments, the SluCas9 or SaCas9 may be fused with two NLSs, one linked at the N-terminus and one at the C-terminus. In some embodiments, the SluCas9 may be fused with 3 NLSs. In some embodiments, the SluCas9 or SaCas9 may be fused with no NLS.

In some embodiments, the heterologous functional domain may be capable of modifying the intracellular half-life of the SluCas9 or SaCas9. In some embodiments, the half-life of the SluCas9 or SaCas9 may be increased. In some embodiments, the half-life of the SluCas9 or SaCas9 may be reduced. In some embodiments, the heterologous functional domain may be capable of increasing the stability of the SluCas9 or SaCas9. In some embodiments, the heterologous functional domain may be capable of reducing the stability of the SluCas9 or SaCas9. In some embodiments, the heterologous functional domain may act as a signal peptide for protein degradation. In some embodiments, the protein degradation may be mediated by proteolytic enzymes, such as, for example, proteasomes, lysosomal proteases, or calpain proteases. In some embodiments, the heterologous functional domain may comprise a PEST sequence. In some embodiments, the SluCas9 may be modified by addition of ubiquitin or a polyubiquitin chain. In some embodiments, the ubiquitin may be a ubiquitin-like protein (UBL). Non-limiting examples of ubiquitin-like proteins include small ubiquitin-like modifier (SUMO), ubiquitin cross-reactive protein (UCRP, also known as interferon-stimulated gene-15 (ISG15)), ubiquitin-related modifier-1 (URM1), neuronal-precursor-cell-expressed developmentally downregulated protein-8 (NEDD8, also called Rub1 in S. cerevisiae), human leukocyte antigen F-associated (FAT10), autophagy-8 (ATG8) and -12 (ATG12), Fau ubiquitin-like protein (FUB1), membrane-anchored UBL (MUB), ubiquitin fold-modifier-1 (UFM1), and ubiquitin-like protein-5 (UBL5).

In some embodiments, the heterologous functional domain may be a marker domain. Non-limiting examples of marker domains include fluorescent proteins, purification tags, epitope tags, and reporter gene sequences. In some embodiments, the marker domain may be a fluorescent protein. Non-limiting examples of suitable fluorescent proteins include green fluorescent proteins (e.g., GFP, GFP-2, tagGFP, turboGFP, sfGFP, EGFP, Emerald, Azami Green, Monomeric Azami Green, CopGFP, AceGFP, ZsGreen1), yellow fluorescent proteins (e.g., YFP, EYFP, Citrine, Venus, YPet, PhiYFP, ZsYellowl), blue fluorescent proteins (e.g., EBFP, EBFP2, Azurite, mKalamal, GFPuv, Sapphire, T-sapphire,), cyan fluorescent proteins (e.g., ECFP, Cerulean, CyPet, AmCyanl, Midoriishi-Cyan), red fluorescent proteins (e.g., mKate, mKate2, mPlum, DsRed monomer, mCherry, mRFP1, DsRed-Express, DsRed2, DsRed-Monomer, HcRed-Tandem, HcRedl, AsRed2, eqFP611, mRasberry, mStrawberry, Jred), and orange fluorescent proteins (mOrange, mKO, Kusabira-Orange, Monomeric Kusabira-Orange, mTangerine, tdTomato) or any other suitable fluorescent protein. In other embodiments, the marker domain may be a purification tag and/or an epitope tag. Non-limiting exemplary tags include glutathione-S-transferase (GST), chitin binding protein (CBP), maltose binding protein (MBP), thioredoxin (TRX), poly(NANP), tandem affinity purification (TAP) tag, myc, AcV5, AU1, AUS, E, ECS, E2, FLAG, HA, nus, Softag 1, Softag 3, Strep, SBP, Glu-Glu, HSV, KT3, S, S1, T7, V5, VSV-G, 6×His, 8×His, biotin carboxyl carrier protein (BCCP), poly-His, and calmodulin. Non-limiting exemplary reporter genes include glutathione-S-transferase (GST), horseradish peroxidase (HRP), chloramphenicol acetyltransferase (CAT), beta-galactosidase, beta-glucuronidase, luciferase, or fluorescent proteins.

In additional embodiments, the heterologous functional domain may target the SluCas9 to a specific organelle, cell type, tissue, or organ. In some embodiments, the heterologous functional domain may target the SluCas9 or SaCas9 to muscle.

In further embodiments, the heterologous functional domain may be an effector domain. When the SluCas9 or SaCas9 is directed to its target sequence, e.g., when SluCas9 or SaCas9 is directed to a target sequence by a gRNA, the effector domain may modify or affect the target sequence. In some embodiments, the effector domain may be chosen from a nucleic acid binding domain or a nuclease domain (e.g., a non-Cas nuclease domain) In some embodiments, the heterologous functional domain is a nuclease, such as a FokI nuclease. See, e.g., U.S. Pat. No. 9,023,649.

In some embodiments, the SluCas9 is any of the modified SluCas9 polypeptides as described in WO2020186059, WO2019118935, or WO2019183150, incorporated herein in their entirety and as discussed in more detail in the definitions section and provided in the Table of Additional Sequences.

Determination of Efficacy of gRNAs

In some embodiments, the efficacy of a gRNA is determined when delivered or expressed together with other components forming an RNP. In some embodiments, the gRNA is expressed together with SluCas9. In some embodiments, the gRNA is delivered to or expressed in a cell line that already stably expresses SluCas9 or SaCas9. In some embodiments the gRNA is delivered to a cell as part of a RNP. In some embodiments, the gRNA is delivered to a cell along with a mRNA encoding SluCas9 or SaCas9.

As described herein, use of SluCas9 or SaCas9 and a pair of guide RNAs disclosed herein can lead to double-stranded breaks in the DNA which can produce excision of a trinucleotide repeat upon repair by cellular machinery. In some embodiments, a pair of guide RNAs can both excise a portion of a genome and function independent of excision such that a pair of guides has both dual and single-cut efficacy.

In some embodiments, the efficacy of particular gRNAs is determined based on in vitro models. In some embodiments, the in vitro model is a cell line containing a target trinucleotide repeat, such as any such cell line described in the Example section below.

In some embodiments, the efficacy of particular gRNAs is determined across multiple in vitro cell models for a gRNA selection process. In some embodiments, a cell line comparison of data with selected gRNAs is performed. In some embodiments, cross screening in multiple cell models is performed.

In some embodiments, the efficacy of particular gRNAs is determined based on in vivo models. In some embodiments, the in vivo model is a rodent model. In some embodiments, the rodent model is a mouse which expresses a gene comprising an expanded trinucleotide repeat. The gene may be the human version or a rodent (e.g., murine) homolog of the DMPK gene. In some embodiments, the gene is human DMPK. In some embodiments, the gene is a rodent (e.g., murine) homolog of DMPK. In some embodiments, the in vivo model is a non-human primate, for example cynomolgus monkey.

In some embodiments, the efficacy of a guide RNA is measured by an amount of excision of a trinucleotide repeat of interest. The amount of excision may be determined by any appropriate method, e.g., quantitative sequencing; quantitative PCR; quantitative analysis of a Southern blot; etc.

### Examples

The following examples are provided to illustrate certain disclosed embodiments and are not to be construed as limiting the scope of this disclosure in any way.

A. Materials and Methods

Guide and Primer sequences. Primer sequences are shown in the Table of Additional Sequences. The crRNA and tracrRNA used for gRNAs with SluCas9 was

(SEQ ID NO: 602)
GUUUUAGUACUCUGGAAACAGAAUCUACUGAAACAAGACAAUAUGUCGU
GUUUAUCCCAUCAAUUUAUUGGUGGGAU.

The crRNA and tracrRNA used for gRNAs with SaCas9

(SEQ ID NO: 97)
GUUUUAGUACUCUGGAAACAGAAUCUACUAAAACAAGGCAAAAUGCCGUG
UUUAUCUCGUCAACUUGUUGGCGAGAU

Preparation and Electroporation of DM1 iPSC Cell Lines. SB1 Cell Line: Cells were isolated from peripheral blood mononuclear cells from an adult female DM1 patient (source of cells from Nicholas E. Johnson (Virginia Commonwealth University)) and reprogrammed with the CytoTune®-iPS Sendai reprogramming kit. Individual iPSC clones were isolated, including clone SB1. The SB1 cell line had a pluripotency signature consistent with an iPSC cell line by Nanostring assay. High resolution aCGH karyotyping revealed no gross genomic abnormalities. Southern analysis confirmed that the SB1 cell line contains a pathogenic CTG repeat expansion (˜300 CTG repeats) (FIG. 1).

Electroporation of DM1 iPSC cells: DM1 iPSC cells (200,000 per reaction) were mixed with RNPs prepared as follows.

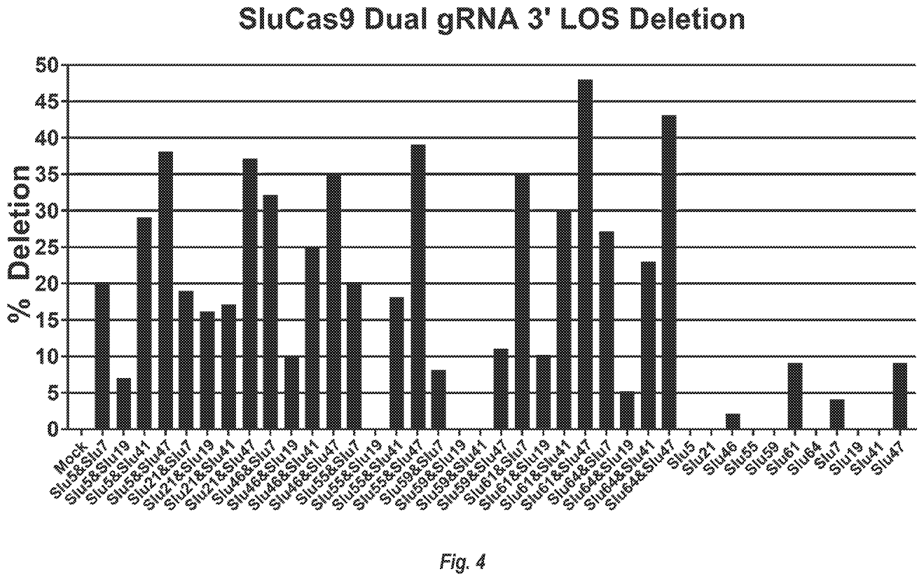

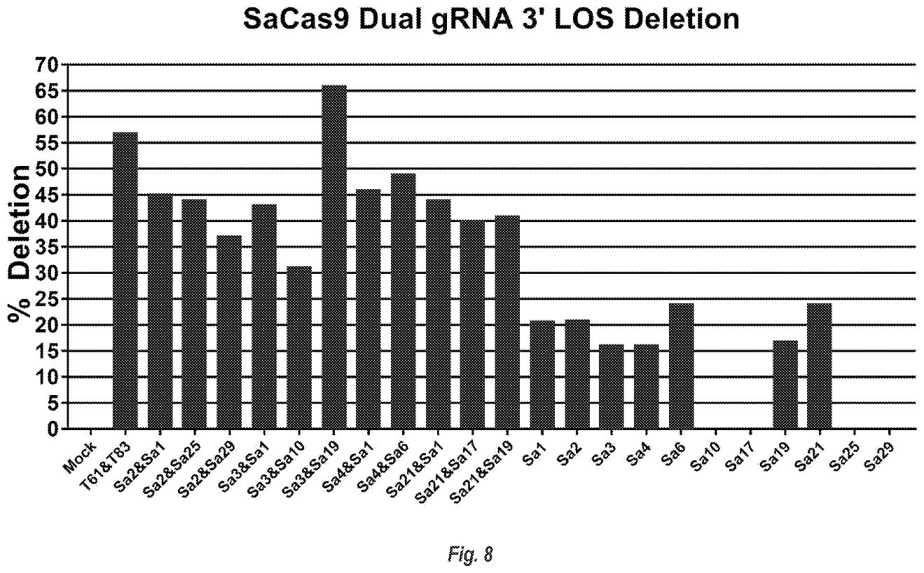

Broadly, RNP complexes for the experiment corresponding to FIG. 4 and FIG. 8 were prepared by assembling 1.5 μg each of the 5′ guide, the 3′ guide, and 3 μg of the SluCas9 or SaCas9 protein. Guide RNAs were diluted to 1.5 μg/μ1 and Cas9 nucleases were diluted to 3 μg/μ1 and 1 μl of each component was combined together and complexed together for a minimum of 10 minutes at room temperature.

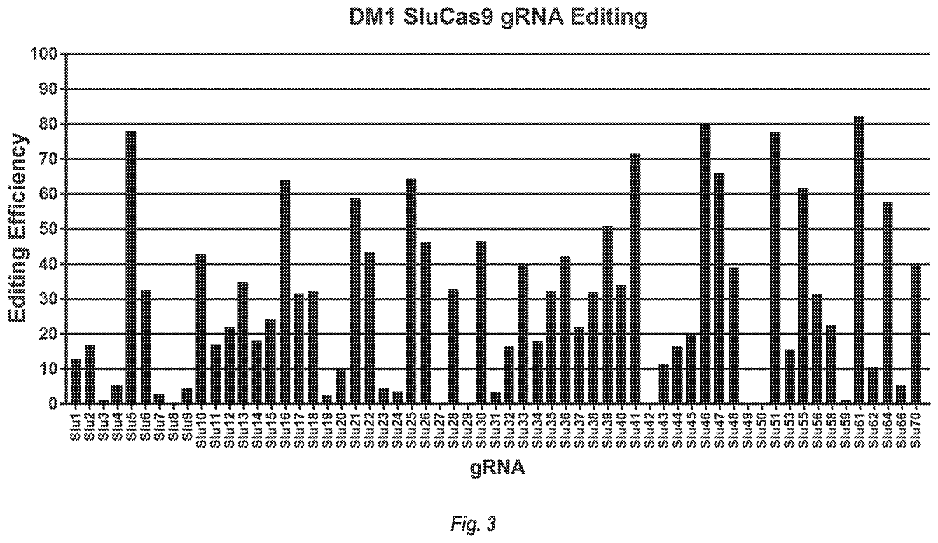

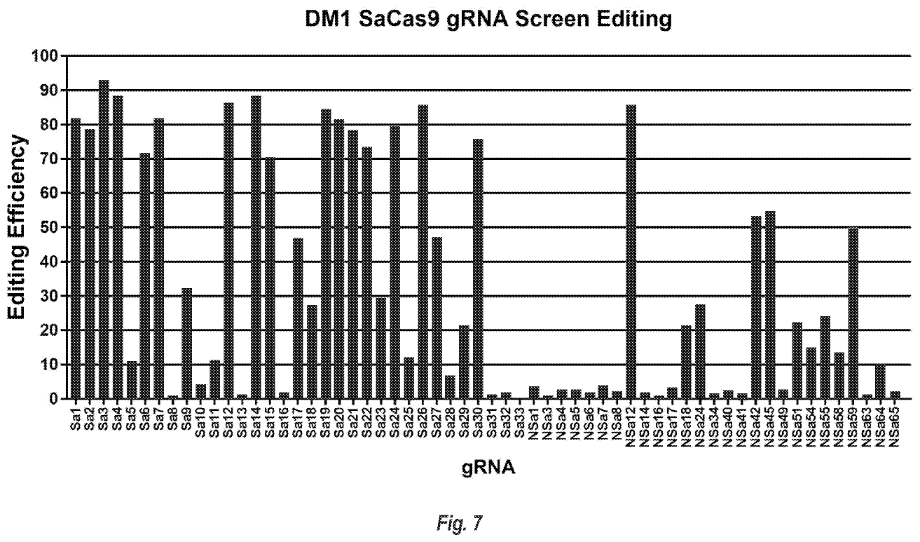

RNP complexes for the experiment corresponding to FIG. 3 and FIG. 7 was prepared by assembling 2 μg guide and 2 μg of the SluCas9 or SaCas9 nuclease. Individual chemically synthesized guide RNAs were diluted to 2 μg/μ1 and Cas9 nucleases were diluted to 2 μg/μ1 and 1 μI of each component was combined together and complexed together for a minimum of 10 minutes at room temperature.

Cells were electroporated with a Lonza Nucleofector (CA-137 setting) and harvested 72 hours post electroporation. Genomic DNA was isolated and used as template for subsequent PCR for TIDE analysis and ddPCR deletion analysis.

Differentiation Protocol for DM1 Cardiomyocytes. DM1 cardiomyocytes were prepared from the DM1 iPSC cell line SB1. Cells were activated with Wnt (4 μM CHIR) for 2 days, followed by Wnt inactivation (4 μM WNT-059) for 2 days. Cells were rested for a recovery period in CDM3 media for 6 days. Cells were then transferred to CDM3-no glucose media for metabolic selection for 2 days.

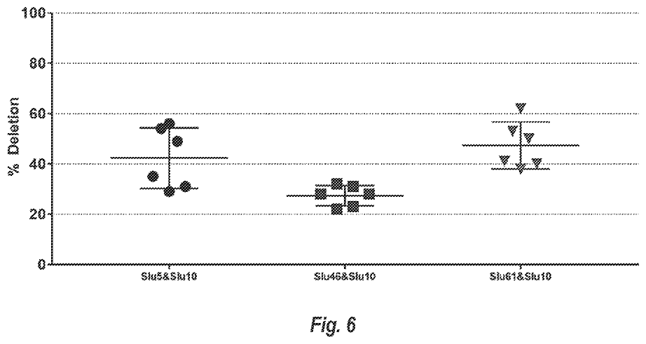

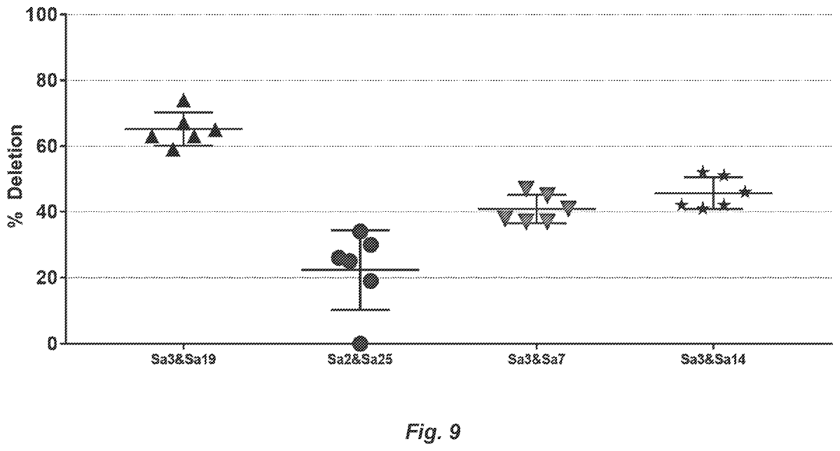

RNP complexes for experiments corresponding to FIG. 6 and FIG. 9 were prepared by assembling 2 μg each of individual chemically synthesized guide RNA and 4 μg of the Cas9 nuclease protein per reaction.

Cells were electroporated a with Lonza Nucleofector (CA-137 setting) and incubated in iCell Maintenance Media. Cells were harvested 72 hours post electroporation. Genomic DNA was isolated and used as template for subsequent PCR for TIDE analysis and ddPCR deletion analysis.

Sequencing and TIDE Analysis. PCR was performed on genomic DNA as follows.

PCR Sample:

| | Volume (μl) |
|---|---|
| Platinum | 45 |
| Enhancer | 5 |
| Primer (10 μM) | 1 |
| DNA | 1 |

PCR Conditions:

| | 34X | | | | |
|---|---|---|---|---|---|
| 94C | 94C | 60C | 68C | 68C | 4C |
| 2 min | 15 sec | 30 sec | 3 min | 10 min | ∞ |

PCR products were cleaned up using AMPure bead-based PCR purification (Beckman Coulter). The AMPure bead bottle was vortexed and aliquoted into a falcon tube. Following incubation for 30 minutes at room temperature, 85 μL of beads were added to each well of PCR products, pipetted up and down 10 times and incubated for 10 minutes. The bead mixture was then placed on a magnet for 5 minutes. Liquid was aspirated, and beads were washed twice with 70% EtOH while keeping the plate on the magnet. The plate was then removed from the magnet and 20 μL of dH2O was added to the beads and pipetted up and down to mix. Following incubation for 5-10 minutes, the plate was placed on the magnet for 1 minute. The dH2O containing the DNA was removed and PCR concentrations were analyzed on by nanodrop.

PCR products were sent for sequenced using Forward Primer (SEQ ID NO: 101) and Reverse Primer (SEQ ID NO: 102). Indel values were estimated using the TIDE analysis algorithm. TIDE is a method based on the recovery of indels' spectrum from the sequencing electrophoretograms to quantify the proportion of template-mediated editing events (Brinkman, E A et al. (2014) Nucleic Acids Res. 42: e168; PMID: 25300484).

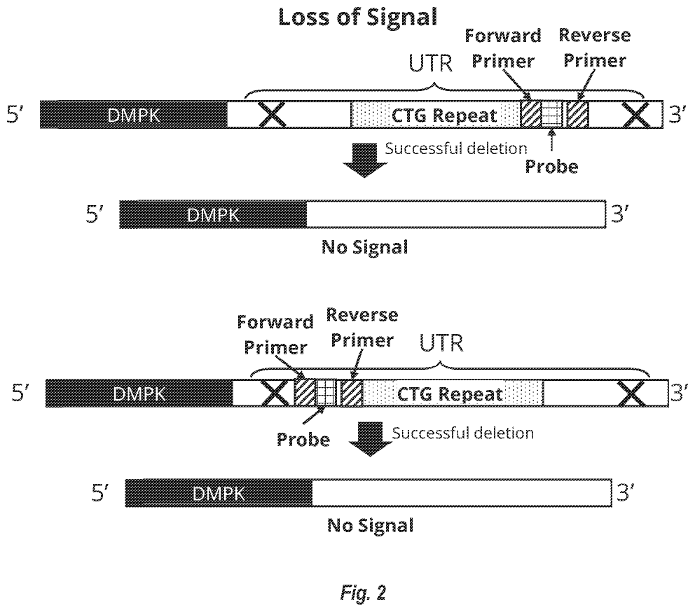

Two Loss-of-Signal (LOS) Droplet Digital PCR (ddPCR) Assay. The loss-of-signal ddPCR assay amplifies a region of the 3′ UTR of DMPK that is 5′ of the CTG repeat region or 3′ of the CTG region and detects the loss-of-signal of a probe targeting the amplified region as a result of successful deletion of the CTG repeat region (see FIG. 2 schematic of assay). The “dual” or “two” LOS ddPCR assay refers to results from both the 5′ LOS and 3′ LOS assays.

For the 5′ LOS ddPCR assay, Forward Primer (SEQ ID NO: 103), Reverse Primer (SEQ ID NO: 104), and Probe (SEQ ID NO: 105) were used.

For the 3′ LOS ddPCR assay, Forward Primer (SEQ ID NO: 106), Reverse Primer (SEQ ID NO: 107), and Probe (SEQ ID NO: 108) were used.

The ddPCR samples were setup at room temperature. DNA samples were diluted to a concentration of 10-20 ng/μL Diluted DNA (4 μL) was added to 21 μL of ddPCR mix.

ddPCR Mix:

| | 1X |
|---|---|
| 2X Droplet PCR Supermix | 12.5 |
| Forward Primer (18 uM) | 1.25 |
| Reverse Primer (18 uM) | 1.25 |
| Probe (5 uM) | 1.25 |
| RPP30 (dHsaCP2500350) | 1 |
| HINDIII | 0.2 |
| H20 | 3.55 |
| Mix volume | 21 |

The plate was sealed with a heat seal and mixed by vortexing, and then centrifuged briefly. The final volume was 25 μL.

The samples were transferred to a 96 well plate for auto digital generation. Droplets (40 μL) were generated and the plate was transferred to the PCR machine.

A three-step cycling protocol was run:

| | | |
|---|---|---|
| # Cycles | Temp | Duration of Cycle |

| | | | | |
|---|---|---|---|---|
| 1 | 95 | C. | 10 | min |
| 40 | 94 | C. | 30 | sec |
| | 60 | C. | 1 | min |
| 1 | 98 | C. | 10 | min |

| | | | |
|---|---|---|---|
| 1 | 4 | C. | forever |

The reference gene used for 5′ and 3′ loss-of-signal (LOS) ddPCRs was RPP30.

B. Results

1. Screening of SluCas9 gRNAs

To assess editing efficiency of individual gRNAs, 61 gRNAs were selected for screening in the wildtype iPSC cell line. The wildtype iPSC cells used, cell line number 0052, is a GMP-grade iPSC line available through Rutgers University Cell and DNA Repository.

Cells were transfected with RNPs containing individual guide RNAs and SluCas9 using electroporation with a Lonza Nucleofector. Genomic DNA was isolated from the cells and amplified by PCR. Sanger sequencing and TIDE analysis were used to quantify the frequency of indels generated by each sgRNA. Results are shown as % editing efficiency by TIDE analysis (Table 3, FIG. 3).

TABLE 3

| SEQ | | | |
|---|---|---|---|
| ID | Guide | | Editing |
| NO | RNA | Guide Sequence | Efficiency (%) |
| 1 | Slu1 | GATGGAGGGCCTTTTATTCGCG | 12.8 |
| 2 | Slu2 | GGCCTTTTATTCGCGAGGGTCG | 16.7 |
| 3 | Slu3 | CAGTTCACAACCGCTCCGAGCG | 0.8 |
| 4 | Slu4 | AATATCCAAACCGCCGAAGCGG | 5.2 |
| 5 | Slu5 | AGGACCCTTCGAGCCCCGTTCG | 77.7 |
| 6 | Slu6 | CCACGCTCGGAGCGGTTGTGAA | 32.3 |
| 7 | Slu7 | CTCCACGCACCCCCACCTATCG | 2.6 |
| 8 | Slu8 | CACCCCCGACCCTCGCGAATAA | 0 |
| 9 | Slu9 | ACCCTAGAACTGTCTTCGACTC | 4.4 |
| 10 | Slu10 | CTTTGCGAACCAACGATAGGTG | 42.6 |
| 11 | Slu11 | GAGGGCCTTTTATTCGCGAGGG | 16.8 |
| 12 | Slu12 | GGGCCTTTTATTCGCGAGGGTC | 21.7 |
| 13 | Slu13 | ACCTCGTCCTCCGACTCGCTGA | 34.5 |
| 14 | Slu14 | TTTGCACTTTGCGAACCAACGA | 17.9 |
| 15 | Slu15 | CGGGATCCCCGAAAAAGCGGGT | 24.2 |
| 16 | Slu16 | CCAGTTCACAACCGCTCCGAGC | 63.7 |
| 17 | Slu17 | ACTTTGCGAACCAACGATAGGT | 31.4 |
| 18 | Slu18 | ATAAATATCCAAACCGCCGAAG | 32 |
| 19 | Slu19 | AGATGGAGGGCCTTTTATTCGC | 2.4 |
| 20 | Slu20 | CGGCTCCGCCCGCTTCGGCGGT | 9.8 |
| 21 | Slu21 | CCCCGGAGTCGAAGACAGTTCT | 58.7 |
| 22 | Slu22 | TGGGCGGAGACCCACGCTCGGA | 42.9 |
| 23 | Slu23 | GCGCGATCTCTGCCTGCTTACT | 4.3 |
| 24 | Slu24 | CACTTTGCGAACCAACGATAGG | 3.5 |
| 25 | Slu25 | GCGGCCGGCGAACGGGGCTCGA | 64.1 |
| 26 | Slu26 | ATCCGGGCCCGCCCCCTAGCGG | 45.9 |
| 27 | Slu27 | CCTGCAGTTTGCCCATCCACGT | 12.6 |
| 28 | Slu28 | GGCGCGATCTCTGCCTGCTTAC | 32.4 |
| 29 | Slu29 | CAAACCGCCGAAGCGGGCGGAG | 0 |
| 30 | Slu30 | TGTCTTCGACTCCGGGGCCCCG | 46.4 |
| 31 | Slu31 | CCCAACAACCCCAATCCACGTT | 3 |
| 32 | Slu32 | GGGCGCGGGATCCCCGAAAAAG | 16.1 |
| 33 | Slu33 | AGGGCCTTTTATTCGCGAGGGT | 39.9 |
| 34 | Slu34 | AATAAATATCCAAACCGCCGAA | 17.6 |
| 35 | Slu35 | GGGGCGCGGGATCCCCGAAAAA | 32 |
| 36 | Slu36 | TGTGATCCGGGCCCGCCCCCTA | 42 |
| 37 | Slu37 | CCTCCGACTCGCTGACAGGCTA | 21.5 |
| 38 | Slu38 | CTTCGAGCCCCGTTCGCCGGCC | 31.6 |
| 39 | Slu39 | GGGCTCGAAGGGTCCTTGTAGC | 50.5 |
| 40 | Slu40 | GCTCGGAGCGGTTGTGAACTGG | 33.8 |
| 41 | Slu41 | CCAGCCGGCTCCGCCCGCTTCG | 71.5 |
| 42 | Slu42 | CTGCAGTTTGCCCATCCACGTC | 6.3 |
| 43 | Slu43 | GGTCCTGTAGCCTGTCAGCGAG | 11.4 |
| 44 | Slu44 | CTCAGTGCATCCAAAACGTGGA | 16.2 |
| 45 | Slu45 | TCAGTGCATCCAAAACGTGGAT | 20 |
| 46 | Slu46 | GCCCCGTTGGAAGACTGAGTGC | 79.6 |
| 47 | Slu47 | TTCTTGTGCATGACGCCCTGCT | 65.5 |
| 48 | Slu48 | TCTTGTGCATGACGCCCTGCTC | 38.8 |
| 49 | Slu49 | TGGAGGATGGAACACGGACGGC | 25.1 |
| 50 | Slu50 | TCGCGCCAGACGCTCCCCAGAG | 0 |
| 51 | Slu51 | CCCCGTTGGAAGACTGAGTGCC | 77.4 |
| 53 | Slu53 | GCCGGGTCCGCGGCCGGCGAAC | 15.6 |
| 55 | Slu55 | GCTAGGGGGCGGGCCCGGATCA | 61.2 |
| 56 | Slu56 | GCCCCGGAGTCGAAGACAGTTC | 31.1 |
| 58 | Slu58 | GCCCCGTTCGCCGGCCGCGGAC | 22.2 |
| 59 | Slu59 | CCCTAGAACTGTCTTCGACTCC | 1 |
| 61 | Slu61 | GGGGCTCGAAGGGTCCTTGTAG | 82.1 |
| 62 | Slu62 | CCCGGGCACTCAGTCTTCCAAC | 10.5 |
| 64 | Slu64 | AGCGGTTGTGAACTGGCAGGCG | 57.6 |
| 66 | Slu66 | GGCGCGGCTTCTGTGCCGTGCC | 5.3 |
| 70 | Slu70 | CGGAGCGGTTGTGAACTGGCAG | 39.9 |

2. Screening of SluCas9 gRNA Pairs in DM1 iPSC Cells

Seven upstream gRNAs (SEQ ID NOs: 5, 21, 46, 55, 59, 61, and 64) and four downstream gRNAs (SEQ ID NOs: 7, 9, 41, and 47) were selected for evaluation of CTG repeat region deletion in DM1 iPSC SB1 cells with SluCas9.

Specifically, the following pairs of gRNAs were tested: SEQ ID NOs: 5 and 7; SEQ ID NOs: 5 and 9; SEQ ID NOs: 5 and 41; SEQ ID NOs: 5 and 47; SEQ ID NOs: 21 and 7; SEQ ID NOs: 21 and 9; SEQ ID NOs: 21 and 41; SEQ ID NOs: 21 and 47; SEQ ID NOs: 46 and 7; SEQ ID NOs: 46 and 9; SEQ ID NOs: 46 and 41; SEQ ID NOs: 46 and 47; SEQ ID NOs: 55 and 7; SEQ ID NOs: and 9; SEQ ID NOs: 55 and 41; SEQ ID NOs: 55 and 47; SEQ ID NOs: 59 and 7; SEQ ID NOs: 59 and 9; SEQ ID NOs: 59 and 41; SEQ ID NOs: 59 and 47; SEQ ID NOs: 61 and 7; SEQ ID NOs: 61 and 9; SEQ ID NOs: 61 and 41; SEQ ID NOs: 61 and 47; SEQ ID NOs: 64 and 7; SEQ ID NOs: 64 and 9; SEQ ID NOs: 64 and 41; and SEQ ID NOs: 64 and 47.

The percentage of CTG repeat region deletion for SluCas9 gRNA pairs and individual SluCas9 gRNAs is shown in FIG. 4 based on results from the 3′ LOS ddPCR assay. The 5′ LOS assay did not accurately portray deletion due to single gRNAs knocking out the ddPCR primer site (n=1). Percent editing efficiencies are shown for individual SluCas9 gRNAs in Table 4. In Table 4, #5 refers to gRNA Slu5, #21 refers to gRNA Slu21, #46 refers to gRNA Slu46, #55 refers to gRNA Slu55, #59 refers to gRNA Slu59, #61 refers to gRNA Slu61, #7 refers to gRNA Slu7, #19 refers to gRNA Slu19, #41 refers to gRNA Slu41, and #47 refers to gRNA Slu47

TABLE 4

| Slu gRNA | Editing Efficiency (%) |
|---|---|
| 5′ - #5 | 78 |
| 5′ - #21 | 60 |
| 5′ - #46 | 80 |
| 5′ - #55 | 60 |
| 5′ - #59 | 5 |
| 5′ - #61 | 82 |
| 3′ - #7 | 5 |
| 3′ - #19 | 5 |
| 3′ - #41 | 72 |
| 3′ - #47 | 65 |

The percentage of dual deletion in SB1 iPSCs for SluCas9 gRNA pairs is shown in Table 5 based on results from MS1 deletion screen.

TABLE 5

| | Single gRNA | | Single gRNA | Dual Deletion |
|---|---|---|---|---|
| 5′ gRNA | Editing | 3′ gRNA | Editing | in SB1 iPSCs |
| Slu5 | 77.7% | Slu10 | 42.6% | 45% |
| Slu46 | 79.6% | Slu10 | 42.6% | 51% |
| Slu61 | 82.1% | Slu10 | 42.6% | 56% |
| Slu64 | 57.6% | Slu47 | 65.5% | 45% |

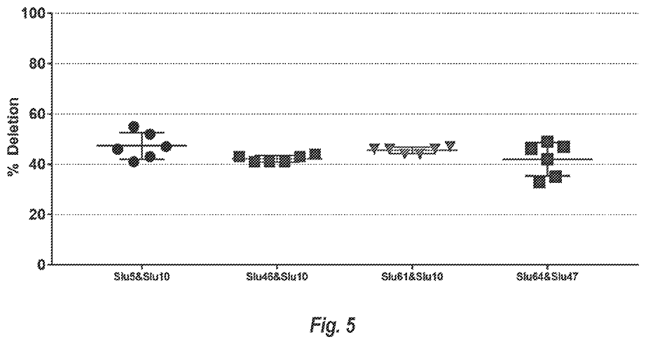

The percentage of CTG repeat region deletion for selected SluCas9 gRNA pairs is shown in Table 6 and 7, and FIG. 5. Table 6 presents results of triplicate testing across two separate experiments of SluCas9 dual gRNA screening in DM1 iPS cells. Table 7 presents the average deletion of the same pairs.

TABLE 6

| | Slu5&Slu10 | Slu46&Slu10 | Slu61&Slu10 | Slu64&Slu47 |
|---|---|---|---|---|
| Exp#1Rep#1 | 55% | 41% | 46% | 49% |
| Exp#1Rep#2 | 52% | 41% | 44% | 46% |
| Exp#1Rep#3 | 43% | 43% | 47% | 47% |
| Exp#2Rep#1 | 47% | 41% | 44% | 42% |
| Exp#2Rep#2 | 46% | 43% | 46% | 33% |
| Exp#2Rep#3 | 41% | 44% | 46% | 35% |

TABLE 7

| Cas9 Format | gRNA Pair | Average Deletion in iPSCs |
|---|---|---|
| SluCas9 | Slu61&Slu10 | 46% |
| SluCas9 | Slu5&Slu10 | 47% |
| SluCas9 | Slu46&Slu10 | 42% |

3. Screening of SluCas9 gRNA Pairs in DM1 Cardiomyocytes

Three upstream gRNAs (SEQ ID NOs: 5, 46, and 61) and one downstream gRNA (SEQ ID NO: 10) were selected for evaluation of CTG repeat region deletion in DM1 cardiomyocyte cells with SluCas9.

Specifically, the following pairs of gRNAs were tested: SEQ ID NOs: 5 and 10; SEQ ID NOs: 46 and 10; and SEQ ID NOs: 61 and 10.

The percentage of CTG repeat region deletion for selected SluCas9 gRNA pairs is shown in FIG. 6 and Table 8. Table 8 presents results of triplicate testing across two separate experiments of SluCas9 dual gRNA screening in DM1 cardiomyocytes.

TABLE 8

| | Slu5&Slu10 | Slu46&Slu10 | Slu61&Slu10 |
|---|---|---|---|
| Exp#1Rep#1 | 29% | 31% | 38% |
| Exp#1Rep#2 | 31% | 32% | 40% |
| Exp#1Rep#3 | 35% | 28% | 41% |
| Exp#2Rep#1 | 49% | 28% | 62% |
| Exp#2Rep#2 | 56% | 23% | 53% |
| Exp#2Rep#3 | 54% | 22% | 50% |

4. Screening of SaCas9 gRNAs

To assess editing efficiency of individual saCas9 gRNAs, 58 saCas9gRNAs were selected for screening in the wildtype iPSC cell line. The wildtype iPSC cells used, cell line number 0052, is a GMP-grade iPSC line available through Rutgers University Cell and DNA Repository.

Cells were transfected with RNPs containing individual guide RNAs and SaCas9 using electroporation with a Lonza Nucleofector. Genomic DNA was isolated from the cells and amplified by PCR. Sanger sequencing and TIDE analysis were used to quantify the frequency of indels generated by each sgRNA. Results are shown as % editing efficiency by TIDE analysis (Table 9, FIG. 7).

TABLE 9

| | | | Editing |
|---|---|---|---|
| Guide | SEQ ID | Guide Sequence | Efficiency |
| RNA | NO. | | (%) |
| Sa1 | 200 | GCGGGATGCGAAGCGGCCGAAT | 81.7 |
| Sa2 | 201 | GCCCCGGAGTCGAAGACAGTTC | 78.5 |
| Sa3 | 202 | CGCGGCCGGCGAACGGGGCTCG | 92.8 |
| Sa4 | 203 | CCAGTTCACAACCGCTCCGAGC | 88.1 |
| Sa5 | 204 | GGGCCTTTTATTCGCGAGGGTC | 10.7 |
| Sa6 | 205 | AGATGGAGGGCCTTTTATTCGC | 71.5 |
| Sa7 | 206 | GAGCTAGCGGGATGCGAAGCGG | 81.7 |
| Sa8 | 207 | CGGCTCCGCCCGCTTCGGCGGT | 0.7 |
| Sa9 | 208 | CAACGATAGGTGGGGGTGCGTG | 32.1 |
| Sa10 | 209 | TGGGGACAGACAATAAATACCG | 4.1 |
| Sa11 | 210 | CCCAACAACCCCAATCCACGTT | 10.9 |
| Sa12 | 211 | ACTCAGTCTTCCAACGGGGCCC | 86.1 |
| Sa13 | 212 | GGGGTCTCAGTGCATCCAAAAC | 1 |
| Sa14 | 213 | ACAACGCAAACCGCGGACACTG | 88.3 |
| Sa15 | 214 | CTTCGGCCGCCTCCACACGCCT | 70.2 |
| Sa16 | 215 | CCCCGGCCGCTAGGGGGCGGGC | 1.8 |
| Sa17 | 216 | GGGGCGCGGGATCCCCGAAAAA | 46.7 |
| Sa18 | 217 | CAAAACGTGGATTGGGGTTGTT | 27.2 |
| Sa19 | 218 | TTGGGGGTCCTGTAGCCTGTCA | 84.4 |
| Sa20 | 219 | TCAGTGCATCCAAAACGTGGAT | 81.1 |
| Sa21 | 220 | ACTCCGGGGCCCCGTTGGAAGA | 78.3 |
| Sa22 | 221 | GACAATAAATACCGAGGAATGT | 73.2 |
| Sa23 | 222 | TCGGCCAGGCTGAGGCCCTGAC | 29 |
| Sa24 | 223 | ACTTTGCGAACCAACGATAGGT | 79.3 |
| Sa25 | 224 | CTTTTGCCAAACCCGCTTTTTC | 12.3 |
| Sa26 | 225 | GGCTCGAAGGGTCCTTGTAGCC | 85.5 |
| Sa27 | 226 | TTTATTCGCGAGGGTCGGGGGT | 47 |
| Sa28 | 227 | CCGAAGGTCTGGGAGGAGCTAG | 6.5 |
| Sa29 | 228 | AGGACCCCCACCCCCGACCCTC | 21.4 |
| Sa30 | 229 | GGGTTTGGCAAAAGCAAATTTC | 75.5 |
| Sa31 | 230 | AGCGCAAGTGAGGAGGGGGGCG | 1 |
| Sa32 | 231 | CTAGCGGCCGGGGAGGGAGGGG | 1.6 |
| Sa33 | 232 | CTGCTGCTGCTGCTGCTGCTGG | Cannot |
| | | | evaluate |
| | | | editing, |
| | | | gRNA cuts on |
| | | | the repeat |
| NSa1 | 233 | CCAGGCTGAGGCCCTGACGTGG | 3.3 |
| NSa3 | 234 | AACCAACGATAGGTGGGGGTGC | 0.7 |
| NSa4 | 235 | TGTCTTCGACTCCGGGGCCCCG | 2.5 |
| NSa5 | 236 | AGGTGGGGACAGACAATAAATA | 2.6 |
| NSa6 | 237 | GCGGGCGGAGCCGGCTGGGGCT | 1.7 |
| NSa7 | 238 | CGCCTGCCAGTTCACAACCGCT | 3.6 |
| NSa8 | 239 | TCGCGCCAGACGCTCCCCAGAG | 1.8 |
| NSa12 | 240 | GCCCCGTTGGAAGACTGAGTGC | 85.6 |
| NSa14 | 241 | CGCCCAGCTCCAGTCCTGTGAT | 1.6 |
| NSa16 | 242 | GGCGCGGCTTCTGTGCCGTGCC | 0.9 |
| NSa17 | 243 | GGGGCGGGCCCGGATCACAGGA | 3 |
| NSa18 | 244 | GGGGCTCGAAGGGTCCTTGTAG | 21.5 |
| NSa24 | 245 | CATTCCCGGCTACAAGGACCCT | 27.4 |
| NSa34 | 246 | CGGCCCCTCCCTCCCCGGCCGC | 1.4 |
| NSa40 | 247 | CGGGCCCGCCCCCTAGCGGCCG | 2.2 |
| NSa41 | 248 | CCCGCCCCCTAGCGGCCGGGGA | 1.5 |
| NSa42 | 249 | CACTCAGTCTTCCAACGGGGCC | 53.2 |
| NSa45 | 250 | GGAGCTGGGCGGAGACCCACGC | 54.5 |
| NSa49 | 251 | GCCCCTCCCTCCCCGGCCGCTA | 2.5 |
| NSa51 | 252 | ACTGAGTGCCCGGGGCACGGCA | 21.9 |
| NSa54 | 253 | GTCCGCGGCCGGCGAACGGGGC | 14.9 |
| NSa55 | 254 | GTCTTCCAACGGGGCCCCGGAG | 24.1 |
| NSa58 | 255 | GAGACCCACGCTCGGAGCGGTT | 13.3 |
| NSa59 | 256 | GTCTTCGACTCCGGGGCCCCGT | 49.8 |
| NSa63 | 257 | ACCCTAGAACTGTCTTCGACTC | 1.2 |
| NSa64 | 258 | CCCCGTTGGAAGACTGAGTGCC | 9.8 |
| NSa65 | 259 | GGCCGGGTCCGCGGCCGGCGAA | 2 |

5. Screening of SaCas9 gRNA Pairs in DM1 iPSC Cells

Two upstream gRNAs (SEQ ID NOs: 201 and 202 (Sa2 and Sa3)) and four downstream gRNAs (SEQ ID NOs: 206 (Sa7), Sa14, Sa19, and Sa25) were selected for evaluation of CTG repeat region deletion in DM1 iPSC SB1 cells with SaCas9.

Specifically, the following pairs of gRNAs were tested: SEQ ID NOs: 202 and 218 (Sa3 and Sa19); SEQ ID NOs: 201 and 224 (Sa2 and Sa25); SEQ ID NOs: 202 and 206 (Sa3 and Sa7); and SEQ ID NOs: 202 and 213 (Sa3 and Sa14).

The percentage of CTG repeat region deletion for SaCas9 gRNA pairs and individual SaCas9 gRNAs is shown in FIG. 8 based on results from the 3′ LOS ddPCR assay. Percent editing efficiencies are shown for individual SaCas9 gRNAs in Table 10.

TABLE 10

| Sa gRNA | SEQ ID No. | Editing Efficiency (%) |
|---|---|---|
| 5′ - Sa2 | 201 | 78 |
| 5′ - Sa3 | 202 | 90 |
| 5′ - Sa4 | 203 | 86 |
| 5′ - Sa21 | 220 | 78 |
| 5′ - Sa1 | 200 | 75 |
| 3′ - Sa10 | 209 | 5 |
| 3′ - Sa17 | 216 | 48 |
| 3′ - Sa19 | 218 | 84 |
| 3′ - Sa25 | 224 | 13 |
| 3′ - Sa29 | 228 | 21 |

The percentage of dual deletion in SB1 iPSCs for SaCas9 gRNA pairs is shown in Table 11 based on results from MS1 deletion screen.

TABLE 11

| | | | | | | Dual |
|---|---|---|---|---|---|---|
| | | Single | | | Single | Deletion |
| | SEQ ID | gRNA | | SEQ ID | gRNA | in SB1 |
| 5′ gRNA | NO. | Editing | 3′ gRNA | NO. | Editing | iPSCs |
| Sa3 | 202 | 92.8% | Sa19 | 218 | 84.4% | 66% |
| Sa2 | 201 | 78.5% | Sa25 | 224 | 12.3% | 44% |
| Sa3 | 202 | 92.8% | Sa14 | 213 | 88.3% | 45% |
| Sa3 | 201 | 92.8% | Sa7 | 206 | 81.7% | 43% |

The percentage of CTG repeat region deletion for selected SaCas9 gRNA pairs is shown in Table 12 and 13, and FIG. 9. Table 12 presents results of triplicate testing across two separate experiments of SaCas9 dual gRNA screening in DM1 iPS cells. Two SaCas9 pairs show greater than 40% deletion in DM1 iPSCs. Table 13 presents the average dual deletion of the same pairs.

TABLE 12

| SEQ ID Nos. | 202 & 218 | 201 & 224 | 202 & 206 | 202 & 213 |
|---|---|---|---|---|
| Exp#1Rep#1 | 63% | 25% | 45% | 52% |
| Exp#1Rep#2 | 63% | 26% | 41% | 46% |
| Exp#1Rep#3 | 65% | 0% | 47% | 51% |
| Exp#2Rep#1 | 74% | 34% | 38% | 42% |
| Exp#2Rep#2 | 67% | 19% | 37% | 42% |
| Exp#2Rep#3 | 59% | 30% | 37% | 41% |

TABLE 13

| Cas9 Format | gRNA Pair | SEQ ID Nos. | Average Deletion in iPSCs |
|---|---|---|---|
| SaCas9 | Sa3&Sa19 | 202 & 218 | 65% |
| SaCas9 | Sa3&Sa14 | 202 & 213 | 46% |

6. Screening of SaCas9 gRNA Pairs in DM1 Cardiomyocytes

One upstream gRNA (SEQ ID NO: 3) and two downstream gRNAs (SEQ ID NOs: 14 and 19) were selected for evaluation of CTG repeat region deletion in DM1 cardiomyocyte cells with SaCas9.

Specifically, the following pairs of gRNAs were tested: SEQ ID NOs: 3 and 19; and SEQ ID NOs: 3 and 14.

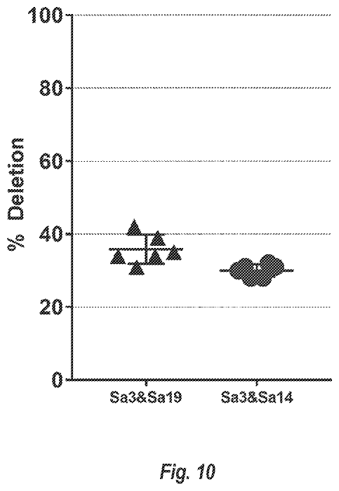

The percentage of CTG repeat region deletion for selected SaCas9 gRNA pairs is shown in FIG. 10 and Table 14. Table 14 presents results of triplicate testing across two separate experiments of SaCas9 dual gRNA screening in DM1 cardiomyocytes.

TABLE 14

| | Sa3&Sa19 (SEQ ID | Sa3&Sa14 (SEQ ID |
|---|---|---|
| | NOs: 202 & 218) | NOs: 202 & 213) |
| Exp#1Rep#1 | 34% | 28% |
| Exp#1Rep#2 | 34% | 32% |
| Exp#1Rep#3 | 42% | 31% |
| Exp#2Rep#1 | 31% | 28% |
| Exp#2Rep#2 | 39% | 31% |
| Exp#2Rep#3 | 35% | 30% |

This description and exemplary embodiments should not be taken as limiting. For the purposes of this specification and appended claims, unless otherwise indicated, all numbers expressing quantities, percentages, or proportions, and other numerical values used in the specification and claims, are to be understood as being modified in all instances by the term “about,” to the extent they are not already so modified. Accordingly, unless indicated to the contrary, the numerical parameters set forth in the following specification and attached claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques.

It is noted that, as used in this specification and the appended claims, the singular forms “a,” “an,” and “the,” and any singular use of any word, include plural referents unless expressly and unequivocally limited to one referent. As used herein, the term “include” and its grammatical variants are intended to be non-limiting, such that recitation of items in a list is not to the exclusion of other like items that can be substituted or added to the listed items.

TABLE OF ADDITIONAL SEQUENCES

| SEQ | | |
|---|---|---|
| ID NO | Description | Sequence |
| 101 | TIDE sequencing | GAGTCCCAGGAGCCAATCA |
| | Forward Primer | |
| 102 | TIDE sequencing | CCCCTCTTCTCGACGCTC |
| | Reverse Primer | |
| 103 | 5′ LOS ddPCR | CTAGCGGCCGGGGAG |
| | Forward Primer | |
| 104 | 5′ LOS ddPCR | AGCAGCATTCCCGGCTA |
| | Reverse Primer | |
| 105 | 5′ LOS ddPCR | CGAACGGGGCTCGAAGGGTCCTTG |
| | Probe | |
| 106 | 3′ LOS ddPCR | GGGGGATCACAGACCATTTCT |
| | Forward Primer | |
| 107 | 3′ LOS ddPCR | CGAACCAACGATAGGTGGGG |
| | Reverse Primer | |
| 108 | 3′ LOS ddPCR | CCTGGGAAGGCAGCAAGCCG |
| | Probe | |
| 800 | M-SluCas9_X | MNQKFILGLDIGITSVGYGLIDYETKNIIDAGVRLFPEANVENNEGRRSKRGSRRL |
| | | KRRRIHRLERVKKLLEDYNLLD |
| | | QSQIPQSTNPYAIRVKGLSEALSKDELVIALLHIAKRRGIHK |
| | | IDVIDSNDDVGNELSTKEQLNKNSKLLKDKFVCQIQLERM |
| | | NEGQVRGEKNRFKTADIIKEIIQLLNVQKNFHQLDENFINK |
| | | YIELVEMRREYFEGPGKGSPYGWEGDPKAWYETLMGHXT |
| | | YFPDELRSVKYAYSADLFNALNDLNNLVIQRDGLSKLEYH |
| | | EKYHIIENVFKQKKKPTLKQIANEINVNPEDIKGYRITKSGK |
| | | PQFTEFKLYHDLKSVLFDQSILENEDVLDQIAEILTIYQDKD |
| | | SIKSKLTELDILLNEEDKENIAQLTGYTGTHRLSLKXIRLVL |
| | | EXQWYSSXNQMXIFTXLNIKPKKINLTAANKIPKAMIDEFI |
| | | LSPWKRTFGQAINLINKIIEKYGVPEDIIIELARENNSKD |
| | | KQKFINEMQKKNENTRKRINEIIGKYGNQNAKRLVEKIRLH |
| | | DEQEGKCLYSLESIPLEDLLNNPNHYEVDHIIPRSVSFDNSY |
| | | HNKVLVKQSENSKKSNLTPYQYFNSGKSKLSYNQFKQHIL |
| | | NLSKSQDRISKKKKEYLLEERDINKFEVQKEFINRNLVDTR |
| | | YATRELTNYLKAYFSANNMNVKVKTINGSFTDYLRKVWK |
| | | FKKERNHGYKHHAEDALIIANADFLFKENKKLKAVNSVLE |
| | | KPEIETKQLDIQVDSEDNYSEMFIIPKQVQDIKDFRNFKYSH |
| | | RVDKKPNRQLINDTLYSTRKKDNSTYIVQTIKDIYAKDNTT |
| | | LKKQFDKSPEKFLMYQHDPRTFEKLEVIMKQYANEKNPLA |
| | | KYHEETGEYLTKYSKKNNGPIVKSLKYIGNKLGSHLDVTH |
| | | QFKSSTKKLVKLSIKPYRFDVYLTDKGYKFITISYLDVLKKD |
| | | NYYYIPEQKYDKLKLGKAIDKNAKFIASFYKNDLIKLDGEIY |
| | | KIIGVNSDTRNMIELDLPDIRYKEYCELNNIKGEPRIKKTIG |
| | | KKVNSIEKLTTDVLGNVFTNTQYTKPQLLFKRGNGG. |
| 801 | M-SluCas9- | MNQKFILGLDIGITSVGYGLIDYETKNIIDAGVRLFPEANVE |
| | R414A | NNEGRRSKRGSRRLKRRRIHRLERVKKLLEDYNLLDQSQIP |
| | | QSTNPYAIRVKGLSEALSKDELVIALLHIAKRRGIHKIDVID |
| | | SNDDVGNELSTKEQLNKNSKLLKDKFVCQIQLERMNEGQV |
| | | RGEKNRFKTADIIKEIIQLLNVQKNFHQLDENFINKYIELVEM |
| | | RREYFEGPGKGSPYGWEGDPKAWYETLMGHCTYFPDELRS |
| | | VKYAYSADLFNALNDLNNLVIQRDGLSKLEYHEKYHIIENV |
| | | FKQKKKPTLKQIANEINVNPEDIKGYRITKSGKPQFTEFKLY |
| | | HDLKSVLFDQSILENEDVLDQIAEILTIYQDKDSIKSKLTELD |
| | | ILLNEEDKENIAQLTGYTGTHRLSLKCIRLVLEEQWYSSAN |
| | | QMEIFTHLNIKPKKINLTAANKIPKAMIDEFILSPWKRTFGQ |
| | | AINLINKIIEKYGVPEDIIIELARENNSKDKQKFINEMQKKNE |
| | | NTRKRINEIIGKYGNQNAKRLVEKIRLHDEQEGKCLYSLESI |
| | | PLEDLLNNPNHYEVDHIIPRSVSFDNSYHNKVLVKQSENSK |
| | | KSNLTPYQYFNSGKSKLSYNQFKQHILNLSKSQDRISKKKK |
| | | EYLLEERDINKFEVQKEFINRNLVDTRYATRELTNYLKAYF |
| | | SANNMNVKVKTINGSFTDYLRKVWKFKKERNHGYKHHAE |
| | | DALIIANADFLFKENKKLKAVNSVLEKPEIETKQLDIQVDSE |
| | | DNYSEMFIIPKQVQDIKDFRNFKYSHRVDKKPNRQLINDTL |
| | | YSTRKKDNSTYIVQTIKDIYAKDNTTLKKQFDKSPEKFLMY |
| | | QHDPRTFEKLEVIMKQYANEKNPLAKYHEETGEYLTKYSK |
| | | KNNGPIVKSLKYIGNKLGSHLDVTHQFKSSTKKLVKLSIKPY |
| | | RFDVYLTDKGYKFITISYLDVLKKDNYYYIPEQKYDKLKLG |
| | | KAIDKNAKFIASFYKNDLIKLDGEIYKIIGVNSDTRNMIELDL |
| | | PDIRYKRYCELNNIKGEPRIKKTIGKKVNSIEKLTTDVLGNVF |
| | | TNTQYTKPQLLFKRGNGG |
| 802 | SluCas9 in | MNQKFILGLDIGITSVGYGLIDYETKNIIDAGVRLFPEANVEN |
| | WO2019/ 118935 | NEGRRSKRGSRRLKRRRIHRLERVKKLLEDYNLLDQSQIPQS |
| | | TNPYAIRVKGLSEALSKDELVIALLHIAKRRGIHKIDVIDSND |
| | | DVGNELSTKEQLNKNSKLLKDKFVCQIQLERMNEGQVRGEK |
| | | NRFKTADIIKEIIQLLNVQKNFHQLDENFINKYIELVEMRREYF |
| | | EGPGKGSPYGWEGDPKAWYETLMGHCTYFPDELRSVKYAY |
| | | SADLFNALNDLNNLVIQRDGLSKLEYHEKYHIIENVFKQKKK |
| | | PTLKQIANEINVNPEDIKGYRITKSGKPQFTEFKLYHDLKSVL |
| | | FDQSILENEDVLDQIAEILTIYQDKDSIKSKLTELDILLNEEDK |
| | | ENIAQLTGYTGTHRLSLKCIRLVLEEQWYSSRNQMEIFTHLN |
| | | IKPKKINLTAANKIPKAMIDEFILSPVVKRTFGQAINLINKIIEK |
| | | YGVPEDIIIELARENNSKDKQKFINEMQKKNENTRKRINEIIGK |
| | | YGNQNAKRLVEKIRLHDEQEGKCLYSLESIPLEDLLNNPNHY |
| | | EVDHIIPRSVSFDNSYHNKVLVKQSENSKKSNLTPYQYFNSGK |
| | | SKLSYNQFKQHILNLSKSQDRISKKKKEYLLEERDINKFEVQK |
| | | EFINRNLVDTRYATRELTNYLKAYFSANNMNVKVKTINGSFT |
| | | DYLRKVWKFKKERNHGYKHHAEDALIIANADFLFKENKKLK |
| | | AVNSVLEKPEIETKQLDIQVDSEDNYSEMFIIPKQVQDIKDER |
| | | NFKYSHRVDKKPNRQLINDTLYSTRKKDNSTYIVQTIKDIYAK |
| | | DNTTLKKQFDKSPEKFLMYQHDPRTFEKLEVIMKQYANEKN |
| | | PLAKYHEETGEYLTKYSKKNNGPIVKSLKYIGNKLGSHLDVT |
| | | HQFKSSTKKLVKLSIKPYRFDVYLTDKGYKFITISYLDVLKKD |
| | | NYYYIPEQKYDKLKLGKAIDKNAKFIASFYKNDLIKLDGEIY |
| | | KIIGVNSDTRNMIELDLPDIRYKEYCELNNIKGEPRIKKTIG |
| | | KKVNSIEKLTTDVLGNVFTNTQYTKPQLLFKRGNGG |
| 803 | Codon optimized | atg aaa cgt ccg gca gca acc aaa aaa gca ggt cag gcc aag |
| | version of a | aaa aaa 48 |
| | polynucleotide | aaa ggt ggt ggt tca ggt aac cag aaa ttt atc ctg ggt ctg |
| | sequence shown | gat att 96 |
| | from position 61 | ggt att acc agc gtt ggt tat ggc ctg att gat tac gaa acc |
| | to 3225 (SEQ ID | aaa aac 144 |
| | NO: 3 in | att att gat gcc ggt gtt cgt ctg ttt ccg gaa gca aat gtt |
| | WO2019/ | gaa aat 192 |
| | 183150) | aat gaa ggt cgt cgt agc aaa cgt ggt agc cgt cgt ctg aaa |
| | | cgt cgt 240 |
| | | cgt att cat cgt ctg gaa cgt gtt aaa aaa ctg ctg gaa gat |
| | | tat aac 288 |
| | | ctg ctg gat cag agc cag att ccg cag agc acc aat ccg tat |
| | | gca att 336 |
| | | cgt gtt aaa ggt ctg agc gaa gca ctg agc aaa gat gaa ctg |
| | | gtt att 384 |
| | | gca ctg ctg cat att gca aaa cgc cgt ggc att cat aaa atc |
| | | gat gtg 432 |
| | | att gat agc aat gac gat gtg ggt aat gaa ctg agc acc aaa |
| | | gaa cag 480 |
| | | ctg aaa aaa aat agc aaa ctg ctg aaa gac aaa ttc gtg tgt |
| | | cag att 528 |
| | | cag ctg gaa cgt atg aat gaa ggc cag gtt cgt ggt gaa aag |
| | | aat cgc 576 |
| | | ttt aaa acc gca gac atc atc aaa gaa att atc cag ctg ctg |
| | | aac gtg 624 |
| | | cag aaa aac ttc cat cag ctg gat gaa aac ttc atc aac aaa |
| | | tac atc 672 |
| | | gag ctg gtt gaa atg cgt cgc gaa tat ttt gaa ggt ccg ggt |
| | | aaa ggt 720 |
| | | agc ccg tat ggt tgg gaa ggt gat ccg aaa gca tgg tat gaa |
| | | acc ctg 768 |
| | | atg ggt cat tgt acc tat ttt ccg gat gaa ctg cgt agc gtt |
| | | aaa tat 816 |
| | | gcc tat agc gca gac ctg ttt aat gca ctg aat gat ctg aat |
| | | aac ctg 864 |
| | | gtg att cag cgt gat ggt ctg agc aaa ctg gaa tat cat gag |
| | | aaa tat 912 |
| | | cac atc atc gaa aac gtg ttc aaa cag aag aag aaa ccg acc |
| | | ctg aaa 960 |
| | | caa atc gcc aac gaa att aat gtg aac ccg gaa gat att aaa |
| | | ggc tac 1008 |
| | | cgt att acc aaa agc ggt aaa ccg cag ttc acc gaa ttt aaa |
| | | ctg tat 1056 |
| | | cac gat ctg aaa agc gtg ctg ttt gat cag agc att ctg gaa |
| | | aat gaa 1104 |
| | | gat gtg ctg gac cag att gca gaa att ctg acc att tat cag |
| | | gac aaa 1152 |
| | | gac agc atc aaa agc aaa ctg acc gaa ctg gat att ctg ctg |
| | | aat gaa 1200 |
| | | gaa gat aaa gag aac att gca cag ctg acc ggt tat acc ggc |
| | | acc cat 1248 |
| | | cgt ctg agc ctg aaa tgt att cgt ctg gta ctg gaa gaa cag |
| | | tgg tat 1296 |
| | | agc agc cgt aat cag atg gaa atc ttt acc cat ctg aac att |
| | | aaa ccg 1344 |
| | | aag aaa atc aat ctg acc gca gcc aac aaa att ccg aaa gcc |
| | | atg att 1392 |
| | | gat gag ttt att ctg agt ccg gtt gtg aaa cgt acc ttt ggt |
| | | cag gca 1440 |
| | | att aac ctg atc aac aaa atc att gaa aaa tat ggc gtg cct |
| | | gag gat 1488 |
| | | atc att att gaa ctg gca cgt gaa aac aac agc aaa gat aaa |
| | | cag aaa 1536 |
| | | ttc atc aac gag atg cag aag aag aac gaa aat acc cgc aaa |
| | | cgg att 1584 |
| | | aac gag atc att ggc aaa tat ggt aat cag aat gcc aaa cgc |
| | | ctg gtg 1632 |
| | | gaa aaa att cgt ctg cat gat gaa caa gag ggc aaa tgt ctg |
| | | tat agc 1680 |
| | | ctg gaa agc att cct ctg gaa gat ctg ctg aac aat ccg aat |
| | | cat tat 1728 |
| | | gaa gtg gat cac att att ccg cgt agc gtg agc ttt gat aat |
| | | tcc tat 1776 |
| | | cat aat aaa gtg ctg gtg aaa cag agc gaa aac tcc aaa aaa |
| | | tcc aac 1824 |
| | | ctg aca ccg tat cag tat ttc aat agc ggc aaa tcc aaa ctg |
| | | agc tac 1872 |
| | | aac cag ttt aaa cag cat att ctg aac ctg agc aaa agc cag |
| | | gat cgc 1920 |
| | | atc agc aag aag aag aag gag tac ctg ctg gaa gaa cgc gac |
| | | atc aac 1968 |
| | | aaa ttt gaa gtg cag aaa gaa ttt atc aac cgc aac ctg gtt |
| | | gat acc 2016 |
| | | cgt tat gca acc cgt gaa ctg acc aat tat ctg aaa gca tat |
| | | ttc agc 2064 |
| | | gcc aac aac atg aac gtg aaa gtg aaa acg att aac ggc agc |
| | | ttt acc 2112 |
| | | gat tat ctg cgt aaa gtg tgg aaa ttc aaa aaa gaa cgc aac |
| | | cac ggc 2160 |
| | | tat aaa cat cat gcc gaa gat gcc ctg att att gca aat gca |
| | | gat ttc 2208 |
| | | ctg ttt aaa gaa aac aaa aaa ctg aaa gcc gtc aac agc gtg |
| | | ctg gaa 2256 |
| | | aaa ccg gaa att gag aca aaa cag ctg gac att cag gtt gat |
| | | agc gaa 2304 |
| | | gat aat tac agc gaa atg ttt atc atc ccg aaa cag gtg cag |
| | | gat atc 2352 |
| | | aaa gat ttt cgc aac ttc aaa tat agc cac cgc gtt gac aaa |
| | | aaa cct 2400 |
| | | aat cgt cag ctg att aac gat acc ctg tat agc acc cgc aaa |
| | | aaa gat 2448 |
| | | aac agc acc tat att gtg cag acc att aaa gac atc tac gcc |
| | | aaa gat 2496 |
| | | aat acc acc ctg aaa aaa cag ttc gac aaa agc cca gaa aaa |
| | | ttt ctg 2544 |
| | | atg tat cag cat gat ccg cgt acc ttc gaa aaa ctg gaa gtt |
| | | att atg 2592 |
| | | aaa cag tat gcc aac gag aaa aat ccg ctg gcc aaa tat cac |
| | | gaa gaa 2640 |
| | | acc ggt gaa tat ctg acc aaa tat tcc aag aag aac aac ggt |
| | | ccg atc 2688 |
| | | gtt aaa tcc ctg aaa tat atc ggt aat aaa ctg ggc agc cat |
| | | ctg gat 2736 |
| | | gtt acc cat cag ttt aaa agc tcc aca aag aag ctg gtt aaa |
| | | ctg tcc 2784 |
| | | atc aaa ccg tat cgc ttt gat gtg tat ctg acc gac aaa ggc |
| | | tat aaa 2832 |
| | | ttc att acc atc agc tat ctg gac gtg ctg aaa aaa gac aac |
| | | tat tat 2880 |
| | | tat atc ccg gaa cag aaa tat gat aaa ctg aaa ctg ggt aaa |
| | | gcc atc 2928 |
| | | gat aaa aac gcc aaa ttt atc gcc agc ttc tac aaa aac gac |
| | | ctg att 2976 |
| | | aaa ctg gat ggc gag atc tat aaa atc atc ggt gtt aat agc |
| | | gac acc 3024 |
| | | cgc aat atg att gag ctg gat ctg ccg gat att cgc tat aaa |
| | | gaa tat 3072 |
| | | tgc gaa ctg aac aac att aaa ggc gaa ccg cgt atc aaa aag |
| | | acc atc 3120 |
| | | ggc aaa aaa gtg aat agc atc gag aaa ctg acc acc gat gtt |
| | | ctg ggt 3168 |
| | | aat gtg ttt acc aat acc cag tat acc aaa cct cag ctg ctg |
| | | ttc aaa 3216 |
| | | cgc ggt aat ggc gga gga tct ggc ccc cct aag aaa aag cgg |
| | | aag gtg 3264 |
| | | ggt gga agc gga ggc agc ggg gga tca ggc cat cat cat cac |
| | | cat cat 3312 |
| | | taa |
| | | 3315 |
| 804 | Codon optimized | atgaaccaaa agttcattct ggggctcgat atcggcatca cctccgtggg |
| | version of a | atatggtctg 60 |
| | polynucleotide | atcgactacg agactaagaa catcatcgac gctggagtgc gactgttccc |
| | sequence shown | ggaagcgaac 120 |
| | from position 61 | gtggagaaca acgaaggccg cagatccaag cgcgggtcca gaaggctcaa |
| | to 3225 (SEQ ID | gaggcggagg 180 |
| | NO: 44 in | atccatagac tcgaaagagt gaagaagctc cttgaagatt acaatctgtt |
| | WO2019/ | ggaccagagc 240 |
| | 183150) | cagattcccc aaagcaccaa cccgtacgcc atcagagtga agggcctgtc |
| | | cgaagccctg 300 |
| | | tcgaaagatg aactggtcat tgccctgctg catattgcca aacggcgcgg |
| | | aatccataag 360 |
| | | atcgacgtga tagactccaa cgatgacgtg ggcaacgaac tgtcaaccaa |
| | | ggagcagctg 420 |
| | | aacaagaact cgaaactgct gaaggacaag ttcgtctgcc aaattcaact |
| | | ggaacggatg 480 |
| | | aacgagggac aagtcagggg agagaaaaac cggttcaaga ccgcggacat |
| | | catcaaggag 540 |
| | | atcatccaac tcctgaatgt gcagaagaac tttcaccagc tggatgaaaa |
| | | cttcattaac 600 |
| | | aagtacattg aactggtgga aatgcggagg gagtacttcg agggacctgg |
| | | aaagggatcc 660 |
| | | ccttacggct gggaagggga ccccaaggct tggtacgaaa cgctcatggg |
| | | ccattgcact 720 |
| | | tactttccgg acgaactccg gtccgtgaag tacgcatact ctgccgatct |
| | | gttcaatgca 780 |
| | | ctcaacgacc ttaacaactt ggtgatccag cgcgatggcc tgtccaagtt |
| | | ggaataccac 840 |
| | | gaaaagtatc acatcatcga gaacgtgttc aagcagaaaa agaagccaac |
| | | tctgaagcag 900 |
| | | attgccaacg aaattaacgt gaaccccgag gatatcaagg gataccggat |
| | | cactaagtcc 960 |
| | | ggcaaaccac agttcaccga gttcaagctg taccacgatc tgaagtcggt |
| | | gctctttgac 1020 |
| | | cagtccatcc tggaaaacga agatgtgctg gaccagattg ctgagatcct |
| | | gaccatctac 1080 |
| | | caggacaagg actcgattaa gagcaagctc acggagctgg acattctgct |
| | | gaacgaagag 1140 |
| | | gataaggaga acatcgcgca gctcactggt tacaccggta cccaccgctt |
| | | gtcccttaag 1200 |
| | | tgcatccggc tggtcctcga ggaacaatgg tactccagcc ggaaccagat |
| | | ggagatcttc 1260 |
| | | acgcacttga acatcaagcc gaagaagatt aacctgaccg ctgcgaacaa |
| | | gatacccaag 1320 |
| | | gccatgatcg acgagtttat cctctcaccg gtggtcaagc gcaccttcgg |
| | | acaagccatc 1380 |
| | | aacctcatca acaagattat cgagaagtac ggcgtgcctg aggatatcat |
| | | catcgagctg 1440 |
| | | gctcgggaga acaactcaaa ggataagcag aagttcatta acgagatgca |
| | | gaaaaagaac 1500 |
| | | gagaacactc gcaagcggat taatgagatc atcggtaaat acgggaacca |
| | | gaacgccaag 1560 |
| | | cggcttgtgg aaaagattcg gctccacgac gagcaggagg gaaagtgtct |
| | | gtactcgctg 1620 |
| | | gagagcattc ccctggagga cctcctgaac aacccaaacc actacgaagt |
| | | ggatcacata 1680 |
| | | atcccccgca gcgtgtcatt cgacaattcc taccataaca aggtcctcgt |
| | | gaagcagtcc 1740 |
| | | gagaatagca agaagtccaa cctgactccg taccagtact tcaactccgg |
| | | caaatccaag 1800 |
| | | ctgtcctaca accagttcaa acagcacatc ctcaacctgt caaagagcca |
| | | ggacaggatc 1860 |
| | | tcgaagaaga agaaggaata ccttctcgag gaacgggata tcaataagtt |
| | | cgaggtgcag 1920 |
| | | aaggagttta tcaatagaaa cctggtggac actcgctatg ccacccgcga |
| | | actgaccaac 1980 |
| | | tacctgaagg cgtacttctc cgccaacaac atgaacgtga aggtcaaaac |
| | | tattaacggc 2040 |
| | | agcttcaccg actatctgcg caaggtctgg aagttcaaga aggaacgcaa |
| | | ccacggttac 2100 |
| | | aagcaccacg cggaagatgc gctgattatc gccaacgctg acttcctgtt |
| | | caaggaaaac 2160 |
| | | aagaagctca aggccgtgaa ctcagtgctc gagaagcctg aaatcgagac |
| | | taagcagctg 2220 |
| | | gacatccagg tcgattcgga agataactac tccgaaatgt tcatcatccc |
| | | taagcaagtg 2280 |
| | | caggacatca aggacttcag gaatttcaag tacagccatc gcgtggacaa |
| | | gaagccaaac 2340 |
| | | agacagctga tcaacgatac actgtattcc acccggaaga aggacaactc |
| | | cacctacatc 2400 |
| | | gtccaaacca ttaaggacat ctacgcaaag gacaacacca cgcttaagaa |
| | | gcagttcgac 2460 |
| | | aagagccccg aaaagttcct catgtaccag cacgacccca gaaccttcga |
| | | gaagcttgaa 2520 |
| | | gtgatcatga agcagtacgc caacgaaaag aacccactgg ctaagtacca |
| | | cgaggaaacc 2580 |
| | | ggcgaatacc tgaccaagta ctccaaaaag aacaacggac cgatcgtcaa |
| | | gtccctgaag 2640 |
| | | tacattggga acaagctcgg ctcgcacctc gatgtgaccc accagttcaa |
| | | gtcctcgacc 2700 |
| | | aaaaagctcg tgaagctgtc catcaagccg taccggttcg acgtgtacct |
| | | gactgacaag 2760 |
| | | ggatataagt tcatcaccat ttcctacctc gacgtgttga agaaggataa |
| | | ctactactac 2820 |
| | | attccggaac agaagtacga caagctcaag ctcggaaagg ccatcgacaa |
| | | aaatgcgaag 2880 |
| | | ttcatcgcga gcttctacaa gaatgacttg atcaagctgg atggcgaaat |
| | | ctacaagatc 2940 |
| | | atcggggtca actccgatac ccgcaacatg attgagctgg atctgcccga |
| | | cattcggtac 3000 |
| | | aaggaatact gcgagctgaa caacatcaag ggagaaccgc ggatcaagaa |
| | | aaccatcgga 3060 |
| | | aagaaagtga acagcatcga gaaactgact actgacgtcc tgggaaacgt |
| | | gttcaccaac 3120 |
| | | acacaataca ccaaacccca gctgctgttt aagcgcggga ac |
| | | 3162 |
| 805 | Codon optimized | atgaaccaga agttcatcct gggcctcgac atcggcatca cctctgttgg |
| | version of a | ctacggcctg 60 |
| | polynucleotide | atcgactacg agacaaagaa catcatcgat gccggcgtgc ggctgttccc |
| | sequence shown | tgaggccaac 120 |
| | from position 61 | gtggaaaaca acgagggccg cagaagcaag agaggcagca gaaggctgaa |
| | to 3225 (SEQ ID | gcggcggaga 180 |
| | NO: 45 in | atccaccggc tggaaagagt gaagaagctg ctcgaggact acaacctgct |
| | WO2019/ | ggaccagtct 240 |
| | 183150) | cagatccctc agagcacaaa cccctacgcc atcagagtga agggcctgtc |
| | | tgaggccctg 300 |
| | | agcaaggacg agctggttat cgccctgctg cacattgcca agcggagagg |
| | | catccacaag 360 |
| | | atcgacgtga tcgacagcaa cgacgacgtg ggcaatgage tgagcaccaa |
| | | agagcagctg 420 |
| | | aacaagaaca gcaagctgct gaaggacaag ttcgtgtgcc agattcagct |
| | | ggaacggatg 480 |
| | | aatgagggcc aagtgcgggg cgagaagaac agattcaaga ccgccgacat |
| | | catcaaagag 540 |
| | | atcatccagc tgctcaacgt gcagaagaac ttccaccagc tggacgagaa |
| | | cttcatcaac 600 |
| | | aagtacatcg agctggtcga gatgcggcgc gagtactttg aaggccctgg |
| | | aaagggcagc 660 |
| | | ccttatggct gggaaggcga toccaaggct tggtacgaga cactgatggg |
| | | ccactgcacc 720 |
| | | tactttcccg acgagctgag aagcgtgaag tacgcctaca gcgccgacct |
| | | gttcaacgcc 780 |
| | | ctgaacgacc tgaacaacct cgtgatccag agagatggcc tgtccaagct |
| | | ggaataccac 840 |
| | | gagaagtacc acatcattga gaacgtgttc aagcagaaga agaagcccac |
| | | actgaagcag 900 |
| | | atcgccaacg agatcaacgt gaaccccgag gacatcaagg gctacagaat |
| | | caccaagagc 960 |
| | | ggcaagcccc agttcaccga gttcaagctg taccacgatc tgaagtccgt |
| | | gctgttcgac 1020 |
| | | cagagcatcc tggaaaacga ggacgtgctg gatcagatcg ccgagatcct |
| | | gaccatctac 1080 |
| | | caggacaagg acagcatcaa gagcaagctg accgagctgg acatcctgct |
| | | gaacgaagag 1140 |
| | | gacaaagaga atatcgccca gctgaccggc tacaccggca cacatagact |
| | | gagcctgaag 1200 |
| | | tgcatccggc tggtgctgga agaacagtgg tactccagcc ggaaccagat |
| | | ggaaatcttc 1260 |
| | | acccacctga acatcaagcc caagaagatc aacctgaccg ccgccaacaa |
| | | gatccccaag 1320 |
| | | gccatgatcg acgagttcat tctgagcccc gtggtcaaga gaaccttcgg |
| | | ccaggccatc 1380 |
| | | aatctgatca acaagattat cgagaagtat ggcgtgcccg aggatatcat |
| | | catcgaactg 1440 |
| | | gccagagaga acaacagcaa ggacaagcaa aagttcatca acgagatgca |
| | | gaaaaagaac 1500 |
| | | gagaacaccc ggaagcggat caacgaaatc atcgggaagt acggcaacca |
| | | gaacgccaag 1560 |
| | | agactggtgg aaaagatccg gctgcacgac gagcaagagg gcaagtgtct |
| | | gtacagcctg 1620 |
| | | gaatctatcc ctctcgagga tctgctgaac aatcccaacc actacgaggt |
| | | ggaccacatt 1680 |
| | | atccccagaa gcgtgtcctt cgacaacagc taccacaaca aggtgctggt |
| | | caagcagagc 1740 |
| | | gagaactcca agaagtccaa tctgacccct taccagtact tcaacagcgg |
| | | caagtctaag 1800 |
| | | ctgagctaca accagtttaa gcagcacatc ctgaacctca gcaagagcca |
| | | ggaccggatc 1860 |
| | | agcaagaaga agaaagagta cctgctcgaa gagagggaca ttaacaagtt |
| | | cgaggtgcag 1920 |
| | | aaagagttta tcaaccggaa cctggtggac accagatacg ccaccagaga |
| | | gctgaccaac 1980 |
| | | tacctgaagg cctacttcag cgccaacaac atgaacgtga aagtcaagac |
| | | catcaacggc 2040 |
| | | agcttcaccg actacctgcg gaaagtgtgg aagtttaaga aagagcggaa |
| | | ccacggctac 2100 |
| | | aagcaccacg ccgaagatgc cctgattatc gccaatgccg acttcctgtt |
| | | caaagagaac 2160 |
| | | aagaaactga aggccgtgaa cagcgtgctg gaaaagcccg agatcgagac |
| | | aaaacagctc 2220 |
| | | gacatccagg tggacagcga ggacaactac agcgagatgt tcatcatccc |
| | | caaacaggtg 2280 |
| | | caggatatca aggacttccg gaacttcaag tacagccacc gcgtggacaa |
| | | gaagcctaac 2340 |
| | | cggcagctga tcaatgacac cctgtacagc acccgcaaga aggacaacag |
| | | cacctacatc 2400 |
| | | gtgcagacga tcaaggacat ctacgccaag gacaatacga ccctgaagaa |
| | | gcagttcgac 2460 |
| | | aagagccccg agaagttcct gatgtaccag cacgacccca ggaccttcga |
| | | gaagctggaa 2520 |
| | | gtgatcatga agcagtacgc taatgagaag aacccgctgg ccaagtacca |
| | | cgaggaaacc 2580 |
| | | ggcgagtacc tgaccaagta ctctaagaag aacaacggcc ccatcgtgaa |
| | | gtccctgaag 2640 |
| | | tatatcggca acaagctggg cagccacctg gacgtgacac accagttcaa |
| | | gagcagcacc 2700 |
| | | aagaagctgg tcaaactgtc catcaagcca taccgcttcg acgtgtacct |
| | | gacagacaag 2760 |
| | | gggtacaagt ttatcaccat cagctacctc gacgtgctga agaaggataa |
| | | ctactactac 2820 |
| | | atccccgagc agaagtacga caagctgaag ctgggaaaag ccatcgacaa |
| | | gaatgccaag 2880 |
| | | ttcattgcca gcttctacaa gaacgacctc atcaagctgg acggcgagat |
| | | ctacaagatc 2940 |
| | | atcggcgtga actccgacac acggaacatg attgagctgg acctgcctga |
| | | catccggtac 3000 |
| | | aaagagtact gcgaactgaa caatatcaag ggcgagcccc ggatcaaaaa |
| | | gacgatcggc 3060 |
| | | aagaaagtga acagcattga gaagctgacc accgatgtgc tgggcaatgt |
| | | gttcaccaac 3120 |
| | | acacagtaca ccaagcctca gctgctgttc aagcggggca at |
| | | 3162 |
| 806 | Staphylococcus | MKEKYILGLDIGITSVGYGIINFETKKIIDAGVRLFPEANVDNNEGRRSKRGS |
| | pasteuri Cas9 | RRLKRRRIHRLERVKLLLTEYDLINKEQIPTSNNPYQIRVKGLSEILSKDELAIA |
| | | LLHLAKRRGIHNINVSSEDEDASNELSTKEQINRNNKLLKDKYVCEVQLQRL |
| | | KEGQIRGEKNRFKTTDILKEIDQLLKVQKDYHNLDIDFINQYKEIVETRREYF |
| | | EGPGQGSPFGWNGDLKKWYEMLMGHCTYFPQELRSVKYAYSADLFNALND |
| | | LNNLIIQRDNSEKLEYHEKYHIIENVFKQKKKPTLKQIAKEIGVNPEDIKGYRI |
| | | TKSGTPQEFKLYHDLKSIVFDKSILENEAILDQIAEILTIYQDEQSIKEELNK |
| | | LPEILNEQDKAEIAKLIGYNGTHRLSLKCIHLINEELWQTSRNQMEIFNYLNIK |
| | | PNKVDLSEQNKIPKDMVNDFILSPVVKRTFIQSINVINKVIEKYGIPEDIIIELAR |
| | | ENNSDDRKKFINNLQKKNEATRKRINEIIGQTGNQNAKRIVEKIRLHDQQEGK |
| | | CLYSLESIALMDLLNNPQNYEVDHIIPRSVAFDNSIHNKVLVKQIENSKKGNR |
| | | TPYQYLNSSDAKLSYNQFKQHILNLSKSKDRISKKKKDYLLEERDINKFEVQ |
| | | KEFINRNLVDTRYATRELTSYLKAYFSANNMDVKVKTINGSFTNHLRKVWR |
| | | FDKYRNHGYKHHAEDALIIANADFLFKENKKLQNTNKILEKPTIENNTKKVT |
| | | VEKEEDYNNVFETPKLVEDIKQYRDYKFSHRVDKKPNRQLINDTLYSTRMK |
| | | DEHDYIVQTITDIYGKDNTNLKKQFNKNPEKFLMYQNDPKTFEKLSIIMKQY |
| | | SDEKNPLAKYYEETGEYLTKYSKKNNGPIVKKIKLLGNKVGNHLDVTNKYE |
| | | NSTKKLVKLSIKNYRFDVYLTEKGYKFVTIAYLNVFKKDNYYYIPKDKYQEL |
| | | KEKKKIKDTDQFIASFYKNDLIKLNGDLYKIIGVNSDDRNIIELDYYDIKYKDY |
| | | CEINNIKGEPRIKKTIGKKTESIEKFTTDVLGNLYLHSTEKAPQLIFKRGL |
| 807 | Staphylococcus | MEKDYILGLDIGIGSVGYGLIDYDTKSIIDAGVRLFPEANADNNLGRRAKRGA |
| | microti Cas9 | RRLKRRRIHRLERVKSLLSEYKIISGLAPTNNQPYNIRVKGLTEQLTKDELAV |
| | | ALLHIAKRRGIHNVDVAADKEETASDSLSTKDQINKNAKFLESRYVCELQKE |
| | | RLENEGHVRGVENRFLTKDIVREAKKIIDTQMQYYPEIDETFKEKYISLVETR |
| | | REYYEGPGKGSPYGWDADVKKWYQLMMGHCTYFPVEFRSVKYAYTADLY |
| | | NALNDLNNLTIARDDNPKLEYHEKYHIIENVFKQKRNPTLKQIAKEIGVNDIN |
| | | ISGYRVTKSGKPQFTSFKLFHDLKKVVKDHAILDDIDLLNQIAEILTIYQDKDS |
| | | IVAELGQLEYLMSEADKQSISELTGYTGTHSLSLKCMNMIIDELWHSSMNQM |
| | | EVFTYLNMRPKKYELKGYQRIPTDMIDDAILSPVVKRSFKQAIGVVNAIIKKY |
| | | GLPKDIIIELARESNSAEKSRYLRAIQKKNEKTRERIEAIIKEYGNENAKGLVQ |
| | | KIKLHDAQEGKCLYSLKDIPLEDLLRNPNNYDIDHIIPRSVSFDDSMHNKVLV |
| | | RREQNAKKNNQTPYQYLTSGYADIKYSVFKQHVLNLAENKDRMTKKKREY |
| | | LLEERNINKYDVQKEFINRNLVDTRYTTRELTTLLKTYFTINNLDVKVKTING |
| | | SFTDFLRKRWGFKKNRDEGYKHHAEDALIIANADYLFKEHKLLKEIKDVSDL |
| | | AGDERNSNVKDEDQYEEVFGGYFKIEDIKKYKIKKFSHRVDKKPNRQLINDT |
| | | IYSTRVKDDKRYLINTLKNLYDKSNGDLKERMQKDPESLLMYHHDPQTFEK |
| | | LKIVMSQYENEKNPLAKYFEETGQYLTKYAKHDNGPAIHKIKYYGNKLYEH |
| | | LDITKNYHNPQNKVVQLSQKSFRFDVYQTDKGYKFISIAYLTLKNEKNYYAI |
| | | SQEKYDQLKSEKKISNNAVFIGSFYTSDIIEINNEKFRVIGVNSDKNNLIEVDRI |
| | | DIRQKEFIELEEEKKNNRIKVTIGRKTTNIEKFHTDILGNMYKSKRPKAPQLVFKK |
| | | G |
| 808 | Staphylococcus | MNNYILGLDIGITSVGYGIVDSDTREIKDAGVRLFPEANVDNNEGRRSKRGA |
| | hyicus Cas9 | RRLKRRRIHRLDRVKHLLAEYDLLDLTNIPKSTNPYQTRVKGLNEKLSKDEL |
| | | VIALLHIAKRRGIHNVNVMMDDNDSGNELSTKDQLKKNAKALSDKYVCELQ |
| | | LERFEQDYKVRGEKNRFKTEDFVREARKLLETQSKFFEIDQTFIMRYIELIETR |
| | | REYFEGPGKGSPFGWEGNIKKWFEQMMGHCTYFPEELRSVKYSYSAELFNA |
| | | LNDLNNLVITRDEDAKLNYGEKFQIIENVFKQKKTPNLKQIAIEIGVHETEIKG |
| | | YRVNKSGKPEFTQFKLYHDLKNIFKDPKYLNDIQLMDNIAEIITIYQDAESIIK |
| | | ELNQLPELLSEREKEKISALSGYSGTHRLSLKCINLLLDDLWESSLNQMELFT |
| | | KLNLKPKKIDLSQQHKIPSKLVDDFILSPVVKRAFIQSIQVVNAIIDKYGLPEDI |
| | | IIELARENNSDDRRKFLNQLQKQNEETRKQVEKVLREYGNDNAKRIVQKIKL |
| | | HNMQEGKCLYSLKDIPLEDLLRNPHHYEVDHIIPRSVAFDNSMHNKVLVRAD |
| | | ENSKKGNRTPYQYLNSSESSLSYNEFKQHILNLSKTKDRITKKKREYLLEERD |
| | | INKFDVQKEFINRNLVDTRYATRELTSLLKAYFSANNLDVKVKTINGSFTNYL |
| | | RKVWKFDKDRNKGYKHHAEDALIIANADFLFKHNKKLRNINKVLDAPSKEV |
| | | DKKRVTVQSEDEYNQIFEDTQKAQAIKKFEIRKFSHRVDKKPNRQLINDTLYS |
| | | TRNIDGIEYVVESIKDIYSVNNDKVKTKFKKDPHRLLMYRNDPQTFEKFEKV |
| | | FKQYESEKNPFAKYYEETGEKIRKFSKTGQGPYINKIKYLRERLGRHCDVTN |
| | | KYINSRNKIVQLKIYSYRFDIYQYGNNYKMITISYIDLEQKSNYYYISREKYEQ |
| | | KKKDKQIDDSYKFIGSFYKNDIINYNGEMYRVIGVNDSEKNKIQLDMIDISIK |
| | | DYMELNNIKKTGVIYKTIGKSTTHIEKYTTDILGNLYKAAPPKKPQLIFK |
| 809 | Staphylococcus | MNQKFILGLDIGITSVGYGLIDYETKNIIDAGVRLFPEANVENNEGRRSKRGS |
| | lugdunensis mini- | RRLKRRRIHRLERV |
| | domain 1 from 8 | |
| | mini-domain | |
| | library | |
| 810 | Staphylococcus | MKEKYILGLDIGITSVGYGIINFETKKIIDAGVRLFPEANVDNNEGRRSKRGS |
| | pasteuri mini- | RRLKRRRIHRLERV |
| | domain 1 from 8 | |
| | mini-domain | |
| | library | |
| 811 | Staphylococcus | MNNYILGLDIGITSVGYGIVDSDTREIKDAGVRLFPEANVDNNEGRRSKRGA |
| | hyicus mini- | RRLKRRRIHRLDRV |
| | domain 1 from 8 | |
| | mini-domain | |
| | library | |
| 812 | Staphylococcus | MEKDYILGLDIGIGSVGYGLIDYDTKSIIDAGVRLFPEANADNNLGRRAKRGA |
| | microti mini- | RRLKRRRIHRLERV |
| | domain 1 from 8 | |
| | mini-domain | |
| | library | |
| 813 | Staphylococcus | KKLLEDYNLLDQSQIPQSTNPY AIRVKGLSEALSKDELVIALLHIAKRRGIH |
| | lugdunensis mini- | |
| | domain 2 from 8 | |
| | mini-domain | |
| | library | |
| 814 | Staphylococcus | KLLLTEYDLINKEQIPTSNNPYQIRVKGLSEILSKDELAIALLHLAKRRGIH |
| | pasteuri mini- | |
| | domain 2 from 8 | |
| | mini-domain | |
| | library | |
| 815 | Staphylococcus | KHLLAEYDLLDLTNIPKSTNPYQTRVKGLNEKLSKDELVIALLHIAKRRGIH |
| | hyicus mini- | |
| | domain 2 from 8 | |
| | mini-domain | |
| | library | |
| 816 | Staphylococcus | KSLLSEYKIISGLAPTNNQPYNIRVKGLTEQLTKDELA V ALLHIAKRRGIH |
| | microti mini- | |
| | domain 2 from 8 | |
| | mini-domain | |
| | library | |
| 817 | Staphylococcus | KIDVIDSNDDVGNELSTKEQLNKNSKLLKDKFVCQIQLERMNEGQVRGEKNRFKT |
| | lugdunensis mini- | ADIIKEIIQLLNVQKNFHQLDENFINKYIELVEMRRE |
| | domain 3 from 8 | |
| | mini-domain | |
| | library | |
| 818 | Staphylococcus | NINVSSEDEDASNELSTKEQINRNNKLLKDKYVCEVQLQRLKEGQIRGEKNR |
| | pasteuri mini- | FKTTDILKEIDQLLKVQKDYHNLDIDFINQYKEIVETRRE |
| | domain 3 from 8 | |
| | mini-domain | |
| | library | |
| 819 | Staphylococcus | NVNVMMDDNDSGNELSTKDQLKKNAKALSDKYVCELQLERFEQDYKVRG |
| | hyicus mini- | EKNRFKTEDFVREARKLLETQSKFFEIDQTFIMRYIELIETRRE |
| | domain 3 from 8 | |
| | mini-domain | |
| | library | |
| 820 | Staphylococcus | NVDVAADKEETASDSLSTKDQINKNAKFLESRYVCELQKERLENEGHVRGV |
| | microti mini- | ENRFLTKDIVREAKKIIDTQMQYYPEIDETFKEKYISLVETRRE |
| | domain 3 from 8 | |
| | mini-domain | |
| | library | |
| 821 | Staphylococcus | YFEGPGKGSPYGWEGDPKAWYETLMGHCTYFPDELRSVKYAYSADLFNAL |
| | lugdunensis mini- | NDLNNLVIQRDGLSKLEYHEKYHIIENVFKQKKKPTLKQIANEINVNPEDIKG |
| | domain 4 from 8 | YRITKSGK |
| | mini-domain | |
| | library | |
| 822 | Staphylococcus | YFEGPGQGSPFGWNGDLKKWYEMLMGHCTYFPQELRSVKYAYSADLFNAL |
| | pasteuri mini- | NDLNNLIIQRDNSEKLEYHEKYHIIENVFKQKKKPTLKQIAKEIGVNPEDIKGY |
| | domain 4 from 8 | RITKSGT |
| | mini-domain | |
| | library | |
| 823 | Staphylococcus | YFEGPGKGSPFGWEGNIKKWFEQMMGHCTYFPEELRSVKYSYSAELFNALNDLNNL |
| | hyicus mini- | VITRDEDAKLNYGEKFQIIENVFKQKKTPNLKQIAIEIGVHETEIKGYRVNKSGK |
| | domain 4 from 8 | |
| | mini-domain | |
| | library | |
| 824 | Staphylococcus | YYEGPGKGSPYGWDADVKKWYQLMMGHCTYFPVEFRSVKYAYTADLYNA |
| | microti mini- | LNDLNNLTIARDDNPKLEYHEKYHIIENVFKQKRNPTLKQIAKEIGVNDINISG |
| | domain 4 from 8 | YRVTKSGK |
| | mini-domain | |
| | library | |
| 825 | Staphylococcus | PQFTEFKLYHDLKSVLFDQSILENEDVLDQIAEILTIYQDKDSIKSKLTELDILL |
| | lugdunensis mini- | NEEDKENIAQLTGYTGTHRLSLKCIRLVLEEQWYSSRNQMEIFTHLNIKPKKINLT |
| | domain 5 from 8 | AANKIPKAMIDEFILSPVVK |
| | mini-domain | |
| | library | |
| 826 | Staphylococcus | PQFTEFKLYHDLKSIVFDKSILENEAILDQIAEILTIYQDEQSIKEELNKLPEILN |
| | pasteuri mini- | EQDKAEIAKLIGYNGTHRLSLKCIHLINEELWQTSRNQMEIFNYLNIKPNKVD |
| | domain 5 from 8 | LSEQNKIPKDMVNDFILSPVVK |
| | mini-domain | |
| | library | |
| 827 | Staphylococcus | PEFTQFKLYHDLKNIFKDPKYLNDIQLMDNIAEIITIYQDAESIIKELNQLPELL |
| | hyicus mini- | SEREKEKISALSGYSGTHRLSLKCINLLLDDLWESSLNQMELFTKLNLKPKKI |
| | domain 5 from 8 | DLSQQHKIPSKLVDDFILSPVVK |
| | mini-domain | |
| | library | |
| 828 | Staphylococcus | PQFTSFKLFHDLKKVVKDHAILDDIDLLNQIAEILTIYQDKDSIVAELGQLEYL |
| | microti mini- | MSEADKQSISELTGYTGTHSLSLKCMNMIIDELWHSSMNQMEVFTYLNMRP |
| | domain 5 from 8 | KKYELKGYQRIPTDMIDDAILSPVVK |
| | mini-domain | |
| | library | |
| 829 | Staphylococcus | RTFGQAINLINKIIEKYGVPEDIIIELARENNSKDKQKFINEMQKKNENTRKRI |
| | lugdunensis mini- | NEIIGKYGNQN AKRLVEKIRLHDEQEGKCLYSLES |
| | domain 6 from 8 | |
| | mini-domain | |
| | library | |
| 830 | Staphylococcus | RTFIQSINVINKVIEKYGIPEDIIIELARENNSDDRKKFINNLQKKNEATRKRINE |
| | pasteuri mini- | IIGQTGNQNAKRIVEKIRLHDQQEGKCLYSLES |
| | domain 6 from 8 | |
| | mini-domain | |
| | library | |
| 831 | Staphylococcus | RAFIQSIQVVNAIIDKYGLPEDIIIELARENNSDDRRKFLNQLQKQNEETRKQV |
| | hyicus mini- | EKVLREYGNDNAKRIVQKIKLHNMQEGKCLYSLKD |
| | domain 6 from 8 | |
| | mini-domain | |
| | library | |
| 832 | Staphylococcus | RSFKQAIGVVNAIIKKYGLPKDIIIELARESNSAEKSRYLRAIQKKNEKTRERIE |
| | microti mini- | AIIKEYGNENAKGLVQKIKLHDAQEGKCLYSLKD |
| | domain 6 from 8 | |
| | mini-domain | |
| | library | |
| 833 | Staphylococcus | IPLEDLLNNPNHYEVDHIIPRSVSFDNSYHNKVLVKQSENSKKSNLTPYQYFN |
| | lugdunensis mini- | SGKSKLSYNQFKQHILNLSKSQDRISKKKKEYLLEERDINKFEVQKEFINRNL |
| | domain 7 from 8 | VDTRYATREL |
| | mini-domain | |
| | library | |
| 834 | Staphylococcus | IALMDLLNNPQNYEVDHIIPRSVAFDNSIHNKVLVKQIENSKKGNRTPYQYLN |
| | pasteuri mini- | SSDAKLSYNQFKQHILNLSKSKDRISKKKKDYLLEERDINKFEVQKEFINRNL |
| | domain 7 from 8 | VDTRYATREL |
| | mini-domain | |
| | library | |
| 835 | Staphylococcus | IPLEDLLRNPHHYEVDHIIPRSVAFDNSMHNKVLVRADENSKKGNRTPYQYL |
| | hyicus mini- | NSSESSLSYNEFKQHILNLSKTKDRITKKKREYLLEERDINKFDVQKEFINRNL |
| | domain 7 from 8 | VDTRYATREL |
| | mini-domain | |
| | library | |
| 836 | Staphylococcus | IPLEDLLRNPNNYDIDHIIPRSVSFDDSMHNKVLVRREQAKKNNQTPYQYLTSGYA |
| | microti mini- | DIKYSVFKQHVLNLAENKDRMTKKKREYLLEERNINKYDVQKEFINR |
| | domain 7 from 8 | NLVDTRYTTREL |
| | mini-domain | |
| | library | |
| 837 | Staphylococcus | TNYLKAYFSANNMNVKVKTINGSFTDYLRKVWKFKKERNHGYKHHAEDAL |
| | lugdunensis mini- | IIANADFLFKENKKLKAVNSVLEKPEIETKQLDIQVDSEDNYSEMFIIPKQVQD |
| | domain 8 from 8 | IKDFRNFKYSHRVDKKPNRQLINDTLYSTRKKDNSTYIVQTIKDIYAKDNTTL |
| | mini-domain | KKQFDKSPEKFLMYQHDPRTFEKLEVIMKQYANEKNPLAKYHEETGEYLTK |
| | library | YSKKNNGPIVKSLKYIGNKLGSHLDVTHQFKSSTKKLVKLSIKPYRFDVYLT |
| | | DKGYKFITISYLDVLKKDNYYYIPEQKYDKLKLGKAIDKNAKFIASFYKNDLI |
| | | KLDGEIYKIIGVNSDTRNMIELDLPDIRYKEYCELNNIKGEPRIKKTIGKKVNSI |
| | | EKLTTDVLGNVFTNTQYTKPQLLFKRGN |
| 838 | Staphylococcus | TSYLKAYFSANNMDVKVKTINGSFTNHLRKVWRFDKYRNHGYKHHAEDAL |
| | pasteuri mini- | IIANADFLFKENKKLQNTNKILEKPTIENNTKKVTVEKEEDYNNVFETPKLVE |
| | domain 8 from 8 | DIKQYRDYKFSHRVDKKPNRQLINDTLYSTRMKDEHDYIVQTITDIYGKDNT |
| | mini-domain | NLKKQFNKNPEKFLMYQNDPKTFEKLSIIMKQYSDEKNPLAKYYEETGEYLT |
| | library | KYSKKNNGPIVKKIKLLGNKVGNHLDVTNKYENSTKKLVKLSIKNYRFDVY |
| | | LTEKGYKFVTIAYLNVFKKDNYYYIPKDKYQELKEKKKIKDTDQFIASFYKN |
| | | DLIKLNGDLYKIIGVNSDDRNIIELDYYDIKYKDYCEINNIKGEPRIKKTIGKKT |
| | | ESIEKFTTDVLGNLYLHSTEKAPQLIFKRGL |
| 839 | Staphylococcus | TSLLKAYFSANNLDVKVKTINGSFTNYLRKVWKFDKDRNKGYKHHAEDALI |
| | hyicus mini- | IANADFLFKHNKKLRNINKVLDAPSKEVDKKRVTVQSEDEYNQIFEDTQKAQ |
| | domain 8 from 8 | AIKKFEIRKFSHRVDKKPNRQLINDTLYSTRNIDGIEYVVESIKDIYSVNNDKV |
| | mini-domain | KTKFKKDPHRLLMYRNDPQTFEKFEKVFKQYESEKNPFAKYYEETGEKIRKE |
| | library | SKTGQGPYINKIKYLRERLGRHCDVTNKYINSRNKIVQLKIYSYRFDIYQYGN |
| | | NYKMITISYIDLEQKSNYYYISREKYEQKKKDKQIDDSYKFIGSFYKNDIINYN |
| | | GEMYRVIGVNDSEKNKIQLDMIDISIKDYMELNNIKKTGVIYKTIGKSTTHIEK |
| | | YTTDILGNLYKAAPPKKPQLIFK |
| 840 | Staphylococcus | TTLLKTYFTINNLDVKVKTINGSFTDFLRKRWGFKKNRDEGYKHHAEDALII |
| | microti mini- | ANADYLFKEHKLLKEIKDVSDLAGDERNSNVKDEDQYEEVFGGYFKIEDIKK |
| | domain 8 from 8 | YKIKKFSHRVDKKPNRQLINDTIYSTRVKDDKRYLINTLKNLYDKSNGDLKE |
| | mini-domain | RMQKDPESLLMYHHDPQTFEKLKIVMSQYENEKNPLAKYFEETGQYLTKYA |
| | library | KHDNGPAIHKIKYYGNKLVEHLDITKNYHNPQNKVVQLSQKSFRFDVYQTD |
| | | KGYKFISIAYLTLKNEKNYYAISQEKYDQLKSEKKISNNAVFIGSFYTSDIIEIN |
| | | NEKFRVIGVNSDKNNLIEVDRIDIRQKEFIELEEEKKNNRIKVTIGRKTTNIEKF |
| | | HTDILGNMYKSKRPKAPQLVFKKG |
| 841 | Staphylococcus | MNQKFILGLDIGITSVGYGLIDYETKNIIDAGVRLFPEANV |
| | lugdunensis mini- | |
| | domain 1 from 12 | |
| | mini-domain | |
| | library | |
| 842 | Staphylococcus | MKEKYILGLDIGITSVGYGIINFETKKIIDAGVRLFPEANV |
| | pasteuri mini- | |
| | domain 1 from 12 | |
| | mini-domain | |
| | library | |
| 843 | Staphylococcus | MNNYILGLDIGITSVGYGIVDSDTREIKDAGVRLFPEANV |
| | hyicus mini- | |
| | domain 1 from 12 | |
| | mini-domain | |
| | library | |
| 844 | Staphylococcus | MEKDYILGLDIGIGSVGYGLIDYDTKSIIDAGVRLFPEANA |
| | microti mini- | |
| | domain 1 from 12 | |
| | mini-domain | |
| | library | |
| 845 | Staphylococcus | ENNEGRRSKRGSRRLKRRRIHRL |
| | lugdunensis mini- | |
| | domain 2 from 12 | |
| | mini-domain | |
| | library | |
| 846 | Staphylococcus | DNNEGRRSKRGSRRLKRRRIHRL |
| | pasteuri mini- | |
| | domain 2from 12 | |
| | mini-domain | |
| | library | |
| 847 | Staphylococcus | DNNEGRRSKRGARRLKRRRIHRL |
| | hyicus mini- | |
| | domain 2 from 12 | |
| | mini-domain | |
| | library | |
| 848 | Staphylococcus | DNNLGRRAKRGARRLKRRRIHRL |
| | microti mini- | |
| | domain 2 from 12 | |
| | mini-domain | |
| | library | |
| 849 | Staphylococcus | ERVKKLLEDYNLLDQSQIPQSTNPYAIRVKGLSEALSKDELVIALLHIAKRRGI H |
| | lugdunensis mini- | |
| | domain 3 from 12 | |
| | mini-domain | |
| | library | |
| 850 | Staphylococcus | ERVKKLLEDYNLLDQSQIPQSTNPYAIRVKGLSEALSKDELVIALLHIAKRRG IH |
| | pasteuri mini- | |
| | domain 3 from 12 | |
| | mini-domain | |
| | library | |
| 851 | Staphylococcus | DRVKHLLAEYDLLDLTNIPKSTNPYQTRVKGLNEKLSKDELVIALLHIAKRR GIH |
| | hyicus mini- | |
| | domain 3 from 12 | |
| | mini-domain | |
| | library | |
| 852 | Staphylococcus | ERVKSLLSEYKIISGLAPTNNQPYNIRVKGLTEQLTKDELAVALLHIAKRRGIH |
| | microti mini- | |
| | domain 3 from 12 | |
| | mini-domain | |
| | library | |
| 853 | Staphylococcus | KIDVIDSNDDVGNELSTKEQLNKNSKLLKDKFVCQIQLERMNEGQVRGEKNRFKT |
| | lugdunensis mini- | ADIIKEIIQLLNVQKNFHQLDENFINKYIELVEMRREY |
| | domain 4 from 12 | |
| | mini-domain | |
| | library | |
| 854 | Staphylococcus | INVSSEDEDASNELSTKEQINRNNKLLKDKYVCEVQLQRLKEGQIRGEKNR |
| | pasteuri mini- | FKTTDILKEIDQLLKVQKDYHNLDIDFINQYKEIVETRREY |
| | domain 4 from 12 | |
| | mini-domain | |
| | library | |
| 855 | Staphylococcus | NVNVMMDDNDSGNELSTKDQLKKNAKALSDKYVCELQLERFEQDYKVRG |
| | hyicus mini- | EKNRFKTEDFVREARKLLETQSKFFEIDQTFIMRYIELIETRREY |
| | domain 4 from 12 | |
| | mini-domain | |
| | library | |
| 856 | Staphylococcus | NVDVAADKEETASDSLSTKDQINKNAKFLESRYVCELQKERLENEGHVRGV |
| | microti mini- | ENRFLTKDIVREAKKIIDTQMQYYPEIDETFKEKYISLVETRREY |
| | domain 4 from 12 | |
| | mini-domain | |
| | library | |
| 857 | Staphylococcus | FEGPGKGSPYGWEGDPKAWYETLMGHCTYFPDELRSVKYAYSADLFNALN |
| | lugdunensis mini- | DLNNLVIQRDGLSKLEYHEKYHIIENVFKQKKKPTLKQIANEINVNPEDIKGY |
| | domain 5 from 12 | RITKSGKPQFT |
| | mini-domain | |
| | library | |
| 858 | Staphylococcus | FEGPGQGSPFGWNGDLKKWYEMLMGHCTYFPQELRSVKYAYSADLFNALN |
| | pasteuri mini- | DLNNLIIQRDNSEKLEYHEKYHIIENVFKQKKKPTLKQIAKEIGVNPEDIKGYR |
| | domain 5 from 12 | ITKSGTPQFf |
| | mini-domain | |
| | library | |
| 859 | Staphylococcus | FEGPGKGSPFGWEGNIKKWFEQMMGHCTYFPEELRSVKYSYSAELFNALNDLNNL |
| | hyicus mini- | VITRDEDAKLNYGEKFQIIENVFKQKKTPNLKQIAIEIGVHETEIKGYRV |
| | domain 5 from 12 | NKSGKPEFT |
| | mini-domain | |
| | library | |
| 860 | Staphylococcus | YEGPGKGSPYGWDADVKKWYQLMMGHCTYFPVEFRSVKYAYTADLYNAL |
| | microti mini- | NDLNNLTIARDDNPKLEYHEKYHIIENVFKQKRNPTLKQIAKEIGVNDINISG |
| | domain 5 from 12 | YRVTKSGKPQFT |
| | mini-domain | |
| | library | |
| 861 | Staphylococcus | EFKLYHDLKSVLFDQSILENEDVLDQIAEILTIYQDKDSIKSKLTELDILLNEED |
| | lugdunensis mini- | KENIAQLTGYTGTHRLSLKCIRLVLEEQWYSSRNQMEIFTHLNIKPKKINLTA |
| | domain 6 from 12 | ANKIPKAMIDEFILSPVVKR |
| | mini-domain | |
| | library | |
| 862 | Staphylococcus | EFKLYHDLKSIVEDKSILENEAILDQIAEILTIYQDEQSIKEELNKLPEILNEQDK |
| | pasteuri mini- | AEIAKLIGYNGTHRLSLKCIHLINEELWQTSRNQMEIFNYLNIKPNKVDLSEQ |
| | domain 6 from 12 | NKIPKDMVNDFILSPVVKR |
| | mini-domain | |
| | library | |
| 863 | Staphylococcus | QFKLYHDLKNIFKDPKYLNDIQLMDNIAEIITIYQDAESIIKELNQLPELLSERE |
| | hyicus mini- | KEKISALSGYSGTHRLSLKCINLLLDDLWESSLNQMELFTKLNLKPKKIDLSQ |
| | domain 6 from 12 | QHKIPSKLVDDFILSPVVKR |
| | mini-domain | |
| 864 | Staphylococcus | SFKLFHDLKKVVKDHAILDDIDLLNQIAEILTIYQDKDSIVAELGQLEYLMSE |
| | microti mini- | ADKQSISELTGYTGTHSLSLKCMNMIIDELWHSSMNQMEVFTYLNMRPKKY |
| | domain 6 from 12 | ELKGYQRIPTDMIDDAILSPVVKR |
| | mini-domain | |
| | library | |
| 865 | Staphylococcus | TFGQAINLINKIIEKYGVPEDIIIELARENNSKDKQKFINEMQKKNENTRKRINE |
| | lugdunensis mini- | IIGKYGNQNAKRL VEKIRLHDEQEGKCLYSL |
| | domain 7 from 12 | |
| | mini-domain | |
| | library | |
| 866 | Staphylococcus | TFIQSINVINKVIEKYGIPEDIIIELARENNSDDRKKFINNLQKKNEATRKRINEI |
| | pasteuri mini- | I GQTGNQNAKRIVEKIRLHDQQEGKCLYSL |
| | domain 7 from 12 | |
| | mini-domain | |
| | library | |
| 867 | Staphylococcus | AFIQSIQVVNAIIDKYGLPEDIIIELARENNSDDRRKFLNQLQKQNEETRKQVE |
| | hyicus mini- | KVLREYGNDNAKRIVQKIKLHNMQEGKCLYSL |
| | domain 7 from 12 | |
| | mini-domain | |
| | library | |
| 868 | Staphylococcus | SFKQAIGVVNAIIKKYGLPKDIIIELARESNSAEKSRYLRAIQKKNEKTRERIEA |
| | microti mini- | IIKEYGNENAKGLVQKIKLHDAQEGKCLYSL |
| | domain 7 from 12 | |
| | mini-domain | |
| | library | |
| 869 | Staphylococcus | ESIPLEDLLNNPNHYEVDHIIPRSVSFDNSYHNKVLVKQSENSKKSNLTPYQY |
| | lugdunensis mini- | FNSGKSKLSYNQFKQHILNLSKSQDRISKKKKEYLLEER |
| | domain 8 from 12 | |
| | mini-domain | |
| | library | |
| 870 | Staphylococcus | ESIALMDLLNNPQNYEVDHIIPRSVAFDNSIHNKVLVKQIENSKKGNRTPYQY |
| | pasteuri mini- | LNSSDAKLSYNQFKQHILNLSKSKDRISKKKKDYLLEER |
| | domain 8 from 12 | |
| | mini-domain | |
| | library | |
| 871 | Staphylococcus | KDIPLEDLLRNPHHYEVDHIIPRSVAFDNSMHNKVLVRADENSKKGNRTPYQ |
| | hyicus mini- | YLNSSESSLSYNEFKQHILNLSKTKDRITKKKREYLLEER |
| | domain 8 from 12 | |
| | mini-domain | |
| | library | |
| 872 | Staphylococcus | KDIPLEDLLRNPNNYDIDHIIPRSVSFDDSMHNKVLVRREQNAKKNNQTPYQ |
| | microti mini- | YLTSGYADIKYSVFKQHVLNLAENKDRMTKKKREYLLEER |
| | domain 8 from 12 | |
| | mini-domain | |
| | library | |
| 873 | Staphylococcus | DINKFEVQKEFINRNLVDTRYATRELT |
| | lugdunensis mini- | |
| | domain 9 from 12 | |
| | mini-domain | |
| | library | |
| 874 | Staphylococcus | DINKFEVQKEFINRNLVDTRYATRELT |
| | pasteuri mini- | |
| | domain 9 from 12 | |
| | mini-domain | |
| | library | |
| 875 | Staphylococcus | DINKFDVQKEFINRNLVDTRYATRELT |
| | hyicus mini- | |
| | domain 9 from 12 | |
| | mini-domain | |
| | library | |
| 876 | Staphylococcus | NINKYDVQKEFINRNLVDTRYTTRELT |
| | microti mini- | |
| | domain 9 from 12 | |
| | mini-domain | |
| | library | |
| 877 | Staphylococcus | NYLKAYFSANNMNVKVKTINGSFTDYLRKVWKFKKERNHGYKHHAEDALII |
| | lugdunensis mini- | ANADFLFKENKKL |
| | domain 10 from | |
| | 12 mini-domain | |
| | library | |
| 878 | Staphylococcus | SYLKAYFSANNMDVKVKTINGSFTNHLRKVWRFDKYRNHGYKHHAEDALII |
| | pasteuri mini- | ANADFLFKENKKL |
| | domain 10 from | |
| | 12 mini-domain | |
| | library | |
| 879 | Staphylococcus | SLLKAYFSANNLDVKVKTINGSFTNYLRKVWKFDKDRNKGYKHHAEDALII |
| | hyicus mini- | ANADFLFKHNKKL |
| | domain 10 from | |
| | 12 mini-domain | |
| | library | |
| 880 | Staphylococcus | TLLKTYFTINNLDVKVKTINGSFTDFLRKRWGFKKNRDEGYKHHAEDALIIA |
| | microti mini- | NADYLFKEHKLL |
| | domain 10 from | |
| | 12 mini-domain | |
| | library | |
| 881 | Staphylococcus | KAVNSVLEKPEIETKQLDIQVDSEDNYSEMFIIPKQVQDIKDERNFKYSHRVD |
| | lugdunensis mini- | KKPNRQLINDTLYSTRKKDNSTYIVQTIKDIYAKDNTTLKKQFDKSPEKFLM |
| | domain 11 from | YQHDPRTFEKLEVIMKQYANEKNPLAKYHEETGEYLTKYSKKNNGPIVKSL |
| | 12 mini-domain | KYIGNKLGSHLDVTHQFKSSTKKLVKLSIK |
| | library | |
| 882 | Staphylococcus | QNTNKILEKPTIENNTKKVTVEKEEDYNNVFETPKLVEDIKQYRDYKFSHRV |
| | pasteuri mini- | DKKPNRQLINDTLYSTRMKDEHDYIVQTITDIYGKDNTNLKKQFNKNPEKFL |
| | domain 11 from | MYQNDPKTFEKLSIIMKQYSDEKNPLAKYYEETGEYLTKYSKKNNGPIVKKI |
| | 12 mini-domain | KLLGNKVGNHLDVTNKYENSTKKL VKLSIK |
| | library | |
| 883 | Staphylococcus | RNINKVLDAPSKEVDKKRVTVQSEDEYNQIFEDTQKAQAIKKFEIRKFSHRV |
| | hyicus mini- | DKKPNRQLINDTLYSTRNIDGIEYVVESIKDIYSVNNDKVKTKFKKDPHRLLM |
| | domain 11 from | YRNDPQTFEKFEKVFKQYESEKNPFAKYYEETGEKIRKFSKTGQGPYINKIKY |
| | 12 mini-domain | LRERLGRHCDVTNKYINSRNKIVQLK |
| | library | |
| 884 | Staphylococcus | KEIKDVSDLAGDERNSNVKDEDQYEEVEGGYFKIEDIKKYKIKKFSHRVDKK |
| | microti mini- | PNRQLINDTIYSTRVKDDKRYLINTLKNLYDKSNGDLKERMQKDPESLLMYH |
| | domain 11 from | HDPQTFEKLKIVMSQYENEKNPLAKYFEETGQYLTKYAKHDNGPAIHKIKYY |
| | 12 mini-domain | GNKL VEHLDITKNYHNPQNKVVQLSQK |
| | library | |
| 885 | Staphylococcus | PYRFDVYLTDKGYKFITISYLDVLKKDNYYYIPEQKYDKLKLGKAIDKNAKF |
| | lugdunensis mini- | IASFYKNDLIKLDGEIYKIIGVNSDTRNMIELDLPDIRYKEYCELNNIKGEPRIK |
| | domain 12 from | KTIGKKVNSIEKLTTDVLGNVFTNTQYTKPQLLFKRGN |
| | 12 mini-domain | |
| | library | |
| 886 | Staphylococcus | NYRFDVYLTEKGYKFVTIAYLNVFKKDNYYYIPKDKYQELKEKKKIKDTDQ |
| | pasteuri mini- | FIASFYKNDLIKLNGDLYKIIGVNSDDRNIIELDYYDIKYKDYCEINNIKGEPRI |
| | domain 12 from | KKTIGKKTESIEKFTTDVLGNLYLHSTEKAPQLIFKRGL |
| | 12 mini-domain | |
| | library | |
| 887 | Staphylococcus | IYSYRFDIYQYGNNYKMITISYIDLEQKSNYYYISREKYEQKKKDKQIDDSYK |
| | hyicus mini- | FIGSFYKNDIINYNGEMYRVIGVNDSEKNKIQLDMIDISIKDYMELNNIKKTG |
| | domain 12 from | VIYKTIGKSTTHIEKYTTDILGNLYKAAPPKKPQLIFK |
| | 12 mini-domain | |
| | library | |
| 888 | Staphylococcus | SFRFDVYQTDKGYKFISIA YLTLKNEKNYY AISQEKYDQLKSEKKISNNA VFI |
| | microti mini- | GSFYTSDIIEINNEKFRVIGVNSDKNNLIEVDRIDIRQKEFIELEEEKKNNRIKV |
| | domain 12 from | TIGRKTTNIEKFHTDILGNMYKSKRPKAPQLVFKKG |
| | 12 mini-domain | |
| | library | |

What is claimed is:

1. A composition comprising:
a. one or more guide RNAs (gRNAs), or a vector encoding one or more gRNAs, wherein each gRNA comprises:
i. a spacer sequence selected from any one of SEQ ID NOs: 1-51, 53, 55-56, 58-59, 61-62, 64, 66, and 70; or
ii. a spacer sequence that is at least 20 contiguous nucleotides of any one of SEQ ID NOs: 1-51, 53, 55-56, 58-59, 61-62, 64, 66, or 70; or
iii. a spacer sequence that is at least 90% identical to any one of SEQ ID NOs: 1-51, 53, 55-56, 58-59, 61-62, 64, 66, or 70;
wherein the gRNAs are for use with a SluCas9; and
optionally a Staphylococcus lugdunensis Cas9 (SluCas9) or a nucleic acid encoding a SluCas9; or
b. one or more guide RNAs (gRNAs), or a vector encoding one or more gRNAs, wherein each gRNA comprises:
i. a spacer sequence selected from any one of SEQ ID NOs: 200-259; or
ii. a spacer sequence that is at least 20 contiguous nucleotides of any one of SEQ ID NOs: 200-259; or
iii. a spacer sequence that is at least 90% identical to any one of SEQ ID NOs: 200-259;
wherein the gRNAs are for use with a Staphylococcus aureus Cas9 (SaCas9); and
optionally a SaCas9 or a nucleic acid encoding a SaCas9.

2. The composition of claim 1, comprising a SluCas9 or a nucleic acid encoding a SluCas9.

3. The composition of claim 1, comprising a SaCas9 or a nucleic acid encoding a SaCas9.

4. A composition comprising:
a pair of guide RNAs comprising a pair of spacer sequences, or one or more vectors encoding the pair of guide RNAs, wherein the pair of spacer sequences comprise:
i. a first spacer sequence selected from SEQ ID NOs: 3, 5, 6, 9, 16, 21, 22, 25, 26, 30, 36, 38, 39, 40, 46, 51, 53, 55, 56, 58, 59, 61, 62, 64, 66, and 70, and a second spacer sequence selected from SEQ ID NOs: 1, 2, 4, 7, 8, 10, 11, 12, 13, 14, 15, 17, 18, 19, 20, 23, 24, 27, 28, 29, 31, 32, 33, 34, 35, 37, 41, 42, 43, 44, 45, 47, 48, 49, and 50; and/or
ii. a first spacer sequence having at least 20 contiguous nucleotides of a sequence selected from SEQ ID NOs: 3, 5, 6, 9, 16, 21, 22, 25, 26, 30, 36, 38, 39, 40, 46, 51, 53, 55, 56, 58, 59, 61, 62, 64, 66, and 70, and a second spacer sequence having at least 20 contiguous nucleotides of a sequence selected from SEQ ID NOs: 1, 2, 4, 7, 8, 10, 11, 12, 13, 14, 15, 17, 18, 19, 20, 23, 24, 27, 28, 29, 31, 32, 33, 34, 35, 37, 41, 42, 43, 44, 45, 47, 48, 49, and 50; and/or
iii. a first spacer sequence that is at least 99%, 98%, 97%, 96%, 95%, 94%, 93%, 92%, 91%, or 90% identical to a sequence selected from SEQ ID NOs: 3, 5, 6, 9, 16, 21, 22, 25, 26, 30, 36, 38, 39, 40, 46, 51, 53, 55, 56, 58, 59, 61, 62, 64, 66, and 70, and a second spacer sequence that is at least 99%, 98%, 97%, 96%, 95%, 94%, 93%, 92%, 91%, or 90% identical to a sequence selected from SEQ ID NOs: 1, 2, 4, 7, 8, 10, 11, 12, 13, 14, 15, 17, 18, 19, 20, 23, 24, 27, 28, 29, 31, 32, 33, 34, 35, 37, 41, 42, 43, 44, 45, 47, 48, 49, and 50, wherein the gRNAs are for use with a SluCas9.

5. A composition comprising:
a pair of guide RNAs comprising a pair of spacer sequences, or one or more vectors encoding the pair of guide RNAs, wherein the pair of spacer sequences comprise:
i. a first spacer sequence selected from SEQ ID NOs: 201-203, 211, 215, 220, 225, 231, 235, 238, and 240-259 and a second spacer sequence selected from SEQ ID NOs: 200, 204-210, 212-214, 216-219, 221-224, 226-230, 232-234, 236-237, and 239; and/or
ii. a first spacer sequence having at least 20 contiguous nucleotides of a sequence selected from SEQ ID NOs: 201-203, 211, 215, 220, 225, 231, 235, 238, and 240-259, and a second spacer sequence having at least 20 contiguous nucleotides of a sequence selected from SEQ ID NOs: 1200, 204-210, 212-214, 216-219, 221-224, 226-230, 232-234, 236-237, and 239; and/or
iii. a first spacer sequence that is at least 99%, 98%, 97%, 96%, 95%, 94%, 93%, 92%, 91%, or 90% identical to a sequence selected from SEQ ID NOs: 201-203, 211, 215, 220, 225, 231, 235, 238, and 240-259, and a second spacer sequence that is at least 99%, 98%, 97%, 96%, 95%, 94%, 93%, 92%, 91%, or 90% identical to a sequence selected from SEQ ID NOs: 200, 204-210, 212-214, 216-219, 221-224, 226-230, 232-234, 236-237, and 239, wherein the gRNAs are for use with a SaCas9.

6. A composition comprising:
a pair of guide RNAs comprising a pair of spacer sequences, or one or more vectors encoding the pair of guide RNAs, wherein the pair of spacer sequences comprise:
a) a first spacer sequence selected from SEQ ID NOs: 5, 21, 46, 55, 59, 61, or 64 and a second spacer sequence selected from SEQ ID NOs: 7, 19, 41, or 47, wherein the gRNAs are for use with a SluCas9;
b) a first spacer sequence selected from SEQ ID NOs: 201-202 and a second spacer sequence selected from SEQ ID NOs: 206, 213, 218, or 224, wherein the gRNAs are for use with a SaCas9;
c) a first and second spacer sequence of SEQ ID NOs: 5 and 7, wherein the gRNAs are for use with a SluCas9;
d) a first and second spacer sequence of SEQ ID NOs: 5 and 10, wherein the gRNAs are for use with a SluCas9;
e) a first and second spacer sequence of SEQ ID NOs: 5 and 19, wherein the gRNAs are for use with a SluCas9;
f) a first and second spacer sequence of SEQ ID NOs: 5 and 41, wherein the gRNAs are for use with a SluCas9;
g) a first and second spacer sequence of SEQ ID NOs: 5 and 47, wherein the gRNAs are for use with a SluCas9;
h) a first and second spacer sequence of SEQ ID NOs: 21 and 7, wherein the gRNAs are for use with a SluCas9;
i) a first and second spacer sequence of SEQ ID NOs: 21 and 19, wherein the gRNAs are for use with a SluCas9;
j) a first and second spacer sequence of SEQ ID NOs: 21 and 41, wherein the gRNAs are for use with a SluCas9;
k) a first and second spacer sequence of SEQ ID NOs: 21 and 47, wherein the gRNAs are for use with a SluCas9;
l) a first and second spacer sequence of SEQ ID NOs: 46 and 7, wherein the gRNAs are for use with a SluCas9;
m) a first and second spacer sequence of SEQ ID NOs: 46 and 10, wherein the gRNAs are for use with a SluCas9;
n) a first and second spacer sequence of SEQ ID NOs: 46 and 19 wherein the gRNAs are for use with a SluCas9;
o) a first and second spacer sequence of SEQ ID NOs: 46 and 41, wherein the gRNAs are for use with a SluCas9;
p) a first and second spacer sequence of SEQ ID NOs: 46 and 47, wherein the gRNAs are for use with a SluCas9;
q) a first and second spacer sequence of SEQ ID NOs: 55 and 7, wherein the gRNAs are for use with a SluCas9;
r) a first and second spacer sequence of SEQ ID NOs: 55 and 19, wherein the gRNAs are for use with a SluCas9;
s) a first and second spacer sequence of SEQ ID NOs: 55 and 41, wherein the gRNAs are for use with a SluCas9;
t) a first and second spacer sequence of SEQ ID NOs: 55 and 47, wherein the gRNAs are for use with a SluCas9;
u) a first and second spacer sequence of SEQ ID NOs: 59 and 7, wherein the gRNAs are for use with a SluCas9;
v) a first and second spacer sequence of SEQ ID NOs: 59 and 19, wherein the gRNAs are for use with a SluCas9;
w) a first and second spacer sequence of SEQ ID NOs: 59 and 41, wherein the gRNAs are for use with a SluCas9;
x) a first and second spacer sequence of SEQ ID NOs: 59 and 47, wherein the gRNAs are for use with a SluCas9;
y) a first and second spacer sequence of SEQ ID NOs: 61 and 7, wherein the gRNAs are for use with a SluCas9;
z) a first and second spacer sequence of SEQ ID NOs: 61 and 10, wherein the gRNAs are for use with a SluCas9;
aa) a first and second spacer sequence of SEQ ID NOs: 61 and 19, wherein the gRNAs are for use with a SluCas9;
bb) a first and second spacer sequence of SEQ ID NOs: 61 and 41, wherein the gRNAs are for use with a SluCas9;
cc) a first and second spacer sequence of SEQ ID NOs: 61 and 47, wherein the gRNAs are for use with a SluCas9;
dd) a first and second spacer sequence of SEQ ID NOs: 64 and 7, wherein the gRNAs are for use with a SluCas9;
ee) a first and second spacer sequence of SEQ ID NOs: 64 and 19, wherein the gRNAs are for use with a SluCas9;
ff) a first and second spacer sequence of SEQ ID NOs: 64 and 41, wherein the gRNAs are for use with a SluCas9;
gg) a first and second spacer sequence of SEQ ID NOs: 64 and 47, wherein the gRNAs are for use with a SluCas9;
hh) a first and second spacer sequence of SEQ ID NOs: 202 and 218, wherein the gRNAs are for use with a SaCas9;
ii) a first and second spacer sequence of SEQ ID NOs: 201 and 224, wherein the gRNAs are for use with a SaCas9;
jj) a first and second spacer sequence of SEQ ID NOs: 202 and 213, wherein the gRNAs are for use with a SaCas9; or
kk) a first and second spacer sequence of SEQ ID NOs: 202 and 206, wherein the gRNAs are for use with a SaCas9.

7. The composition of claim 4, further comprising a SluCas9, or a nucleic acid encoding the SluCas9.

8. The composition of claim 5, further comprising a SaCas9, or a nucleic acid encoding the SaCas9.

9. The composition of any one of the preceding claims, wherein the guide RNA comprises a crRNA and/or a tracrRNA sequence.

10. The composition of any one of claims 1a, 4, 6a, and 6c-6gg, wherein the guide RNA comprises any one of SEQ ID NOs: 1-51, 53, 55-56, 58-59, 61-62, 64, 66, and 70, and further comprises:
a. a sequence selected from SEQ ID NOs: 600-604;
b. a sequence that is at least 80%, 85%, 90%, 91%, 92%, 93%, 94%, 95%, 96%, 97%, 98%, 99% or 100% identical to SEQ ID NOs: 600-604; or
c. a sequence that differs from SEQ ID NOs: 600-604 by no more than 1, 2, 3, 4, 5, 10, 20, or 25 nucleotides.

11. The composition of any one of claims 1a, 4, 6a, and 6c-6gg, wherein the SluCas9 comprises SEQ ID NO: 712.

12. The composition of any one of claims 1a, 4, 6a, and 6c-6gg, wherein the SluCas9 comprises a sequence that is at least 80%, 85%, 90%, 91%, 92%, 93%, 94%, 95%, 96%, 97%, 98%, 99% or 100% identical to the sequence of SEQ ID NO: 712.

13. The composition of any one of claims 1b, 5, 6b, and 6hh-6kk, wherein the SaCas9 comprises SEQ ID NO: 711.

14. The composition of any one of claims 1b, 5, 6b, and 6hh-6kk, wherein the SaCas9 comprises a sequence that is at least 80%, 85%, 90%, 91%, 92%, 93%, 94%, 95%, 96%, 97%, 98%, 99% or 100% identical to the sequence of SEQ ID NO: 711.

15. The composition of any one of claims 1a, 4, 6a, and 6c-6gg, wherein the SluCas9 comprises:
a. a sequence selected from SEQ ID NOs: 800-805 and 809-888;
b. a chimeric SluCas9 protein comprising a SluCas9 PAM interacting domain.

16. The composition of any one of claims 1a, 4, 6a, and 6c-6gg, wherein the SluCas9 or nucleic acid encoding SluCas9 comprises one or more of the following mutations to SEQ ID NO: 712:
a. a mutation at any one of, or combination of, positions R246, N414, T420, or R655;
b. a mutation at the position corresponding to position R246 of SEQ ID NO: 712 (e.g., R246A);
c. a mutation at the position corresponding to position N414 of SEQ ID NO: 712 (e.g., N414A);
d. a mutation at the position corresponding to position T420 of SEQ ID NO: 712 (e.g., T420A);
e. a mutation at the position corresponding to position R655 of SEQ ID NO: 712 (e.g., R655A);
f. a combination of mutations at the positions corresponding to position R246 of SEQ ID NO: 712 (e.g., R246A), position N414 of SEQ ID NO: 712 (e.g., N414A), position T420 of SEQ ID NO: 712 (e.g., T420A), and position R655 of SEQ ID NO: 712 (e.g., R655A);
g. a mutation at the position corresponding to position Q781 of SEQ ID NO: 712 (e.g., Q781K);
h. a mutation at the position corresponding to position R1013 of SEQ ID NO: 712 (e.g., R1013H); and
i. a combination of mutations at the positions corresponding to position Q781 of SEQ ID NO: 712 (e.g., Q781K) and position R1013 of SEQ ID NO: 712 (e.g., R1013H).

17. The composition of any one of the preceding claims, wherein the guide RNA is an sgRNA.

18. The composition of claim 17, wherein the sgRNA is modified.

19. The composition of claim 18, wherein the modifications alter one or more 2′ positions and/or phosphodiester linkages.

20. The composition of any one of claims 18-19, wherein the modifications alter one or more, or all, of the first three nucleotides of the sgRNA.

21. The composition of any one of claims 18-20, wherein the modifications alter one or more, or all, of the last three nucleotides of the sgRNA.

22. The composition of any one of claims 18-21, wherein the modifications include one or more of a phosphorothioate modification, a 2′-OMe modification, a 2′-O-MOE modification, a 2′-F modification, a 2′-O-methine-4′ bridge modification, a 3′-thiophosphonoacetate modification, and a 2′-deoxy modification.

23. The composition of any one of the preceding claims, wherein the composition further comprises a pharmaceutically acceptable excipient.

24. The composition of any one of the preceding claims, wherein the guide RNA or nucleic acid encoding the guide RNA is associated with a lipid nanoparticle (LNP).

25. The composition of any one of the preceding claims, wherein the guide RNA or nucleic acid encoding the guide RNA is associated with a viral vector.

26. The composition of claim 25, wherein the viral vector is an adeno-associated virus vector, a lentiviral vector, an integrase-deficient lentiviral vector, an adenoviral vector, a vaccinia viral vector, an alphaviral vector, or a herpes simplex viral vector.

27. The composition of claim 26, wherein the viral vector is an adeno-associated virus (AAV) vector.

28. The composition of claim 27, wherein the AAV vector is an AAV1, AAV2, AAV3, AAV4, AAV5, AAV6, AAV7, AAV8, AAVrh10, AAVrh74, or AAV9 vector, wherein the number following AAV indicates the AAV serotype.

29. The composition of claim 28, wherein the AAV vector is an AAV serotype 9 vector.

30. The composition of any one of claims 25-28, wherein the viral vector comprises a tissue-specific promoter.

31. The composition of any one of claims 25-30, wherein the viral vector comprises a muscle-specific promoter, optionally wherein the muscle-specific promoter is a muscle creatine kinase promoter, a desmin promoter, an MHCK7 promoter, or an SPc5-12 promoter.

32. The composition of any one of claims 25-31, wherein the viral vector comprises a neuron-specific promoter, optionally wherein the neuron-specific promoter is an enolase promoter.

33. The composition of any one of the preceding claims comprising a spacer sequence having the sequence of SEQ ID NO: 1.

34. The composition of any one of the preceding claims comprising a spacer sequence having the sequence of SEQ ID NO: 2.

35. The composition of any one of the preceding claims comprising a spacer sequence having the sequence of SEQ ID NO: 3.

36. The composition of any one of the preceding claims comprising a spacer sequence having the sequence of SEQ ID NO: 4.

37. The composition of any one of the preceding claims comprising a spacer sequence having the sequence of SEQ ID NO: 5.

38. The composition of any one of the preceding claims comprising a spacer sequence having the sequence of SEQ ID NO: 6.

39. The composition of any one of the preceding claims comprising a spacer sequence having the sequence of SEQ ID NO: 7.

40. The composition of any one of the preceding claims comprising a spacer sequence having the sequence of SEQ ID NO: 8.

41. The composition of any one of the preceding claims comprising a spacer sequence having the sequence of SEQ ID NO: 9.

42. The composition of any one of the preceding claims comprising a spacer sequence having the sequence of SEQ ID NO: 10.

43. The composition of any one of the preceding claims comprising a spacer sequence having the sequence of SEQ ID NO: 11.

44. The composition of any one of the preceding claims comprising a spacer sequence having the sequence of SEQ ID NO: 12.

45. The composition of any one of the preceding claims comprising a spacer sequence having the sequence of SEQ ID NO: 13.

46. The composition of any one of the preceding claims comprising a spacer sequence having the sequence of SEQ ID NO: 14.

47. The composition of any one of the preceding claims comprising a spacer sequence having the sequence of SEQ ID NO: 15.

48. The composition of any one of the preceding claims comprising a spacer sequence having the sequence of SEQ ID NO: 16.

49. The composition of any one of the preceding claims comprising a spacer sequence having the sequence of SEQ ID NO: 17.

50. The composition of any one of the preceding claims comprising a spacer sequence having the sequence of SEQ ID NO: 18.

51. The composition of any one of the preceding claims comprising a spacer sequence having the sequence of SEQ ID NO: 19.

52. The composition of any one of the preceding claims comprising a spacer sequence having the sequence of SEQ ID NO: 20.

53. The composition of any one of the preceding claims comprising a spacer sequence having the sequence of SEQ ID NO: 21.

54. The composition of any one of the preceding claims comprising a spacer sequence having the sequence of SEQ ID NO: 22.

55. The composition of any one of the preceding claims comprising a spacer sequence having the sequence of SEQ ID NO: 23.

56. The composition of any one of the preceding claims comprising a spacer sequence having the sequence of SEQ ID NO: 24.

57. The composition of any one of the preceding claims comprising a spacer sequence having the sequence of SEQ ID NO: 25.

58. The composition of any one of the preceding claims comprising a spacer sequence having the sequence of SEQ ID NO: 26.

59. The composition of any one of the preceding claims comprising a spacer sequence having the sequence of SEQ ID NO: 27.

60. The composition of any one of the preceding claims comprising a spacer sequence having the sequence of SEQ ID NO: 28.

61. The composition of any one of the preceding claims comprising a spacer sequence having the sequence of SEQ ID NO: 29.

62. The composition of any one of the preceding claims comprising a spacer sequence having the sequence of SEQ ID NO: 30.

63. The composition of any one of the preceding claims comprising a spacer sequence having the sequence of SEQ ID NO: 31.

64. The composition of any one of the preceding claims comprising a spacer sequence having the sequence of SEQ ID NO: 32.

65. The composition of any one of the preceding claims comprising a spacer sequence having the sequence of SEQ ID NO: 33.

66. The composition of any one of the preceding claims comprising a spacer sequence having the sequence of SEQ ID NO: 34.

67. The composition of any one of the preceding claims comprising a spacer sequence having the sequence of SEQ ID NO: 35.

68. The composition of any one of the preceding claims comprising a spacer sequence having the sequence of SEQ ID NO: 36.

69. The composition of any one of the preceding claims comprising a spacer sequence having the sequence of SEQ ID NO: 37.

70. The composition of any one of the preceding claims comprising a spacer sequence having the sequence of SEQ ID NO: 38.

71. The composition of any one of the preceding claims comprising a spacer sequence having the sequence of SEQ ID NO: 39.

72. The composition of any one of the preceding claims comprising a spacer sequence having the sequence of SEQ ID NO: 40.

73. The composition of any one of the preceding claims comprising a spacer sequence having the sequence of SEQ ID NO: 41.

74. The composition of any one of the preceding claims comprising a spacer sequence having the sequence of SEQ ID NO: 42.

75. The composition of any one of the preceding claims comprising a spacer sequence having the sequence of SEQ ID NO: 43.

76. The composition of any one of the preceding claims comprising a spacer sequence having the sequence of SEQ ID NO: 44.

77. The composition of any one of the preceding claims comprising a spacer sequence having the sequence of SEQ ID NO: 45.

78. The composition of any one of the preceding claims comprising a spacer sequence having the sequence of SEQ ID NO: 46.

79. The composition of any one of the preceding claims comprising a spacer sequence having the sequence of SEQ ID NO: 47.

80. The composition of any one of the preceding claims comprising a spacer sequence having the sequence of SEQ ID NO: 48.

81. The composition of any one of the preceding claims comprising a spacer sequence having the sequence of SEQ ID NO: 49.

82. The composition of any one of the preceding claims comprising a spacer sequence having the sequence of SEQ ID NO: 50.

83. The composition of any one of the preceding claims comprising a spacer sequence having the sequence of SEQ ID NO: 51.

84. The composition of any one of the preceding claims comprising a spacer sequence having the sequence of SEQ ID NO: 53.

85. The composition of any one of the preceding claims comprising a spacer sequence having the sequence of SEQ ID NO: 55.

86. The composition of any one of the preceding claims comprising a spacer sequence having the sequence of SEQ ID NO: 56.

87. The composition of any one of the preceding claims comprising a spacer sequence having the sequence of SEQ ID NO: 58.

88. The composition of any one of the preceding claims comprising a spacer sequence having the sequence of SEQ ID NO: 59.

89. The composition of any one of the preceding claims comprising a spacer sequence having the sequence of SEQ ID NO: 61.

90. The composition of any one of the preceding claims comprising a spacer sequence having the sequence of SEQ ID NO: 62.

91. The composition of any one of the preceding claims comprising a spacer sequence having the sequence of SEQ ID NO: 64.

92. The composition of any one of the preceding claims comprising a spacer sequence having the sequence of SEQ ID NO: 66.

93. The composition of any one of the preceding claims comprising a spacer sequence having the sequence of SEQ ID NO: 70.

94. Use of a composition of any one of the preceding claims for the preparation of a medicament for treating a human subject having DM1.

95. Use of a composition of any one of the preceding claims for treating a human subject having DM1.

96. A method of treating a muscular dystrophy characterized by a trinucleotide repeat (TNR) in the 3′ UTR of the DMPK gene, the method comprising delivering to a cell that comprises a TNR in the 3′ UTR of the DMPK gene:
a. the composition of any one of claims 1a, 4, 6a, 6c-6gg, 9-12, and 15-95; or
b. a pair of guide RNAs comprising a pair of spacer sequences, or one or more vectors encoding the pair of guide RNAs, wherein the pair of spacer sequences comprise a first spacer sequence selected from SEQ ID NOs: 3, 5, 6, 9, 16, 21, 22, 25, 26, 30, 36, 38, 39, 40, 46, 51, 53, 55, 56, 58, 59, 61, 62, 64, 66, and 70, and a second spacer sequence selected from SEQ ID NOs: 1, 2, 4, 7, 8, 10, 11, 12, 13, 14, 15, 17, 18, 19, 23, 24, 27, 28, 29, 31, 32, 33, 34, 35, 37, 41, 42, 43, 44, 45, 47, 48, 49, and 50, or a nucleic acid encoding the guide RNA;
c. a pair of guide RNAs comprising a pair of spacer sequences, or one or more vectors encoding the pair of guide RNAs, wherein the pair of spacer sequences comprises SEQ ID NO: 5 and SEQ ID NO: 10;
d. a pair of guide RNAs comprising a pair of spacer sequences, or one or more vectors encoding the pair of guide RNAs, wherein the pair of spacer sequences comprises SEQ ID NO: 46 and SEQ ID NO: 10;
e. a pair of guide RNAs comprising a pair of spacer sequences, or one or more vectors encoding the pair of guide RNAs, wherein the pair of spacer sequences comprises SEQ ID NO: 61 and SEQ ID NO: 10; or
f. a pair of guide RNAs comprising a pair of spacer sequences, or one or more vectors encoding the pair of guide RNAs, wherein the pair of spacer sequences comprises SEQ ID NO: 64 and SEQ ID NO: 47; and
SluCas9 or a nucleic acid encoding the SluCas9.

97. A method of excising a trinucleotide repeat (TNR) in the 3′ UTR of the DMPK gene comprising delivering to a cell that comprises the TNR in the 3′ UTR of the DMPK gene a pair of guide RNAs comprising a pair of spacer sequences, or one or more vectors encoding the pair of guide RNAs, wherein the pair of spacer sequences comprise:
i. a first spacer sequence selected from SEQ ID NOs: 3, 5, 6, 9, 16, 21, 22, 25, 26, 30, 36, 38, 39, 40, 46, 51, 53, 55, 56, 58, 59, 61, 62, 64, 66, and 70, and a second spacer sequence selected from SEQ ID NOs: 1, 2, 4, 7, 8, 10, 11, 12, 13, 14, 15, 17, 18, 19, 20, 23, 24, 27, 28, 29, 31, 32, 33, 34, 35, 37, 41, 42, 43, 44, 45, 47, 48, 49, and 50, or a nucleic acid encoding the guide RNA; and
ii. SluCas9 or a nucleic acid encoding the SluCas9, wherein at least one TNR is excised.

98. The method of any one of claims 96-97, wherein a pair of guide RNAs that comprises a first and second spacer sequence that guide the SluCas9 to or near a TNR, or one or more vectors encoding the pair of guide RNAs, are delivered to the cell.

99. The method of any one of claims 96-98, wherein the first and second spacer sequences have the sequences of SEQ ID NOs: 5 and 7.

100. The method of any one of claims 96-98, wherein the first and second spacer sequences have the sequences of SEQ ID NOs: 5 and 10.

101. The method of any one of claims 96-98, wherein the first and second spacer sequences have the sequences of SEQ ID NOs: 5 and 19.

102. The method of any one of claims 96-98, wherein the first and second spacer sequences have the sequences of SEQ ID NOs: 5 and 41.

103. The method of any one of claims 96-98, wherein the first and second spacer sequences have the sequences of SEQ ID NOs: 5 and 47.

104. The method of any one of claims 96-98, wherein the first and second spacer sequences have the sequences of SEQ ID NOs: 21 and 7.

105. The method of any one of claims 96-98, wherein the first and second spacer sequences have the sequences of SEQ ID NOs: 21 and 19.

106. The method of any one of claims 96-98, wherein the first and second spacer sequences have the sequences of SEQ ID NOs: 21 and 41.

107. The method of any one of claims 96-98, wherein the first and second spacer sequences have the sequences of SEQ ID NOs: 21 and 47.

108. The method of any one of claims 96-98, wherein the first and second spacer sequences have the sequences of SEQ ID NOs: 46 and 7.

109. The method of any one of claims 96-98, wherein the first and second spacer sequences have the sequences of SEQ ID NOs: 46 and 10.

110. The method of any one of claims 96-98, wherein the first and second spacer sequences have the sequences of SEQ ID NOs: 46 and 19.

111. The method of any one of claims 96-98, wherein the first and second spacer sequences have the sequences of SEQ ID NOs: 46 and 41.

112. The method of any one of claims 96-98, wherein the first and second spacer sequences have the sequences of SEQ ID NOs: 46 and 47.

113. The method of any one of claims 96-98, wherein the first and second spacer sequences have the sequences of SEQ ID NOs: 55 and 7.

114. The method of any one of claims 96-98, wherein the first and second spacer sequences have the sequences of SEQ ID NOs: 55 and 19.

115. The method of any one of claims 96-98, wherein the first and second spacer sequences have the sequences of SEQ ID NOs: 55 and 41.

116. The method of any one of claims 96-98, wherein the first and second spacer sequences have the sequences of SEQ ID NOs: 55 and 47.

117. The method of any one of claims 96-98, wherein the first and second spacer sequences have the sequences of SEQ ID NOs: 59 and 7.

118. The method of any one of claims 96-98, wherein the first and second spacer sequences have the sequences of SEQ ID NOs: 59 and 19.

119. The method of any one of claims 96-98, wherein the first and second spacer sequences have the sequences of SEQ ID NOs: 59 and 41.

120. The method of any one of claims 96-98, wherein the first and second spacer sequences have the sequences of SEQ ID NOs: 59 and 47.

121. The method of any one of claims 96-98, wherein the first and second spacer sequences have the sequences of SEQ ID NOs: 61 and 7.

122. The method of any one of claims 96-98, wherein the first and second spacer sequences have the sequences of SEQ ID NOs: 61 and 10.

123. The method of any one of claims 96-98, wherein the first and second spacer sequences have the sequences of SEQ ID NOs: 61 and 19.

124. The method of any one of claims 96-98, wherein the first and second spacer sequences have the sequences of SEQ ID NOs: 61 and 41.

125. The method of any one of claims 96-98, wherein the first and second spacer sequences have the sequences of SEQ ID NOs: 61 and 47.

126. The method of any one of claims 96-98, wherein the first and second spacer sequences have the sequences of SEQ ID NOs: 64 and 7.

127. The method of any one of claims 96-98, wherein the first and second spacer sequences have the sequences of SEQ ID NOs: 64 and 19.

128. The method of any one of claims 96-98, wherein the first and second spacer sequences have the sequences of SEQ ID NOs: 64 and 41.

129. The method of any one of claims 96-98, wherein the first and second spacer sequences have the sequences of SEQ ID NOs: 64 and 47.

130. The method of any one of claims 96-129, further comprising SluCas9, or a nucleic acid encoding the SluCas9.

131. The method of any one of claims 96-130, wherein the guide RNA further comprises a SluCas9 crRNA and/or a tracrRNA sequence.

132. The method of any one of claims 96-131, wherein the guide RNA further comprises:
a. a sequence selected from SEQ ID NOs: 600-603;
b. a sequence that is at least 80%, 85%, 90%, 91%, 92%, 93%, 94%, 95%, 96%, 97%, 98%, 99% or 100% identical to SEQ ID NOs: 600-603; or
c. a sequence that differs from SEQ ID NOs: 600-603 by no more than 1, 2, 3, 4, 5, 10, 15, 20, or 25 nucleotides.

133. The method of any one of claims 96-132, wherein the SluCas9 or nucleic acid encoding SluCas9 comprises SEQ ID NO: 712.

134. The method of any one of claims 96-133, wherein the SluCas9 or nucleic acid encoding SluCas9 comprises a sequence that is at least 80%, 85%, 90%, 91%, 92%, 93%, 94%, 95%, 96%, 97%, 98%, 99% or 100% identical to the sequence of SEQ ID NO: 712.

135. The method of any one of claims 96-134, wherein the SluCas9 or nucleic acid encoding SluCas9 comprises:
a. a sequence selected from SEQ ID NOs: 800-805 and 809-888;
b. a chimeric SaCas9 protein comprising a SluCas9 PAM interacting domain.

136. The method of any one of the claims 96-135, wherein the SluCas9 or nucleic acid encoding SluCas9 comprises one or more of the following mutations to SEQ ID NO: 712:
a. a mutation at any one of, or combination of, positions R246, N414, T420, or R655;
b. a mutation at the position corresponding to position R246 of SEQ ID NO: 712 (e.g., R246A);
c. a mutation at the position corresponding to position N414 of SEQ ID NO: 712 (e.g., N414A);
d. a mutation at the position corresponding to position T420 of SEQ ID NO: 712 (e.g., T420A);
e. a mutation at the position corresponding to position R655 of SEQ ID NO: 712 (e.g., R655A);
f. a combination of mutations at the positions corresponding to position R246 of SEQ ID NO: 712 (e.g., R246A), position N414 of SEQ ID NO: 712 (e.g., N414A), position T420 of SEQ ID NO: 712 (e.g., T420A), and position R655 of SEQ ID NO: 712 (e.g., R655A);
g. a mutation at the position corresponding to position Q781 of SEQ ID NO: 712 (e.g., Q781K);
h. a mutation at the position corresponding to position R1013 of SEQ ID NO: 712 (e.g., R1013H); and
i. a combination of mutations at the positions corresponding to position Q781 of SEQ ID NO: 712 (e.g., Q781K) and position R1013 of SEQ ID NO: 712 (e.g., R1013H).

137. The method of any one of claims 96-136, wherein the guide RNA is an sgRNA.

138. The method of claim 137, wherein the sgRNA is modified.

139. The method of claim 138, wherein the modifications alter one or more 2′ positions and/or phosphodiester linkages.

140. The method of claims 138-139, wherein the modifications alter one or more, or all, of the first three nucleotides of the sgRNA.

141. The method of claims 138-140, wherein the modifications alter one or more, or all, of the last three nucleotides of the sgRNA.

142. The method of claims 138-141, wherein the modifications include one or more of a phosphorothioate modification, a 2′-OMe modification, a 2′-O-MOE modification, a 2′-F modification, a 2′-O-methine-4′ bridge modification, a 3′-thiophosphonoacetate modification, and a 2′-deoxy modification.

143. The method of any one of claims 96-142, wherein the composition further comprises a pharmaceutically acceptable excipient.

144. The method of any one of claims 96-143, wherein the guide RNA is associated with a lipid nanoparticle (LNP), or encoded by a viral vector.

145. The method of claim 144, wherein the viral vector is an adeno-associated virus vector, a lentiviral vector, an integrase-deficient lentiviral vector, an adenoviral vector, a vaccinia viral vector, an alphaviral vector, or a herpes simplex viral vector.

146. The method of claim 145, wherein the viral vector is an adeno-associated virus (AAV) vector.

147. The method of claim 146, wherein the AAV vector is an AAV1, AAV2, AAV3, AAV4, AAV5, AAV6, AAV7, AAV8, AAVrh10, AAVrh74, or AAV9 vector, wherein the number following AAV indicates the AAV serotype.

148. The method of claim 147, wherein the AAV vector is an AAV serotype 9 vector.

149. The method of any one of claims 144-148, wherein the viral vector comprises a tissue-specific promoter.

150. The method of any one of claims 144-147, wherein the viral vector comprises a muscle-specific promoter, optionally wherein the muscle-specific promoter is a muscle creatine kinase promoter, a desmin promoter, an MHCK7 promoter, or an SPc5-12 promoter. The method of any one of claims 135-141, wherein the viral vector comprises a neuron-specific promoter, optionally wherein the neuron-specific promoter is an enolase promoter.

151. A method of treating a muscular dystrophy characterized by a trinucleotide repeat (TNR) in the 3′ UTR of the DMPK gene, the method comprising delivering to a cell that comprises a TNR in the 3′ UTR of the DMPK gene:
a. the composition of any one of 1b, 5, 6b, 6hh-6kk, 13-14, and 15-95; or
b. a pair of guide RNAs comprising a pair of spacer sequences, or one or more vectors encoding the pair of guide RNAs, wherein the pair of spacer sequences comprise a first spacer sequence selected from SEQ ID NOs: 201-203, 211, 215, 220, 225, 231, 235, 238, and 240-259 and a second spacer sequence selected from SEQ ID NOs: 200, 204-210, 212-214, 216-219, 221-224, 226-230, 232-234, 236-237, and 239;
g. a pair of guide RNAs comprising a pair of spacer sequences, or one or more vectors encoding the pair of guide RNAs, wherein the pair of spacer sequences comprises SEQ ID NO: 201 and SEQ ID NO: 206;
h. a pair of guide RNAs comprising a pair of spacer sequences, or one or more vectors encoding the pair of guide RNAs, wherein the pair of spacer sequences comprises SEQ ID NO: 201 and SEQ ID NO: 224;
i. a pair of guide RNAs comprising a pair of spacer sequences, or one or more vectors encoding the pair of guide RNAs, wherein the pair of spacer sequences comprises SEQ ID NO: 202 and SEQ ID NO: 213;
j. a pair of guide RNAs comprising a pair of spacer sequences, or one or more vectors encoding the pair of guide RNAs, wherein the pair of spacer sequences comprises SEQ ID NO: 202 and SEQ ID NO: 218; and
SluCas9 or a nucleic acid encoding the SaCas9.

152. A method of excising a trinucleotide repeat (TNR) in the 3′ UTR of the DMPK gene comprising delivering to a cell that comprises the TNR in the 3′ UTR of the DMPK gene a pair of guide RNAs comprising a pair of spacer sequences, or one or more vectors encoding the pair of guide RNAs, wherein the pair of spacer sequences comprise:
i. a first spacer sequence selected from SEQ ID NOs: 201-203, 211, 215, 220, 225, 231, 235, 238, and 240-259, and a second spacer sequence selected from SEQ ID NOs: 200, 204-210, 212-214, 216-219, 221-224, 226-230, 232-234, 236-237, and 239, or a nucleic acid encoding the guide RNA; and
ii. SaCas9 or a nucleic acid encoding the SaCas9, wherein at least one TNR is excised.

153. The method of any one of claims 151-152, wherein a pair of guide RNAs that comprises a first and second spacer sequence that guide the SaCas9 to or near a TNR, or one or more vectors encoding the pair of guide RNAs, are delivered to the cell.

154. The method of any one of claims 151-153, wherein the first and second spacer sequences have the sequences of SEQ ID NOs: 201 and 206.

155. The method of any one of claims 151-153, wherein the first and second spacer sequences have the sequences of SEQ ID NOs: 201 and 224.

156. The method of any one of claims 151-153, wherein the first and second spacer sequences have the sequences of SEQ ID NOs: 202 and 213.

157. The method of any one of claims 151-153, wherein the first and second spacer sequences have the sequences of SEQ ID NOs: 202 and 218.

158. The method of any one of claims 151-157, further comprising SaCas9, or a nucleic acid encoding the SaCas9.

159. The method of any one of claim 151-158, wherein the guide RNA further comprises a SaCas9 crRNA and/or a tracrRNA sequence.

160. The method of any one of claims 96-128, wherein the guide RNA further comprises:
a. a sequence selected from SEQ ID NO: 500;
b. a sequence that is at least 80%, 85%, 90%, 91%, 92%, 93%, 94%, 95%, 96%, 97%, 98%, 99% or 100% identical to SEQ ID NO: 500; or
c. a sequence that differs from SEQ ID NO: 500 by no more than 1, 2, 3, 4, 5, 10, 15, or 25 nucleotides.

161. The method of any one of claims 151-160, wherein the SaCas9 or nucleic acid encoding SaCas9 comprises SEQ ID NO: 711.

162. The method of any one of claims 151-161, wherein the SaCas9 or nucleic acid encoding SaCas9 comprises a sequence that is at least 80%, 85%, 90%, 91%, 92%, 93%, 94%, 95%, 96%, 97%, 98%, 99% or 100% identical to the sequence of SEQ ID NO: 711.

163. The method of any one of claims 151-162, wherein the guide RNA is an sgRNA.

164. The method of claim 163, wherein the sgRNA is modified.

165. The method of claim 164, wherein the modifications alter one or more 2′ positions and/or phosphodiester linkages.

166. The method of claims 164-165, wherein the modifications alter one or more, or all, of the first three nucleotides of the sgRNA.

167. The method of claims 164-166, wherein the modifications alter one or more, or all, of the last three nucleotides of the sgRNA.

168. The method of claims 164-167, wherein the modifications include one or more of a phosphorothioate modification, a 2′-OMe modification, a 2′-O-MOE modification, a 2′-F modification, a 2′-O-methine-4′ bridge modification, a 3′-thiophosphonoacetate modification, and a 2′-deoxy modification.

169. The method of any one of claims 151-168, wherein the composition further comprises a pharmaceutically acceptable excipient.

170. The method of any one of claims 151-169, wherein the guide RNA is associated with a lipid nanoparticle (LNP), or encoded by a viral vector.

171. The method of claim 170, wherein the viral vector is an adeno-associated virus vector, a lentiviral vector, an integrase-deficient lentiviral vector, an adenoviral vector, a vaccinia viral vector, an alphaviral vector, or a herpes simplex viral vector.

172. The method of claim 171, wherein the viral vector is an adeno-associated virus (AAV) vector.

173. The method of claim 172, wherein the AAV vector is an AAV1, AAV2, AAV3, AAV4, AAV5, AAV6, AAV7, AAV8, AAVrh10, AAVrh74, or AAV9 vector, wherein the number following AAV indicates the AAV serotype.

174. The method of claim 173, wherein the AAV vector is an AAV serotype 9 vector.

175. The method of any one of claims 170-173, wherein the viral vector comprises a tissue-specific promoter.

176. The method of any one of claims 170-175, wherein the viral vector comprises a muscle-specific promoter, optionally wherein the muscle-specific promoter is a muscle creatine kinase promoter, a desmin promoter, an MHCK7 promoter, or an SPc5-12 promoter.

177. The method of any one of claims 170-176, wherein the viral vector comprises a neuron-specific promoter, optionally wherein the neuron-specific promoter is an enolase promoter.
